SEQUENCE LISTING (34 sequences)

SEQ ID NO: 1 - protein, 701 aa, Homo sapiens
MAAPMTPAARPEDYEAALNAALADVPELARLLEIDPYLKPYAVDFQRRYKQFSQILKNIGENEGGIDKFSRGYESFGVHRCADGGLYCKEWAPGAEGVFLTGDFNGWNPFSYPYKKLDYGKWELYIPPKQNKSVLVPHGSKLKVVITSKSGEILYRISPWAKYVVREGDNVNYDWIHWDPEHSYEFKHSPKKPRSLRIYESHVGISSHEGKVASYKHFTCNVLPRIKGLGYNCIQLMAIMEHAYYASFGYQITSFFAASSRYGTPEELQELVDTAHSMGIIVLLDVVHSHASKNSADGLNMFDGTDSCYFHSGPRGTHDLWDSRLFAYSSWEILRFLLSNIRWWLEEYRFDGFRFDGVTSMLYHHHGVGQGFSGDYSEYFGLQVDEDALTYLMLANHLVHTLCPDSITIAEDVSGMPALCSPISQGGGGFDYRLAMAIPDKWIQLLKEFKDEDWNMGDIVYTLTNRRYLEKCIAYAESHDQALVGDKSLAFWLMDAEMYTNMSVLTPFTPVIDRGIQLHKMIRLITHGLGGEGYLNFMGNEFGHPEWLDFPRKGNNESYHYARRQFHLTDDDLLRYKFLNNFDRDMNRLEERYGWLAAPQAYVSEKHEGNKIIAFERAGLLFIFNFHPSKSYTDYRVGTALPGKFKIVLDSDAAEYGGHQRLDHSTDFFSEAFEHNGRPYSLLVYIPSRVALILQNVDLPN

SEQ ID NO: 2 - DNA, 2109 bp, Homo sapiens
atggcggctccgatgactcccgcggctcggcccgaggactacgaggcggcgctcaatgccgccctggctgacgtgcccgaactggccagactcctggagatcgacccgtacttgaagccctacgccgtggacttccagcgcaggtataagcagtttagccaaattttgaagaacattggagaaaatgaaggtggtattgataagttttccagaggctatgaatcatttggcgtccacagatgtgctgatggtggtttatactgcaaagaatgggccccgggagcagaaggagtttttcttactggagattttaatggttggaatccattttcgtacccatacaaaaaactggattatggaaaatgggagctgtatatcccaccaaagcagaataaatctgtactcgtgcctcatggatccaaattaaaggtagttattactagtaaaagcggagagatcttgtatcgtatttcaccgtgggcaaagtatgtggttcgtgaaggtgataatgtgaattatgattggatacactgggatccagaacactcatatgagtttaagcattccagaccaaagaagccacggagtctaagaatttatgaatctcatgtgggaatttcttcccatgaaggaaaagtagcttcttataaacattttacatgcaatgtactaccaagaatcaaaggccttggatacaactgcattcagttgatggcaatcatggagcatgcttactatgccagctttggttaccaaatcacaagcttctttgcagcttccagccgttatggaacacctgaagagctacaagaactggtagacacagctcattccatgggtatcatagtcctcttagatgtggtacacagccatgcttcaaaaaattcagcagatggattgaatatgtttgatgggacagattcctgttattttcattctggacctagagggactcatgatctttgggatagcagattgtttgcctactccagctgggaaattttaagattccttctgtcaaacataagatggtggttggaagaatatcgctttgatggatttcgttttgatggtgttacgtccatgctttatcatcaccatggagtgggtcaaggtttctcaggtgattacagtgaatatttcggactacaagtagatgaagatgccttgacttacctcatgttggcaaatcatttggttcacacgctgtgtcccgattctataacaatagctgaggatgtatcaggaatgccagctctgtgctctccaatttcccagggagggggtggttttgactatcgactagccatggcaattccagataagtggattcagctacttaaagagtttaaagatgaagactggaacatgggcgatatagtatacacgctcacaaacaggcgctaccttgaaaagtgcattgcttatgcagagagccatgatcaggcattggttggggataagtcgctggcattttggttgatggatgccgaaatgtatacaaacatgagtgtcctgactccttttactccagttattgatcgtggaatacagcttcataaaatgattcgactcattacgcatgggcttggtggagaaggctatctcaatttcatgggtaatgaatttgggcatcctgaatggttagacttcccaagaaaaggaaataatgagagttaccattatgccaggcggcagtttcatttaactgacgacgaccttcttcgctacaagttcctaaataattttgacagggatatgaatagattggaagaaagatatggttggcttgcagctccacaggcctacgtgagtgaaaaacatgaaggcaataagatcattgcttttgaaagagcaggtcttcttttcattttcaacttccatccaagcaagagctacactgactaccgagttggaacagcattgccagggaaattcaaaattgtgctagattcagatgcagcggaatatggagggcatcagagactggaccacagcactgactttttttctgaggcttttgaacataatgggcgtccctattctcttttggtgtacattccaagcagagtggccctcatccttcagaatgtggatctgccgaattga

SEQ ID NO: 3 - DNA, 2109 bp, Homo sapiens
atggctgcaccaatgactccagcagccagaccagaagactatgaggcagctctcaatgctgcactggctgatgtcccagagctggccagactcctggaaattgatccctacctgaagccttatgcagtggactttcagagaaggtataagcagttctcccaaattctgaaaaatattggagagaatgaaggaggcattgataagttcagcagagggtatgaatcctttggagtgcataggtgtgctgatggaggcctgtattgcaaggaatgggccccaggagcagagggagtgtttctgacaggggactttaatgggtggaatcctttctcttatccttataagaagctggattatggcaaatgggaactgtatattccacccaaacagaacaaatctgtgctggtcccacatggcagtaagctgaaagtggtcattactagtaagagtggagaaatcctgtatagaataagtccttgggcaaagtatgtggttagagagggagacaatgttaactatgattggatccactgggaccctgagcatagctatgaatttaaacacagcaggcctaagaaacctagaagcctgaggatttatgagtcccatgtgggaataagctcccatgagggaaaggttgcatcctataaacattttacatgcaatgtgctgccaaggatcaaaggactgggctacaactgcatccaactgatggctatcatggaacatgcttactatgcatcctttggataccagattacaagcttctttgcagcctctagcagatatggcacacctgaagagctgcaggaactggtggatacagcccattccatgggtatcattgttctcctggatgttgtccattctcatgccagcaaaaactctgctgatgggctgaatatgtttgatggaacagattcttgctattttcactctggaccaagaggaacccatgacctctgggattccagactgtttgcttactcctcctgggaaatcctgaggtttctcctgagcaacattagatggtggctggaagagtacagatttgatggcttcagatttgatggagtgacctccatgctgtatcaccaccatggggtgggacaaggattctctggagactactctgaatactttggactgcaagtggatgaggatgctctgacatatctgatgctggcaaatcacctggtccacacactgtgtcctgattctatcactattgctgaagatgtgtcaggaatgcctgcactgtgtagccctatcagccagggtggaggagggtttgattatagactggctatggctattcctgacaagtggattcagctgctgaaggagtttaaagatgaggactggaacatgggagatattgtgtatacactcaccaatagaaggtatctggagaaatgtattgcatatgctgagagccatgaccaggcactggtgggagataagtcactggcattttggctgatggatgctgagatgtacactaatatgagtgttctcacacctttcacaccagtgattgacagaggaatccagctccacaagatgattagattgattacacatggactgggaggtgaggggtatctgaactttatgggaaatgaatttggacacccagagtggctggactttcccagaaagggcaacaatgaatcttaccactatgctaggagacagtttcatctcacagatgatgacctcctcaggtacaaatttttgaacaactttgacagagacatgaacagactggaagagaggtatggctggctggcagctccacaggcttatgtttctgaaaaacatgaaggaaataaaatcattgcatttgaaagagcaggactcctgttcatcttcaactttcatcctagcaaaagctatacagattatagggtgggaacagcactccctgggaagttcaagattgtgctggactctgatgctgcagagtatggaggacaccagaggctggaccacagcacagacttcttttctgaagcctttgaacataatggcagaccctactccctcctggtgtacatcccttccagggtggcactcattctgcaaaatgtggatctgccaaactga

SEQ ID NO: 4 - DNA, 2109 bp, Homo sapiens
atggctgctcccatgactcctgctgctagacctgaggactatgaggctgccctcaatgctgccctggctgatgtgcctgaactggccagactcctggagattgacccctacttgaagccctatgctgtggacttccagagaaggtataagcagtttagccaaattttgaagaacattggagaaaatgaaggtggtattgataagttttccagaggctatgaatcatttggagtccacagatgtgctgatggtggtttatactgcaaagaatgggcccctggagcagaaggagtttttcttactggagattttaatggttggaatccatttagctacccatacaaaaaactggattatggaaaatgggagctgtatatcccaccaaagcagaataaatctgtactggtgcctcatggatccaaattaaaggtagttattactagtaaatctggagagatcttgtatagaatttcaccctgggcaaagtatgtggttagagaaggtgataatgtgaattatgattggatacactgggatccagaacactcatatgagtttaagcattccagaccaaagaagccaagaagtctaagaatttatgaatctcatgtgggaatttcttcccatgaaggaaaagtagcttcttataaacattttacatgcaatgtactaccaagaatcaaaggccttggatacaactgcattcagttgatggcaatcatggagcatgcttactatgccagctttggttaccaaatcacaagcttctttgcagcttccagcagatatggaacacctgaagagctacaagaactggtagacacagctcattccatgggtatcatagtcctcttagatgtggtacacagccatgcttcaaaaaattcagcagatggattgaatatgtttgatgggacagattcctgttattttcattctggacctagagggactcatgatctttgggatagcagattgtttgcctactccagctgggaagttttaagattccttctgtcaaacataagatggtggttggaagaatatagatttgatggatttagatttgatggtgttacctccatgctttatcatcaccatggagtgggtcaaggtttctcaggtgattacagtgaatattttggactacaagtagatgaagatgccttgacttacctcatgttggcaaatcatttggttcacaccctgtgtcctgattctataacaatagctgaggatgtatcaggaatgccagctctgtgctctccaatttcccagggagggggtggttttgactatagactagccatggcaattccagataagtggattcagctacttaaagagtttaaagatgaagactggaacatgggagatatagtatacaccctcacaaacaggagataccttgaaaagtgcattgcttatgcagagagccatgatcaggcattggttggggataagagcctggcattttggttgatggatgctgaaatgtatacaaacatgagtgtcctgactccttttactccagttattgatagaggaatacagcttcataaaatgattagactcattacccatgggcttggtggagaaggctatctcaatttcatgggtaatgaatttgggcatcctgaatggttagacttcccaagaaaaggaaataatgagagttaccattatgccaggagacagtttcatttaactgatgatgaccttcttagatacaagttcctaaataattttgacagggatatgaatagattggaagaaagatatggttggcttgcagctccacaggcctatgtgagtgaaaaacatgaaggcaataagatcattgcttttgaaagagcaggtcttcttttcattttcaacttccatccaagcaagagctacactgactacagagttggaacagcattgccagggaaattcaaaattgtgctagattcagatgcagctgaatatggagggcatcagagactggaccacagcactgactttttttctgaggcttttgaacataatgggagaccctattctcttttggtgtacattccaagcagagtggccctcatccttcagaatgtggatctgcccaattga

SEQ ID NO: 5 - DNA, 1265 bp, Artificial Sequence
  Dual promoter comprising a liver-specific promoter and a CMV enhancer/chicken beta-actin (CB) promoter
gagttaatttttaaaaagcagtcaaaagtccaagtggcccttgcgagcatttactctctctgtttgctctggttaataatctcaggagcacaaaattccttactagtcctagaagttaatttttaaaaagcagtcaaaagtccaagtccaagtggcccttgcgagcatttactctctctgtttgctctggttaataatctcaggagcacaaacattccttactagttctagagcggccgccagtgtgctggaattcggcttttttagggctggaagctacctttgacatcatctcctctgcgaatgcatgtataatttctacagaacctattagaaaggatcacccagcctctgcttttgtacaactttcccttaaaaaactgccaatcccactgctgtttggcccaatagtgagaactttttctgctgcctcttggtgcttttgcctatggcccctattctgctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactgagagtgctctagttttgcaatacaggacatgctataaaaatggcttaaggttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgcatggtcgaggtgagccccacgttctgcttcactctccccatctcccccccctccccacccccaattttgtatttatttattttttaattattttgtgcagcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 6 - DNA, 1372 bp, Artificial Sequence
  Note Dual promoter comprising a liver specific promoter and a mCMV/hEF1alphaCpG-free promoter
gagttaatttttaaaaagcagtcaaaagtccaagtggcccttgcgagcatttactctctctgtttgctctggttaataatctcaggagcacaaaattccttactagtcctagaagttaatttttaaaaagcagtcaaaagtccaagtccaagtggcccttgcgagcatttactctctctgtttgctctggttaataatctcaggagcacaaacattccttactagttctagagcggccgccagtgtgctggaattcggcttttttagggctggaagctacctttgacatcatctcctctgcgaatgcatgtataatttctacagaacctattagaaaggatcacccagcctctgcttttgtacaactttcccttaaaaaactgccaatcccactgctgtttggcccaatagtgagaacttttttctgctgcctcttggtgcttttgcctatggcccctattctgctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactgagagtgctctagttttgcaatacaggacatgctataaaaatggcttaaggagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattc

SEQ ID NO: 7 - protein, 511 aa, Homo sapiens
MKLFWLLFTIGFCWAQYSSNTQQGRTSIVHLFEWRWVDIALECERYLAPKGFGGVQVSPPNENVAIHNPFRPWWERYQPVSYKLCTRSGNEDEFRNMVTRCNNVGVRIYVDAVINHMCGNAVSAGTSSTCGSYFNPGSRDFPAVPYSGWDFNDGKCKTGSGDIENYNDATQVRDCRLSGLLDLALGKDYVRSKIAEYMNHLIDIGVAGFRIDASKHMWPGDIKAILDKLHNLNSNWFPEGSKPFIYQEVIDLGGEPIKSSDYFGNGRVTEFKYGAKLGTVIRKWNGEKMSYLKNWGEGWGFMPSDRALVFVDNHDNQRGHGAGGASILTFWDARLYKMAVGFMLAHPYGFTRVMSSYRWPRYFENGKDVNDWVGPPNDNGVTKEVTINPDTTCGNDWVCEHRWRQIRNMVNFRNVVDGQPFTNWYDNGSNQVAFGRGNRGFIVFNNDDWTFSLTLQTGLPAGTYCDVISGDKINGNCTGIKIYVSDDGKAHFSISNSAEDPFIAIHAESKL

SEQ ID NO: 8 - protein, 511 aa, Homo sapiens
MKFFLLLFTIGFCWAQYSPNTQQGRTSIVHLFEWRWVDIALECERYLAPKGFGGVQVSPPNENVAIYNPFRPWWERYQPVSYKLCTRSGNEDEFRNMVTRCNNVGVRIYVDAVINHMCGNAVSAGTSSTCGSYFNPGSRDFPAVPYSGWDFNDGKCKTGSGDIENYNDATQVRDCRLTGLLDLALEKDYVRSKIAEYMNHLIDIGVAGFRLDASKHMWPGDIKAILDKLHNLNSNWFPAGSKPFIYQEVIDLGGEPIKSSDYFGNGRVTEFKYGAKLGTVIRKWNGEKMSYLKNWGEGWGFVPSDRALVFVDNHDNQRGHGAGGASILTFWDARLYKMAVGFMLAHPYGFTRVMSSYRWPRQFQNGNDVNDWVGPPNNNGVIKEVTINPDTTCGNDWVCEHRWRQIRNMVIFRNVVDGQPFTNWYDNGSNQVAFGRGNRGFIVFNNDDWSFSLTLQTGLPAGTYCDVISGDKINGNCTGIKIYVSDDGKAHFSISNSAEDPFIAIHAESKL

SEQ ID NO: 9 - protein, 1053 aa, Artificial Sequence
  Cas9 in Staphylococcus Aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 10 - protein, 1368 aa, Artificial Sequence
  Cas9 in Streptococcus pyogenes
MDKKYSIGLDIGTNSVGWAVITDDYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRLNSEITKAPLSASMIKRYDEHHQDLTLLKALVRQQLPEKYKEIFFDQSKNGYAGYIDGGASQEEFYKFIKPILEKMDGTEELLAKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFIKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGLTIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 11 - DNA, 3162 bp, Artificial Sequence
  Cas9 in Staphylococcus Aureus (45349..48510 in ORF139)
atgaaaaggaattatatcttaggattagatatcggaattacatcagtgggttatggaattattgattatgaaactagagatgtcatagatgcgggcgtacgtttatttaaagaggctaatgttgaaaataatgaaggacgacgatcaaaaagaggtgccagaaggcttaagaggcgtcgtagacatagaatacaaagagtaaagaaacttttatttgattacaatttgttgacagatcatagtgagctaagtggaatcaatccttacgaggcgcgcgtaaagggattaagtcaaaaattaagtgaagaggaattttctgcggcattgctacatttagcaaagcgtagaggtgtacataatgttaatgaagtggaagaagatacaggtaatgaattatccactaaagaacaaatttcaagaaatagtaaagcgttagaagagaagtatgttgcagaattacagttggaacgtttgaaaaaagacggtgaagtgagaggttcgattaaccgtttcaaaacatctgactatgtaaaagaagcaaagcagttattaaaagtacaaaaagcatatcatcaacttgatcaatcatttatagacacttatattgatttattggaaacaagaagaacatattatgagggaccaggtgaaggtagcccatttggatggaaagatattaaagaatggtatgaaatgttaatgggacattgtacgtatttcccagaagaattacgtagtgtgaaatatgcctataatgctgatttatataatgcgctgaatgatttgaacaacttggttattacacgagatgagaatgagaagctagagtattatgaaaaattccaaattatcgagaatgtctttaaacaaaagaaaaagccgacgcttaaacaaattgcgaaggaaatcttggtgaatgaagaagacatcaaaggctatcgtgtcacaagtacaggtaaaccagaatttacaaacttgaaagtttatcacgatatcaaagatattacagcaagaaaagaaattatcgagaatgcagagctactcgatcaaatagctaaaatattaactatttaccagtcatcagaagatatacaagaagaattaacaaacctaaattcagaattgacacaagaagagattgaacaaatttcaaacttgaaaggttatacaggaactcataacctttcactaaaggcaataaatttaatattagacgaattgtggcatacgaacgataatcaaatagctattttcaatcgtttgaaacttgtacctaaaaaggtagatttaagccaacaaaaagaaattcctactactttagttgatgattttatactgtctccagtagtgaaacgttcatttatacaatctattaaagttattaacgctattattaaaaaatacggtttgccaaatgatattattattgaacttgcgagagaaaagaattctaaagatgcacaaaaaatgattaatgaaatgcagaagagaaatcgtcaaacgaatgaacgtattgaggaaattataagaacgacaggtaaagaaaatgctaaatatttaattgaaaaaattaagctgcacgatatgcaagaagggaaatgtttatactcgttagaagcaatccctctagaagatttacttaataatccattcaattacgaagtagaccatatcattccacgttctgtttctttcgataactctttcaataataaagtgttggtgaaacaagaagaaaatagtaaaaaaggtaatagaacgccatttcaatatttaagttcttcagattctaaaataagttatgagacattcaaaaagcatattttaaatcttgctaaaggcaaaggtagaatctctaagacgaaaaaagaatatttgttagaagaacgagatatcaatcgcttcagtgtccaaaaagattttattaaccgtaacttagtagatacacgctatgcgacaagagggttaatgaacttattaagatcttattttagagtgaataacttagatgtcaaagtgaaatcgattaatggcggattcacaagtttcttaagaaggaaatggaagttcaaaaaagaaagaaataagggctacaaacaccatgctgaagatgcactgattattgcgaacgctgattttattttcaaagaatggaaaaaactagataaagctaaaaaagtgatggaaaatcaaatgtttgaagaaaagcaagctgaaagtatgcctgaaattgagactgagcaagagtataaagaaatttttataacgcctcatcaaattaaacatattaaggattttaaagattataaatattcacatagagttgataaaaagccgaatagagagttaataaatgatacattatattctacgagaaaagatgacaagggtaatacattaatcgttaataacttaaatggtttatacgataaagataatgataaattgaaaaaattaattaataaatcacctgaaaaattattgatgtatcatcatgatccacaaacatatcaaaaattaaaattgatcatggaacaatatggcgatgagaaaaatccgctttataaatattatgaagaaacaggcaattacttaacaaaatatagtaaaaaagataacggaccagtcatcaaaaaaattaaatattatggtaacaagctaaatgcgcatttagatattacggatgattatccaaatagcagaaataaagtagtaaaactttcattaaaaccatatcgctttgatgtttatttagataatggggtatataaatttgtgacagttaaaaatttagatgttatcaaaaaagaaaactactatgaagttaattcaaagtgttatgaagaagcaaaaaaactgaagaaaattagtaatcaagcagaatttatcgcaagtttttacaataatgacttgattaagattaacggagaattatatagagtcataggtgtaaataatgatctacttaacagaattgaagtaaatatgatagacatcacatatagagaatatttagagaacatgaatgataaaagaccacctagaataattaaaacaatagcaagcaaaacacaatctattaaaaagtattctacagatattctaggcaatctttatgaagtgaagagtaaaaagcatcctcaaatcataaagaaaggatga

SEQ ID NO: 12 - protein, 673 aa, Artificial Sequence
  GYS1 human
MPLNRTLSMSSLPGLEDWEDEFDLENAVLFEVAWEVANKVGGIYTVLQTKAKVTGDEWGDNYFLVGPYTEQGVRTQVELLEAPTPALKRTLDSMNSKGCKFLAQSEEKPHVVAHFHEWLAGVGLCLCRARRLPVATIFTTHATLLGRYLCAGAVDFYNNLENFNVDKEAGERQIYHRYCMERAAAHCAHVFTTVSQITAIEAQHLLKRKPDIVTPNGLNVKKFSAMHEFQNLHAQSKARIQEFVRGHFYGHLDFNLDKTLYFFIAGRYEFSNKGADVFLEALARLNYLLRVNGSEQTVVAFFIMPARTNNFNVETLKGQAVRKQLWDTANTVKEKFGRKLYESLLVGSLPDMNKMLDKEDFTMMKRAIFATQRQSFPPVCTHNMLDDSSDPILTTIRRIGLFNSSADRVKVIFHPEFLSSTSPLLPVDYEEFVRGCHLGVFPSYYEPWGYTPAECTVMGIPSISTNLSGFGCFMEEHIADPSAYGIYILDRRFRSLDDSCSQLTSFLYSFCQQSRRQRIIQRNRTERLSDLLDWKYLGRYYMSARHMALSKAFPEHFTYEPNEADAAQGYRYPRPASVPPSPSLSRHSSPHQSEDEEDPRNGPLEEDGERYDEDEEAAKDRRNIRAPEWPRRASCTSSTSGSKRNSVDTATSSSLSTPSEPLSPTSSLGEERN

SEQ ID NO: 13 - protein, 737 aa, Artificial Sequence
  GYS1 human
MPLNRTLSMSSLPGLEDWEDEFDLENAVLFEVAWEVANKVGGIYTVLQTKAKVTGDEWGDNYFLVGPYTEQGVRTQVELLEAPTPALKRTLDSMNSKGCKVYFGRWLIEGGPLVVLLDVGASAWALERWKGELWDTCNIGVPWYDREANDAVLFGFLTTWFLGEFLAQSEEKPHVVAHFHEWLAGVGLCLCRARRLPVATIFTTHATLLGRYLCAGAVDFYNNLENFNVDKEAGERQIYHRYCMERAAAHCAHVFTTVSQITAIEAQHLLKRKPDIVTPNGLNVKKFSAMHEFQNLHAQSKARIQEFVRGHFYGHLDFNLDKTLYFFIAGRYEFSNKGADVFLEALARLNYLLRVNGSEQTVVAFFIMPARTNNFNVETLKGQAVRKQLWDTANTVKEKFGRKLYESLLVGSLPDMNKMLDKEDFTMMKRAIFATQRQSFPPVCTHNMLDDSSDPILTTIRRIGLFNSSADRVKVIFHPEFLSSTSPLLPVDYEEFVRGCHLGVFPSYYEPWGYTPAECTVMGIPSISTNLSGFGCFMEEHIADPSAYGIYILDRRFRSLDDSCSQLTSFLYSFCQQSRRQRIIQRNRTERLSDLLDWKYLGRYYMSARHMALSKAFPEHFTYEPNEADAAQGYRYPRPASVPPSPSLSRHSSPHQSEDEEDPRNGPLEEDGERYDEDEEAAKDRRNIRAPEWPRRASCTSSTSGSKRNSVDTATSSSLSTPSEPLSPTSSLGEERN

SEQ ID NO: 14 - protein, 703 aa, Artificial Sequence
  GYS2 human
MLRGRSLSVTSLGGLPQWEVEELPVEELLLFEVAWEVTNKVGGIYTVIQTKAKTTADEWGENYFLIGPYFEHNMKTQVEQCEPVNDAVRRAVDAMNKHGCQVHFGRWLIEGSPYVVLFDIGYSAWNLDRWKGDLWEACSVGIPYHDREANDMLIFGSLTAWFLKEVTDHADGKYVVAQFHEWQAGIGLILSRARKLPIATIFTTHATLLGRYLCAANIDFYNHLDKFNIDKEAGERQIYHRYCMERASVHCAHVFTTVSEITAIEAEHMLKRKPDVVTPNGLNVKKFSAVHEFQNLHAMYKARIQDFVRGHFYGHLDFDLEKTLFLFIAGRYEFSNKGADIFLESLSRLNFLLRMHKSDITVMVFFIMPAKTNNFNVETLKGQAVRKQLWDVAHSVKEKFGKKLYDALLRGEIPDLNDILDRDDLTIMKRAIFSTQRQSLPPVTTHNMIDDSTDPILSTIRRIGLFNNRTDRVKVILHPEFLSSTSPLLPMDYEEFVRGCHLGVFPSYYEPWGYTPAECTVMGIPSVTTNLSGFGCFMQEHVADPTAYGIYIVDRRFRSPDDSCNQLTKFLYGFCKQSRRQRIIQRNRTERLSDLLDWRYLGRYYQHARHLTLSRAFPDKFHVELTSPPTTEGFKYPRPSSVPPSPSGSQASSPQSSDVEDEVEDERYDEEEEAERDRLNIKSPFSLSHVPHGKKKLHGEYKN

SEQ ID NO: 15 - protein, 952 aa, Artificial Sequence
  GAA human
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPRVHSRAPSPLYSVEFSEEPFGVIVHRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 16 - protein, 952 aa, Artificial Sequence [sequence omitted]

SEQ ID NO: 17 - protein, 952 aa, Artificial Sequence
  GAA human
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 18 - protein, 951 aa, Artificial Sequence
  GAA human
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGPVSNFTYSPDTKVLDICVSLLMGEQFLVSWC

SEQ ID NO: 19 - protein, 883 aa, Artificial Sequence
  GAA human
AHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 20 - DNA, 2877 bp, Artificial Sequence
  GAA human
ctcgagcaaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcgtgtccacagccgggcaccgtccccactctacagcgtggagttctctgaggagcccttcggggtgatcgtgcaccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccaatggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacgctgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggagaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttaaactcgag

SEQ ID NO: 21 - DNA, 3846 bp, Artificial Sequence
  GAA human
gcgcctgcgcgggaggccgcgtcacgtgacccaccgcggccccgccccgcgacgagctcccgccggtcacgtgacccgcctctgcgcgcccccgggcacgaccccggagtctccgcgggcggccagggcgcgcgtgcgcggaggtgagccgggccggggctgcggggcttccctgagcgcgggccgggtcggtggggcggtcggctgcccgcgccggcctctcagttgggaaagctgaggttgtcgccggggccgcgggtggaggtcggggatgaggcagcaggtaggacagtgacctcggtgacgcgaaggaccccggccacctctaggttctcctcgtccgcccgttgttcagcgagggaggctctgggcctgccgcagctgacggggaaactgaggcacggagcgggcctgtaggagctgtccaggccatctccaaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcgtgtccacagccgggcaccgtccccactctacagcgtggagttctccgaggagcccttcggggtgatcgtgcaccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccaatggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcagaggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggagaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagccgggcggagtgtgttagtctctccagagggaggctggttccccagggaagcagagcctgtgtgcgggcagcagctgtgtgcgggcctgggggttgcatgtgtcacctggagctgggcactaaccattccaagccgccgcatcgcttgtttccacctcctgggccggggctctggcccccaacgtgtctaggagagctttctccctagatcgcactgtgggccggggcctggagggctgctctgtgttaataagattgtaaggtttgccctcctcacctgttgccggcatgcgggtagtattagccacccccctccatctgttcccagcaccggagaagggggtgctcaggtggaggtgtggggtatgcacctgagctcctgcttcgcgcctgctgctctgccccaacgcgaccgcttcccggctgcccagagggctggatgcctgccggtccccgagcaagcctgggaactcaggaaaattcacaggacttgggagattctaaatcttaagtgcaattattttaataaaaggggcatttggaatc

SEQ ID NO: 22 - protein, 702 aa, Artificial Sequence
  Synthetic
MAAPAAPAAGETGPDARLEAALADVPELARLLEIDPYLKPFAADFQRRYKKFSQVLHDIGENEGGIDKFSRGYESFGIHRCSDGGIYCKEWAPGAEGVFLTGEFSGWNPFSHPYKKLEYGKWELYIPPKQNKSPLIPHGSKLKVVITSKSGEILYRISPWAKYVVRENNNVNYDWIHWAPEDPYKFKHSRPKKPRSLRIYESHVGISSHEGKIASYKHFTSNVLPRIKDLGYNCIQLMAIMEHAYYASFGYQITSFFAASSRYGTPEELKELVDTAHSMGIVVLLDVVHSHASKNSEDGLNMFDGTDSCYFHSGPRGTHDLWDSRLFIYSSWEVLRFLLSNIRWWLEEYCFDGFRFDGVTSMLYHHHGMGQGFSGDYNEYFGLQVDEDALIYLMLANHLAHTLYPDSITIAEDVSGMPALCSPTSQGGGGFDYRLAMAIPDKWIQLLKEFKDEDWNMGNIVYTLTNRRYLEKCVAYAESHDQALVGDKTLAFWLMDAEMYTNMSVLAPFTPVIDRGIQLHKMIRLITHGLGGEGYLNFMGNEFGHPEWLDFPRKGNNESYHYARRQFNLTDDDLLRYKFLNNFDRDMNRLEERCGWLSAPQAYVSEKHEANKTITFERAGLLFIFNFHPSKSYTDYRVGTATPGKFKIVLDSDAAEYGGHQRLDHNTNYFAEAFEHNGRPYSLLVYIPSRVALILQNVDLQN

SEQ ID NO: 23 - DNA, 5384 bp, Artificial Sequence
  Synthetic
cagatctttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcggccgtctagaatatatagttgctctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgcatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcgggcgaggcggagaggtgcggcggcagccaatcagagcggcgcgctccgaaagtttccttttatggcgaggcggcggcggcggcggccctataaaaagcgaagcgcgcggcgggcgggagtcgctgcgaccgtgccttcgccccgtggtaccgtcgactgctgcggcgaagatggcggcgccggcggctcctgcggcaggggaaacgggccccgatgctcgtctggaggccgccttggccgacgtgccggagctggcccgactcctggagattgacccgtacctgaagcccttcgccgcggacttccagcgcaggtataagaagtttagccaggttttgcatgacattggagagaatgaaggtgggatcgataagttctccagaggctatgagtcatttggcatccacagatgttctgatggtggcatctactgcaaagaatgggccccaggagcagaaggagtttttcttactggagaattcagtggttggaatccattttctcacccatataaaaagctggaatacggaaaatgggagctgtatatcccacccaagcagaacaaatcccccctgatacctcatgggtccaagctgaaggtagttattactagtaagagtggcgagatactgtatcgcatttccccatgggcaaagtatgtggttcgtgagaacaacaacgtgaactatgattggatacactgggctccagaagacccctataaattcaagcattccagacctaagaaaccaagaagcctaagaatttatgaatctcatgtgggaatttcttcccacgaaggaaaaatagcttcttacaaacattttaccagcaatgtactaccaagaatcaaagaccttgggtataactgcattcagttgatggcgatcatggaacatgcttactatgccagttttgggtaccaaatcacgagcttttttgcagcttcaagtcgttatggaactcctgaagagctgaaagaacttgttgacacagcacattcgatgggcattgtagtcctcctagatgtggttcatagccatgcttcaaaaaattcagaagatgggttaaacatgtttgatgggaccgattcctgttattttcactctggaccccgagggacccatgatctttgggacagtagattatttatctattccagctgggaagttttaagattccttctgtcgaacataagatggtggttggaggagtactgctttgatggcttccgttttgatggtgtcacctctatgctctatcatcaccacggaatgggtcaaggtttttcgggtgactataatgaatattttggactacaagtagatgaagatgctttgatttatctcatgttggcaaatcatttggctcacacgttgtacccagactcgataacaatagcagaggatgtatcagggatgccggctctctgttctccaacttcccagggaggcggtggttttgactacagattagcaatggctattccagataaatggatccaattacttaaggaatttaaagatgaagactggaatatgggcaatatagtgtatactctcaccaatcgacgctaccttgaaaaatgtgtggcttatgcagagagtcatgatcaggcattggttggtgacaagacactggcgttttggttgatggacgctgagatgtacacgaacatgagcgttctggcccctttcactccagtaattgatcgaggaatacagcttcataagatgattcgtctcatcactcacgggctcggtggagaaggctatctcaatttcatgggtaatgagtttgggcatcctgaatggttggacttcccaaggaaaggaaataatgagagttaccattatgccagaaggcagtttaatttaaccgacgatgaccttcttcgctataagttcctaaataactttgacagagatatgaatagactggaagaaagatgtggttggctttcagctccacaggcctacgtgagtgaaaaacatgaagccaataagacaatcacctttgagagagcaggacttctctttattttcaacttccacccaagcaagagctatacggactaccgagtcgggacagcaacaccagggaagttcaaaattgtactagattctgacgcagcggagtatggaggtcatcagagactggaccacaacaccaactactttgctgaggcttttgaacataatgggcgcccctattctcttctggtgtacattccaagccgagtggctctcatccttcagaatgtggatctgcaaaactgaagatcctgaattctgcagatctgtggcttctagctgcccgggtggcatccctgtgacccctccccagtgcctctcctggccttggaagttgccactccagtgcccaccagccttgtcctaataaaattaagttgcatcattttgtctgactaggtgtcctctataatattatggggtggaggggggtggtttggagcaaggggcccaagttgggaagacacaccatattggtcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaagatctgcgacgcgaggctggatggccttccccattatgattcttctcgcttccggcggcatcgggatgcccgcgttgcaggccatgctgtccaggcaggtagatgacgaccatcagggacagcttcagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 24 - DNA, 6175 bp, Artificial Sequence
  Synthetic
aacgcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctctagaaatatatagttgctctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgcatggtcgaggtgagccccacgttctgcttcactctccccatctcccccccctccccacccccaattttgtatttatttattttttaattattttgtgcagcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctagcggtaccgtcgacgccatggcggctccgatgactcccgcggctcggcccgaggactacgaggcggcgctcaatgccgccctggctgacgtgcccgaactggccagactcctggagatcgacccgtacttgaagccctacgccgtggacttccagcgcaggtataagcagtttagccaaattttgaagaacattggagaaaatgaaggtggtattgataagttttccagaggctatgaatcatttggcgtccacagatgtgctgatggtggtttatactgcaaagaatgggccccgggagcagaaggagtttttcttactggagattttaatggttggaatccattttcgtacccatacaaaaaactggattatggaaaatgggagctgtatatcccaccaaagcagaataaatctgtactcgtgcctcatggatccaaattaaaggtagttattactagtaaaagcggagagatcttgtatcgtatttcaccgtgggcaaagtatgtggttcgtgaaggtgataatgtgaattatgattggatacactgggatccagaacactcatatgagtttaagcattccagaccaaagaagccacggagtctaagaatttatgaatctcatgtgggaatttcttcccatgaaggaaaagtagcttcttataaacattttacatgcaatgtactaccaagaatcaaaggccttggatacaactgcattcagttgatggcaatcatggagcatgcttactatgccagctttggttaccaaatcacaagcttctttgcagcttccagccgttatggaacacctgaagagctacaagaactggtagacacagctcattccatgggtatcatagtcctcttagatgtggtacacagccatgcttcaaaaaattcagcagatggattgaatatgtttgatgggacagattcctgttattttcattctggacctagagggactcatgatctttgggatagcagattgtttgcctactccagctgggaagttttaagattccttctgtcaaacataagatggtggttggaagaatatcgctttgatggatttcgttttgatggtgttacgtccatgctttatcatcaccatggagtgggtcaaggtttctcaggtgattacagtgaatatttcggactacaagtagatgaagatgccttgacttacctcatgttggcaaatcatttggttcacacgctgtgtcccgattctataacaatagctgaggatgtatcaggaatgccagctctgtgctctccaatttcccagggagggggtggttttgactatcgactagccatggcaattccagataagtggattcagctacttaaagagtttaaagatgaagactggaacatgggcgatatagtatacacgctcacaaacaggcgctaccttgaaaagtgcattgcttatgcagagagccatgatcaggcattggttggggataagtcgctggcattttggttgatggatgccgaaatgtatacaaacatgagtgtcctgactccttttactccagttattgatcgtggaatacagcttcataaaatgattcgactcattacgcatgggcttggtggagaaggctatctcaatttcatgggtaatgaatttgggcatcctgaatggttagacttcccaagaaaaggaaataatgagagttaccattatgccaggcggcagtttcatttaactgacgacgaccttcttcgctacaagttcctaaataattttgacagggatatgaatagattggaagaaagatatggttggcttgcagctccacaggcctacgtgagtgaaaaacatgaaggcaataagatcattgcttttgaaagagcaggtcttcttttcattttcaacttccatccaagcaagagctacactgactaccgagttggaacagcattgccagggaaattcaaaattgtgctagattcagatgcagcggaatatggagggcatcagagactggaccacagcactgactttttttctgaggcttttgaacataatgggcgtccctattctcttttggtgtacattccaagcagagtggccctcatccttcagaatgtggatctgccgaattgagaattctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 25 - DNA, 6645 bp, Artificial Sequence
  Synthetic
aacgcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctctagagcggccgccagtgtgctggaattcggcttttttagggctggaagctacctttgacatcatttcctctgcgaatgcatgtataatttctacagaacctattagaaaggatcacccagcctctgcttttgtacaactttcccttaaaaaatgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaaggttcaaaccttatcattttttgctttgttcctcttggccttggttttgtacatcagctttgaaaataccatcccagggttaatgctggggttaatttataactaagagtgctctagttttgcaatacaggacatgctataaaaatggcttaaggttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgcatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcgggcgaggcggagaggtgcggcggcagccaatcagagcggcgcgctccgaaagtttccttttatggcgaggcggcggcggcggcggccctataaaaagcgaagcgcgcggcgggcgggagtcgctgcgcggtaccgtcgacgccatggcggctccgatgactcccgcggctcggcccgaggactacgaggcggcgctcaatgccgccctggctgacgtgcccgaactggccagactcctggagatcgacccgtacttgaagccctacgccgtggacttccagcgcaggtataagcagtttagccaaattttgaagaacattggagaaaatgaaggtggtattgataagttttccagaggctatgaatcatttggcgtccacagatgtgctgatggtggtttatactgcaaagaatgggccccgggagcagaaggagtttttcttactggagattttaatggttggaatccattttcgtacccatacaaaaaactggattatggaaaatgggagctgtatatcccaccaaagcagaataaatctgtactcgtgcctcatggatccaaattaaaggtagttattactagtaaaagcggagagatcttgtatcgtatttcaccgtgggcaaagtatgtggttcgtgaaggtgataatgtgaattatgattggatacactgggatccagaacactcatatgagtttaagcattccagaccaaagaagccacggagtctaagaatttatgaatctcatgtgggaatttcttcccatgaaggaaaagtagcttcttataaacattttacatgcaatgtactaccaagaatcaaaggccttggatacaactgcattcagttgatggcaatcatggagcatgcttactatgccagctttggttaccaaatcacaagcttctttgcagcttccagccgttatggaacacctgaagagctacaagaactggtagacacagctcattccatgggtatcatagtcctcttagatgtggtacacagccatgcttcaaaaaattcagcagatggattgaatatgtttgatgggacagattcctgttattttcattctggacctagagggactcatgatctttgggatagcagattgtttgcctactccagctgggaagttttaagattccttctgtcaaacataagatggtggttggaagaatatcgctttgatggatttcgttttgatggtgttacgtccatgctttatcatcaccatggagtgggtcaaggtttctcaggtgattacagtgaatatttcggactacaagtagatgaagatgccttgacttacctcatgttggcaaatcatttggttcacacgctgtgtcccgattctataacaatagctgaggatgtatcaggaatgccagctctgtgctctccaatttcccagggagggggtggttttgactatcgactagccatggcaattccagataagtggattcagctacttaaagagtttaaagatgaagactggaacatgggcgatatagtatacacgctcacaaacaggcgctaccttgaaaagtgcattgcttatgcagagagccatgatcaggcattggttggggataagtcgctggcattttggttgatggatgccgaaatgtatacaaacatgagtgtcctgactccttttactccagttattgatcgtggaatacagcttcataaaatgattcgactcattacgcatgggcttggtggagaaggctatctcaatttcatgggtaatgaatttgggcatcctgaatggttagacttcccaagaaaaggaaataatgagagttaccattatgccaggcggcagtttcatttaactgacgacgaccttcttcgctacaagttcctaaataattttgacagggatatgaatagattggaagaaagatatggttggcttgcagctccacaggcctacgtgagtgaaaaacatgaaggcaataagatcattgcttttgaaagagcaggtcttcttttcattttcaacttccatccaagcaagagctacactgactaccgagttggaacagcattgccagggaaattcaaaattgtgctagattcagatgcagcggaatatggagggcatcagagactggaccacagcactgactttttttctgaggcttttgaacataatgggcgtccctattctcttttggtgtacattccaagcagagtggccctcatccttcagaatgtggatctgccgaattgagaattctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 26 - DNA, 6267 bp, Artificial Sequence
  Synthetic
tctagaggtcggccgtctaggagttaatttttaaaaagcagtcaaaagtccaagtgcccttgcgagcatttactctctctgtttgctctggttaataatctcaggagcacaaacattccttactagttctaggagttaatttttaaaaagcagtcaaaagtccaagtgcccttgcgagcatttactctctctgtttgctctggttaataatctcaggagcacaaacattccttactagttctagctagggctggaagctacctttgacatcatttcctctgcgaatgcatgtataatttctacagaacctattagaaaggatcacccagcctctgcttttgtacaactttcccttaaaaaactgccaatcccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactgagagtgctctagttctgcaatacaggacatgctataaaaatggaaagatgttgctttctgagagatcagcttacatgtgctagagagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttggtaagtcactgactgtctatgcctgggaaagggtgggcaggaggtggggcagtgcaggaaaagtggcactgtgaaccctgcagccctagacaattgtactaaccttcttctctttcctctcctgacaggttggtgtacagtagcttccaggtaccgtcgacaccatggctgcaccaatgactccagcagccagaccagaagactatgaggcagctctcaatgctgcactggctgatgtcccagagctggccagactcctggaaattgatccctacctgaagccttatgcagtggactttcagagaaggtataagcagttctcccaaattctgaaaaatattggagagaatgaaggaggcattgataagttcagcagagggtatgaatcctttggagtgcataggtgtgctgatggaggcctgtattgcaaggaatgggccccaggagcagagggagtgtttctgacaggggactttaatgggtggaatcctttctcttatccttataagaagctggattatggcaaatgggaactgtatattccacccaaacagaacaaatctgtgctggtcccacatggcagtaagctgaaagtggtcattactagtaagagtggagaaatcctgtatagaataagtccttgggcaaagtatgtggttagagagggagacaatgttaactatgattggatccactgggaccctgagcatagctatgaatttaaacacagcaggcctaagaaacctagaagcctgaggatttatgagtcccatgtgggaataagctcccatgagggaaaggttgcatcctataaacattttacatgcaatgtgctgccaaggatcaaaggactgggctacaactgcatccaactgatggctatcatggaacatgcttactatgcatcctttggataccagattacaagcttctttgcagcctctagcagatatggcacacctgaagagctgcaggaactggtggatacagcccattccatgggtatcattgttctcctggatgttgtccattctcatgccagcaaaaactctgctgatgggctgaatatgtttgatggaacagattcttgctattttcactctggaccaagaggaacccatgacctctgggattccagactgtttgcttactcctcctgggaaatcctgaggtttctcctgagcaacattagatggtggctggaagagtacagatttgatggcttcagatttgatggagtgacctccatgctgtatcaccaccatggggtgggacaaggattctctggagactactctgaatactttggactgcaagtggatgaggatgctctgacatatctgatgctggcaaatcacctggtccacacactgtgtcctgattctatcactattgctgaagatgtgtcaggaatgcctgcactgtgtagccctatcagccagggtggaggagggtttgattatagactggctatggctattcctgacaagtggattcagctgctgaaggagtttaaagatgaggactggaacatgggagatattgtgtatacactcaccaatagaaggtatctggagaaatgtattgcatatgctgagagccatgaccaggcactggtgggagataagtcactggcattttggctgatggatgctgagatgtacactaatatgagtgttctcacacctttcacaccagtgattgacagaggaatccagctccacaagatgattagattgattacacatggactgggaggtgaggggtatctgaactttatgggaaatgaatttggacacccagagtggctggactttcccagaaagggcaacaatgaatcttaccactatgctaggagacagtttcatctcacagatgatgacctcctcaggtacaaatttttgaacaactttgacagagacatgaacagactggaagagaggtatggctggctggcagctccacaggcttatgtttctgaaaaacatgaaggaaataaaatcattgcatttgaaagagcaggactcctgttcatcttcaactttcatcctagcaaaagctatacagattatagggtgggaacagcactccctgggaagttcaagattgtgctggactctgatgctgcagagtatggaggacaccagaggctggaccacagcacagacttcttttctgaagcctttgaacataatggcagaccctactccctcctggtgtacatcccttccagggtggcactcattctgcaaaatgtggatctgccaaactgaattcaagcttagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgagttgttgggattccaggcatcgagtagataagtagcatggcgggttaatcattagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcc

SEQ ID NO: 27 - DNA, 5700 bp, Artificial Sequence
  Synthetic
cagctgcgcgctcgctcgctcactgaggccgcccgggcaaagcccgggcgtcgggcgacctttggtcgcccggcctcagtgagcgagcgagcgcgcagagagggagtggccaactccatcactaggggttcctagaagctttatccacagaatcagggttcggctgcgtcctcgctgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcaggccaccatggctgcaccaatgactccagcagccagaccagaagactatgaggcagctctcaatgctgcactggctgatgtcccagagctggccagactcctggaaattgatccctacctgaagccttatgcagtggactttcagagaaggtataagcagttctcccaaattctgaaaaatattggagagaatgaaggaggcattgataagttcagcagagggtatgaatcctttggagtgcataggtgtgctgatggaggcctgtattgcaaggaatgggccccaggagcagagggagtgtttctgacaggggactttaatgggtggaatcctttctcttatccttataagaagctggattatggcaaatgggaactgtatattccacccaaacagaacaaatctgtgctggtcccacatggcagtaagctgaaagtggtcattactagtaagagtggagaaatcctgtatagaataagtccttgggcaaagtatgtggttagagagggagacaatgttaactatgattggatccactgggaccctgagcatagctatgaatttaaacacagcaggcctaagaaacctagaagcctgaggatttatgagtcccatgtgggaataagctcccatgagggaaaggttgcatcctataaacattttacatgcaatgtgctgccaaggatcaaaggactgggctacaactgcatccaactgatggctatcatggaacatgcttactatgcatcctttggataccagattacaagcttctttgcagcctctagcagatatggcacacctgaagagctgcaggaactggtggatacagcccattccatgggtatcattgttctcctggatgttgtccattctcatgccagcaaaaactctgctgatgggctgaatatgtttgatggaacagattcttgctattttcactctggaccaagaggaacccatgacctctgggattccagactgtttgcttactcctcctgggaaatcctgaggtttctcctgagcaacattagatggtggctggaagagtacagatttgatggcttcagatttgatggagtgacctccatgctgtatcaccaccatggggtgggacaaggattctctggagactactctgaatactttggactgcaagtggatgaggatgctctgacatatctgatgctggcaaatcacctggtccacacactgtgtcctgattctatcactattgctgaagatgtgtcaggaatgcctgcactgtgtagccctatcagccagggtggaggagggtttgattatagactggctatggctattcctgacaagtggattcagctgctgaaggagtttaaagatgaggactggaacatgggagatattgtgtatacactcaccaatagaaggtatctggagaaatgtattgcatatgctgagagccatgaccaggcactggtgggagataagtcactggcattttggctgatggatgctgagatgtacactaatatgagtgttctcacacctttcacaccagtgattgacagaggaatccagctccacaagatgattagattgattacacatggactgggaggtgaggggtatctgaactttatgggaaatgaatttggacacccagagtggctggactttcccagaaagggcaacaatgaatcttaccactatgctaggagacagtttcatctcacagatgatgacctcctcaggtacaaatttttgaacaactttgacagagacatgaacagactggaagagaggtatggctggctggcagctccacaggcttatgtttctgaaaaacatgaaggaaataaaatcattgcatttgaaagagcaggactcctgttcatcttcaactttcatcctagcaaaagctatacagattatagggtgggaacagcactccctgggaagttcaagattgtgctggactctgatgctgcagagtatggaggacaccagaggctggaccacagcacagacttcttttctgaagcctttgaacataatggcagaccctactccctcctggtgtacatcccttccagggtggcactcattctgcaaaatgtggatctgccaaactgaatccgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgaggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgatactcgagaaggatctaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctctattacgc

SEQ ID NO: 28 - DNA, 7671 bp, Artificial Sequence
  Synthetic
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtacccctttgagaatccacggtgtctcgatgcagtcagctttctaacaagctggggcctcacctgttttcccacggataaaaacgtgctggaggaagcagaaaggggctggcaggtggaaagatgaggaccagctcatcgtctcatgactatgaggttgctctgatccagagggtccccctgcctggtggcccaccgccaggaagactcccactgtccctggatgcccagagtgggatgtcaactccatcacttatcaactccttatccataggggtattcttcctgaggcgtctcagaaaacagggccctccccatatgctgaccacataatagaacccctcccaactcagagaccctggctgctagctgccctggcatgacccagacagtggcctttgtatatgtttttagactcaccttgactcacctctgaccatagaaactctcatcccagaggtcactgcaatagttactccacaacagaggcttatctgggtagagggaggctccctacctatggcccagcagccctgacagtgcagatcacatataccccacgccccagcactgcctgccacgcatgggcttactttacacccacccacagtcaccaacacattacctgctctccaaggttaggcgtggcaggagaagtttgcttggaccagcagaaaccatgcagtcaaggacaactggagtcagcatgggctgggtgcgagcccttggtggggtggggaggagactccaggtcatacctcctggaggatgttttaatcatttccagcatggaatgctgtcaacttttgccacagattcattagctctgagtttcttttttctgtccccagctaccccttacatgtcaatatggacttaatgatgggaaattcaggcaagtttttaaacattttattccccctggctcttatcctcaaaaaatgcatgaatttggaggcagtggctcatgcctgtaatcccaatgctttgctaggttgaggcgggaggatcacttgaagccaggaatttgagaccagcctgggccgcatagtgagaccccgtttctacaaaaataaataaataaataataaataatagtgatatgaagcatgattaaatagccctattttttaaaatgcatgagttcgttacctgattcattccctggttcctttcacagtcctccgtgacccaagtgttagggttttggtctctctactatttgtaggctgatatatagtatacacacacacacacacacacatatacacacacacagtgtatcttgagctttcttttgtatatctacacacatatgtataagaaagctcaagatatagaagccctttttcaaaaataactgaaagtttcaaactctttaagtctccagttaccattttgctggtattcttatttggaaccatacattcatcatattgttgcacagtaagactatacattcattattttgcttaaacgtatgagttaaaacacttggccaggcatggtggttcacacctgtaatcccagagctttgggaagccaagactggcagatctcttgagctcaggaattcaagaccagcctgggcaacatggaaaaaccccatctctacaaaagatagaaaaattagccaggcatggtggcgtgtgcctgtggtcccagctactcaggaggctgaggtgggaggatcacattagcccaggaggttgaggctgcagtgagccgtgattatgccactgcactccagcctgggagacagagtgagaccctgtttcaaaaaaaagagagagaaaatttaaaaaagaaaacaacaccaagggctgtaactttaaggtcattaaatgaattaatcactgcattcaaaaacgattactttctggccctaagagacatgaggccaataccaggaagggggttgatctcccaaaccagaggcagaccctagactctaatacagttaaggaaagaccagcaagatgatagtccccaatacaatagaagttactatattttatttgttgtttttcttttgttttgttttgttttgttttgttttgttttagagactggggtcttgctcgattgcccaggctgtagtgcagcggtgggacaatagctcactgcagactccaactcctgggctcaagcaatcctcctgcctcagcctcctgaatagctgggactacaagggtacaccatcacacacaccaaaacaattttttaaatttttgtgtagaaacgagggtcttgctttgttgcccaggctggtctccaactcctggcttcaagggatcctcccacctcagcctcccaaattgctgggattacaggtgtgagccaccacaaccagccagaactttactaattttaaaattaagaacttaaaacttgaatagctagagcaccaagatttttctttgtccccaaataagtgcagttgcaggcatagaaaatctgacatctttgcaagaatcatcgtggatgtagactctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcagagcaatcaccaccaagcctggaataactgcaagggctctgctgacatcttcctgaggtgccaaggaaatgaggtctagagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggccctgcaggccaccatggaggaaggaatgaatgttctccatgactttgggatccagtcaacacattacctccaggtgaattaccaagactcccaggactggttcatcttggtgtccgtgatcgcagacctcaggaatgccttctacgtcctcttccccatctggttccatcttcaggaagctgtgggcattaaactcctttgggtagctgtgattggagactggctcaacctcgtctttaagtggattctctttggacagcgtccatactggtgggttttggatactgactactacagcaacacttccgtgcccctgataaagcagttccctgtaacctgtgagactggaccagggagcccctctggccatgccatgggcacagcaggtgtatactacgtgatggtcacatctactctttccatctttcagggaaagataaagccgacctacagatttcggtgcttgaatgtcattttgtggttgggattctgggctgtgcagctgaatgtctgtctgtcacgaatctaccttgctgctcattttcctcatcaagttgttgctggagtcctgtcaggcattgctgttgcagaaactttcagccacatccacagcatctataatgccagcctcaagaaatattttctcattaccttcttcctgttcagcttcgccatcggattttatctgctgctcaagggactgggtgtagacctcctgtggactctggagaaagcccagaggtggtgcgagcagccagaatgggtccacattgacaccacaccctttgccagcctcctcaagaacctgggcacgctctttggcctggggctggctctcaactccagcatgtacagggagagctgcaaggggaaactcagcaagtggctcccattccgcctcagctgcattgtagcctccctcgtcctcctgcacgtctttgactccttgaaacccccatcccaagtcgagctggtcttctacgtcttgtccttctgcaagagtgcggtagtgcccctggcatccgtcagtgtcatcccctactgcctcgcccaggtcctgggccagccgcacaagaagtcgttgtaagcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgaccatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgca

SEQ ID NO: 29 - DNA, 7671 bp, Artificial Sequence
  Synthetic
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtacccctttgagaatccacggtgtctcgatgcagtcagctttctaacaagctggggcctcacctgttttcccacggataaaaacgtgctggaggaagcagaaaggggctggcaggtggaaagatgaggaccagctcatcgtctcatgactatgaggttgctctgatccagagggtccccctgcctggtggcccaccgccaggaagactcccactgtccctggatgcccagagtgggatgtcaactccatcacttatcaactccttatccataggggtattcttcctgaggcgtctcagaaaacagggccctccccatatgctgaccacataatagaacccctcccaactcagagaccctggctgctagctgccctggcatgacccagacagtggcctttgtatatgtttttagactcaccttgactcacctctgaccatagaaactctcatcccagaggtcactgcaatagttactccacaacagaggcttatctgggtagagggaggctccctacctatggcccagcagccctgacagtgcagatcacatataccccacgccccagcactgcctgccacgcatgggcttactttacacccacccacagtcaccaacacattacctgctctccaaggttaggcgtggcaggagaagtttgcttggaccagcagaaaccatgcagtcaaggacaactggagtcagcatgggctgggtgcgagcccttggtggggtggggaggagactccaggtcatacctcctggaggatgttttaatcatttccagcatggaatgctgtcaacttttgccacagattcattagctctgagtttcttttttctgtccccagctaccccttacatgtcaatatggacttaatgatgggaaattcaggcaagtttttaaacattttattccccctggctcttatcctcaaaaaatgcatgaatttggaggcagtggctcatgcctgtaatcccaatgctttgctaggttgaggcgggaggatcacttgaagccaggaatttgagaccagcctgggccgcatagtgagaccccgtttctacaaaaataaataaataaataataaataatagtgatatgaagcatgattaaatagccctattttttaaaatgcatgagttcgttacctgattcattccctggttcctttcacagtcctccgtgacccaagtgttagggttttggtctctctactatttgtaggctgatatatagtatacacacacacacacacacacatatacacacacacagtgtatcttgagctttcttttgtatatctacacacatatgtataagaaagctcaagatatagaagccctttttcaaaaataactgaaagtttcaaactctttaagtctccagttaccattttgctggtattcttatttggaaccatacattcatcatattgttgcacagtaagactatacattcattattttgcttaaacgtatgagttaaaacacttggccaggcatggtggttcacacctgtaatcccagagctttgggaagccaagactggcagatctcttgagctcaggaattcaagaccagcctgggcaacatggaaaaaccccatctctacaaaagatagaaaaattagccaggcatggtggcgtgtgcctgtggtcccagctactcaggaggctgaggtgggaggatcacattagcccaggaggttgaggctgcagtgagccgtgattatgccactgcactccagcctgggagacagagtgagaccctgtttcaaaaaaaagagagagaaaatttaaaaaagaaaacaacaccaagggctgtaactttaaggtcattaaatgaattaatcactgcattcaaaaacgattactttctggccctaagagacatgaggccaataccaggaagggggttgatctcccaaaccagaggcagaccctagactctaatacagttaaggaaagaccagcaagatgatagtccccaatacaatagaagttactatattttatttgttgtttttcttttgttttgttttgttttgttttgttttgttttagagactggggtcttgctcgattgcccaggctgtagtgcagcggtgggacaatagctcactgcagactccaactcctgggctcaagcaatcctcctgcctcagcctcctgaatagctgggactacaagggtacaccatcacacacaccaaaacaattttttaaatttttgtgtagaaacgagggtcttgctttgttgcccaggctggtctccaactcctggcttcaagggatcctcccacctcagcctcccaaattgctgggattacaggtgtgagccaccacaaccagccagaactttactaattttaaaattaagaacttaaaacttgaatagctagagcaccaagatttttctttgtccccaaataagtgcagttgcaggcatagaaaatctgacatctttgcaagaatcatcgtggatgtagactctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcagagcaatcaccaccaagcctggaataactgcaagggctctgctgacatcttcctgaggtgccaaggaaatgaggtctagagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggccctgcaggccaccatggaagagggcatgaacgtgctgcacgacttcggcatccagagcacccactatctgcaggtcaactaccaggacagccaggactggttcatcctggtgtccgtgatcgccgacctgcggaacgccttctacgtgctgttccccatctggttccatctgcaagaagccgtcggcatcaagctgctgtgggtggccgtgatcggcgattggctgaacctggtgttcaagtggatcctgttcggccagcggccctattggtgggtgctggacaccgactactacagcaacaccagcgtgcccctgatcaagcagttccccgtgacctgcgagacaggccctggctctccttctggccacgccatgggaacagccggcgtgtactacgtgatggtcaccagcaccctgagcatcttccagggcaagatcaagcccacctaccggttccggtgcctgaacgtgatcctgtggctgggcttctgggccgtgcagctgaacgtgtgcctgagccggatctacctggccgcccacttcccacatcaagtggtggccggcgtgctgagcggaatcgccgtggccgagacattcagccacatccacagcatctacaacgccagcctgaagaagtacttcctgatcacattctttctgttcagcttcgccatcggcttctacctgctgctgaagggcctgggcgtggacctgctgtggaccctggaaaaggcccagcggtggtgcgagcagcccgagtgggtgcacatcgacaccacccccttcgccagcctgctgaagaacctgggcaccctgtttggactgggcctggccctgaacagcagcatgtacagagagagctgcaagggcaagctgagcaagtggctgcccttccggctgagctgcatcgtggccagcctggtgctgctgcacgtgttcgacagcctgaagccccccagccaggtggaactggtgttttacgtgctgagcttctgcaagagcgccgtggtgcccctggcctccgtgtctgtgatcccctactgcctggctcaggtgctgggccagccccacaagaagtccctctgagcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgaccatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgca

SEQ ID NO: 30 - DNA, 5531 bp, Artificial Sequence
  Synthetic
tctagagagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttggtaagtcactgactgtctatgcctgggaaagggtgggcaggaggtggggcagtgcaggaaaagtggcactgtgaaccctgcagccctagacaattgtactaaccttcttctctttcctctcctgacaggttggtgtacagtagcttccaggtaccgtcgacaccatggctgcaccaatgactccagcagccagaccagaagactatgaggcagctctcaatgctgcactggctgatgtcccagagctggccagactcctggaaattgatccctacctgaagccttatgcagtggactttcagagaaggtataagcagttctcccaaattctgaaaaatattggagagaatgaaggaggcattgataagttcagcagagggtatgaatcctttggagtgcataggtgtgctgatggaggcctgtattgcaaggaatgggccccaggagcagagggagtgtttctgacaggggactttaatgggtggaatcctttctcttatccttataagaagctggattatggcaaatgggaactgtatattccacccaaacagaacaaatctgtgctggtcccacatggcagtaagctgaaagtggtcattactagtaagagtggagaaatcctgtatagaataagtccttgggcaaagtatgtggttagagagggagacaatgttaactatgattggatccactgggaccctgagcatagctatgaatttaaacacagcaggcctaagaaacctagaagcctgaggatttatgagtcccatgtgggaataagctcccatgagggaaaggttgcatcctataaacattttacatgcaatgtgctgccaaggatcaaaggactgggctacaactgcatccaactgatggctatcatggaacatgcttactatgcatcctttggataccagattacaagcttctttgcagcctctagcagatatggcacacctgaagagctgcaggaactggtggatacagcccattccatgggtatcattgttctcctggatgttgtccattctcatgccagcaaaaactctgctgatgggctgaatatgtttgatggaacagattcttgctattttcactctggaccaagaggaacccatgacctctgggattccagactgtttgcttactcctcctgggaaatcctgaggtttctcctgagcaacattagatggtggctggaagagtacagatttgatggcttcagatttgatggagtgacctccatgctgtatcaccaccatggggtgggacaaggattctctggagactactctgaatactttggactgcaagtggatgaggatgctctgacatatctgatgctggcaaatcacctggtccacacactgtgtcctgattctatcactattgctgaagatgtgtcaggaatgcctgcactgtgtagccctatcagccagggtggaggagggtttgattatagactggctatggctattcctgacaagtggattcagctgctgaaggagtttaaagatgaggactggaacatgggagatattgtgtatacactcaccaatagaaggtatctggagaaatgtattgcatatgctgagagccatgaccaggcactggtgggagataagtcactggcattttggctgatggatgctgagatgtacactaatatgagtgttctcacacctttcacaccagtgattgacagaggaatccagctccacaagatgattagattgattacacatggactgggaggtgaggggtatctgaactttatgggaaatgaatttggacacccagagtggctggactttcccagaaagggcaacaatgaatcttaccactatgctaggagacagtttcatctcacagatgatgacctcctcaggtacaaatttttgaacaactttgacagagacatgaacagactggaagagaggtatggctggctggcagctccacaggcttatgtttctgaaaaacatgaaggaaataaaatcattgcatttgaaagagcaggactcctgttcatcttcaactttcatcctagcaaaagctatacagattatagggtgggaacagcactccctgggaagttcaagattgtgctggactctgatgctgcagagtatggaggacaccagaggctggaccacagcacagacttcttttctgaagcctttgaacataatggcagaccctactccctcctggtgtacatcccttccagggtggcactcattctgcaaaatgtggatctgccaaactgaattcaagcttagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgagttgttgggattccaggcatcgagtagataagtagcatggcgggttaatcattagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcc

SEQ ID NO: 31 - DNA, 6173 bp, Artificial Sequence
  Synthetic
aacgcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctctagaaatatatagttgctctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgcatggtcgaggtgagccccacgttctgcttcactctccccatctcccccccctccccacccccaattttgtatttatttattttttaattattttgtgcagcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctagcggtaccgtcgacaccatggctgcaccaatgactccagcagccagaccagaagactatgaggcagctctcaatgctgcactggctgatgtcccagagctggccagactcctggaaattgatccctacctgaagccttatgcagtggactttcagagaaggtataagcagttctcccaaattctgaaaaatattggagagaatgaaggaggcattgataagttcagcagagggtatgaatcctttggagtgcataggtgtgctgatggaggcctgtattgcaaggaatgggccccaggagcagagggagtgtttctgacaggggactttaatgggtggaatcctttctcttatccttataagaagctggattatggcaaatgggaactgtatattccacccaaacagaacaaatctgtgctggtcccacatggcagtaagctgaaagtggtcattactagtaagagtggagaaatcctgtatagaataagtccttgggcaaagtatgtggttagagagggagacaatgttaactatgattggatccactgggaccctgagcatagctatgaatttaaacacagcaggcctaagaaacctagaagcctgaggatttatgagtcccatgtgggaataagctcccatgagggaaaggttgcatcctataaacattttacatgcaatgtgctgccaaggatcaaaggactgggctacaactgcatccaactgatggctatcatggaacatgcttactatgcatcctttggataccagattacaagcttctttgcagcctctagcagatatggcacacctgaagagctgcaggaactggtggatacagcccattccatgggtatcattgttctcctggatgttgtccattctcatgccagcaaaaactctgctgatgggctgaatatgtttgatggaacagattcttgctattttcactctggaccaagaggaacccatgacctctgggattccagactgtttgcttactcctcctgggaaatcctgaggtttctcctgagcaacattagatggtggctggaagagtacagatttgatggcttcagatttgatggagtgacctccatgctgtatcaccaccatggggtgggacaaggattctctggagactactctgaatactttggactgcaagtggatgaggatgctctgacatatctgatgctggcaaatcacctggtccacacactgtgtcctgattctatcactattgctgaagatgtgtcaggaatgcctgcactgtgtagccctatcagccagggtggaggagggtttgattatagactggctatggctattcctgacaagtggattcagctgctgaaggagtttaaagatgaggactggaacatgggagatattgtgtatacactcaccaatagaaggtatctggagaaatgtattgcatatgctgagagccatgaccaggcactggtgggagataagtcactggcattttggctgatggatgctgagatgtacactaatatgagtgttctcacacctttcacaccagtgattgacagaggaatccagctccacaagatgattagattgattacacatggactgggaggtgaggggtatctgaactttatgggaaatgaatttggacacccagagtggctggactttcccagaaagggcaacaatgaatcttaccactatgctaggagacagtttcatctcacagatgatgacctcctcaggtacaaatttttgaacaactttgacagagacatgaacagactggaagagaggtatggctggctggcagctccacaggcttatgtttctgaaaaacatgaaggaaataaaatcattgcatttgaaagagcaggactcctgttcatcttcaactttcatcctagcaaaagctatacagattatagggtgggaacagcactccctgggaagttcaagattgtgctggactctgatgctgcagagtatggaggacaccagaggctggaccacagcacagacttcttttctgaagcctttgaacataatggcagaccctactccctcctggtgtacatcccttccagggtggcactcattctgcaaaatgtggatctgccaaactgaattctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 32 - DNA, 6267 bp, Artificial Sequence; identical to SEQ ID NO: 26
  Synthetic

SEQ ID NO: 33 - DNA, 6643 bp, Artificial Sequence
  Synthetic
aacgcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctctagagcggccgccagtgtgctggaattcggcttttttagggctggaagctacctttgacatcatttcctctgcgaatgcatgtataatttctacagaacctattagaaaggatcacccagcctctgcttttgtacaactttcccttaaaaaatgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaaggttcaaaccttatcattttttgctttgttcctcttggccttggttttgtacatcagctttgaaaataccatcccagggttaatgctggggttaatttataactaagagtgctctagttttgcaatacaggacatgctataaaaatggcttaaggttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgcatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcgggcgaggcggagaggtgcggcggcagccaatcagagcggcgcgctccgaaagtttccttttatggcgaggcggcggcggcggcggccctataaaaagcgaagcgcgcggcgggcgggagtcgctgcgcggtaccgtcgacaccatggctgcaccaatgactccagcagccagaccagaagactatgaggcagctctcaatgctgcactggctgatgtcccagagctggccagactcctggaaattgatccctacctgaagccttatgcagtggactttcagagaaggtataagcagttctcccaaattctgaaaaatattggagagaatgaaggaggcattgataagttcagcagagggtatgaatcctttggagtgcataggtgtgctgatggaggcctgtattgcaaggaatgggccccaggagcagagggagtgtttctgacaggggactttaatgggtggaatcctttctcttatccttataagaagctggattatggcaaatgggaactgtatattccacccaaacagaacaaatctgtgctggtcccacatggcagtaagctgaaagtggtcattactagtaagagtggagaaatcctgtatagaataagtccttgggcaaagtatgtggttagagagggagacaatgttaactatgattggatccactgggaccctgagcatagctatgaatttaaacacagcaggcctaagaaacctagaagcctgaggatttatgagtcccatgtgggaataagctcccatgagggaaaggttgcatcctataaacattttacatgcaatgtgctgccaaggatcaaaggactgggctacaactgcatccaactgatggctatcatggaacatgcttactatgcatcctttggataccagattacaagcttctttgcagcctctagcagatatggcacacctgaagagctgcaggaactggtggatacagcccattccatgggtatcattgttctcctggatgttgtccattctcatgccagcaaaaactctgctgatgggctgaatatgtttgatggaacagattcttgctattttcactctggaccaagaggaacccatgacctctgggattccagactgtttgcttactcctcctgggaaatcctgaggtttctcctgagcaacattagatggtggctggaagagtacagatttgatggcttcagatttgatggagtgacctccatgctgtatcaccaccatggggtgggacaaggattctctggagactactctgaatactttggactgcaagtggatgaggatgctctgacatatctgatgctggcaaatcacctggtccacacactgtgtcctgattctatcactattgctgaagatgtgtcaggaatgcctgcactgtgtagccctatcagccagggtggaggagggtttgattatagactggctatggctattcctgacaagtggattcagctgctgaaggagtttaaagatgaggactggaacatgggagatattgtgtatacactcaccaatagaaggtatctggagaaatgtattgcatatgctgagagccatgaccaggcactggtgggagataagtcactggcattttggctgatggatgctgagatgtacactaatatgagtgttctcacacctttcacaccagtgattgacagaggaatccagctccacaagatgattagattgattacacatggactgggaggtgaggggtatctgaactttatgggaaatgaatttggacacccagagtggctggactttcccagaaagggcaacaatgaatcttaccactatgctaggagacagtttcatctcacagatgatgacctcctcaggtacaaatttttgaacaactttgacagagacatgaacagactggaagagaggtatggctggctggcagctccacaggcttatgtttctgaaaaacatgaaggaaataaaatcattgcatttgaaagagcaggactcctgttcatcttcaactttcatcctagcaaaagctatacagattatagggtgggaacagcactccctgggaagttcaagattgtgctggactctgatgctgcagagtatggaggacaccagaggctggaccacagcacagacttcttttctgaagcctttgaacataatggcagaccctactccctcctggtgtacatcccttccagggtggcactcattctgcaaaatgtggatctgccaaactgaattctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 34 - DNA, 755 bp, Artificial Sequence
  Synthetic
gagttaatttttaaaaagcagtcaaaagtccaagtggcccttgcgagcatttactctctctgtttgctctggttaataatctcaggagcacaaaattccttactagtcctagaagttaatttttaaaaagcagtcaaaagtccaagtccaagtggcccttgcgagcatttactctctctgtttgctctggttaataatctcaggagcacaaacattccttactagttctagagcggccgccagtgtgctggaattcggcttttttagggctggaagctacctttgacatcatttcctctgcgaatgcatgtataatttctacagaacctattagaaaggatcacccagcctctgcttttgtacaactttcccttaaaaaatgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaaggttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagatcagcttacatgt

(19) United States
(12) Patent Application Publication
(10) Pub. No.: US 2025/0186618 A1
(43) Pub. Date: Jun. 12, 2025

(54) COMPOSITIONS AND METHODS FOR TREATING AND/OR PREVENTING GLYCOGEN STORAGE DISEASES

(71) Applicant: DUKE UNIVERSITY, Durham, NC (US)

(72) Inventors: Baodong Sun, Durham, NC (US); Priya Kishnani, Durham, NC (US)

(73) Assignee: DUKE UNIVERSITY, Durham, NC (US)

(21) Appl. No.: 18/269,622

(22) PCT Filed: Dec. 23, 2021
(86) PCT No.: PCT/US21/65160

§ 371 Date: Jun. 26, 2023

Related U.S. Application Data
(60) Provisional application No. 63/130,687, filed on Dec. 26, 2020.
(60) Provisional application No. 63/243,127, filed on Sep. 11, 2021.

(51) Int. Cl. A61K 48/00; A61K 31/085; A61K 38/45; A61K 45/06; A61P 21/00; C12N 9/10; C12N 15/86

(52) U.S. Cl. CPC A61K 48/0058; A61K 31/085; A61K 38/45; A61K 45/06; A61P 21/00; C12N 9/107; C12N 15/86; C12Y 204/01018; C12N 2750/14143; C12N 2830/008

(57) ABSTRACT

Disclosed herein are compositions for and gene therapy methods of treating and/or preventing glycogen storage disease progression including the progression of GSD IV and/or APBD. Also disclosed herein are compositions for and gene therapy methods of preventing glycogen accumulation and/or degrading accumulated glycogen.

### I. CROSS-REFERENCE TO RELATED APPLICATIONS

This application claim priority to U.S. Provisional Application No. 63/130,687 filed 26 Dec. 2020 and U.S. Provisional Application No. 63/243,127 filed 11 Sep. 2021, both of which are incorporated herein in their entirety.

### II. REFERENCE TO THE SEQUENCE LISTING

The Sequence Listing submitted 23 Dec. 2021 as a text file named “20_2004_WO_Sequence_Listing”, created on 23 Dec. 2021 and having a size of 133 kilobytes is hereby incorporated by reference pursuant to 37 C.F.R. § 1.52(e)(5).

### III. BACKGROUND

Glycogen branching enzyme (GBE) is the enzyme that introduces branches to the growing glycogen molecule during the synthesis of glycogen. Mutations in the GBE1 gene cause GBE deficiency in glycogen storage disease type IV (GSD IV), resulting in pathogenic deposition of soluble glycogen and a poorly soluble, amylopectin-like glycogen, called polyglucosan bodies, in liver, skeletal and smooth muscle, heart, and the central and peripheral nervous system (CNS and PNS). (Levin B, et al. (1968) Arch Dis Child. 43(231):548-555) GSD IV is clinically variable. The classical form of GSD IV is characterized by failure to thrive, hepatosplenomegaly, and progressive liver cirrhosis that normally leads to death by 5 years of age. In addition to the hepatic presentation, four neuromuscular forms can be distinguished based on the ages at onset: fatal perinatal neuromuscular type, congenital muscular type, childhood neuromuscular type, and adult neuromuscular type. (Bruno C, et al. (2004) Neurology. 63(6):1053-1058). Most early onset GSD IV patients die in infancy or early childhood of severe hypotonia, respiratory distress, cardiomyopathy and/or liver dysfunction. Adult onset GSD IV constitutes the majority of adult polyglucosan body disease (APBD). (Bruno C, et al., 2004). Y329S is the most common mutation in the GBE1 gene in APBD patients of Ashkenazi Jewish ancestry. (Lossos A, et al. (1998) Ann Neurol. 44(6):867-872). APBD can present as an isolated myopathy or as a multi-system disorder with intracellular accumulation of polyglucosan bodies in the CNS and PNS, and in muscles, heart, and/or liver. (Mochel F, et al. (2012) Ann Neurol. 72(3):433-441; Klein C J, et al. (2004) Muscle Nerve. 29(2):323-328). Patients with APBD suffer from a poor quality of life and endure high rates of disability from progressive neuropathy and muscle weakness and some patients may develop mild cognitive impairment that can lead to progressive loss of memory and intellectual abilities. (Colombo I, et al. (2015) Neuromuscul Disord. 25(5):423-428; Mochel F, et al. (2012) Ann Neurol. 72(3):433-441; Soffer D, et al. (1997) Brain Pathol. 7(4):1342-1342).

Although liver transplantation has been successful in some cases to alleviate liver symptoms, there is currently no definitive treatment for GSD IV. (Ban H R, et al. (2009) Gut Liver. 3(1):60-63). Despite liver transplant, extrahepatic manifestations can occur such as cardiac involvement and CNS disease progression. GSD IV is often misdiagnosed and underdiagnosed. Potential treatment modalities include either a method to correct GBE enzyme deficiency or to inhibit glycogen synthase (liver/muscle—GYS1 and GYS2) in those affected with GSD IV. (Oldfors A, et al. (2013) Curr Opin Neurol. 26(5):544-553; Sun B, et al. (2015) Curr Gene Ther. 15(4):338-347).

Adeno-associated virus (AAV) mediated gene therapy has shown promise for treating human inherited disorders with successful translation to clinical trials. However, the induction of immune responses against gene therapy vectors remains a major challenge to successful gene therapy. Transgene-induced cytotoxic T lymphocyte (CTL) response is a major cause of the loss of short-term and long-term efficacy of gene therapy.

There is a need for a minimally invasive, definitive therapy to address the underlying cause of as well as the sequelae of symptoms associated with glycogen storage diseases including GSD IV and APBD. This disclosure provides methods of gene therapy for the treatment and mitigation of GSDs including GSD IV and APBD, which can be used alone or in combination with other therapies such as SRT.

### IV. BRIEF DESCRIPTION OF THE FIGURES

FIG. 1A shows a schematic of disease progression as it relates to residual GBE activity and FIG. 1B shows a schematic of disease progression in a mouse model of adult-form GSD IV (Gbe1^{ys/ys} mice).

FIG. 2 provides an illustrative example of the metabolic pathways of glycogen metabolism and glycogenolysis including the sites of enzymatic defects that result in clinical GSDs.

FIG. 3 shows a schematic of both an AAV-CB-hGBE vector containing a ubiquitous CMV enhancer/chicken β-actin (CB) promoter and an AAV-Dual-hGBE vector containing a tandem human alfa-antitrypsin (hAAT)-derived liver-specific promoter (LSP) and the CB fusion dual promoter (Dual). Both AAV vectors carry an unmodified human GBE (hGBE) open reading frame (ORF).

FIG. 4 shows immunohistochemical detection of CD8⁺ and CD4⁺ lymphocytes in the livers of untreated GSD IV mice (UT) and the GSD IV mice two weeks after treatment with either AAV-CB-hGBE (CB) or AAV-Dual-hGBE (Dual) at a dose of 2.5×10¹³ vector genomes (vg/kg). Both AAV vectors were packaged as AAV9.

FIG. 5 shows the GBE activities in major tissues of untreated (UT) GSD IV mice and in the GSD IV mice two weeks after treatment with either AAV-CB-hGBE (CB) or AAV-Dual-hGBE (Dual) at 2.5×10¹³ vg/kg. Both the AAV vectors were packaged as AAV9.

FIG. 6A shows GBE activities and FIG. 6B shows glycogen contents in tissues of untreated (UT) GSD IV mice and the GSD IV mice six weeks after treatment with AAV-Dual-hGBE (AAV) packaged as AAV9 at 1×10¹⁴ vg/kg.

FIG. 7A shows the results of the wire-hang test and FIG. 7B shows the treadmill test in the untreated (UT) GSD IV mice and the GSD IV mice six weeks after treatment with AAV-Dual-hGBE (AAV) packaged as AAV9 at 1×10¹⁴ vg/kg.

FIG. 8A-FIG. 8B show a schematic of two new AAV constructs carrying a CpG-free hGBE ORF under the control of either the CB promoter (AAV-CB-hGBE^{CpG-free}) (FIG. 8A) or the LSP-CB dual promoter (AAV-Dual-hGBE^{CpG-free}) (FIG. 8B).

FIG. 9 shows a comparison of GBE enzyme activity in HEK293T cells transfected with AAV vector plasmids containing either the unmodified hGBE ORF or CpG-free ORF driven by either the CB promoter or the LSP-CB Dual promoter. The GBE expression levels between the unmodified hGBE ORF and the CpG-free hGBE ORF were similar driven by the same promoter.

FIG. 10 shows GBE activity in HEK293T following transfection with equal amount of the 4 AAV vector plasmids having CpG-free ORFs driven by either the CB promoter, a CpG-free hEF1α (human translation elongation factor 1 alpha) promoter, a new dual promoter comprising the LSP and hEF1α, or the original dual promoter comprising the LSP and CB.

FIG. 11 shows functional expression of human GBE in adult GSD IV mice from AAV9 vectors carrying the CpG-free hGBE ORF under CB or LSP-CB dual promoter. Three-month-old GSD IV mice were intravenously injected with AAV9 vectors (2.5×10¹³ vg/kg) carrying various expression cassettes and euthanized one month later. Tissues were homogenized in cold water and GBE activities were measured in tissue lysates of liver (left panel), skeletal muscle (quadriceps) (middle panel), and heart (right panel).

FIG. 12 (which is modified from Hanlon K S, et al. (2019) Mol Ther Methods Clin Dev. 15:320-332) shows robust transgene (GFP) expression in the brain (top panels) and spinal cord (bottom panels) in adult C57BL/6 mice following intravenous injection of AAV-CB-GFP packaged as AAV9 (left panels) or AAV-F (right panels) at the same dose of 3.2×10¹³ vg/kg.

FIG. 13A-FIG. 13C show dose-dependent activity across tissues following administration of an AAV9-CB-mGBE vector expressing mouse GBE (mGBE). FIG. 13A shows AAV biodistribution in the liver, heart, quadriceps muscle, and brain following 3 different doses of AAV9-CB-mGBE. FIG. 13B shows the GBE activity level across these tissues following 3 different doses of AAV9-CB-mGBE as well as in UT and wild-type (WT) animals. FIG. 13C shows that AAV9-CB-mGBE treatment generated clinically meaningful reduction in glycogen levels in liver and the quadriceps muscle.

FIG. 14 shows that AAV9-CB-mGBE treated mice had reduced levels of polyglucosan body accumulation in the liver and quadriceps muscle as evidenced by PAS-D staining.

FIG. 15 shows that AAV9-CB-mGBE treated mice had reduced levels of polyglucosan body accumulation in the spinal cord, diaphragm, tongue, and bladder as evidenced by PAS-D staining.

FIG. 16A-FIG. 16D show a dose-dependent improvement of neurological and neuromuscular phenotypes following systemic AAV9-CB-mGBE administration. FIG. 16A shows the Rota-rod results in mice treated with 3 different doses of AAV9-CB-mGBE as well as UT and WT mice. The same mice were also evaluated using the wire-hang test (FIG. 16B), the treadmill test (FIG. 16C), and the pain test (FIG. 16D).

FIG. 17A-FIG. 17D show that glycogen synthase 1 (Gys1) expression was restored in the quadriceps muscle by the high-dose (1×10¹⁴ vg/kg) AAV9-CB-mGBE treatment. For WT, UT, and AAV-treated mice, FIG. 17A shows a series of Western blots against Gys1 and GBE, FIG. 17B quantifies the relative GBE expression, FIG. 17C shows quantifies the relative Gys1 expression, and FIG. 17D shows the Gys1/GBE ratio.

FIG. 18A-FIG. 18D show GBE expression and glycogen reduction across tissues following AAVF-CB-mGBE administration at a dose of 2.5×10¹³ vg/kg. FIG. 18A shows AAV biodistribution in the liver, quadriceps muscle, and brain following AAVF-CB-mGBE treatment while FIG. 18B shows the GBE activity level across these tissues. FIG. 18C shows a series of Western blots against Gys1 and GBE in these 3 tissues while FIG. 18D shows glycogen levels in the liver, quadriceps muscle, and brain following AAVF-CB-mGBE treatment.

FIG. 19A-FIG. 19B show that AAVF-CB-mGBE treated mice had reduced levels of polyglucosan body accumulation in the spinal cord and cerebellum as evidenced by PAS-D staining (FIG. 19A) and quantified in FIG. 19B (spinal cord only).

FIG. 20 shows that mice had significant improvements on the pain test following AAVF-CB-mGBE treatment.

FIG. 21 shows a schematic of an AAV-Dual-hGBE vector containing a dual promoter (i.e., a liver-specific promoter (LSP) and a hEF1α promoter) and a CpG-free hGBE transgene.

FIG. 22A-FIG. 22C show comparison of GBE expression and glycogen reduction across tissues following administration of the same dose (2.5×10¹³ vg/kg) of AAV9-LSP-hEF1α-hGBE^{CpG-free} and AAVF-LSP-hEF1α-hGBE^{CpG-free}. FIG. 22A shows AAV biodistribution in the liver, quadriceps muscle, and brain following AAV treatment while FIG. 22B shows the GBE activity level across these tissues. FIG. 22C shows the resulting glycogen levels in the liver, quadriceps muscle, and brain following AAV treatment.

FIG. 23A shows the generation of a construct of CpG-free hGBE with a neuron-specific promoter (i.e., synapsin) while FIG. 23B shows Western blots of HEK293 cells which demonstrated increased expression of hGBE with synapsin plasmid (Syn1-hGBE) transfection. Synapsin plasmid transfection is compared to transfection with GFP plasmid and no plasmid (negative control), with β-actin as the housekeeping protein internal controls. FIG. 23C shows the quantification of Western blots in FIG. 23B which confirms the increased expression of hGBE via transfection with the Syn1-hGBE plasmid compared to the negative control.

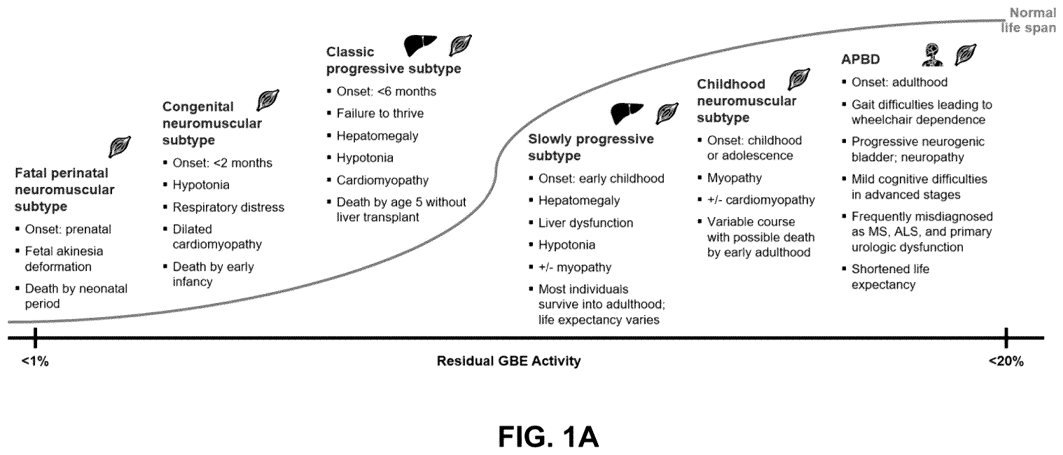

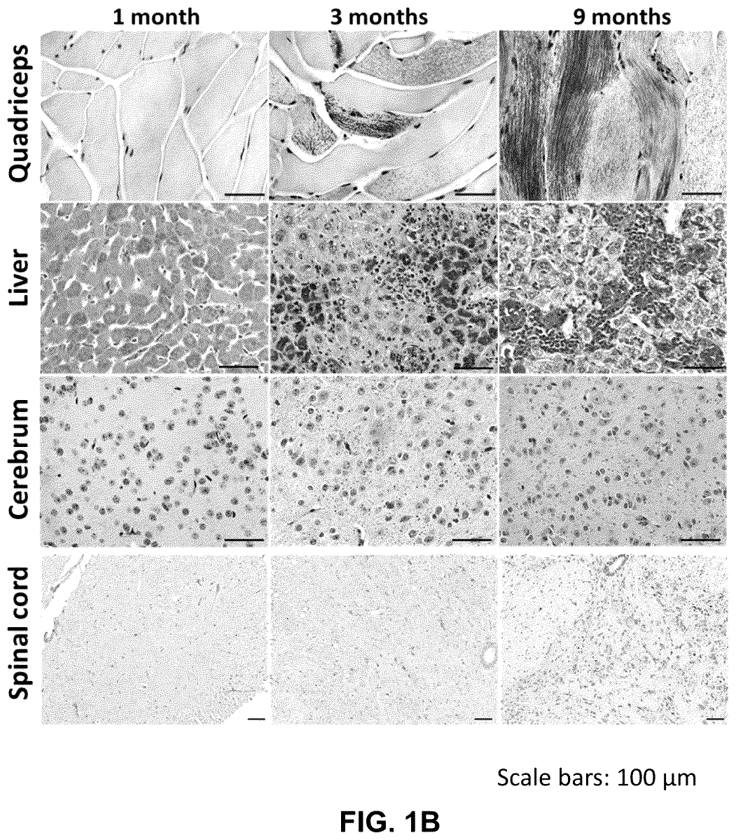

### V. BRIEF SUMMARY

Disclosed herein is an isolated nucleic acid molecule comprising a nucleic acid sequence encoding a polypeptide for preventing glycogen accumulation and/or degrading accumulated glycogen, wherein the nucleic acid sequence is CpG-depleted and codon-optimized for expression in a human or a mammalian cell.

Disclosed herein is a vector comprising an isolated nucleic acid molecule comprising a nucleic acid sequence encoding a polypeptide for preventing glycogen accumulation and/or degrading accumulated glycogen, wherein the nucleic acid sequence is CpG-depleted and codon-optimized for expression in a human or a mammalian cell.

Disclosed herein is an AAV vector comprising an isolated nucleic acid molecule, wherein the isolated nucleic acid sequence encodes a human glycogen branching enzyme, and wherein the isolated nucleic acid sequence is CpG-depleted and codon-optimized for expression in a human or a mammalian cell.

Disclosed herein is vector comprising a gene expression cassette comprising an isolated nucleic acid molecule comprising a nucleic acid sequence encoding a polypeptide for preventing glycogen accumulation and/or degrading accumulated glycogen under the control of a ubiquitous promoter, a tissue-specific promoter, or an immunotolerant dual promoter comprising a liver-specific promoter and a ubiquitous promoter, wherein the nucleic acid sequence is CpG-depleted and codon-optimized for expression in a human or a mammalian cell.

Disclosed herein is a method of treating GSD IV and/or APBD disease progression comprising administering to a subject in need thereof a therapeutically effective amount of a disclosed isolated nucleic acid molecule, a disclosed vector, a disclosed pharmaceutical formulation, or a combination thereof.

Disclosed herein is a method of treating GSD IV and/or APBD disease progression comprising administering to a subject in need thereof a therapeutically effective amount of a vector comprising an isolated nucleic acid molecule comprising a nucleic acid sequence encoding a polypeptide for preventing glycogen accumulation and/or degrading accumulated glycogen, wherein the nucleic acid sequence is CpG-depleted and codon-optimized for expression in a human or a mammalian cell, wherein glycogen accumulation is prevented and/or accumulated glycogen is degraded in the subject.

Disclosed herein is a method of treating GSD IV and/or APBD disease progression comprising preventing glycogen accumulation and/or degrading accumulated glycogen in a subject in need thereof by administering to the subject a therapeutically effective amount of a vector comprising an isolated nucleic acid molecule comprising a nucleic acid sequence encoding a polypeptide for preventing glycogen accumulation and/or degrading accumulated glycogen, wherein the nucleic acid sequence is CpG-depleted and codon-optimized for expression in a human or a mammalian cell.

Disclosed herein is a method of treating GSD IV and/or APBD disease progression comprising administering to a subject in need thereof a therapeutically effective amount of a vector comprising an isolated nucleic acid molecule comprising a nucleic acid sequence encoding a polypeptide for preventing glycogen accumulation and/or degrading accumulated glycogen, wherein the nucleic acid sequence is CpG-depleted and codon-optimized for expression in a human or a mammalian cell, thereby preventing glycogen accumulation and/or degrading accumulated glycogen in the subject.

Disclosed herein is a method of preventing glycogen accumulation and/or degrading accumulated glycogen, comprising: administering to a subject having GSD IV and/or APBD a therapeutically effective amount of a vector comprising an isolated nucleic acid molecule comprising a nucleic acid sequence encoding a polypeptide for preventing glycogen accumulation and/or degrading accumulated glycogen, wherein the nucleic acid sequence is CpG-depleted and codon-optimized for expression in a human or a mammalian cell.

Disclosed herein is a method of treating GSD IV and/or APBD disease progression comprising administering to a subject in need thereof a therapeutically effective amount of a disclosed isolated nucleic acid molecule.

Disclosed herein is a method of treating GSD IV and/or APBD disease progression comprising administering to a subject in need thereof an isolated nucleic acid molecule comprising a nucleic acid sequence encoding a therapeutic polypeptide, wherein glycogen accumulation is prevented and/or accumulated glycogen is degraded in the subject.

Disclosed herein is a method of treating GSD IV and/or APBD disease progression comprising preventing glycogen accumulation and/or degrading accumulated glycogen in a subject in need thereof by administering to the subject an isolated nucleic acid molecule comprising a nucleic acid sequence encoding a therapeutic polypeptide.

Disclosed herein is a method of treating GSD IV and/or APBD disease progression comprising administering to a subject in need thereof an isolated nucleic acid molecule comprising a nucleic acid sequence encoding a therapeutic polypeptide, thereby preventing glycogen accumulation and/or degrading accumulated glycogen in the subject.

Disclosed herein is a method of preventing glycogen accumulation and/or degrading accumulated glycogen comprising administering to a subject having GSD IV and/or APBD disease progression an isolated nucleic acid molecule comprising a nucleic acid sequence encoding a therapeutic polypeptide.

Disclosed herein is a method of treating and/or preventing a disease comprising administering to a subject in need thereof a therapeutically effective amount of a disclosed isolated nucleic acid molecule, a disclosed vector, disclosed pharmaceutical formulation, or a combination thereof, wherein the disease is a GSD (such as GSD IV and/or APBD), Lafora disease (including those diseases caused by mutations in the EPM2A gene (glucan phosphatase, laforin) or the NHLRC1 gene (NHL repeat containing E3 ubiquitin protein ligase 1 or EPM2B), polyglucosan body myopathy-1, polyglucosan body myopathy-2, or any disease or pathology caused by a mutation in a GYG1 gene, a RBCK1 gene, a PRKAG2 gene, or any disease resulting in polyglucosan body accumulation.

Disclosed herein is a method of treating and/or preventing a disease comprising administering to a subject in need thereof a therapeutically effective amount of a vector comprising an isolated nucleic acid molecule comprising a nucleic acid sequence encoding a polypeptide for preventing glycogen accumulation and/or degrading accumulated glycogen, wherein the nucleic acid sequence is CpG-depleted and codon-optimized for expression in a human or a mammalian cell, and preventing glycogen accumulation and/or degrading accumulated glycogen in the subject, wherein the disease is a GSD (such as GSD IV and/or APBD), Lafora disease (including those diseases caused by mutations in the EPM2A gene (glucan phosphatase, laforin) or the NHLRC1 gene (NHL repeat containing E3 ubiquitin protein ligase 1 or EPM2B)), polyglucosan body myopathy-1, polyglucosan body myopathy-2, or any disease or pathology caused by a mutation in a GYG1 gene, a RBCK1 gene, or a PRKAG2 gene.

Disclosed herein is a method of treating and/or preventing a disease comprising preventing glycogen accumulation and/or degrading accumulated glycogen in a subject in need thereof by administering to the subject a therapeutically effective amount of a vector comprising an isolated nucleic acid molecule comprising a nucleic acid sequence encoding a polypeptide for preventing glycogen accumulation and/or degrading accumulated glycogen, wherein the nucleic acid sequence is CpG-depleted and codon-optimized for expression in a human or a mammalian cell, wherein the disease is a GSD (such as GSD IV and/or APBD), Lafora disease (including those diseases caused by mutations in the EPM2A gene (glucan phosphatase, laforin) or the NHLRC1 gene (NHL repeat containing E3 ubiquitin protein ligase 1 or EPM2B)), polyglucosan body myopathy-1, polyglucosan body myopathy-2, or any disease or pathology caused by a mutation in a GYG1 gene, a RBCK1 gene, or a PRKAG2 gene.

Disclosed herein is a method of treating and/or preventing a disease comprising administering to a subject in need thereof a therapeutically effective amount of a vector comprising an isolated nucleic acid molecule comprising a nucleic acid sequence encoding a polypeptide for preventing glycogen accumulation and/or degrading accumulated glycogen, wherein the nucleic acid sequence is CpG-free and codon-optimized for expression in a human or a mammalian cell, thereby preventing glycogen accumulation and/or degrading accumulated glycogen in the subject, wherein the disease is a GSD (such as GSD IV and/or APBD), Lafora disease (including those diseases caused by mutations in the EPM2A gene (glucan phosphatase, laforin) or the NHLRC1 gene (NHL repeat containing E3 ubiquitin protein ligase 1 or EPM2B)), polyglucosan body myopathy-1, polyglucosan body myopathy-2, or any disease or pathology caused by a mutation in a GYG1 gene, a RBCK1 gene, or a PRKAG2 gene.

Disclosed herein is a method of preventing glycogen accumulation and/or degrading accumulated glycogen comprising administering to a subject having a disease a therapeutically effective amount of a vector comprising an isolated nucleic acid molecule comprising a nucleic acid sequence encoding a polypeptide for preventing glycogen accumulation and/or degrading accumulated glycogen, wherein the nucleic acid sequence is CpG-depleted and codon-optimized for expression in a human or a mammalian cell, wherein the disease is a GSD (such as GSD IV and/or APBD), Lafora disease (including those diseases caused by mutations in the EPM2A gene (glucan phosphatase, laforin) or the NHLRC1 gene (NHL repeat containing E3 ubiquitin protein ligase 1 or EPM2B)), polyglucosan body myopathy-1, polyglucosan body myopathy-2, or any disease or pathology caused by a mutation in a GYG1 gene, a RBCK1 gene, or a PRKAG2 gene.

Disclosed herein is a method of treating and/or preventing GSD IV and/or APBD disease progression comprising administering to a subject in need thereof a therapeutically effective amount of a disclosed isolated nucleic acid molecule, a disclosed vector, and/or a disclosed pharmaceutical formulation, and administering to the subject an RNA therapeutic to reduce or inhibit the expression level and/or activity level of glycogen synthase, wherein the RNA therapeutic comprises RNAi or antisense oligonucleotides or wherein the RNA therapeutic comprises miRNA.

Disclosed herein is a method of treating and/or preventing GSD IV and/or APBD disease progression comprising administering to a subject in need thereof a therapeutically effective amount of a disclosed isolated nucleic acid molecule, a disclosed vector, and/or a disclosed pharmaceutical formulation, and administering to the subject a small molecule to reduce or inhibit the expression level and/or activity level of glycogen synthase, wherein the small molecule targets GYS1 and/or GYS2, transcription of GYS1 and/or GYS2, and/or translation of GYS1 and/or GYS2.

Disclosed herein is a method of treating and/or preventing GSD IV and/or APBD disease progression comprising administering to a subject in need thereof a therapeutically effective amount of a disclosed isolated nucleic acid molecule, a disclosed vector, and/or a disclosed pharmaceutical formulation, and using a gene editing system to reduce or inhibit the expression level and/or activity level of glycogen synthase, wherein the gene editing system comprises a Cas9 enzyme sequence (or a derivative thereof) and a guide RNA (gRNA).

Disclosed herein is a method of treating and/or preventing GSD IV and/or APBD disease progression comprising administering to a subject in need thereof a therapeutically effective amount of a disclosed isolated nucleic acid molecule, a disclosed vector, and/or a disclosed pharmaceutical formulation, and reducing or inhibiting the expression level and/or activity level of glycogen synthase, wherein reducing or inhibiting the expression level and/or activity level of glycogen synthase comprises any means known to and practiced by the art and/or disclosed herein.

Disclosed herein is a method of preventing glycogen accumulation and/or degrading accumulated glycogen comprising administering to a subject in need thereof a therapeutically effective amount of a disclosed isolated nucleic acid molecule, a disclosed vector, a disclosed pharmaceutical formulation, or a combination thereof, and a therapeutically effective amount of an agent for reducing the expression level and/or activity level of glycogen synthase.

### VI. DETAILED DESCRIPTION

The present disclosure describes formulations, compounded compositions, kits, capsules, containers, and/or methods thereof. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. Definitions

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase “consisting essentially of” limits the scope of a claim to the recited components in a composition or the recited steps in a method as well as those that do not materially affect the basic and novel characteristic or characteristics of the claimed composition or claimed method. The phrase “consisting of” excludes any component, step, or element that is not recited in the claim. The phrase “comprising” is synonymous with “including”, “containing”, or “characterized by”, and is inclusive or open-ended. “Comprising” does not exclude additional, unrecited components or steps.

As used herein, when referring to any numerical value, the term “about” means a value falling within a range that is ±10% of the stated value.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5, and are present in such ratio regardless of whether additional components are contained in the compound.

As used herein, the terms “optional” or “optionally” means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

As used herein, the term “subject” refers to the target of administration, e.g., a human being. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). The subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, adult and child subjects, as well as fetuses, whether male or female, are intended to be covered. In an aspect, a subject can be a human patient. In an aspect, a subject can have a glycogen storage disease, be suspected of having a glycogen storage disease, or be at risk of developing a glycogen storage disease. In an aspect, a glycogen storage disease can be a GSD (such as GSD IV and/or APBD), Lafora disease (including those diseases caused by mutations in the EPM2A gene (glucan phosphatase, laforin) or the NHLRC1 gene (NHL repeat containing E3 ubiquitin protein ligase 1 or EPM2B)), polyglucosan body myopathy-1, polyglucosan body myopathy-2, or any disease or pathology caused by a mutation in a GYG1 gene, a RBCK1 gene, or a PRKAG2 gene. In an aspect, a GSD can be GSD IV. In an aspect, a GSD can be APBD.

As used herein, the term “diagnosed” means having been subjected to an examination by a person of skill, for example, a physician, and found to have a condition that can be diagnosed or treated by one or more of the disclosed isolated nucleic acid molecules, disclosed vectors, disclosed pharmaceutical formulations, or a combination thereof, or by one or more of the disclosed methods. For example, “diagnosed with a glycogen storage disease” means having been subjected to an examination by a person of skill, for example, a physician, and found to have a condition that can be treated by one or more of the disclosed isolated nucleic acid molecules, disclosed vectors, disclosed pharmaceutical formulations, or a combination thereof, or by one or more of the disclosed methods. For example, “suspected of having a glycogen storage disease” can mean having been subjected to an examination by a person of skill, for example, a physician, and found to have a condition that can likely be treated by one or more of by one or more of the disclosed isolated nucleic acid molecules, disclosed vectors, disclosed pharmaceutical formulations, or a combination thereof, or by one or more of the disclosed methods. In an aspect, an examination can be physical, can involve various tests (e.g., blood tests, genotyping, biopsies, etc.) and assays (e.g., enzymatic assay), or a combination thereof.

A “patient” refers to a subject afflicted with a glycogen storage disease. In an aspect, a patient can refer to a subject that has been diagnosed with or is suspected of having a glycogen storage disease, Lafora disease (including those diseases caused by mutations in the EPM2A gene (glucan phosphatase, laforin) or the NHLRC1 gene (NHL repeat containing E3 ubiquitin protein ligase 1 or EPM2B)), polyglucosan body myopathy-1, polyglucosan body myopathy-2, APBD, or any disease or pathology caused by a mutation in a GYG1 gene, a RBCK1 gene, or a PRKAG2 gene. In an aspect, a patient can refer to a subject that has been diagnosed with or is suspected of having a glycogen storage disease (GSD) and is seeking treatment or receiving treatment for a GSD (such as GSD IV and/or APBD). In an aspect, a patient can refer to a subject that has been diagnosed with or is suspected of having Lafora disease (including those diseases caused by mutations in the EPM2A gene (glucan phosphatase, laforin) or the NHLRC1 gene (NHL repeat containing E3 ubiquitin protein ligase 1 or EPM2B)), polyglucosan body myopathy-1, polyglucosan body myopathy-2, APBD, or any disease or pathology caused by a mutation in a GYG1 gene, a RBCK1 gene, or a PRKAG2 gene and is seeking treatment or receiving treatment. In an aspect, a patient can refer to a subject that has been diagnosed with or is suspected of having GSD IV and/or APBD and is seeking treatment or receiving treatment.

As used herein, the phrase “identified to be in need of treatment for a disorder,” or the like, refers to selection of a subject based upon need for treatment of the disorder. For example, a subject can be identified as having a need for treatment of a disorder (e.g., a glycogen storage disease such as GSD IV and/or APBD, Lafora disease (including those diseases caused by mutations in the EPM2A gene (glucan phosphatase, laforin) or the NHLRC1 gene (NHL repeat containing E3 ubiquitin protein ligase 1 or EPM2B)), polyglucosan body myopathy-1, polyglucosan body myopathy-2, or any disease or pathology caused by a mutation in a GYG1 gene, a RBCK1 gene, or a PRKAG2 gene) based upon an earlier diagnosis by a person of skill and thereafter subjected to treatment for the disorder. In an aspect, a subject can be identified as having a need for treatment of GSD IV and/or APBD based upon an earlier diagnosis by a person of skill and thereafter subjected to treatment for the disorder. In an aspect, the identification can be performed by a person different from the person making the diagnosis. In an aspect, the administration can be performed by one who performed the diagnosis.

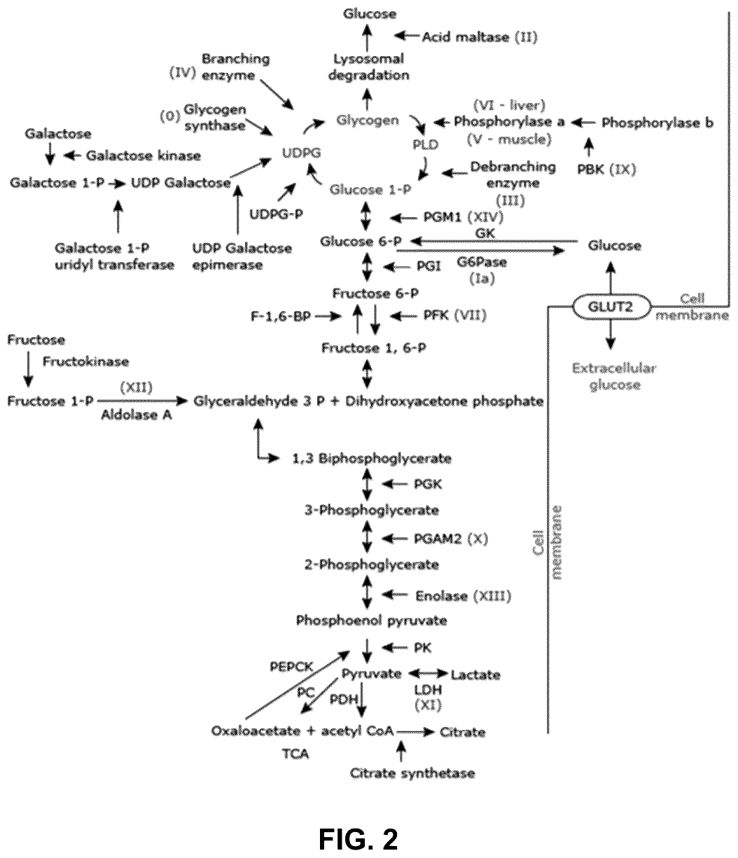

As used herein, “glycogen” refers to a branched polysaccharide with a molecular weight of 9-10 million Daltons. The average glycogen molecule contains about 55,000 glucosyl residues linked by α-1,4 (92%) and α-1,6 (8%) glycosidic bonds. Glycogen synthesis is catalyzed by the actions of 3 enzymes: (a) glycogenin (GYG), the initiating enzyme that starts a primer of glucose chain attached to itself; (b) glycogen synthase (GYS), which strings glucose to extend linear chains; and (c) glycogen-branching enzyme (GBE), which attaches a short new branch to a linear chain (see FIG. 2). In an aspect, glycogen can comprise glycogen, polyglucosan bodies, Lafora bodies, amylopectin-like glycogen, or any combination thereof.

As used herein, “inhibit,” “inhibiting”, and “inhibition” mean to diminish or decrease an activity, level, response, condition, severity, disease, or other biological parameter. This can include, but is not limited to, the complete ablation of the activity, level, response, condition, severity, disease, or other biological parameter. This can also include, for example, a 10% inhibition or reduction in the activity, level, response, condition, severity, disease, or other biological parameter as compared to the native or control level (e.g., a subject not having a GSD such as GSD IV and/or APBD, Lafora disease (including those diseases caused by mutations in the EPM2A gene (glucan phosphatase, laforin) or the NHLRC1 gene (NHL repeat containing E3 ubiquitin protein ligase 1 or EPM2B)), polyglucosan body myopathy-1, polyglucosan body myopathy-2, or any disease or pathology caused by a mutation in a GYG1 gene, a RBCK1 gene, or a PRKAG2 gene). Thus, in an aspect, the inhibition or reduction can be a 10%, 20%, 30%, 40%, 50%, 60%, 70%, 80%, 90%, 100%, or any amount of reduction in between as compared to native or control levels. In an aspect, the inhibition or reduction can be 10-20%, 20-30%, 30-40%, 40-50%, 50-60%, 60-70%, 70-80%, 80-90%, or 90-100% as compared to native or control levels. In an aspect, the inhibition or reduction can be 0-25%, 25-50%, 50-75%, or 75-100% as compared to native or control levels.

The words “treat” or “treating” or “treatment” include palliative treatment, that is, treatment designed for the relief of symptoms rather than the curing of the disease, pathological condition, or disorder; preventative treatment, that is, treatment directed to minimizing or partially or completely inhibiting, or mitigating the development of the associated disease, pathological condition, or disorder; and supportive treatment, that is, treatment employed to supplement another specific therapy directed toward the improvement of the associated disease, pathological condition, or disorder. In an aspect, the terms cover any treatment of a subject, including a mammal (e.g., a human), and includes: (i) preventing the undesired physiological change, disease, pathological condition, or disorder from occurring in a subject that can be predisposed to the disease but has not yet been diagnosed as having it; (ii) inhibiting the physiological change, disease, pathological condition, or disorder, i.e., arresting its development; or (iii) relieving the physiological change, disease, pathological condition, or disorder, i.e., causing regression of the disease. For example, in an aspect, treating a GSD such as GSD IV and/or APBD, Lafora disease (including those diseases caused by mutations in the EPM2A gene (glucan phosphatase, laforin) or the NHLRC1 gene (NHL repeat containing E3 ubiquitin protein ligase 1 or EPM2B)), polyglucosan body myopathy-1, polyglucosan body myopathy-2, or any disease or pathology caused by a mutation in a GYG1 gene, a RBCK1 gene, or a PRKAG2 gene) can reduce the severity of an established GSD in a subject by 1%-100% as compared to a control (such as, for example, an individual not having a glycogen storage disease). In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of a GSD (such as GSD IV and/or APBD). For example, treating a GSD can reduce one or more symptoms of a GSD in a subject by 1%-100% as compared to a control (such as, for example, an individual not having a glycogen storage disease). In an aspect, treating can refer to 1%, 2%, 3%, 4%, 5%, 6%, 7%, 8%, 9% 10%, 20%, 30%, 40%, 50%, 60%, 70%, 80%, 90%, 100% reduction of one or more symptoms of an established GSD. It is understood that treatment does not necessarily refer to a cure or complete ablation or eradication of a GSD such as GSD IV and/or APBD, Lafora disease (including those diseases caused by mutations in the EPM2A gene (glucan phosphatase, laforin) or the NHLRC1 gene (NHL repeat containing E3 ubiquitin protein ligase 1 or EPM2B)), polyglucosan body myopathy-1, polyglucosan body myopathy-2, or any disease or pathology caused by a mutation in a GYG1 gene, a RBCK1 gene, or a PRKAG2 gene). However, in an aspect, treatment can refer to a cure or complete ablation or eradication of a GSD (such as GSD IV) or any condition which results in polyglucosan body accumulation (such as APBD). In an aspect, treating can refer to the minimizing or reversing polyglucosan body accumulation in the subject.

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing a GSD (such as GSD IV and/or APBD) is intended. The words “prevent” and “preventing” and “prevention” can also refer to prophylactic or preventative measures for protecting or precluding a subject (e.g., an individual) not having a given GSD or GSD-related complication from progressing to that complication. In an aspect, a GSD can be GSD IV and/or APBD, Lafora disease (including those diseases caused by mutations in the EPM2A gene (glucan phosphatase, laforin) or the NHLRC1 gene (NHL repeat containing E3 ubiquitin protein ligase 1 or EPM2B)), polyglucosan body myopathy-1, polyglucosan body myopathy-2, or any disease or pathology caused by a mutation in a GYG1 gene, a RBCK1 gene, or a PRKAG2 gene. However, in an aspect, prevention can refer to a cure or complete ablation or eradication of a GSD (such as GSD IV) or any condition which results in polyglucosan body accumulation (such as APBD). In an aspect, preventing can refer to the minimizing or reversing polyglucosan body accumulation in the subject.

As used herein, the term “glycogen” can refer to glycogen, polyglucosan bodies, amylopectin-like glycogen, Lafora bodies, or any combination thereof. For example, as used herein, the phrase “preventing glycogen accumulation and/or degrading accumulated glycogen” can also be read to include polyglucosan bodies, amylopectin-like glycogen, Lafora bodies, or any combination thereof in addition to glycogen.

As used herein, the terms “administering” and “administration” refer to any method of providing one or more of the disclosed isolated nucleic acid molecules, disclosed vectors, disclosed pharmaceutical formulations, or a combination thereof to a subject. Such methods are well known to those skilled in the art and include, but are not limited to, the following: oral administration, transdermal administration, administration by inhalation, nasal administration, topical administration, intravaginal administration, in utero administration, ophthalmic administration, intraaural administration, otic administration, intracerebral administration, rectal administration, sublingual administration, buccal administration, and parenteral administration, including injectable such as intravenous administration, intra-CSF administration, intracerebroventricular (ICV) administration, intraventricular administration, intra-cistema magna (ICM) administration, intraparenchymal administration, intrathecal (lumbar, cistemal, or both) administration, intra-lumber administration, intra-arterial administration, intramuscular administration, and subcutaneous administration. In an aspect, administration can comprise one or more modes of administration, such as, for example, IV administration and intra-CSF administration. In an aspect, any combination of administration can be used such as intra-hepatic administration and IV administration. Administration of a disclosed isolated nucleic acid molecule, a disclosed vector, a disclosed pharmaceutical composition, a disclosed therapeutic agent, a disclosed immune modulator, a disclosed proteasome inhibitor, a disclosed small molecule, a disclosed endonuclease, a disclosed oligonucleotide, and/or a disclosed RNA therapeutic can comprise administration directly into the CNS or the PNS. Administration can be continuous or intermittent and can comprise a combination of one or more routes of administration. In an aspect, a disclosed small molecule that inhibits glycogen synthase (GYS1) can be orally delivered.

In an aspect, the skilled person can determine an efficacious dose, an efficacious schedule, and an efficacious route of administration for one or more of the disclosed isolated nucleic acid molecules, disclosed vectors, disclosed pharmaceutical formulations, or a combination thereof so as to treat or prevent a GSD such as GSD IV and/or APBD, Lafora disease (including those diseases caused by mutations in the EPM2A gene (glucan phosphatase, laforin) or the NHLRC1 gene (NHL repeat containing E3 ubiquitin protein ligase 1 or EPM2B)), polyglucosan body myopathy-1, polyglucosan body myopathy-2, or any disease or pathology caused by a mutation in a GYG1 gene, a RBCK1 gene, or a PRKAG2 gene.). In an aspect, the skilled person can also alter, change, or modify an aspect of an administering step to improve efficacy of one or more of the disclosed isolated nucleic acid molecules, disclosed vectors, disclosed pharmaceutical formulations, or a combination thereof.

As used herein, “modifying the method” can comprise modifying or changing one or more features or aspects of one or more steps of a disclosed method. For example, in an aspect, a method can be altered by changing the amount of one or more of the disclosed isolated nucleic acid molecules, disclosed vectors, disclosed pharmaceutical formulations, or a combination thereof administered to a subject, or by changing the frequency of administration of one or more of the disclosed isolated nucleic acid molecules, disclosed vectors, disclosed pharmaceutical formulations, or a combination thereof to a subject, or by changing the duration of time one or more of the disclosed isolated nucleic acid molecules, disclosed vectors, disclosed pharmaceutical formulations, or a combination are administered to a subject.

As used herein, “concurrently” means (1) simultaneously in time, or (2) at different times during the course of a common treatment schedule.

The term “contacting” as used herein refers to bringing one or more of the disclosed isolated nucleic acid molecules, disclosed vectors, disclosed pharmaceutical formulations, or a combination thereof together with a target area or intended target area in such a manner that the one or more of the disclosed isolated nucleic acid molecules, disclosed vectors, disclosed pharmaceutical formulations, or a combination thereof exert an effect on the intended target or targeted area either directly or indirectly. A target area or intended target area can be one or more of a subject's organs (e.g., lungs, heart, liver, kidney, brain, etc.). In an aspect, a target area or intended target area can be any cell or any organ infected by a GSD such as GSD IV and/or APBD, Lafora disease (including those diseases caused by mutations in the EPM2A gene (glucan phosphatase, laforin) or the NHLRC1 gene (NHL repeat containing E3 ubiquitin protein ligase 1 or EPM2B)), polyglucosan body myopathy-1, polyglucosan body myopathy-2, or any disease or pathology caused by a mutation in a GYG1 gene, a RBCK1 gene, or a PRKAG2 gene). In an aspect, a target area or intended target area can be the liver.

As used herein, “determining” can refer to measuring or ascertaining the presence and severity of a glycogen storage disease, such as, for example, GSD IV and/or APBD, Lafora disease (including those diseases caused by mutations in the EPM2A gene (glucan phosphatase, laforin) or the NHLRC1 gene (NHL repeat containing E3 ubiquitin protein ligase 1 or EPM2B)), polyglucosan body myopathy-1, polyglucosan body myopathy-2, or any disease or pathology caused by a mutation in a GYG1 gene, a RBCK1 gene, or a PRKAG2 gene. Methods and techniques used to determine the presence and/or severity of a GSD are typically known to the medical arts. For example, the art is familiar with the ways to identify and/or diagnose the presence, severity, or both of a GSD.

As used herein, “effective amount” and “amount effective” can refer to an amount that is sufficient to achieve the desired result such as, for example, the treatment and/or prevention of a glycogen storage disease (e.g., GSD IV and/or APBD, Lafora disease (including those diseases caused by mutations in the EPM2A gene (glucan phosphatase, laforin) or the NHLRC1 gene (NHL repeat containing E3 ubiquitin protein ligase 1 or EPM2B)), polyglucosan body myopathy-1, polyglucosan body myopathy-2, or any disease or pathology caused by a mutation in a GYG1 gene, a RBCK1 gene, or a PRKAG2 gene) or a suspected a glycogen storage disease (e.g., GSD IV, Lafora disease (including those diseases caused by mutations in the EPM2A gene (glucan phosphatase, laforin) or the NHLRC1 gene (NHL repeat containing E3 ubiquitin protein ligase 1 or EPM2B)), polyglucosan body myopathy-1, polyglucosan body myopathy-2, or any disease or pathology caused by a mutation in a GYG1 gene, a RBCK1 gene, or a PRKAG2 gene).

As used herein, the terms “effective amount” and “amount effective” can refer to an amount that is sufficient to achieve the desired an effect on an undesired condition (e.g., a GSD). For example, a “therapeutically effective amount” refers to an amount that is sufficient to achieve the desired therapeutic result or to have an effect on undesired symptoms but is generally insufficient to cause adverse side effects. In an aspect, “therapeutically effective amount” means an amount of a disclosed composition that (i) treats the particular disease, condition, or disorder (e.g., a GSD), (ii) attenuates, ameliorates, or eliminates one or more symptoms of the particular disease, condition, or disorder e.g., a glycogen storage disease), or (iii) delays the onset of one or more symptoms of the particular disease, condition, or disorder described herein (e.g., a GSD). The specific therapeutically effective dose level for any particular patient will depend upon a variety of factors including the disorder being treated and the severity of the disorder; the disclosed isolated nucleic acid molecules, disclosed vectors, disclosed pharmaceutical formulations employed; the disclosed methods employed; the age, body weight, general health, sex and diet of the patient; the time of administration; the route of administration; the rate of excretion of the disclosed isolated nucleic acid molecules, disclosed vectors, or disclosed pharmaceutical formulations employed; the duration of the treatment; drugs used in combination or coincidental with the disclosed isolated nucleic acid molecules, disclosed vectors, or disclosed pharmaceutical formulations employed, and other like factors well known in the medical arts. For example, it is well within the skill of the art to start doses of the disclosed isolated nucleic acid molecules, disclosed vectors, or disclosed pharmaceutical formulations at levels lower than those required to achieve the desired therapeutic effect and to gradually increase the dosage until the desired effect is achieved. If desired, then the effective daily dose can be divided into multiple doses for purposes of administration. Consequently, a single dose of the disclosed isolated nucleic acid molecules, disclosed vectors, or disclosed pharmaceutical formulations can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition, such as, for example, a glycogen storage disease (e.g., GSD IV and/or APBD, Lafora disease (including those diseases caused by mutations in the EPM2A gene (glucan phosphatase, laforin) or the NHLRC1 gene (NHL repeat containing E3 ubiquitin protein ligase 1 or EPM2B)), polyglucosan body myopathy-1, polyglucosan body myopathy-2, or any disease or pathology caused by a mutation in a GYG1 gene, a RBCK1 gene, or a PRKAG2 gene).

As used herein, “enzyme replacement therapy” (ERT) refers to an attempt to supplement the deficient enzyme activity with exogenously supplied enzyme. In an aspect, the enzyme can be a human enzyme or a non-human enzyme. In an aspect, ERT can refer to any effort to correct one or more aspects of a dysregulated glycogen metabolism pathway, such as glycogen synthesis or glycogenolysis, by supplying a deficient enzyme or precursor of a deficient enzyme. In an aspect, such an enzyme can be any enzyme encoded by one or more of the GYG1 gene, the RBCK1 gene, the PRKAG2 gene, or the GBE gene, or a combination thereof. In an aspect, such an enzyme can replace any enzyme in a dysregulated or dysfunctional glycogen metabolism pathway (see, e.g., FIG. 2). The art is familiar with the methodology of ERT and the skilled person can determine the dose and frequency of ERT necessary to obtain a desired clinical effect.

As used herein, the term “pharmaceutically acceptable carrier” refers to sterile aqueous or nonaqueous solutions, dispersions, suspensions, or emulsions, as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. In an aspect, a pharmaceutical carrier employed can be a solid, liquid, or gas. In an aspect, examples of solid carriers can include lactose, terra alba, sucrose, talc, gelatin, agar, pectin, acacia, magnesium stearate, and stearic acid. In an aspect, examples of liquid carriers can include sugar syrup, peanut oil, olive oil, and water. In an aspect, examples of gaseous carriers can include carbon dioxide and nitrogen. In preparing a disclosed composition for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, for reference, Remington's Pharmaceutical Sciences, (1990) Mack Publishing Co., Easton, Pa., which is hereby incorporated by reference in its entirety.

In an aspect, a disclosed promoter can be a promoter for the G6PC gene, which is the gene that encodes glucose-6-phosphatase-α (G6Pase-α or G6PC) gene. In an aspect, a disclosed promoter can comprise nucleotides 182-3045 in pTR-GPE-human G6PC-S298C (SEQ ID NO:28). In an aspect, a disclosed promoter can comprise nucleotides 182-3045 in pTR-GPE-codon optimized (co) G6PC-S298C (SEQ ID NO:29). In an aspect, a disclosed promoter corresponding to nucleotides 182-3045 of either SEQ ID NO:28 or SEQ ID NO:29 can drive expression of a disclosed a nucleic acid sequence encoding a polypeptide capable of preventing glycogen accumulation and/or degrading accumulated glycogen (such as, for example, GBE).

As used herein, “adult polyglucosan body disease” or “APBD” refers to late adult onset GSD IV. APBD is a neurodegenerative disorder that simulates amyotrophic lateral sclerosis (ALS), but is often associated with bladder dysfunction and dementia (about 50% of the time). APBD is characterized by dysfunction of the central (CNS) and peripheral nervous systems (PNS). In individuals with APBD, associated symptoms and findings may include sensory loss in the legs; progressive muscle weakness of the arms and legs; walking (gait) disturbances; progressive urinary difficulties; occasionally mild cognitive impairment or dementia; deficiencies in the autonomic nervous system; and/or other abnormalities. Symptoms and severity can vary greatly from one person to another. Typically, symptoms develop around the fifth decade of life. The initial sign is often times related to neurogenic bladder: specifically, an increased need to urinate that may eventually progress to cause a near complete loss of bladder control (urinary incontinence). In some cases, urinary difficulties may precede other symptoms by one or two decades. Another common early sign of APBD disease can be a feeling of numbness or weakness in the hands and feet (paresthesia). Affected individuals may experience an inability to lift the front part of the foot (foot drop), which results in the need to drag the front of the foot on the ground when walking. Affected individuals may experience weakness in the arms and legs. Eventually, affected individuals may develop progressively increased muscle tone and stiffness of the legs (spasticity), causing difficulty walking. Most individuals may eventually need assistance walking (e.g., cane or walker), and ultimately the use of a wheelchair may be required. Some affected individuals may develop mild cognitive impairment, most commonly, mild attention and memory deficits. In some cases, cognitive problems may worsen, resulting in progressive loss of memory and intellectual abilities (dementia).

As used herein, “CpG-free” can mean completely free of CpGs or partially free of CpGs. In an aspect, “CpG-free” can mean “CpG-depleted”. In an aspect, “CpG-depleted” can mean completely depleted of CpGs or partially depleted of CpGs. In an aspect, “CpG-free” can mean “CpG-optimized” for a desired and/or ideal expression level. CpG depletion and/or optimization is known to the skilled person in the art.

As used herein, “small molecule” can refer to any organic or inorganic material that is not a polymer. Small molecules exclude large macromolecules, such as large proteins (e.g., proteins with molecular weights over 2,000, 3,000, 4,000, 5,000, 6,000, 7,000, 8,000, 9,000, or 10,000), large nucleic acids (e.g., nucleic acids with molecular weights of over 2,000, 3,000, 4,000, 5,000, 6,000, 7,000, 8,000, 9,000, or 10,000), or large polysaccharides (e.g., polysaccharides with a molecular weight of over 2,000, 3,000, 4,000, 5,000, 6,000, 7,000, 8,000, 9,000, or 10,000). In an aspect, a “small molecule”, for example, can be a drug that can enter cells easily because it has a low molecular weight.

As used herein, “guaiacol” refers to a small molecule having a MW of 124.14. Guaiacol is a monomethoxybenzene comprising phenol with a methoxy substituent at the ortho position (C₇H₈O₂). In an aspect, guaiacol can increase inactivating GYS1 phosphorylation and/or can increase phosphorylation of the master activator of catabolism, AMP-dependent protein kinase. In an aspect, guaiacol can be a competitive inhibitor of purified GYS1 and GYS2 and a mixed inhibitor of the enzymes in cell lysates.

As used herein, “RNA therapeutics” can refer to the use of oligonucleotides to target RNA. RNA therapeutics can offer the promise of uniquely targeting the precise nucleic acids involved in a particular disease with greater specificity, improved potency, and decreased toxicity. This could be particularly powerful for genetic diseases where it is most advantageous to aim for the RNA as opposed to the protein. In an aspect, a therapeutic RNA can comprise one or more expression sequences. As known to the art, expression sequences can comprise an RNAi, shRNA, mRNA, non-coding RNA (ncRNA), an antisense such as an antisense RNA, miRNA, morpholino oligonucleotide, peptide-nucleic acid (PNA) or ssDNA (with natural, and modified nucleotides, including but not limited to, LNA, BNA, 2′-O-Me-RNA, 2′-MEO-RNA, 2′-F-RNA), or analog or conjugate thereof. In an aspect, a disclosed therapeutic RNA can comprise one or more long non-coding RNA (lncRNA), such as, for example, a long intergenic non-coding RNA (lincRNA), pre-transcript, pre-miRNA, pre-mRNA, competing endogenous RNA (ceRNA), small nuclear RNA (snRNA), small nucleolar RNA (snoRNA), pseudo-gene, rRNA, or tRNA. In an aspect, ncRNA can be piwi-interacting RNA (piRNA), primary miRNA (pri-miRNA), or premature miRNA (pre-miRNA). In an aspect, a therapeutic RNA or RNA therapeutic can comprise antisense oligonucleotides (ASOs) that inhibit mRNA translation, oligonucleotides that function via RNA interference (RNAi) pathway, RNA molecules that behave like enzymes (ribozymes), RNA oligonucleotides that bind to proteins and other cellular molecules, and ASOs that bind to mRNA and form a structure that is recognized by RNase H resulting in cleavage of the mRNA target. In an aspect, RNA therapeutics can comprise RNAi and ASOs that inhibit mRNA translation of liver or muscle glycogen synthase (e.g., GYS1 and/or GYS2). Generally speaking, as known to the art, RNAi operates sequence specifically and post-transcriptionally by activating ribonucleases which, along with other enzymes and complexes, coordinately degrade the RNA after the original RNA target has been cut into smaller pieces while antisense oligonucleotides bind to their target nucleic acid via Watson-Crick base pairing, and inhibit or alter gene expression via steric hindrance, splicing alterations, initiation of target degradation, or other events.

As known to the art, miRNAs are small non-coding RNAs that are about 17 to about 25 nucleotide bases (nt) in length in their biologically active form. In an aspect, a disclosed miRNA can regulate gene expression post transcriptionally by decreasing target mRNA translation. In an aspect, a disclosed miRNA can function as a negative regulator. In an aspect, a disclosed miRNA is about 17 to about 25, about 17 to about 24, about 17 to about 23, about 17 to about 22, about 17 to about 21, about 17 to about 20, about 17 to about 19, about 18 to about 25, about 18 to about 24, about 18 to about 23, about 18 to about 22, about 18 to about 21, about 18 to about 20, about 19 to about 25, about 19 to about 24, about 19 to about 23, about 19 to about 22, about 19 to about 21, about 20 to about 25, about 20 to about 24, about 20 to about 23, about 20 to about 22, about 21 to about 25, about 21 to about 24, about 21 to about 23, about 22 to about 25, about 22 to about 24, or about 22 nt in length. Generally, there are three forms of miRNAs: primary miRNAs (pri-miRNAs), premature miRNAs (pre-miRNAs), and mature miRNAs, all of which are within the scope of the present disclosure.

As used herein, “promoter” or “promoters” are known to the art. Depending on the level and tissue-specific expression desired, a variety of promoter elements can be used. A promoter can be tissue-specific or ubiquitous and can be constitutive or inducible, depending on the pattern of the gene expression desired. A promoter can be native or foreign and can be a natural or a synthetic sequence. By foreign, it is intended that the transcriptional initiation region is not found in the wild-type host into which the transcriptional initiation region is introduced.

“Tissue-specific promoters” are known to the art and include, but are not limited to, neuron-specific promoters, muscle-specific promoters, liver-specific promoters, skeletal muscle-specific promoters, and heart-specific promoters.

“Neuron-specific promoters” are known to the art and include, but are not limited to, the synapsin I (SYN) promoter, the neuron-specific enolase promoter, the calcium/calmodulin-dependent protein kinase II promoter, the tubulin alpha I promoter, and the platelet-derived growth factor beta chain promoter.

“Liver-specific promoters” are known to the art and include, but are not limited to, the al-microglobulin/bikunin enhancer/thyroid hormone-binding globulin promoter, the human albumin (hALB) promoter, the thyroid hormone-binding globulin promoter, thyroxin binding globulin promoter, the α-1-anti-trypsin promoter, the bovine albumin (bAlb) promoter, the murine albumin (mAlb) promoter, the human α1-antitrypsin (hAAT) promoter, the ApoEhAAT promoter composed of the ApoE enhancer and the hAAT promoter, the transthyretin (TTR) promoter, the liver fatty acid binding protein promoter, the hepatitis B virus (HBV) promoter, the DC172 promoter consisting of the hAAT promoter and the α1-microglobulin enhancer, the DC190 promoter containing the human albumin promoter and the prothrombin enhancer, and other natural and synthetic liver-specific promoters.

In an aspect, a disclosed liver-specific promoter can comprise any liver-specific promoter known to the art. In an aspect, a liver specific promoter can comprise about 845-bp and comprise the thyroid hormone-binding globulin promoter sequences (2382 to 13), two copies of α1-microglobulinybikunin enhancer sequences (22,804 through 22,704), and a 71-bp leader sequence (Ill C R, et al. (1997) Blood Coagul Fibrinolysis. 8 Suppl 2:S23-S30). In an aspect, a disclosed liver-specific promoter can comprise the sequence set froth in SEQ ID NO:34. In an aspect, a disclosed liver-specific promoter can comprise a sequence having at least 40%, 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:34. In an aspect, a disclosed liver-specific promoter can comprise a sequence having at least 40%-60%, at least 60%-80%, at least 80%-90%, or at least 90%-100% identity to the sequence set forth in SEQ ID NO:34.

In an aspect, a disclosed promoter can be an endogenous promoter. In an aspect, a disclosed endogenous promoter can generally be obtained from a non-coding region upstream of a transcription initiation site of a gene (such as, for example, a disclosed GBE or some other enzyme involved in the glycogen signaling pathway). In an aspect, a disclosed engoengous promoter can be used for constitutive and efficient expression of a disclosed transgene (e.g., a nucleic acid sequence encoding a polypeptide capable of preventing glycogen accumulation and/or degrading accumulated glycogen). The skilled person is familiar with the methods and tools to identify an endogenous promoter such as, for example, the endogenous promoter for GBE.

“Muscle-specific promoters” are known to the art and include, but are not limited to, the MHCK7 promoter, the muscle creatine kinase (MCK) promoter/enhancer, the slow isoform of troponin I (TnIS) promoter, the MYODI promoter, the MYLK2 promoter, the SPc5-12 promoter, the desmin (Des) promoter, the unc45b promoter, and other natural and synthetic muscle-specific promoters.

“Skeletal muscle-specific promoters” are known to the art and include, but are not limited to, the HSA promoter, the human α-skeletal actin promoter.

“Heart-specific promoters” are known to the art and include, but art not limited to, the MYH6 promoter, the TNNI3 promoter, the cardiac troponin C (cTnC) promoter, the alpha-myosin heavy chain (α-MHC) promoter, myosin light chain 2 (MLC-2), and the MYBPC3 promoter.

As used herein, a “ubiquitous/constitutive promoter” refer to a promoter that allows for continual transcription of its associated gene. A ubiquitous/constitutive promoter is always active and can be used to express genes in a wide range of cells and tissues, including, but not limited to, the liver, kidney, skeletal muscle, cardiac muscle, smooth muscle, diaphragm muscle, brain, spinal cord, endothelial cells, intestinal cells, pulmonary cells (e.g., smooth muscle or epithelium), peritoneal epithelial cells, and fibroblasts. Ubiquitous/constitutive promoters include, but are not limited to, a CMV major immediate-early enhancer/chicken beta-actin promoter, a cytomegalovirus (CMV) major immediate-early promoter, an Elongation Factor 1-α (EF1-α) promoter, a simian vacuolating virus 40 (SV40) promoter, an AmpR promoter, a PyK promoter, a human ubiquitin C gene (Ubc) promoter, a MFG promoter, a human beta actin promoter, a CAG promoter, a EGR1 promoter, a FerH promoter, a FerL promoter, a GRP78 promoter, a GRP94 promoter, a HSP70 promoter, a β-kin promoter, a murine phosphoglycerate kinase (mPGK) or human PGK (hPGK) promoter, a ROSA promoter, human Ubiquitin B promoter, a Rous sarcoma virus promoter, or any other natural or synthetic ubiquitous/constitutive promoters.

As used herein, an “inducible promoter” refers to a promoter that can be regulated by positive or negative control. Factors that can regulate an inducible promoter include, but are not limited to, chemical agents (e.g., the metallothionein promoter or a hormone inducible promoter), temperature, and light.

As used herein, the term “serotype” is a distinction used to refer to an AAV having a capsid that is serologically distinct from other AAV serotypes. Serologic distinctiveness can be determined on the basis of the lack of cross-reactivity between antibodies to one AAV as compared to another AAV. Such cross-reactivity differences are usually due to differences in capsid protein sequences/antigenic determinants (e.g., due to VP1, VP2, and/or VP3 sequence differences of AAV serotypes).

As used herein, “tropism” refers to the specificity of an AAV capsid protein present in an AAV viral particle, for infecting a particular type of cell or tissue. The tropism of an AAV capsid for a particular type of cell or tissue may be determined by measuring the ability of AAV vector particles comprising the hybrid AAV capsid protein to infect or to transduce a particular type of cell or tissue, using standard assays that are well-known in the art such as those disclosed in the examples of the present application. As used herein, the term “liver tropism” or “hepatic tropism” refers to the tropism for liver or hepatic tissue and cells, including hepatocytes.

“Sequence identity” and “sequence similarity” can be determined by alignment of two peptide or two nucleotide sequences using global or local alignment algorithms. Sequences may then be referred to as “substantially identical” or “essentially similar” when they are optimally aligned. For example, sequence similarity or identity can be determined by searching against databases such as FASTA, BLAST, etc., but hits should be retrieved and aligned pairwise to compare sequence identity. Two proteins or two protein domains, or two nucleic acid sequences can have “substantial sequence identity” if the percentage sequence identity is at least 70%, 75%, 80%, 85%, 90%, 95%, 98%, 99% or more, preferably 90%, 95%, 98%, 99% or more. Such sequences are also referred to as “variants” herein, e.g., other variants of glycogen branching enzymes and amylases. It should be understood that sequence with substantial sequence identity do not necessarily have the same length and may differ in length. For example, sequences that have the same nucleotide sequence, but of which one has additional nucleotides on the 3′- and/or 5′-side are 100% identical.

As used herein, “codon optimization” can refer to a process of modifying a nucleic acid sequence for enhanced expression in the host cells of interest by replacing one or more codons or more of the native sequence with codons that are more frequently or most frequently used in the genes of that host cell while maintaining the native amino acid sequence. Various species exhibit particular bias for certain codons of a particular amino acid. As contemplated herein, genes can be tailored for optimal gene expression in a given organism based on codon optimization. Codon usage tables are readily available, for example, at the “Codon Usage Database.” Many methods and software tools for codon optimization have been reported previously. (See, for example, genomes.urv.es/OPTIMIZER/).

As used herein, “substrate reduction therapy” or “SRT” refers to methods of reducing the level of the substrate to a point where residual degradative activity of one or more enzymes is sufficient to prevent substrate accumulation. Generally, SRT aims to use small molecule inhibitors of biosynthesis to reduce the concentration of accumulating substrate to a level where the residual degradative enzymes can maintain homeostasis. For example, in an aspect, SRT refers to a method of inhibiting glycogen synthase (i.e., GYS1 and/or GYS2) in a cell or a subject to reduce glycogen synthesis and/or glycogen accumulation in cells and tissues (e.g., skeletal muscle, lung tissue, liver tissue, brain tissue, or any other tissue having glycogen accumulation) when GAA and/or GBE activity and/or expression levels are reduced. In an aspect, “glycogen” can refer to glycogen, polyglucosan bodies, amylopectin-like glycogen, Lafora bodies, or any combination thereof. For example, the term “glycogen accumulation” can comprise accumulation of glycogen, polyglucosan bodies, amylopectin-like glycogen, Lafora bodies, or any combination thereof in addition to the accumulation of glycogen. In an aspect, “accumulation” can refer to accumulation of glycogen, polyglucosan bodies, amylopectin-like glycogen, Lafora bodies, or any combination thereof.

In an aspect, SRT can be used to reduce activity and/or expression of GYS1 in view of the reduced activity and/or expression level of GBE or one or more other enzymes in the metabolic pathways of glycogen metabolism, synthesis, and glycolysis. In an aspect, SRT can comprise siRNA-based therapies, shRNA-based therapies, antisense therapies, gene-editing therapies, and therapies using one or more small molecules or peptide drugs. In an aspect, SRT can comprise administration of one or more small molecules that can traverse the blood-brain barrier in quantities that are therapeutic for a subject having neuropathic glycogen storage disease. In an aspect, SRT can comprise administration of one or more small molecules that do not traverse the blood-brain barrier in quantities but are nonetheless therapeutic for a subject having neuropathic glycogen storage disease. In an aspect, a disclosed small molecule that inhibits glycogen synthase (GYS1) in SRT can be orally delivered.

As used herein, “GYS1” refers to glycogen synthase (muscle), which is an enzyme that transfers the glycosyl residue from UDP-Glc to the non-reducing end of alpha-1,4-glucan while “GYS2” refers to glycogen synthase (liver), which is an enzyme that transfers the glycosyl residue from UDP-Glc to the non-reducing end of alpha-1,4-glucan.

In an aspect, the level of glycogen synthase (GYS1) in a subject or in a tissue and/or organ in a subject can be restored to normal or near normal. In an aspect, the level of GBE in a subject or in a tissue and/or organ in a subject can be restored to normal or near normal. In an aspect, the ratio of GYS1 and GBE in a subject or in a tissue and/or organ in a subject can be restored to normal or near normal.

As used herein, “CRISPR or clustered regularly interspaced short palindromic repeat” is an ideal tool for correction of genetic abnormalities as the system can be designed to target genomic DNA directly. A CRISPR system involves two main components: a Cas9 enzyme and a guide (gRNA). The gRNA contains a targeting sequence for DNA binding and a scaffold sequence for Cas9 binding. Cas9 nuclease is often used to “knockout” target genes hence it can be applied for deletion or suppression of oncogenes that are essential for cancer initiation or progression. Similar to ASOs and siRNAs, CRISPR offers a great flexibility in targeting any gene of interest hence, potential CRISPR based therapies can be designed based on the genetic mutation in individual patients. An advantage of CRISPR is its ability to completely ablate the expression of disease genes which can only be suppressed partially by RNA interference methods with ASOs or siRNAs. Furthermore, multiple gRNAs can be employed to suppress or activate multiple genes simultaneously, hence increasing the treatment efficacy and reducing resistance potentially caused by new mutations in the target genes.

As used herein, “CRISPR-based endonucleases” include RNA-guided endonucleases that comprise at least one nuclease domain and at least one domain that interacts with a guide RNA. As known to the art, a guide RNA directs the CRISPR-based endonucleases to a targeted site in a nucleic acid at which site the CRISPR-based endonucleases cleaves at least one strand of the targeted nucleic acid sequence. As the guide RNA provides the specificity for the targeted cleavage, the CRISPR-based endonuclease is universal and can be used with different guide RNAs to cleave different target nucleic acid sequences. CRISPR-based endonucleases are RNA-guided endonucleases derived from CRISPR/Cas systems. Bacteria and archaea have evolved an RNA-based adaptive immune system that uses CRISPR (clustered regularly interspersed short palindromic repeat) and Cas (CRISPR-associated) proteins to detect and destroy invading viruses or plasmids. CRISPR/Cas endonucleases can be programmed to introduce targeted site-specific double-strand breaks by providing target-specific synthetic guide RNAs (Jinek M, et al. (2012) Science. 337:816-821).

In an aspect, a disclosed CRISPR-based endonuclease can be derived from a CRISPR/Cas type I, type II, or type III system. Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966.

In an aspect, a disclosed CRISPR-based endonuclease can be derived from a type II CRISPR/Cas system. For example, in an aspect, a CRISPR-based endonuclease can be derived from a Cas9 protein. The Cas9 protein can be from Streptococcus pyogenes, Streptococcus thermophilus, Streptococcus sp, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina. In an aspect, the CRISPR-based nuclease can be derived from a Cas9 protein from Streptococcus pyogenes (SEQ ID NO:11).

In general, CRISPR/Cas proteins can comprise at least one RNA recognition and/or RNA binding domain. RNA recognition and/or RNA binding domains can interact with the guide RNA such that the CRISPR/Cas protein is directed to a specific genomic or genomic sequence. CRISPR/Cas proteins can also comprise nuclease domains (i.e., DNase or RNase domains), DNA binding domains, helicase domains, protein-protein interaction domains, dimerization domains, as well as other domains.

The CRISPR-based endonuclease can be a wild type CRISPR/Cas protein (such as for example, SEQ ID NO:09 and SEQ ID NO:10), a modified CRISPR/Cas protein, or a fragment of a wild type or modified CRISPR/Cas protein. The CRISPR/Cas protein can be modified to increase nucleic acid binding affinity and/or specificity, alter an enzymatic activity, and/or change another property of the protein. For example, in an aspect, nuclease (i.e., DNase, RNase) domains of the CRISPR/Cas protein can be modified, deleted, or inactivated. A CRISPR/Cas protein can be truncated to remove domains that are not essential for the function of the protein. A CRISPR/Cas protein also can be truncated or modified to optimize the activity of the protein or an effector domain fused with a CRISPR/Cas protein.

In an aspect, a disclosed CRISPR-based endonuclease can be derived from a wild type Cas9 protein or fragment thereof. In an aspect, a disclosed CRISPR-based endonuclease can be derived from a modified Cas9 protein. For example, the amino acid sequence of a disclosed Cas9 protein can be modified to alter one or more properties (e.g., nuclease activity, affinity, stability, etc.) of the protein. Alternatively, domains of the Cas9 protein not involved in RNA-guided cleavage can be eliminated from the protein such that the modified Cas9 protein is smaller than the wild type Cas9 protein.

As used herein, “immune tolerance,” “immunological tolerance,” and “immunotolerance” refers to a state of unresponsiveness or blunted response of the immune system to substances (e.g., a disclosed isolated nucleic acid molecule, a disclosed vector, a disclosed transgene product, a disclosed pharmaceutical formulation, a disclosed therapeutic agent, etc.) that have the capacity to elicit an immune response in a subject. Immune tolerance is induced by prior exposure to a specific antigen. Immune tolerance can be determined in a subject by measuring antibodies against a particular antigen or by liver-restricted transgene expression with an AAV vector. Low or absent antibody titers over time is an indicator of immune tolerance. For example, in some embodiments, immune tolerance can be established by having IgG antibody titers of less than or equal to about 12,000, 11,500, 11,000, 10,500, 10,000, 9,500, 9,000, 8,500, 8,000, 7,500, 7,000, 6,500, or 6,000 within following gene therapy (such as the administration of the transgene encoding, for example, a glycogen branching enzyme, a salivary alpha-amylase 1A precursor, or a pancreatic alpha-amylase, or a truncated variant thereof).

As used herein, “immune-modulating” refers to the ability of a disclosed isolated nucleic acid molecules, a disclosed vector, a disclosed pharmaceutical formulation, or a disclosed agent to alter (modulate) one or more aspects of the immune system. The immune system functions to protect the organism from infection and from foreign antigens by cellular and humoral mechanisms involving lymphocytes, macrophages, and other antigen-presenting cells that regulate each other by means of multiple cell-cell interactions and by elaborating soluble factors, including lymphokines and antibodies, that have autocrine, paracrine, and endocrine effects on immune cells.

As known to the art, antibodies (Abs) can mitigate AAV infection through multiple mechanisms by binding to AAV capsids and blocking critical steps in transduction such as cell surface attachment and uptake, endosomal escape, productive trafficking to the nucleus, or uncoating as well as promoting AAV opsonization by phagocytic cells, thereby mediating their rapid clearance from the circulation. For example, in humans, serological studies reveal a high prevalence of NAbs in the worldwide population, with about 67% of people having antibodies against AAV1, 72% against AAV2, and approximately 40% against AAV serotypes 5 through 9. Vector immunogenicity represents a major challenge in re-administration of AAV vectors.

As used herein, “immune modulator” refers to an agent that is capable of adjusting a given immune response to a desired level (e.g. as in immunopotentiation, immunosuppression, or induction of immunologic tolerance). Examples of immune modulators include but are not limited to, a disclosed immune modulator can comprise aspirin, azathioprine, belimumab, betamethasone dipropionate, betamethasone valerate, bortezomib, bredinin, cyazathioprine, cyclophosphamide, cyclosporine, deoxyspergualin, didemnin B, fluocinolone acetonide, folinic acid, ibuprofen, IL6 inhibitors (such as sarilumab) indomethacin, inebilizumab, intravenous gamma globulin (IVIG), methotrexate, methylprednisolone, mycophenolate mofetil, naproxen, prednisolone, prednisone, prednisolone indomethacin, rapamycin, rituximab, sirolimus, sulindac, synthetic vaccine particles containing rapamycin (SVP-Rapamycin or ImmTOR), thalidomide, tocilizumab, tolmetin, triamcinolone acetonide, anti-CD3 antibodies, anti-CD4 antibodies, anti-CD19 antibodies, anti-CD20 antibodies, anti-CD22 antibodies, anti-CD40 antibodies, anti-FcRN antibodies, anti-IL6 antibodies, anti-IGF1R antibodies, an IL2 mutein, a BTK inhibitor, or a combination thereof. In an aspect, a disclosed immune modulator can comprise one or more Treg (regulatory T cells) infusions (e.g., antigen specific Treg cells to AAV). In an aspect, a disclosed immune modulator can be bortezomib or SVP-Rapamycin. In an aspect, an immune modulator can be administered by any suitable route of administration including, but not limited to, in utero, intra-CSF, intrathecally, intravenously, subcutaneously, transdermally, intradermally, intramuscularly, orally, transcutaneously, intraperitoneally (IP), or intravaginally. In an aspect, a disclosed immune modulator can be administered using a combination of routes.

As used herein, the term “immunotolerant” refers to unresponsiveness to an antigen (e.g., a vector, a therapeutic protein derived from a human, a non-human animal, a plant, or a microorganism, such as, for example, a microbial GBE. An immunotolerant promoter can reduce, ameliorate, or prevent transgene-induced immune responses that can be associated with gene therapy. Assays known in the art to measure immune responses, such as immunohistochemical detection of cytotoxic T cell responses, can be used to determine whether one or more promoters can confer immunotolerant properties.

As used herein, the term “package insert” is used to refer to instructions customarily included in commercial packages of therapeutic products, that contain information about the indications, usage, dosage, administration, contraindications and/or warnings concerning the use of such therapeutic products.

As used herein, the term “in combination” in the context of the administration of other therapies (e.g., other agents) includes the use of more than one therapy (e.g., drug therapy). Administration “in combination with” one or more further therapeutic agents includes simultaneous (e.g., concurrent) and consecutive administration in any order. The use of the term “in combination” does not restrict the order in which therapies are administered to a subject. By way of non-limiting example, a first therapy (e.g., a disclosed isolated nucleic acid molecule, a disclosed vector, a disclosed pharmaceutical formulation, or a combination thereof) may be administered before (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy (e.g., agent) to a subject having or diagnosed with a GSD (such as, for example, GSD IV, Lafora disease (including those diseases caused by mutations in the EPM2A gene (glucan phosphatase, laforin) or the NHLRC1 gene (NHL repeat containing E3 ubiquitin protein ligase 1 or EPM2B)), polyglucosan body myopathy-1, polyglucosan body myopathy-2, APBD, or any disease or pathology caused by a mutation in a GYG1 gene, a RBCK1 gene, or a PRKAG2 gene). Disclosed are the components to be used to prepare the disclosed isolated nucleic acid molecules, disclosed vectors, or disclosed pharmaceutical formulations as well as the disclosed isolated nucleic acid molecules, disclosed vectors, or disclosed pharmaceutical formulations used within the methods disclosed herein.

These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

### B. Compositions for Treating and/or Preventing GSD IV and/or APBD Disease Progression

### 1. Nucleic Acid Molecules

Disclosed herein is an isolated nucleic acid molecule comprising a nucleic acid sequence encoding a polypeptide for preventing glycogen accumulation and/or degrading accumulated glycogen, wherein the nucleic acid sequence is CpG-depleted and codon-optimized for expression in a human or a mammalian cell.

Disclosed herein is an isolated nucleic acid molecule comprising the sequence set forth in SEQ ID NO:03 or SEQ ID NO:04 and the sequence set forth in SEQ ID NO:05 or SEQ ID NO: 06. Disclosed herein is an isolated nucleic acid molecule comprising the sequence set forth in SEQ ID NO:03 and the sequence set forth in SEQ ID NO:05. Disclosed herein is an isolated nucleic acid molecule comprising the sequence set forth in SEQ ID NO:03 and the sequence set forth in SEQ ID NO:06. Disclosed herein is an isolated nucleic acid molecule comprising the sequence set forth in SEQ ID NO:04 and the sequence set forth in SEQ ID NO:05. Disclosed herein is an isolated nucleic acid molecule comprising the sequence set forth in SEQ ID NO:04 and the sequence set forth in SEQ ID NO:06. Disclosed herein is an isolated nucleic acid molecule comprising a sequence having at least 30%, at least 40%, at least 50%, at least 60%, at least 70%, at least 80%, or at least 90% identity to the sequence set forth in SEQ ID NO:03 or SEQ ID NO:04 and a sequence having at least 30%, at least 40%, at least 50%, at least 60%, at least 70%, at least 80%, or at least 90% identity to the sequence set forth in SEQ ID NO:05 or SEQ ID NO:06.

In an aspect, a mammalian cell can be a cell from any non-human species, such as, for example, a cell from a gorilla, a chimpanzee, a Rhesus monkey, a dog, a cow, a mouse, and a rat.

In an aspect, the glycogen can be amylopectin-like glycogen. In an aspect, the glycogen can be Lafora bodies, polyglucosan bodies, or any form of accumulated glycogen.

In an aspect, a disclosed encoded polypeptide can be a human glycogen branching enzyme. In an aspect, the original (non-CpG-depleted) polynucleotide open reading frame (ORF) sequence of human glycogen branching enzyme can comprise the sequence set forth in SEQ ID NO:2. In an aspect, the original (non-CpG-depleted) polynucleotide open reading frame (ORF) sequence of human glycogen branching enzyme can comprise a sequence having at least 50-69%, at least 70-89%, or at least 90-99% identity to the sequence set forth in SEQ ID NO:02.

In an aspect, a disclosed encoded polypeptide can comprise the sequence set forth in SEQ ID NO:01. In an aspect, a disclosed encoded polypeptide can comprise a sequence having at least 30%, at least 40%, at least 50%, at least 60%, at least 70%, at least 80%, or at least 90% identity to the sequence set forth in SEQ ID NO:01. In an aspect, a disclosed encoded polypeptide can degrade insoluble amylopectin-like glycogen, Lafora bodies, polyglucosan bodies, or any form of accumulated glycogen. In an aspect, a disclosed encoded polypeptide can be a human salivary or pancreatic amylase. In an aspect, a disclosed encoded polypeptide can be derived from plant, bacteria, or another microorganism. In an aspect, a disclosed encoded polypeptide can be derived from any non-human species, such as, for example, gorilla, chimpanzee, Rhesus monkey, dog, cow, mouse, rat, chicken, zebrafish, fruit fly, mosquito, C. elegans, and frog.

In an aspect, a disclosed nucleic acid sequence can comprise the sequence set forth in SEQ ID NO:03 or SEQ ID NO:04. In an aspect, a disclosed nucleic acid sequence can comprise a sequence having at least 50-69%, at least 70-89%, or at least 90-99% identity to the sequence set forth in SEQ ID NO:03. In an aspect, a disclosed nucleic acid sequence can comprise a sequence having at least 50-69%, at least 70-89%, or at least 90-99% identity to the sequence set forth in SEQ ID NO:04. In an aspect, a disclosed nucleic acid sequence can comprise a sequence having at least 80% identity to the sequence set forth in SEQ ID NO:03 or SEQ ID NO:04. In an aspect, a disclosed nucleic acid sequence can comprise a coding sequence that is less than about 4.5 kilobases.

a. Nucleotide Sequences

In an aspect, an disclosed original polynucleotide open reading frame (ORF) sequence of human glycogen branching enzyme can have the sequence set forth in NCBI Reference Sequence No. NM_000158.4.

In an aspect, a disclosed original polynucleotide open reading frame (ORF) sequence of human glycogen branching enzyme can comprise the following sequence:

(SEQ ID NO: 02)
ATGGCGGCTCCGATGACTCCCGCGGCTCGGCCCGAGGACTACGAG
GCGGCGCTCAATGCCGCCCTGGCTGACGTGCCCGAACTGGCCAGA
CTCCTGGAGATCGACCCGTACTTGAAGCCCTACGCCGTGGACTTC
CAGCGCAGGTATAAGCAGTTTAGCCAAATTTTGAAGAACATTGGA
GAAAATGAAGGTGGTATTGATAAGTTTTCCAGAGGCTATGAATCA
TTTGGCGTCCACAGATGTGCTGATGGTGGTTTATACTGCAAAGAA
TGGGCCCCGGGAGCAGAAGGAGTTTTTCTTACTGGAGATTTTAAT
GGTTGGAATCCATTTTCGTACCCATACAAAAAACTGGATTATGGA
AAATGGGAGCTGTATATCCCACCAAAGCAGAATAAATCTGTACTC
GTGCCTCATGGATCCAAATTAAAGGTAGTTATTACTAGTAAAAGC
GGAGAGATCTTGTATCGTATTTCACCGTGGGCAAAGTATGTGGTT
CGTGAAGGTGATAATGTGAATTATGATTGGATACACTGGGATCCA
GAACACTCATATGAGTTTAAGCATTCCAGACCAAAGAAGCCACGG
AGTCTAAGAATTTATGAATCTCATGTGGGAATTTCTTCCCATGAA
GGAAAAGTAGCTTCTTATAAACATTTTACATGCAATGTACTACCA
AGAATCAAAGGCCTTGGATACAACTGCATTCAGTTGATGGCAATC
ATGGAGCATGCTTACTATGCCAGCTTTGGTTACCAAATCACAAGC
TTCTTTGCAGCTTCCAGCCGTTATGGAACACCTGAAGAGCTACAA
GAACTGGTAGACACAGCTCATTCCATGGGTATCATAGTCCTCTTA
GATGTGGTACACAGCCATGCTTCAAAAAATTCAGCAGATGGATTG
AATATGTTTGATGGGACAGATTCCTGTTATTTTCATTCTGGACCT
AGAGGGACTCATGATCTTTGGGATAGCAGATTGTTTGCCTACTCC
AGCTGGGAAATTTTAAGATTCCTTCTGTCAAACATAAGATGGTGG
TTGGAAGAATATCGCTTTGATGGATTTCGTTTTGATGGTGTTACG
TCCATGCTTTATCATCACCATGGAGTGGGTCAAGGTTTCTCAGGT
GATTACAGTGAATATTTCGGACTACAAGTAGATGAAGATGCCTTG
ACTTACCTCATGTTGGCAAATCATTTGGTTCACACGCTGTGTCCC
GATTCTATAACAATAGCTGAGGATGTATCAGGAATGCCAGCTCTG
TGCTCTCCAATTTCCCAGGGAGGGGGTGGTTTTGACTATCGACTA
GCCATGGCAATTCCAGATAAGTGGATTCAGCTACTTAAAGAGTTT
AAAGATGAAGACTGGAACATGGGCGATATAGTATACACGCTCACA
AACAGGCGCTACCTTGAAAAGTGCATTGCTTATGCAGAGAGCCAT
GATCAGGCATTGGTTGGGGATAAGTCGCTGGCATTTTGGTTGATG
GATGCCGAAATGTATACAAACATGAGTGTCCTGACTCCTTTTACT
CCAGTTATTGATCGTGGAATACAGCTTCATAAAATGATTCGACTC
ATTACGCATGGGCTTGGTGGAGAAGGCTATCTCAATTTCATGGGT
AATGAATTTGGGCATCCTGAATGGTTAGACTTCCCAAGAAAAGGA
AATAATGAGAGTTACCATTATGCCAGGCGGCAGTTTCATTTAACT
GACGACGACCTTCTTCGCTACAAGTTCCTAAATAATTTTGACAGG
GATATGAATAGATTGGAAGAAAGATATGGTTGGCTTGCAGCTCCA
CAGGCCTACGTGAGTGAAAAACATGAAGGCAATAAGATCATTGCT
TTTGAAAGAGCAGGTCTTCTTTTCATTTTCAACTTCCATCCAAGC
AAGAGCTACACTGACTACCGAGTTGGAACAGCATTGCCAGGGAAA
TTCAAAATTGTGCTAGATTCAGATGCAGCGGAATATGGAGGGCAT
CAGAGACTGGACCACAGCACTGACTTTTTTTCTGAGGCTTTTGAA
CATAATGGGCGTCCCTATTCTCTTTTGGTGTACATTCCAAGCAGA
GTGGCCCTCATCCTTCAGAATGTGGATCTGCCGAATTGA.

In an aspect, a disclosed CpG-free polynucleotide ORF sequence #1 for expressing human glycogen branching enzyme can comprise the following sequence or a fragment thereof:

(SEQ ID NO: 03)
ATGGCTGCACCAATGACTCCAGCAGCCAGACCAGAAGACTATGAG
GCAGCTCTCAATGCTGCACTGGCTGATGTCCCAGAGCTGGCCAGA
CTCCTGGAAATTGATCCCTACCTGAAGCCTTATGCAGTGGACTTT
CAGAGAAGGTATAAGCAGTTCTCCCAAATTCTGAAAAATATTGGA
GAGAATGAAGGAGGCATTGATAAGTTCAGCAGAGGGTATGAATCC
TTTGGAGTGCATAGGTGTGCTGATGGAGGCCTGTATTGCAAGGAA
TGGGCCCCAGGAGCAGAGGGAGTGTTTCTGACAGGGGACTTTAAT
GGGTGGAATCCTTTCTCTTATCCTTATAAGAAGCTGGATTATGGC
AAATGGGAACTGTATATTCCACCCAAACAGAACAAATCTGTGCTG
GTCCCACATGGCAGTAAGCTGAAAGTGGTCATTACTAGTAAGAGT
GGAGAAATCCTGTATAGAATAAGTCCTTGGGCAAAGTATGTGGTT
AGAGAGGGAGACAATGTTAACTATGATTGGATCCACTGGGACCCT
GAGCATAGCTATGAATTTAAACACAGCAGGCCTAAGAAACCTAGA
AGCCTGAGGATTTATGAGTCCCATGTGGGAATAAGCTCCCATGAG
GGAAAGGTTGCATCCTATAAACATTTTACATGCAATGTGCTGCCA
AGGATCAAAGGACTGGGCTACAACTGCATCCAACTGATGGCTATC
ATGGAACATGCTTACTATGCATCCTTTGGATACCAGATTACAAGC
TTCTTTGCAGCCTCTAGCAGATATGGCACACCTGAAGAGCTGCAG
GAACTGGTGGATACAGCCCATTCCATGGGTATCATTGTTCTCCTG
GATGTTGTCCATTCTCATGCCAGCAAAAACTCTGCTGATGGGCTG
AATATGTTTGATGGAACAGATTCTTGCTATTTTCACTCTGGACCA
AGAGGAACCCATGACCTCTGGGATTCCAGACTGTTTGCTTACTCC
TCCTGGGAAATCCTGAGGTTTCTCCTGAGCAACATTAGATGGTGG
CTGGAAGAGTACAGATTTGATGGCTTCAGATTTGATGGAGTGACC
TCCATGCTGTATCACCACCATGGGGTGGGACAAGGATTCTCTGGA
GACTACTCTGAATACTTTGGACTGCAAGTGGATGAGGATGCTCTG
ACATATCTGATGCTGGCAAATCACCTGGTCCACACACTGTGTCCT
GATTCTATCACTATTGCTGAAGATGTGTCAGGAATGCCTGCACTG
TGTAGCCCTATCAGCCAGGGTGGAGGAGGGTTTGATTATAGACTG
GCTATGGCTATTCCTGACAAGTGGATTCAGCTGCTGAAGGAGTTT
AAAGATGAGGACTGGAACATGGGAGATATTGTGTATACACTCACC
AATAGAAGGTATCTGGAGAAATGTATTGCATATGCTGAGAGCCAT
GACCAGGCACTGGTGGGAGATAAGTCACTGGCATTTTGGCTGATG
GATGCTGAGATGTACACTAATATGAGTGTTCTCACACCTTTCACA
CCAGTGATTGACAGAGGAATCCAGCTCCACAAGATGATTAGATTG
ATTACACATGGACTGGGAGGTGAGGGGTATCTGAACTTTATGGGA
AATGAATTTGGACACCCAGAGTGGCTGGACTTTCCCAGAAAGGGC
AACAATGAATCTTACCACTATGCTAGGAGACAGTTTCATCTCACA
GATGATGACCTCCTCAGGTACAAATTTTTGAACAACTTTGACAGA
GACATGAACAGACTGGAAGAGAGGTATGGCTGGCTGGCAGCTCCA
CAGGCTTATGTTTCTGAAAAACATGAAGGAAATAAAATCATTGCA
TTTGAAAGAGCAGGACTCCTGTTCATCTTCAACTTTCATCCTAGC
AAAAGCTATACAGATTATAGGGTGGGAACAGCACTCCCTGGGAAG
TTCAAGATTGTGCTGGACTCTGATGCTGCAGAGTATGGAGGACAC
CAGAGGCTGGACCACAGCACAGACTTCTTTTCTGAAGCCTTTGAA
CATAATGGCAGACCCTACTCCCTCCTGGTGTACATCCCTTCCAGG
GTGGCACTCATTCTGCAAAATGTGGATCTGCCAAACTGA.

In an aspect, a disclosed CpG-free polynucleotide ORF sequence #2 for expressing human glycogen branching enzyme can comprise the following sequence or a fragment thereof:

(SEQ ID NO: 04)
ATGGCTGCTCCCATGACTCCTGCTGCTAGACCTGAGGACTATGAG
GCTGCCCTCAATGCTGCCCTGGCTGATGTGCCTGAACTGGCCAGA
CTCCTGGAGATTGACCCCTACTTGAAGCCCTATGCTGTGGACTTC
CAGAGAAGGTATAAGCAGTTTAGCCAAATTTTGAAGAACATTGGA
GAAAATGAAGGTGGTATTGATAAGTTTTCCAGAGGCTATGAATCA
TTTGGAGTCCACAGATGTGCTGATGGTGGTTTATACTGCAAAGAA
TGGGCCCCTGGAGCAGAAGGAGTTTTTCTTACTGGAGATTTTAAT
GGTTGGAATCCATTTAGCTACCCATACAAAAAACTGGATTATGGA
AAATGGGAGCTGTATATCCCACCAAAGCAGAATAAATCTGTACTG
GTGCCTCATGGATCCAAATTAAAGGTAGTTATTACTAGTAAATCT
GGAGAGATCTTGTATAGAATTTCACCCTGGGCAAAGTATGTGGTT
AGAGAAGGTGATAATGTGAATTATGATTGGATACACTGGGATCCA
GAACACTCATATGAGTTTAAGCATTCCAGACCAAAGAAGCCAAGA
AGTCTAAGAATTTATGAATCTCATGTGGGAATTTCTTCCCATGAA
GGAAAAGTAGCTTCTTATAAACATTTTACATGCAATGTACTACCA
AGAATCAAAGGCCTTGGATACAACTGCATTCAGTTGATGGCAATC
ATGGAGCATGCTTACTATGCCAGCTTTGGTTACCAAATCACAAGC
TTCTTTGCAGCTTCCAGCAGATATGGAACACCTGAAGAGCTACAA
GAACTGGTAGACACAGCTCATTCCATGGGTATCATAGTCCTCTTA
GATGTGGTACACAGCCATGCTTCAAAAAATTCAGCAGATGGATTG
AATATGTTTGATGGGACAGATTCCTGTTATTTTCATTCTGGACCT
AGAGGGACTCATGATCTTTGGGATAGCAGATTGTTTGCCTACTCC
AGCTGGGAAGTTTTAAGATTCCTTCTGTCAAACATAAGATGGTGG
TTGGAAGAATATAGATTTGATGGATTTAGATTTGATGGTGTTACC
TCCATGCTTTATCATCACCATGGAGTGGGTCAAGGTTTCTCAGGT
GATTACAGTGAATATTTTGGACTACAAGTAGATGAAGATGCCTTG
ACTTACCTCATGTTGGCAAATCATTTGGTTCACACCCTGTGTCCT
GATTCTATAACAATAGCTGAGGATGTATCAGGAATGCCAGCTCTG
TGCTCTCCAATTTCCCAGGGAGGGGGTGGTTTTGACTATAGACTA
GCCATGGCAATTCCAGATAAGTGGATTCAGCTACTTAAAGAGTTT
AAAGATGAAGACTGGAACATGGGAGATATAGTATACACCCTCACA
AACAGGAGATACCTTGAAAAGTGCATTGCTTATGCAGAGAGCCAT
GATCAGGCATTGGTTGGGGATAAGAGCCTGGCATTTTGGTTGATG
GATGCTGAAATGTATACAAACATGAGTGTCCTGACTCCTTTTACT
CCAGTTATTGATAGAGGAATACAGCTTCATAAAATGATTAGACTC
ATTACCCATGGGCTTGGTGGAGAAGGCTATCTCAATTTCATGGGT
AATGAATTTGGGCATCCTGAATGGTTAGACTTCCCAAGAAAAGGA
AATAATGAGAGTTACCATTATGCCAGGAGACAGTTTCATTTAACT
GATGATGACCTTCTTAGATACAAGTTCCTAAATAATTTTGACAGG
GATATGAATAGATTGGAAGAAAGATATGGTTGGCTTGCAGCTCCA
CAGGCCTATGTGAGTGAAAAACATGAAGGCAATAAGATCATTGCT
TTTGAAAGAGCAGGTCTTCTTTTCATTTTCAACTTCCATCCAAGC
AAGAGCTACACTGACTACAGAGTTGGAACAGCATTGCCAGGGAAA
TTCAAAATTGTGCTAGATTCAGATGCAGCTGAATATGGAGGGCAT
CAGAGACTGGACCACAGCACTGACTTTTTTTCTGAGGCTTTTGAA
CATAATGGGAGACCCTATTCTCTTTTGGTGTACATTCCAAGCAGA
GTGGCCCTCATCCTTCAGAATGTGGATCTGCCCAATTGA.

In an aspect, a disclosed LSP promoter can have the following sequence or a fragment thereof:

(SEQ ID NO: 34)
GAGTTAATTTTTAAAAAGCAGTCAAAAGTCCAAGTGGCCCTTGCG
AGCATTTACTCTCTCTGTTTGCTCTGGTTAATAATCTCAGGAGCA
CAAAATTCCTTACTAGTCCTAGAAGTTAATTTTTAAAAAGCAGTC
AAAAGTCCAAGTCCAAGTGGCCCTTGCGAGCATTTACTCTCTCTG
TTTGCTCTGGTTAATAATCTCAGGAGCACAAACATTCCTTACTAG
TTCTAGAGCGGCCGCCAGTGTGCTGGAATTCGGCTTTTTTAGGGC
TGGAAGCTACCTTTGACATCATTTCCTCTGCGAATGCATGTATAA
TTTCTACAGAACCTATTAGAAAGGATCACCCAGCCTCTGCTTTTG
TACAACTTTCCCTTAAAAAATGCCAATTCCACTGCTGTTTGGCCC
AATAGTGAGAACTTTTTCCTGCTGCCTCTTGGTGCTTTTGCCTAT
GGCCCCTATTCTGCCTGCTGAAGACACTCTTGCCAGCATGGACTT
AAACCCCTCCAGCTCTGACAATCCTCTTTCTCTTTTGTTTTACAT
GAAGGGTCTGGCAGCCAAAGCAATCACTCAAAGGTTCAAACCTTA
TCATTTTTTGCTTTGTTCCTCTTGGCCTTGGTTTTGTACATCAGC
TTTGAAAATACCATCCCAGGGTTAATGCTGGGGTTAATTTATAAC
TAAGAGTGCTCTAGTTTTGCAATACAGGACATGCTATAAAAATGG
AAAGATGTTGCTTTCTGAGAGATCAGCTTACATGT.

In an aspect, a disclosed LSP-CB dual promoter can comprise the following sequence.

(SEQ ID NO: 05)
GAGTTAATTTTTAAAAAGCAGTCAAAAGTCCAAGTGGCCCTTGCG
AGCATTTACTCTCTCTGTTTGCTCTGGTTAATAATCTCAGGAGCA
CAAAATTCCTTACTAGTCCTAGAAGTTAATTTTTAAAAAGCAGTC
AAAAGTCCAAGTCCAAGTGGCCCTTGCGAGCATTTACTCTCTCTG
TTTGCTCTGGTTAATAATCTCAGGAGCACAAACATTCCTTACTAG
TTCTAGAGCGGCCGCCAGTGTGCTGGAATTCGGCTTTTTTAGGGC
TGGAAGCTACCTTTGACATCATCTCCTCTGCGAATGCATGTATAA
TTTCTACAGAACCTATTAGAAAGGATCACCCAGCCTCTGCTTTTG
TACAACTTTCCCTTAAAAAACTGCCAATCCCACTGCTGTTTGGCC
CAATAGTGAGAACTTTTTCTGCTGCCTCTTGGTGCTTTTGCCTAT
GGCCCCTATTCTGCTGCTGAAGACACTCTTGCCAGCATGGACTTA
AACCCCTCCAGCTCTGACAATCCTCTTTCTCTTTTGTTTTACATG
AAGGGTCTGGCAGCCAAAGCAATCACTCAAAGTTCAAACCTTATC
ATTTTTTGCTTTGTTCCTCTTGGCCTTGGTTTTGTACATCAGCTT
TGAAAATACCATCCCAGGGTTAATGCTGGGGTTAATTTATAACTG
AGAGTGCTCTAGTTTTGCAATACAGGACATGCTATAAAAATGGCT
TAAGGTTCCGCGTTACATAACTTACGGTAAATGGCCCGCCTGGCT
GACCGCCCAACGACCCCCGCCCATTGACGTCAATAATGACGTATG
TTCCCATAGTAACGCCAATAGGGACTTTCCATTGACGTCAATGGG
TGGAGTATTTACGGTAAACTGCCCACTTGGCAGTACATCAAGTGT
ATCATATGCCAAGTACGCCCCCTATTGACGTCAATGACGGTAAAT
GGCCCGCCTGGCATTATGCCCAGTACATGACCTTATGGGACTTTC
CTACTTGGCAGTACATCTACGTATTAGTCATCGCTATTACCATGC
ATGGTCGAGGTGAGCCCCACGTTCTGCTTCACTCTCCCCATCTCC
CCCCCCTCCCCACCCCCAATTTTGTATTTATTTATTTTTTAATTA
TTTTGTGCAGCGAGGGGCGGGGCGGGGCGAGGCGGAGAGGTGCGG
CGGCAGCCAATCAGAGCGGCGCGCTCCGAAAGTTTCCTTTTATGG
CGAGGCGGCGGCGGCGGCGGCCCTATAAAAAGCGAAGCGCGCGGC
GGGCG.

In an aspect, a disclosed LSP-mCMV/hEF1α^{CpG-free} dual promoter can comprise the following sequence or a fragment thereof:

(SEQ ID NO: 06)
GAGTTAATTTTTAAAAAGCAGTCAAAAGTCCAAGTGGCCCTTGCG
AGCATTTACTCTCTCTGTTTGCTCTGGTTAATAATCTCAGGAGCA
CAAAATTCCTTACTAGTCCTAGAAGTTAATTTTTAAAAAGCAGTC
AAAAGTCCAAGTCCAAGTGGCCCTTGCGAGCATTTACTCTCTCTG
TTTGCTCTGGTTAATAATCTCAGGAGCACAAACATTCCTTACTAG
TTCTAGAGCGGCCGCCAGTGTGCTGGAATTCGGCTTTTTTAGGGC
TGGAAGCTACCTTTGACATCATCTCCTCTGCGAATGCATGTATAA
TTTCTACAGAACCTATTAGAAAGGATCACCCAGCCTCTGCTTTTG
TACAACTTTCCCTTAAAAAACTGCCAATCCCACTGCTGTTTGGCC
CAATAGTGAGAACTTTTTTCTGCTGCCTCTTGGTGCTTTTGCCTA
TGGCCCCTATTCTGCTGCTGAAGACACTCTTGCCAGCATGGACTT
AAACCCCTCCAGCTCTGACAATCCTCTTTCTCTTTTGTTTTACAT
GAAGGGTCTGGCAGCCAAAGCAATCACTCAAAGTTCAAACCTTAT
CATTTTTTGCTTTGTTCCTCTTGGCCTTGGTTTTGTACATCAGCT
TTGAAAATACCATCCCAGGGTTAATGCTGGGGTTAATTTATAACT
GAGAGTGCTCTAGTTTTGCAATACAGGACATGCTATAAAAATGGC
TTAAGGAGTCAATGGGAAAAACCCATTGGAGCCAAGTACACTGAC
TCAATAGGGACTTTCCATTGGGTTTTGCCCAGTACATAAGGTCAA
TAGGGGGTGAGTCAACAGGAAAGTCCCATTGGAGCCAAGTACATT
GAGTCAATAGGGACTTTCCAATGGGTTTTGCCCAGTACATAAGGT
CAATGGGAGGTAAGCCAATGGGTTTTTCCCATTACTGACATGTAT
ACTGAGTCATTAGGGACTTTCCAATGGGTTTTGCCCAGTACATAA
GGTCAATAGGGGTGAATCAACAGGAAAGTCCCATTGGAGCCAAGT
ACACTGAGTCAATAGGGACTTTCCATTGGGTTTTGCCCAGTACAA
AAGGTCAATAGGGGGTGAGTCAATGGGTTTTTCCCATTATTGGCA
CATACATAAGGTCAATAGGGGTGACTAGTGGAGAAGAGCATGCTT
GAGGGCTGAGTGCCCCTCAGTGGGCAGAGAGCACATGGCCCACAG
TCCCTGAGAAGTTGGGGGGAGGGGTGGGCAATTGAACTGGTGCCT
AGAGAAGGTGGGGCTTGGGTAAACTGGGAAAGTGATGTGGTGTAC
TGGCTCCACCTTTTTCCCCAGGGTGGGGGAGAACCATATATAAGT
GCAGTAGTCTCTGTGAACATTC.

In an aspect, a disclosed nucleic acid sequence for Cas9 can comprise the sequence set forth in SEQ ID NO: 11 or a fragment thereof. In an aspect, a disclosed nucleic acid sequence for Cas9 can comprise a sequence having at least 80%, at least 85%, at least 90%, or at least 95% identity to the sequence set forth in SE ID NO: 11 or a fragment thereof.

(SEQ ID NO: 11)
ATGAAAAGGAATTATATCTTAGGATTAGATATCGGAATTACATCA
GTGGGTTATGGAATTATTGATTATGAAACTAGAGATGTCATAGAT
GCGGGCGTACGTTTATTTAAAGAGGCTAATGTTGAAAATAATGAA
GGACGACGATCAAAAAGAGGTGCCAGAAGGCTTAAGAGGCGTCGT
AGACATAGAATACAAAGAGTAAAGAAACTTTTATTTGATTACAAT
TTGTTGACAGATCATAGTGAGCTAAGTGGAATCAATCCTTACGAG
GCGCGCGTAAAGGGATTAAGTCAAAAATTAAGTGAAGAGGAATTT
TCTGCGGCATTGCTACATTTAGCAAAGCGTAGAGGTGTACATAAT
GTTAATGAAGTGGAAGAAGATACAGGTAATGAATTATCCACTAAA
GAACAAATTTCAAGAAATAGTAAAGCGTTAGAAGAGAAGTATGTT
GCAGAATTACAGTTGGAACGTTTGAAAAAAGACGGTGAAGTGAGA
GGTTCGATTAACCGTTTCAAAACATCTGACTATGTAAAAGAAGCA
AAGCAGTTATTAAAAGTACAAAAAGCATATCATCAACTTGATCAA
TCATTTATAGACACTTATATTGATTTATTGGAAACAAGAAGAACA
TATTATGAGGGACCAGGTGAAGGTAGCCCATTTGGATGGAAAGAT
ATTAAAGAATGGTATGAAATGTTAATGGGACATTGTACGTATTTC
CCAGAAGAATTACGTAGTGTGAAATATGCCTATAATGCTGATTTA
TATAATGCGCTGAATGATTTGAACAACTTGGTTATTACACGAGAT
GAGAATGAGAAGCTAGAGTATTATGAAAAATTCCAAATTATCGAG
AATGTCTTTAAACAAAAGAAAAAGCCGACGCTTAAACAAATTGCG
AAGGAAATCTTGGTGAATGAAGAAGACATCAAAGGCTATCGTGTC
ACAAGTACAGGTAAACCAGAATTTACAAACTTGAAAGTTTATCAC
GATATCAAAGATATTACAGCAAGAAAAGAAATTATCGAGAATGCA
GAGCTACTCGATCAAATAGCTAAAATATTAACTATTTACCAGTCA
TCAGAAGATATACAAGAAGAATTAACAAACCTAAATTCAGAATTG
ACACAAGAAGAGATTGAACAAATTTCAAACTTGAAAGGTTATACA
GGAACTCATAACCTTTCACTAAAGGCAATAAATTTAATATTAGAC
GAATTGTGGCATACGAACGATAATCAAATAGCTATTTTCAATCGT
TTGAAACTTGTACCTAAAAAGGTAGATTTAAGCCAACAAAAAGAA
ATTCCTACTACTTTAGTTGATGATTTTATACTGTCTCCAGTAGTG
AAACGTTCATTTATACAATCTATTAAAGTTATTAACGCTATTATT
AAAAAATACGGTTTGCCAAATGATATTATTATTGAACTTGCGAGA
GAAAAGAATTCTAAAGATGCACAAAAAATGATTAATGAAATGCAG
AAGAGAAATCGTCAAACGAATGAACGTATTGAGGAAATTATAAGA
ACGACAGGTAAAGAAAATGCTAAATATTTAATTGAAAAAATTAAG
CTGCACGATATGCAAGAAGGGAAATGTTTATACTCGTTAGAAGCA
ATCCCTCTAGAAGATTTACTTAATAATCCATTCAATTACGAAGTA
GACCATATCATTCCACGTTCTGTTTCTTTCGATAACTCTTTCAAT
AATAAAGTGTTGGTGAAACAAGAAGAAAATAGTAAAAAAGGTAAT
AGAACGCCATTTCAATATTTAAGTTCTTCAGATTCTAAAATAAGT
TATGAGACATTCAAAAAGCATATTTTAAATCTTGCTAAAGGCAAA
GGTAGAATCTCTAAGACGAAAAAAGAATATTTGTTAGAAGAACGA
GATATCAATCGCTTCAGTGTCCAAAAAGATTTTATTAACCGTAAC
TTAGTAGATACACGCTATGCGACAAGAGGGTTAATGAACTTATTA
AGATCTTATTTTAGAGTGAATAACTTAGATGTCAAAGTGAAATCG
ATTAATGGCGGATTCACAAGTTTCTTAAGAAGGAAATGGAAGTTC
AAAAAAGAAAGAAATAAGGGCTACAAACACCATGCTGAAGATGCA
CTGATTATTGCGAACGCTGATTTTATTTTCAAAGAATGGAAAAAA
CTAGATAAAGCTAAAAAAGTGATGGAAAATCAAATGTTTGAAGAA
AAGCAAGCTGAAAGTATGCCTGAAATTGAGACTGAGCAAGAGTAT
AAAGAAATTTTTATAACGCCTCATCAAATTAAACATATTAAGGAT
TTTAAAGATTATAAATATTCACATAGAGTTGATAAAAAGCCGAAT
AGAGAGTTAATAAATGATACATTATATTCTACGAGAAAAGATGAC
AAGGGTAATACATTAATCGTTAATAACTTAAATGGTTTATACGAT
AAAGATAATGATAAATTGAAAAAATTAATTAATAAATCACCTGAA
AAATTATTGATGTATCATCATGATCCACAAACATATCAAAAATTA
AAATTGATCATGGAACAATATGGCGATGAGAAAAATCCGCTTTAT
AAATATTATGAAGAAACAGGCAATTACTTAACAAAATATAGTAAA
AAAGATAACGGACCAGTCATCAAAAAAATTAAATATTATGGTAAC
AAGCTAAATGCGCATTTAGATATTACGGATGATTATCCAAATAGC
AGAAATAAAGTAGTAAAACTTTCATTAAAACCATATCGCTTTGAT
GTTTATTTAGATAATGGGGTATATAAATTTGTGACAGTTAAAAAT
TTAGATGTTATCAAAAAAGAAAACTACTATGAAGTTAATTCAAAG
TGTTATGAAGAAGCAAAAAAACTGAAGAAAATTAGTAATCAAGCA
GAATTTATCGCAAGTTTTTACAATAATGACTTGATTAAGATTAAC
GGAGAATTATATAGAGTCATAGGTGTAAATAATGATCTACTTAAC
AGAATTGAAGTAAATATGATAGACATCACATATAGAGAATATTTA
GAGAACATGAATGATAAAAGACCACCTAGAATAATTAAAACAATA
GCAAGCAAAACACAATCTATTAAAAAGTATTCTACAGATATTCTA
GGCAATCTTTATGAAGTGAAGAGTAAAAAGCATCCTCAAATCATA
AAGAAAGGATGA.

In an aspect, a disclosed nucleic acid sequence for GYS1 can comprise the sequence set forth in Accession No. NM_001161587.2, NM_002103.5, or a fragment thereof. In an aspect, a disclosed nucleic acid sequence for GYS1 can comprise a sequence having at least 30%, at least 40%, at least 50%, at least 60%, at least 70%, at least 80%, or at least 90% identity to the sequence set forth in Accession No. NM_001161587.2, NM_002103.5, or a fragment thereof.

In an aspect, a disclosed nucleic acid sequence for GYS2 can comprise the sequence set forth in Accession No. NM_021957.4, XM_024448960.1, XM_006719063.3, XM_017019245.2, or a fragment thereof. In an aspect, a disclosed nucleic acid sequence for GYS2 can comprise a sequence having at least 30%, at least 40%, at least 50%, at least 60%, at least 70%, at least 80%, or at least 90% identity to the sequence set forth in Accession No. NM_021957.4, XM_024448960.1, XM_006719063.3, XM_017019245.2, or a fragment thereof.

In an aspect, a disclosed nucleic acid sequence for GAA can comprise the sequence set forth in SEQ ID NO:20 or a fragment thereof. In an aspect, a disclosed nucleic acid sequence for GAA can comprise a sequence having at least 80%, at least 85%, at least 90%, or at least 95% identity to the sequence set forth in SEQ ID NO:20 or a fragment thereof.

CTCGAGCAACCATGGGAGTGAGGCACCCGCCCTGCTCCCACCGGC
TCCTGGCCGTCTGCGCCCTCGTGTCCTTGGCAACCGCTGCACTCC
TGGGGCACATCCTACTCCATGATTTCCTGCTGGTTCCCCGAGAGC
TGAGTGGCTCCTCCCCAGTCCTGGAGGAGACTCACCCAGCTCACC
AGCAGGGAGCCAGCAGACCAGGGCCCCGGGATGCCCAGGCACACC
CCGGCCGTCCCAGAGCAGTGCCCACACAGTGCGACGTCCCCCCCA
ACAGCCGCTTCGATTGCGCCCCTGACAAGGCCATCACCCAGGAAC
AGTGCGAGGCCCGCGGCTGCTGCTACATCCCTGCAAAGCAGGGGC
TGCAGGGAGCCCAGATGGGGCAGCCCTGGTGCTTCTTCCCACCCA
GCTACCCCAGCTACAAGCTGGAGAACCTGAGCTCCTCTGAAATGG
GCTACACGGCCACCCTGACCCGTACCACCCCCACCTTCTTCCCCA
AGGACATCCTGACCCTGCGGCTGGACGTGATGATGGAGACTGAGA
ACCGCCTCCACTTCACGATCAAAGATCCAGCTAACAGGCGCTACG
AGGTGCCCTTGGAGACCCCGCGTGTCCACAGCCGGGCACCGTCCC
CACTCTACAGCGTGGAGTTCTCTGAGGAGCCCTTCGGGGTGATCG
TGCACCGGCAGCTGGACGGCCGCGTGCTGCTGAACACGACGGTGG
CGCCCCTGTTCTTTGCGGACCAGTTCCTTCAGCTGTCCACCTCGC
TGCCCTCGCAGTATATCACAGGCCTCGCCGAGCACCTCAGTCCCC
TGATGCTCAGCACCAGCTGGACCAGGATCACCCTGTGGAACCGGG
ACCTTGCGCCCACGCCCGGTGCGAACCTCTACGGGTCTCACCCTT
TCTACCTGGCGCTGGAGGACGGCGGGTCGGCACACGGGGTGTTCC
TGCTAAACAGCAATGCCATGGATGTGGTCCTGCAGCCGAGCCCTG
CCCTTAGCTGGAGGTCGACAGGTGGGATCCTGGATGTCTACATCT
TCCTGGGCCCAGAGCCCAAGAGCGTGGTGCAGCAGTACCTGGACG
TTGTGGGATACCCGTTCATGCCGCCATACTGGGGCCTGGGCTTCC
ACCTGTGCCGCTGGGGCTACTCCTCCACCGCTATCACCCGCCAGG
TGGTGGAGAACATGACCAGGGCCCACTTCCCCCTGGACGTCCAAT
GGAACGACCTGGACTACATGGACTCCCGGAGGGACTTCACGTTCA
ACAAGGATGGCTTCCGGGACTTCCCGGCCATGGTGCAGGAGCTGC
ACCAGGGCGGCCGGCGCTACATGATGATCGTGGATCCTGCCATCA
GCAGCTCGGGCCCTGCCGGGAGCTACAGGCCCTACGACGAGGGTC
TGCGGAGGGGGGTTTTCATCACCAACGAGACCGGCCAGCCGCTGA
TTGGGAAGGTATGGCCCGGGTCCACTGCCTTCCCCGACTTCACCA
ACCCCACAGCCCTGGCCTGGTGGGAGGACATGGTGGCTGAGTTCC
ATGACCAGGTGCCCTTCGACGGCATGTGGATTGACATGAACGAGC
CTTCCAACTTCATCAGGGGCTCTGAGGACGGCTGCCCCAACAATG
AGCTGGAGAACCCACCCTACGTGCCTGGGGTGGTTGGGGGGACCC
TCCAGGCGGCCACCATCTGTGCCTCCAGCCACCAGTTTCTCTCCA
CACACTACAACCTGCACAACCTCTACGGCCTGACCGAAGCCATCG
CCTCCCACAGGGCGCTGGTGAAGGCTCGGGGGACACGCCCATTTG
TGATCTCCCGCTCGACCTTTGCTGGCCACGGCCGATACGCCGGCC
ACTGGACGGGGGACGTGTGGAGCTCCTGGGAGCAGCTCGCCTCCT
CCGTGCCAGAAATCCTGCAGTTTAACCTGCTGGGGGTGCCTCTGG
TCGGGGCCGACGTCTGCGGCTTCCTGGGCAACACCTCAGAGGAGC
TGTGTGTGCGCTGGACCCAGCTGGGGGCCTTCTACCCCTTCATGC
GGAACCACAACAGCCTGCTCAGTCTGCCCCAGGAGCCGTACAGCT
TCAGCGAGCCGGCCCAGCAGGCCATGAGGAAGGCCCTCACCCTGC
GCTACGCACTCCTCCCCCACCTCTACACGCTGTTCCACCAGGCCC
ACGTCGCGGGGGAGACCGTGGCCCGGCCCCTCTTCCTGGAGTTCC
CCAAGGACTCTAGCACCTGGACTGTGGACCACCAGCTCCTGTGGG
GGGAGGCCCTGCTCATCACCCCAGTGCTCCAGGCCGGGAAGGCCG
AAGTGACTGGCTACTTCCCCTTGGGCACATGGTACGACCTGCAGA
CGGTGCCAATAGAGGCCCTTGGCAGCCTCCCACCCCCACCTGCAG
CTCCCCGTGAGCCAGCCATCCACAGCGAGGGGCAGTGGGTGACGC
TGCCGGCCCCCCTGGACACCATCAACGTCCACCTCCGGGCTGGGT
ACATCATCCCCCTGCAGGGCCCTGGCCTCACAACCACAGAGTCCC
GCCAGCAGCCCATGGCCCTGGCTGTGGCCCTGACCAAGGGTGGAG
AGGCCCGAGGGGAGCTGTTCTGGGACGATGGAGAGAGCCTGGAAG
TGCTGGAGCGAGGGGCCTACACACAGGTCATCTTCCTGGCCAGGA
ATAACACGATCGTGAATGAGCTGGTACGTGTGACCAGTGAGGGAG
CTGGCCTGCAGCTGCAGAAGGTGACTGTCCTGGGCGTGGCCACGG
CGCCCCAGCAGGTCCTCTCCAACGGTGTCCCTGTCTCCAACTTCA
CCTACAGCCCCGACACCAAGGTCCTGGACATCTGTGTCTCGCTGT
TGATGGGAGAGCAGTTTCTCGTCAGCTGGTGTTAAACTCGAG

In an aspect, a disclosed nucleic acid sequence for GAA can comprise the sequence set forth in SEQ ID NO:21 or a fragment thereof. In an aspect, a disclosed nucleic acid sequence for GAA can comprise a sequence having at least 80%, at least 85%, at least 90%, or at least 95% identity to the sequence set forth in SEQ ID NO:21 or a fragment thereof.

GCGCCTGCGCGGGAGGCCGCGTCACGTGACCCACCGCGGCCCCGC
CCCGCGACGAGCTCCCGCCGGTCACGTGACCCGCCTCTGCGCGCC
CCCGGGCACGACCCCGGAGTCTCCGCGGGCGGCCAGGGCGCGCGT
GCGCGGAGGTGAGCCGGGCCGGGGCTGCGGGGCTTCCCTGAGCGC
GGGCCGGGTCGGTGGGGCGGTCGGCTGCCCGCGCCGGCCTCTCAG
TTGGGAAAGCTGAGGTTGTCGCCGGGGCCGCGGGTGGAGGTCGGG
GATGAGGCAGCAGGTAGGACAGTGACCTCGGTGACGCGAAGGACC
CCGGCCACCTCTAGGTTCTCCTCGTCCGCCCGTTGTTCAGCGAGG
GAGGCTCTGGGCCTGCCGCAGCTGACGGGGAAACTGAGGCACGGA
GCGGGCCTGTAGGAGCTGTCCAGGCCATCTCCAACCATGGGAGTG
AGGCACCCGCCCTGCTCCCACCGGCTCCTGGCCGTCTGCGCCCTC
GTGTCCTTGGCAACCGCTGCACTCCTGGGGCACATCCTACTCCAT
GATTTCCTGCTGGTTCCCCGAGAGCTGAGTGGCTCCTCCCCAGTC
CTGGAGGAGACTCACCCAGCTCACCAGCAGGGAGCCAGCAGACCA
GGGCCCCGGGATGCCCAGGCACACCCCGGCCGTCCCAGAGCAGTG
CCCACACAGTGCGACGTCCCCCCCAACAGCCGCTTCGATTGCGCC
CCTGACAAGGCCATCACCCAGGAACAGTGCGAGGCCCGCGGCTGC
TGCTACATCCCTGCAAAGCAGGGGCTGCAGGGAGCCCAGATGGGG
CAGCCCTGGTGCTTCTTCCCACCCAGCTACCCCAGCTACAAGCTG
GAGAACCTGAGCTCCTCTGAAATGGGCTACACGGCCACCCTGACC
CGTACCACCCCCACCTTCTTCCCCAAGGACATCCTGACCCTGCGG
CTGGACGTGATGATGGAGACTGAGAACCGCCTCCACTTCACGATC
AAAGATCCAGCTAACAGGCGCTACGAGGTGCCCTTGGAGACCCCG
CGTGTCCACAGCCGGGCACCGTCCCCACTCTACAGCGTGGAGTTC
TCCGAGGAGCCCTTCGGGGTGATCGTGCACCGGCAGCTGGACGGC
CGCGTGCTGCTGAACACGACGGTGGCGCCCCTGTTCTTTGCGGAC
CAGTTCCTTCAGCTGTCCACCTCGCTGCCCTCGCAGTATATCACA
GGCCTCGCCGAGCACCTCAGTCCCCTGATGCTCAGCACCAGCTGG
ACCAGGATCACCCTGTGGAACCGGGACCTTGCGCCCACGCCCGGT
GCGAACCTCTACGGGTCTCACCCTTTCTACCTGGCGCTGGAGGAC
GGCGGGTCGGCACACGGGGTGTTCCTGCTAAACAGCAATGCCATG
GATGTGGTCCTGCAGCCGAGCCCTGCCCTTAGCTGGAGGTCGACA
GGTGGGATCCTGGATGTCTACATCTTCCTGGGCCCAGAGCCCAAG
AGCGTGGTGCAGCAGTACCTGGACGTTGTGGGATACCCGTTCATG
CCGCCATACTGGGGCCTGGGCTTCCACCTGTGCCGCTGGGGCTAC
TCCTCCACCGCTATCACCCGCCAGGTGGTGGAGAACATGACCAGG
GCCCACTTCCCCCTGGACGTCCAATGGAACGACCTGGACTACATG
GACTCCCGGAGGGACTTCACGTTCAACAAGGATGGCTTCCGGGAC
TTCCCGGCCATGGTGCAGGAGCTGCACCAGGGCGGCCGGCGCTAC
ATGATGATCGTGGATCCTGCCATCAGCAGCTCGGGCCCTGCCGGG
AGCTACAGGCCCTACGACGAGGGTCTGCGGAGGGGGGTTTTCATC
ACCAACGAGACCGGCCAGCCGCTGATTGGGAAGGTATGGCCCGGG
TCCACTGCCTTCCCCGACTTCACCAACCCCACAGCCCTGGCCTGG
TGGGAGGACATGGTGGCTGAGTTCCATGACCAGGTGCCCTTCGAC
GGCATGTGGATTGACATGAACGAGCCTTCCAACTTCATCAGAGGC
TCTGAGGACGGCTGCCCCAACAATGAGCTGGAGAACCCACCCTAC
GTGCCTGGGGTGGTTGGGGGGACCCTCCAGGCGGCCACCATCTGT
GCCTCCAGCCACCAGTTTCTCTCCACACACTACAACCTGCACAAC
CTCTACGGCCTGACCGAAGCCATCGCCTCCCACAGGGCGCTGGTG
AAGGCTCGGGGGACACGCCCATTTGTGATCTCCCGCTCGACCTTT
GCTGGCCACGGCCGATACGCCGGCCACTGGACGGGGGACGTGTGG
AGCTCCTGGGAGCAGCTCGCCTCCTCCGTGCCAGAAATCCTGCAG
TTTAACCTGCTGGGGGTGCCTCTGGTCGGGGCCGACGTCTGCGGC
TTCCTGGGCAACACCTCAGAGGAGCTGTGTGTGCGCTGGACCCAG
CTGGGGGCCTTCTACCCCTTCATGCGGAACCACAACAGCCTGCTC
AGTCTGCCCCAGGAGCCGTACAGCTTCAGCGAGCCGGCCCAGCAG
GCCATGAGGAAGGCCCTCACCCTGCGCTACGCACTCCTCCCCCAC
CTCTACACACTGTTCCACCAGGCCCACGTCGCGGGGGAGACCGTG
GCCCGGCCCCTCTTCCTGGAGTTCCCCAAGGACTCTAGCACCTGG
ACTGTGGACCACCAGCTCCTGTGGGGGGAGGCCCTGCTCATCACC
CCAGTGCTCCAGGCCGGGAAGGCCGAAGTGACTGGCTACTTCCCC
TTGGGCACATGGTACGACCTGCAGACGGTGCCAATAGAGGCCCTT
GGCAGCCTCCCACCCCCACCTGCAGCTCCCCGTGAGCCAGCCATC
CACAGCGAGGGGCAGTGGGTGACGCTGCCGGCCCCCCTGGACACC
ATCAACGTCCACCTCCGGGCTGGGTACATCATCCCCCTGCAGGGC
CCTGGCCTCACAACCACAGAGTCCCGCCAGCAGCCCATGGCCCTG
GCTGTGGCCCTGACCAAGGGTGGAGAGGCCCGAGGGGAGCTGTTC
TGGGACGATGGAGAGAGCCTGGAAGTGCTGGAGCGAGGGGCCTAC
ACACAGGTCATCTTCCTGGCCAGGAATAACACGATCGTGAATGAG
CTGGTACGTGTGACCAGTGAGGGAGCTGGCCTGCAGCTGCAGAAG
GTGACTGTCCTGGGCGTGGCCACGGCGCCCCAGCAGGTCCTCTCC
AACGGTGTCCCTGTCTCCAACTTCACCTACAGCCCCGACACCAAG
GTCCTGGACATCTGTGTCTCGCTGTTGATGGGAGAGCAGTTTCTC
GTCAGCTGGTGTTAGCCGGGCGGAGTGTGTTAGTCTCTCCAGAGG
GAGGCTGGTTCCCCAGGGAAGCAGAGCCTGTGTGCGGGCAGCAGC
TGTGTGCGGGCCTGGGGGTTGCATGTGTCACCTGGAGCTGGGCAC
TAACCATTCCAAGCCGCCGCATCGCTTGTTTCCACCTCCTGGGCC
GGGGCTCTGGCCCCCAACGTGTCTAGGAGAGCTTTCTCCCTAGAT
CGCACTGTGGGCCGGGGCCTGGAGGGCTGCTCTGTGTTAATAAGA
TTGTAAGGTTTGCCCTCCTCACCTGTTGCCGGCATGCGGGTAGTA
TTAGCCACCCCCCTCCATCTGTTCCCAGCACCGGAGAAGGGGGTG
CTCAGGTGGAGGTGTGGGGTATGCACCTGAGCTCCTGCTTCGCGC
CTGCTGCTCTGCCCCAACGCGACCGCTTCCCGGCTGCCCAGAGGG
CTGGATGCCTGCCGGTCCCCGAGCAAGCCTGGGAACTCAGGAAAA
TTCACAGGACTTGGGAGATTCTAAATCTTAAGTGCAATTATTTTA
ATAAAAGGGGCATTTGGAATC.

b. Polypeptide Sequences

In an aspect, a disclosed human glycogen branching enzyme can comprise the sequence set forth in NCBI Reference Sequence No. NP_000149.4.

In an aspect, a disclosed human glycogen branching enzyme can comprise the following sequence:

(SEQ ID NO: 01)
MAAPMTPAARPEDYEAALNAALADVPELARLLEIDPYLKPYAVDF
QRRYKQFSQILKNIGENEGGIDKFSRGYESFGVHRCADGGLYCKE
WAPGAEGVFLTGDFNGWNPFSYPYKKLDYGKWELYIPPKQNKSVL
VPHGSKLKVVITSKSGEILYRISPWAKYVVREGDNVNYDWIHWDP
EHSYEFKHSPKKPRSLRIYESHVGISSHEGKVASYKHFTCNVLPR
IKGLGYNCIQLMAIMEHAYYASFGYQITSFFAASSRYGTPEELQE
LVDTAHSMGIIVLLDVVHSHASKNSADGLNMFDGTDSCYFHSGPR
GTHDLWDSRLFAYSSWEILRFLLSNIRWWLEEYRFDGFRFDGVTS
MLYHHHGVGQGFSGDYSEYFGLQVDEDALTYLMLANHLVHTLCPD
SITIAEDVSGMPALCSPISQGGGGFDYRLAMAIPDKWIQLLKEFK
DEDWNMGDIVYTLTNRRYLEKCIAYAESHDQALVGDKSLAFWLMD
AEMYTNMSVLTPFTPVIDRGIQLHKMIRLITHGLGGEGYLNFMGN
EFGHPEWLDFPRKGNNESYHYARRQFHLTDDDLLRYKFLNNFDRD
MNRLEERYGWLAAPQAYVSEKHEGNKIIAFERAGLLFIFNFHPSK
SYTDYRVGTALPGKFKIVLDSDAAEYGGHQRLDHSTDFFSEAFEH
NGRPYSLLVYIPSRVALILQNVDLPN.

In an aspect, a disclosed human salivary alpha-amylase 1A precursor can comprise the following sequence:

(SEQ ID NO: 07)
MKLFWLLFTIGFCWAQYSSNTQQGRTSIVHLFEWRWVDIALECER
YLAPKGFGGVQVSPPNENVAIHNPFRPWWERYQPVSYKLCTRSGN
EDEFRNMVTRCNNVGVRIYVDAVINHMCGNAVSAGTSSTCGSYFN
PGSRDFPAVPYSGWDFNDGKCKTGSGDIENYNDATQVRDCRLSGL
LDLALGKDYVRSKIAEYMNHLIDIGVAGFRIDASKHMWPGDIKAI
LDKLHNLNSNWFPEGSKPFIYQEVIDLGGEPIKSSDYFGNGRVTE
FKYGAKLGTVIRKWNGEKMSYLKNWGEGWGFMPSDRALVFVDNHD
NQRGHGAGGASILTFWDARLYKMAVGFMLAHPYGFTRVMSSYRWP
RYFENGKDVNDWVGPPNDNGVTKEVTINPDTTCGNDWVCEHRWRQ
IRNMVNFRNVVDGQPFTNWYDNGSNQVAFGRGNRGFIVFNNDDWT
FSLTLQTGLPAGTYCDVISGDKINGNCTGIKIYVSDDGKAHFSIS
NSAEDPFIAIHAESKL.

In an aspect, a disclosed human salivary amylase can have the sequence set forth in NCBI Reference Sequence No. NP_001008222. In an aspect, a disclosed human pancreatic alpha-amylase can comprise the following sequence:

(SEQ ID NO: 08)
MKFFLLLFTIGFCWAQYSPNTQQGRTSIVHLFEWRWVDIALECER
YLAPKGFGGVQVSPPNENVAIYNPFRPWWERYQPVSYKLCTRSGN
EDEFRNMVTRCNNVGVRIYVDAVINHMCGNAVSAGTSSTCGSYFN
PGSRDFPAVPYSGWDFNDGKCKTGSGDIENYNDATQVRDCRLTGL
LDLALEKDYVRSKIAEYMNHLIDIGVAGFRLDASKHMWPGDIKAI
LDKLHNLNSNWFPAGSKPFIYQEVIDLGGEPIKSSDYFGNGRVTE
FKYGAKLGTVIRKWNGEKMSYLKNWGEGWGFVPSDRALVFVDNHD
NQRGHGAGGASILTFWDARLYKMAVGFMLAHPYGFTRVMSSYRWP
RQFQNGNDVNDWVGPPNNNGVIKEVTINPDTTCGNDWVCEHRWRQ
IRNMVIFRNVVDGQPFTNWYDNGSNQVAFGRGNRGFIVFNNDDWS
FSLTLQTGLPAGTYCDVISGDKINGNCTGIKIYVSDDGKAHFSIS
NSAEDPFIAIHAESKL.

In an aspect, a disclosed human pancreatic alpha-amylase can comprise the sequence set forth in NCBI Reference Sequence No. NP_000690.

In an aspect, a disclosed endonuclease can be Cas9. In an aspect, a disclosed Cas9 can be from Staphylococcus aureus or Streptococcus pyogenes. A disclosed Cas9 can comprise the sequence set forth in SEQ ID NO:09 or a fragment thereof. In an aspect, a disclosed Cas9 can comprise a sequence having at least 75%, at least 80%, at least 85%, at least 90%, or at least 95% identity to the sequence set forth in SEQ ID NO:09 or a fragment thereof.

(SEQ ID NO: 09)
MKRNYILGLDIGITSVGYGIIDYETRDVIDAGVRLFKEANVENNE
GRRSKRGARRLKRRRRHRIQRVKKLLFDYNLLTDHSELSGINPYE
ARVKGLSQKLSEEEFSAALLHLAKRRGVHNVNEVEEDTGNELSTK
EQISRNSKALEEKYVAELQLERLKKDGEVRGSINRFKTSDYVKEA
KQLLKVQKAYHQLDQSFIDTYIDLLETRRTYYEGPGEGSPFGWKD
IKEWYEMLMGHCTYFPEELRSVKYAYNADLYNALNDLNNLVITRD
ENEKLEYYEKFQIIENVFKQKKKPTLKQIAKEILVNEEDIKGYRV
TSTGKPEFTNLKVYHDIKDITARKEIIENAELLDQIAKILTIYQS
SEDIQEELTNLNSELTQEEIEQISNLKGYTGTHNLSLKAINLILD
ELWHTNDNQIAIFNRLKLVPKKVDLSQQKEIPTTLVDDFILSPVV
KRSFIQSIKVINAIIKKYGLPNDIIIELAREKNSKDAQKMINEMQ
KRNRQTNERIEEIIRTTGKENAKYLIEKIKLHDMQEGKCLYSLEA
IPLEDLLNNPFNYEVDHIIPRSVSFDNSFNNKVLVKQEENSKKGN
RTPFQYLSSSDSKISYETFKKHILNLAKGKGRISKTKKEYLLEER
DINRFSVQKDFINRNLVDTRYATRGLMNLLRSYFRVNNLDVKVKS
INGGFTSFLRRKWKFKKERNKGYKHHAEDALIIANADFIFKEWKK
LDKAKKVMENQMFEEKQAESMPEIETEQEYKEIFITPHQIKHIKD
FKDYKYSHRVDKKPNRELINDTLYSTRKDDKGNTLIVNNLNGLYD
KDNDKLKKLINKSPEKLLMYHHDPQTYQKLKLIMEQYGDEKNPLY
KYYEETGNYLTKYSKKDNGPVIKKIKYYGNKLNAHLDITDDYPNS
RNKVVKLSLKPYRFDVYLDNGVYKFVTVKNLDVIKKENYYEVNSK
CYEEAKKLKKISNQAEFIASFYNNDLIKINGELYRVIGVNNDLLN
RIEVNMIDITYREYLENMNDKRPPRIIKTIASKTQSIKKYSTDIL
GNLYEVKSKKHPQIIKKG.

In an aspect, a disclosed Cas9 can comprise the sequence set forth in SEQ ID NO: 10 or a fragment thereof. In an aspect, a disclosed Cas9 can comprise a sequence having at least 75%, at least 80%, at least 85%, at least 90%, or at least 95% identity to the sequence set forth in SEQ ID NO:10 or a fragment thereof.

(SEQ ID NO: 10)
MDKKYSIGLDIGTNSVGWAVITDDYKVPSKKFKVLGNTDRHSIKK
NLIGALLFDSGETAEATRLKRTARRRYTRRKNRICYLQEIFSNEM
AKVDDSFFHRLEESFLVEEDKKHERHPIFGNIVDEVAYHEKYPTI
YHLRKKLVDSTDKADLRLIYLALAHMIKFRGHFLIEGDLNPDNSD
VDKLFIQLVQTYNQLFEENPINASGVDAKAILSARLSKSRRLENL
IAQLPGEKKNGLFGNLIALSLGLTPNFKSNFDLAEDAKLQLSKDT
YDDDLDNLLAQIGDQYADLFLAAKNLSDAILLSDILRLNSEITKA
PLSASMIKRYDEHHQDLTLLKALVRQQLPEKYKEIFFDQSKNGYA
GYIDGGASQEEFYKFIKPILEKMDGTEELLAKLNREDLLRKQRTF
DNGSIPHQIHLGELHAILRRQEDFYPFLKDNREKIEKILTFRIPY
YVGPLARGNSRFAWMTRKSEETITPWNFEEVVDKGASAQSFIERM
TNFDKNLPNEKVLPKHSLLYEYFTVYNELTKVKYVTEGMRKPAFL
SGEQKKAIVDLLFKTNRKVTVKQLKEDYFKKIECFDSVEISGVED
RFNASLGTYHDLLKIIKDKDFLDNEENEDILEDIVLTLTLFEDRE
MIEERLKTYAHLFDDKVMKQLKRRRYTGWGRLSRKLINGIRDKQS
GKTILDFLKSDGFANRNFMQLIHDDSLTFKEDIQKAQVSGQGDSL
HEHIANLAGSPAIKKGILQTVKVVDELVKVMGRHKPENIVIEMAR
ENQTTQKGQKNSRERMKRIEEGIKELGSQILKEHPVENTQLQNEK
LYLYYLQNGRDMYVDQELDINRLSDYDVDHIVPQSFIKDDSIDNK
VLTRSDKNRGKSDNVPSEEVVKKMKNYWRQLLNAKLITQRKFDNL
TKAERGGLSELDKAGFIKRQLVETRQITKHVAQILDSRMNTKYDE
NDKLIREVKVITLKSKLVSDFRKDFQFYKVREINNYHHAHDAYLN
AVVGTALIKKYPKLESEFVYGDYKVYDVRKMIAKSEQEIGKATAK
YFFYSNIMNFFKTEITLANGEIRKRPLIETNGETGEIVWDKGRDF
ATVRKVLSMPQVNIVKKTEVQTGGFSKESILPKRNSDKLIARKKD
WDPKKYGGFDSPTVAYSVLVVAKVEKGKSKKLKSVKELLGLTIME
RSSFEKNPIDFLEAKGYKEVKKDLIIKLPKYSLFELENGRKRMLA
SAGELQKGNELALPSKYVNFLYLASHYEKLKGSPEDNEQKQLFVE
QHKHYLDEIIEQISEFSKRVILADANLDKVLSAYNKHRDKPIREQ
AENIIHLFTLTNLGAPAAFKYFDTTIDRKRYTSTKEVLDATLIHQ
SITGLYETRIDLSQLGGD.

In an aspect, a disclosed GYS1 can comprise the sequence set forth in SEQ ID NO:12 or a fragment thereof. In an aspect, a disclosed GYS1 can comprise a sequence having at least 75%, at least 80%, at least 85%, at least 90%, or at least 95% identity to the sequence set forth in SEQ ID NO: 12 or a fragment thereof.

(SEQ ID NO: 12)
MPLNRTLSMSSLPGLEDWEDEFDLENAVLFEVAWEVANKVGGIYT
VLQTKAKVTGDEWGDNYFLVGPYTEQGVRTQVELLEAPTPALKRT
LDSMNSKGCKFLAQSEEKPHVVAHFHEWLAGVGLCLCRARRLPVA
TIFTTHATLLGRYLCAGAVDFYNNLENFNVDKEAGERQIYHRYCM
ERAAAHCAHVFTTVSQITAIEAQHLLKRKPDIVTPNGLNVKKFSA
MHEFQNLHAQSKARIQEFVRGHFYGHLDFNLDKTLYFFIAGRYEF
SNKGADVFLEALARLNYLLRVNGSEQTVVAFFIMPARTNNFNVET
LKGQAVRKQLWDTANTVKEKFGRKLYESLLVGSLPDMNKMLDKED
FTMMKRAIFATQRQSFPPVCTHNMLDDSSDPILTTIRRIGLFNSS
ADRVKVIFHPEFLSSTSPLLPVDYEEFVRGCHLGVFPSYYEPWGY
TPAECTVMGIPSISTNLSGFGCFMEEHIADPSAYGIYILDRRFRS
LDDSCSQLTSFLYSFCQQSRRQRIIQRNRTERLSDLLDWKYLGRY
YMSARHMALSKAFPEHFTYEPNEADAAQGYRYPRPASVPPSPSLS
RHSSPHQSEDEEDPRNGPLEEDGERYDEDEEAAKDRRNIRAPEWP
RRASCTSSTSGSKRNSVDTATSSSLSTPSEPLSPTSSLGEERN.

In an aspect, a disclosed GYS1 can comprise the sequence set forth in SEQ ID NO:13 or a fragment thereof. In an aspect, a disclosed GYS1 can comprise a sequence having at least 75%, at least 80%, at least 85%, at least 90%, or at least 95% identity to the sequence set forth in SEQ ID NO: 13 or a fragment thereof.

(SEQ ID NO: 13)
MPLNRTLSMSSLPGLEDWEDEFDLENAVLFEVAWEVANKVGGIYT
VLQTKAKVTGDEWGDNYFLVGPYTEQGVRTQVELLEAPTPALKRT
LDSMNSKGCKVYFGRWLIEGGPLVVLLDVGASAWALERWKGELWD
TCNIGVPWYDREANDAVLFGFLTTWFLGEFLAQSEEKPHVVAHFH
EWLAGVGLCLCRARRLPVATIFTTHATLLGRYLCAGAVDFYNNLE
NFNVDKEAGERQIYHRYCMERAAAHCAHVFTTVSQITAIEAQHLL
KRKPDIVTPNGLNVKKFSAMHEFQNLHAQSKARIQEFVRGHFYGH
LDFNLDKTLYFFIAGRYEFSNKGADVFLEALARLNYLLRVNGSEQ
TVVAFFIMPARTNNENVETLKGQAVRKQLWDTANTVKEKFGRKLY
ESLLVGSLPDMNKMLDKEDFTMMKRAIFATQRQSFPPVCTHNMLD
DSSDPILTTIRRIGLFNSSADRVKVIFHPEFLSSTSPLLPVDYEE
FVRGCHLGVFPSYYEPWGYTPAECTVMGIPSISTNLSGFGCFMEE
HIADPSAYGIYILDRRFRSLDDSCSQLTSFLYSFCQQSRRQRIIQ
RNRTERLSDLLDWKYLGRYYMSARHMALSKAFPEHFTYEPNEADA
AQGYRYPRPASVPPSPSLSRHSSPHQSEDEEDPRNGPLEEDGERY
DEDEEAAKDRRNIRAPEWPRRASCTSSTSGSKRNSVDTATSSSLS
TPSEPLSPTSSLGEERN.

In an aspect, a disclosed GYS2 can comprise the sequence set forth in SEQ ID NO: 14 or a fragment thereof. In an aspect, a disclosed GYS2 can comprise a sequence having at least 75%, at least 80%, at least 85%, at least 90%, or at least 95% identity to the sequence set forth in SEQ ID NO: 14 or a fragment thereof.

(SEQ ID NO: 14)
MLRGRSLSVTSLGGLPQWEVEELPVEELLLFEVAWEVTNKVGGIY
TVIQTKAKTTADEWGENYFLIGPYFEHNMKTQVEQCEPVNDAVRR
AVDAMNKHGCQVHFGRWLIEGSPYVVLFDIGYSAWNLDRWKGDLW
EACSVGIPYHDREANDMLIFGSLTAWFLKEVTDHADGKYVVAQFH
EWQAGIGLILSRARKLPIATIFTTHATLLGRYLCAANIDFYNHLD
KFNIDKEAGERQIYHRYCMERASVHCAHVFTTVSEITAIEAEHML
KRKPDVVTPNGLNVKKFSAVHEFQNLHAMYKARIQDFVRGHFYGH
LDFDLEKTLFLFIAGRYEFSNKGADIFLESLSRLNFLLRMHKSDI
TVMVFFIMPAKTNNFNVETLKGQAVRKQLWDVAHSVKEKFGKKLY
DALLRGEIPDLNDILDRDDLTIMKRAIFSTQRQSLPPVTTHNMID
DSTDPILSTIRRIGLENNRTDRVKVILHPEFLSSTSPLLPMDYEE
FVRGCHLGVFPSYYEPWGYTPAECTVMGIPSVTTNLSGFGCFMQE
HVADPTAYGIYIVDRRFRSPDDSCNQLTKFLYGFCKQSRRQRIIQ
RNRTERLSDLLDWRYLGRYYQHARHLTLSRAFPDKFHVELTSPPT
TEGFKYPRPSSVPPSPSGSQASSPQSSDVEDEVEDERYDEEEEAE
RDRLNIKSPFSLSHVPHGKKKLHGEYKN.

In an aspect, a disclosed GAA can comprise the sequence set forth in SEQ ID NO:15 or a fragment thereof. In an aspect, a disclosed GAA can comprise a sequence having at least 75%, at least 80%, at least 85%, at least 90%, or at least 95% identity to the sequence set forth in SEQ ID NO: 15 or a fragment thereof.

(SEQ ID NO: 15)
MGVRHPPCSHRLLAVCALVSLATAALLGHILLHDFLLVPRELSGS
SPVLEETHPAHQQGASRPGPRDAQAHPGRPRAVPTQCDVPPNSRF
DCAPDKAITQEQCEARGCCYIPAKQGLQGAQMGQPWCFFPPSYPS
YKLENLSSSEMGYTATLTRTTPTFFPKDILTLRLDVMMETENRLH
FTIKDPANRRYEVPLETPRVHSRAPSPLYSVEFSEEPFGVIVHRQ
LDGRVLLNTTVAPLFFADQFLQLSTSLPSQYITGLAEHLSPLMLS
TSWTRITLWNRDLAPTPGANLYGSHPFYLALEDGGSAHGVFLLNS
NAMDVVLQPSPALSWRSTGGILDVYIFLGPEPKSVVQQYLDVVGY
PFMPPYWGLGFHLCRWGYSSTAITRQVVENMTRAHFPLDVQWNDL
DYMDSRRDFTFNKDGFRDFPAMVQELHQGGRRYMMIVDPAISSSG
PAGSYRPYDEGLRRGVFITNETGQPLIGKVWPGSTAFPDFTNPTA
LAWWEDMVAEFHDQVPFDGMWIDMNEPSNFIRGSEDGCPNNELEN
PPYVPGVVGGTLQAATICASSHQFLSTHYNLHNLYGLTEAIASHR
ALVKARGTRPFVISRSTFAGHGRYAGHWTGDVWSSWEQLASSVPE
ILQFNLLGVPLVGADVCGFLGNTSEELCVRWTQLGAFYPFMRNHN
SLLSLPQEPYSFSEPAQQAMRKALTLRYALLPHLYTLFHQAHVAG
ETVARPLFLEFPKDSSTWTVDHQLLWGEALLITPVLQAGKAEVTG
YFPLGTWYDLQTVPIEALGSLPPPPAAPREPAIHSEGQWVTLPAP
LDTINVHLRAGYIIPLQGPGLTTTESRQQPMALAVALTKGGEARG
ELFWDDGESLEVLERGAYTQVIFLARNNTIVNELVRVTSEGAGLQ
LQKVTVLGVATAPQQVLSNGVPVSNFTYSPDTKVLDICVSLLMGE
QFLVSWC.

In an aspect, a disclosed GAA can comprise the sequence set forth in SEQ ID NO: 16 or a fragment thereof. In an aspect, a disclosed GAA can comprise a sequence having at least 75%, at least 80%, at least 85%, at least 90%, or at least 95% identity to the sequence set forth in SEQ ID NO: 16 or a fragment thereof.

(SEQ ID NO: 16)
MGVRHPPCSHRLLAVCALVSLATAALLGHILLHDFLLVPRELSGS
SPVLEETHPAHQQGASRPGPRDAQAHPGRPRAVPTQCDVPPNSRF
DCAPDKAITQEQCEARGCCYIPAKQGLQGAQMGQPWCFFPPSYPS
YKLENLSSSEMGYTATLTRTTPTFFPKDILTLRLDVMMETENRLH
FTIKDPANRRYEVPLETPRVHSRAPSPLYSVEFSEEPFGVIVHRQ
LDGRVLLNTTVAPLFFADQFLQLSTSLPSQYITGLAEHLSPLMLS
TSWTRITLWNRDLAPTPGANLYGSHPFYLALEDGGSAHGVFLLNS
NAMDVVLQPSPALSWRSTGGILDVYIFLGPEPKSVVQQYLDVVGY
PFMPPYWGLGFHLCRWGYSSTAITRQVVENMTRAHFPLDVQWNDL
DYMDSRRDFTFNKDGFRDFPAMVQELHQGGRRYMMIVDPAISSSG
PAGSYRLYDEGLRRGVFITNETGQPLIGKVWPGSTAFPDFTNPTA
LAWWEDMVAEFHDQVPFDGMWIDMNEPSNFIRGSEDGCPNNELEN
PPYVPGVVGGTLQAATICASSHQFLSTHYNLHNLYGLTEAIASHR
ALVKARGTRPFVISRSTFAGHGRYAGHWTGDVWSSWEQLASSVPE
ILQFNLLGVPLVGADVCGFLGNTSEELCVRWTQLGAFYPFMRNHN
SLLSLPQEPYSFSEPAQQAMRKALTLRYALLPHLYTLFHQAHVAG
ETVARPLFLEFPKDSSTWTVDHQLLWGEALLITPVLQAGKAEVTG
YFPLGTWYDLQTVPIEALGSLPPPPAAPREPAIHSEGQWVTLPAP
LDTINVHLRAGYIIPLQGPGLTTTESRQQPMALAVALTKGGEARG
ELFWDDGESLEVLERGAYTQVIFLARNNTIVNELVRVTSEGAGLQ
LQKVTVLGVATAPQQVLSNGVPVSNFTYSPDTKVLDICVSLLMGE
QFLVSWC.

In an aspect, a disclosed GAA can comprise the sequence set forth in SEQ ID NO: 17 or a fragment thereof. In an aspect, a disclosed GAA can comprise a sequence having at least 75%, at least 80%, at least 85%, at least 90%, or at least 95% identity to the sequence set forth in SEQ ID NO: 17 or a fragment thereof.

(SEQ ID NO: 17)
MGVRHPPCSHRLLAVCALVSLATAALLGHILLHDFLLVPRELSGS
SPVLEETHPAHQQGASRPGPRDAQAHPGRPRAVPTQCDVPPNSRF
DCAPDKAITQEQCEARGCCYIPAKQGLQGAQMGQPWCFFPPSYPS
YKLENLSSSEMGYTATLTRTTPTFFPKDILTLRLDVMMETENRLH
FTIKDPANRRYEVPLETPHVHSRAPSPLYSVEFSEEPFGVIVRRQ
LDGRVLLNTTVAPLFFADQFLQLSTSLPSQYITGLAEHLSPLMLS
TSWTRITLWNRDLAPTPGANLYGSHPFYLALEDGGSAHGVFLLNS
NAMDVVLQPSPALSWRSTGGILDVYIFLGPEPKSVVQQYLDVVGY
PFMPPYWGLGFHLCRWGYSSTAITRQVVENMTRAHFPLDVQWNDL
DYMDSRRDFTFNKDGFRDFPAMVQELHQGGRRYMMIVDPAISSSG
PAGSYRPYDEGLRRGVFITNETGQPLIGKVWPGSTAFPDFTNPTA
LAWWEDMVAEFHDQVPFDGMWIDMNEPSNFIRGSEDGCPNNELEN
PPYVPGVVGGTLQAATICASSHQFLSTHYNLHNLYGLTEAIASHR
ALVKARGTRPFVISRSTFAGHGRYAGHWTGDVWSSWEQLASSVPE
ILQFNLLGVPLVGADVCGFLGNTSEELCVRWTQLGAFYPFMRNHN
SLLSLPQEPYSFSEPAQQAMRKALTLRYALLPHLYTLFHQAHVAG
ETVARPLFLEFPKDSSTWTVDHQLLWGEALLITPVLQAGKAEVTG
YFPLGTWYDLQTVPVEALGSLPPPPAAPREPAIHSEGQWVTLPAP
LDTINVHLRAGYIIPLQGPGLTTTESRQQPMALAVALTKGGEARG
ELFWDDGESLEVLERGAYTQVIFLARNNTIVNELVRVTSEGAGLQ
LQKVTVLGVATAPQQVLSNGVPVSNFTYSPDTKVLDICVSLLMGE
QFLVSWC.

In an aspect, a disclosed GAA can comprise the sequence set forth in SEQ ID NO:18 or a fragment thereof. In an aspect, a disclosed GAA can comprise a sequence having at least 75%, at least 80%, at least 85%, at least 90%, or at least 95% identity to the sequence set forth in SEQ ID NO: 18 or a fragment thereof.

(SEQ ID NO: 18)
MGVRHPPCSHRLLAVCALVSLATAALLGHILLHDFLLVPRELSGS
SPVLEETHPAHQQGASRPGPRDAQAHPGRPRAVPTQCDVPPNSRF
DCAPDKAITQEQCEARGCCYIPAKQGLQGAQMGQPWCFFPPSYPS
YKLENLSSSEMGYTATLTRTTPTFFPKDILTLRLDVMMETENRLH
FTIKDPANRRYEVPLETPHVHSRAPSPLYSVEFSEEPFGVIVRRQ
LDGRVLLNTTVAPLFFADQFLQLSTSLPSQYITGLAEHLSPLMLS
TSWTRITLWNRDLAPTPGANLYGSHPFYLALEDGGSAHGVFLLNS
NAMDVVLQPSPALSWRSTGGILDVYIFLGPEPKSVVQQYLDVVGY
PFMPPYWGLGFHLCRWGYSSTAITRQVVENMTRAHFPLDVQWNDL
DYMDSRRDFTFNKDGFRDFPAMVQELHQGGRRYMMIVDPAISSSG
PAGSYRPYDEGLRRGVFITNETGQPLIGKVWPGSTAFPDFTNPTA
LAWWEDMVAEFHDQVPFDGMWIDMNEPSNFIRGSEDGCPNNELEN
PPYVPGVVGGTLQAATICASSHQFLSTHYNLHNLYGLTEAIASHR
ALVKARGTRPFVISRSTFAGHGRYAGHWTGDVWSSWEQLASSVPE
ILQFNLLGVPLVGADVCGFLGNTSEELCVRWTQLGAFYPFMRNHN
SLLSLPQEPYSFSEPAQQAMRKALTLRYALLPHLYTLFHQAHVAG
ETVARPLFLEFPKDSSTWTVDHQLLWGEALLITPVLQAGKAEVTG
YFPLGTWYDLQTVPIEALGSLPPPPAAPREPAIHSEGQWVTLPAP
LDTINVHLRAGYIIPLQGPGLTTTESRQQPMALAVALTKGGEARG
ELFWDDGESLEVLERGAYTQVIFLARNNTIVNELVRVTSEGAGLQ
LQKVTVLGVATAPQQVLSNGPVSNFTYSPDTKVLDICVSLLMGEQ
FLVSWC.

In an aspect, a disclosed GAA can comprise the sequence set forth in SEQ ID NO:19 or a fragment thereof. In an aspect, a disclosed GAA can comprise a sequence having at least 75%, at least 80%, at least 85%, at least 90%, or at least 95% identity to the sequence set forth in SEQ ID NO:19 or a fragment thereof.

(SEQ ID NO: 19)
AHPGRPRAVPTQCDVPPNSRFDCAPDKAITQEQCEARGCCYIPAK
QGLQGAQMGQPWCFFPPSYPSYKLENLSSSEMGYTATLTRTTPTF
FPKDILTLRLDVMMETENRLHFTIKDPANRRYEVPLETPHVHSRA
PSPLYSVEFSEEPFGVIVRRQLDGRVLLNTTVAPLFFADQFLQLS
TSLPSQYITGLAEHLSPLMLSTSWTRITLWNRDLAPTPGANLYGS
HPFYLALEDGGSAHGVFLLNSNAMDVVLQPSPALSWRSTGGILDV
YIFLGPEPKSVVQQYLDVVGYPFMPPYWGLGFHLCRWGYSSTAIT
RQVVENMTRAHFPLDVQWNDLDYMDSRRDFTFNKDGFRDFPAMVQ
ELHQGGRRYMMIVDPAISSSGPAGSYRPYDEGLRRGVFITNETGQ
PLIGKVWPGSTAFPDFTNPTALAWWEDMVAEFHDQVPFDGMWIDM
NEPSNFIRGSEDGCPNNELENPPYVPGVVGGTLQAATICASSHQF
LSTHYNLHNLYGLTEAIASHRALVKARGTRPFVISRSTFAGHGRY
AGHWTGDVWSSWEQLASSVPEILQFNLLGVPLVGADVCGFLGNTS
EELCVRWTQLGAFYPFMRNHNSLLSLPQEPYSFSEPAQQAMRKAL
TLRYALLPHLYTLFHQAHVAGETVARPLFLEFPKDSSTWTVDHQL
LWGEALLITPVLQAGKAEVTGYFPLGTWYDLQTVPVEALGSLPPP
PAAPREPAIHSEGQWVTLPAPLDTINVHLRAGYIIPLQGPGLTTT
ESRQQPMALAVALTKGGEARGELFWDDGESLEVLERGAYTQVIFL
ARNNTIVNELVRVTSEGAGLQLQKVTVLGVATAPQQVLSNGVPVS
NFTYSPDTKVLDICVSLLMGEQFLVSWC.

### 2. Vectors

Disclosed herein is a vector comprising an isolated nucleic acid molecule comprising a nucleic acid sequence encoding a polypeptide for preventing glycogen accumulation and/or degrading accumulated glycogen, wherein the nucleic acid sequence is CpG-depleted and codon-optimized for expression in a human or a mammalian cell. In an aspect, a disclosed nucleic acid sequence can have a coding sequence that is less than about 4.5 kilobases.

Disclosed herein is a vector comprising a disclosed isolated nucleic acid molecule. For example, in an aspect, a disclosed vector can comprise a disclosed isolated nucleic acid molecule comprising the sequence set forth in SEQ ID NO:03 or SEQ ID NO:04 and the sequence set forth in SEQ ID NO:05 or SEQ ID NO:06, or a disclosed isolated nucleic acid molecule comprising a sequence having at least 30%, at least 40%, at least 50%, at least 60%, at least 70%, at least 80%, or at least 90% identity to the sequence set forth in SEQ ID NO:03 or SEQ ID NO:04 and a sequence having at least 30%, at least 40%, at least 50%, at least 60%, at least 70%, at least 80%, or at least 90% identity to the sequence set forth in SEQ ID NO:05 or SEQ ID NO:06.

In an aspect, a therapeutically effective amount of disclosed vector can be delivered via intravenous (IV) administration and can comprise a range of 1×10¹⁰ to 2×10¹⁴·vg/kg. In an aspect, a therapeutically effective amount of disclosed vector can be delivered via intra-cistem magna (ICM) administration and can comprise a range of 1×10⁹ to 2×10¹⁴·vg. In an aspect, a therapeutically effective amount of disclosed vector can be delivered via intrathecal (ITH) administration and can comprise a range of 1×10⁹ to 2×10¹⁴·vg. In an aspect, a therapeutically effective amount of disclosed vector can be delivered via intracerebroventricular (ICV) administration and can comprise a range of 1×10⁹ to 2×10¹⁴·vg. In an aspect, a therapeutically effective amount of a disclosed vector can comprise a single dose or a series of doses totalling the desired effective amount.

In an aspect, “CpG-free” can mean completely free of CpGs or partially free of CpGs. In an aspect, “CpG-free” can mean “CpG-depleted”. In an aspect, “CpG-depleted” can mean completely depleted of CpGs or partially depleted of CpGs. In an aspect, “CpG-free” can mean “CpG-optimized” for a desired and/or ideal expression level. CpG depletion and/or optimization is known to the skilled person in the art.

In an aspect, a mammalian cell can be a cell from any non-human species, such as, for example, a cell from a gorilla, a chimpanzee, a Rhesus monkey, a dog, a cow, a mouse, and a rat.

In an aspect, a disclosed vector can be a viral vector or a non-viral vector. In an aspect, a disclosed viral vector can be an adenovirus vector, an adeno-associated virus vector, a herpes simplex virus vector, a retrovirus vector, a lentivirus vector, and alphavirus vector, a flavivirus vector, a rhabdovirus vector, a measles virus vector, a Newcastle disease viral vector, a poxvirus vector, or a picomavirus vector. In an aspect, a disclosed non-viral vector can be a polymer based vector, a peptide based vector, a lipid nanoparticle, a solid lipid nanoparticle, or a cationic lipid based vector.

In an aspect, a disclosed viral vector can be an AAV vector. AAV vectors include, but are not limited to, AAV1, AAV2, AAV3 (including 3a and 3b), AAV4, AAV5, AAV6, AAV7, AAV8, AAVrh8, AAV9, AAV10, AAVrh10, AAV11, AAV12, AAV13, AAVrh39, AAVrh43, AAVcy.7 as well as bovine AAV, caprine AAV, canine AAV, equine AAV, ovine AAV, avian AAV, primate AAV, non-primate AAV, and any other virus classified by the International Committee on Taxonomy of Viruses (ICTV) as an AAV. In an aspect, AAV capsids can be chimeras either created by capsid evolution or by rational capsid engineering from the naturally isolated AAV variants to capture desirable serotype features such as enhanced or specific tissue tropism and host immune response escape, including but not limited to AAV-DJ, AAV-HAE1, AAV-HAE2, AAVM41, AAV-1829, AAV2 Y/F, AAV2 TN, AAV2i8, AAV2.5, AAV9.45, AAV9.61, AAV-B1, AAV-AS, AAV9.45A-String (e.g., AAV9.45-AS), AAV9.45Angiopep, AAV9.47-Angiopep, and AAV9.47-AS, AAV-PHP.B, AAV-PHP.eB, AAV-PHP.S, AAV-F, AAVcc.47, and AAVcc.81. In an aspect, an AAV vector can be AAV9, AAVF, AAVcc.47, or AAVcc.81. In an aspect, a disclosed AAV vector can be AAV-Rh74 or a related variant (e.g., capsid variants like RHM4-1).

In an aspect, a disclosed vector can comprise a ubiquitous promoter operably linked to the isolated nucleic acid molecule. In an aspect, the term “operably linked” means joined as part of the same nucleic acid molecule, suitably positioned and oriented for transcription to be initiated from the promoter. In an aspect, a disclosed ubiquitous promoter can drive the expression of the encoded polypeptide. In an aspect, a disclosed ubiquitous promoter can be a CMV enhancer/chicken β-actin (CB) promoter. In an aspect, a disclosed ubiquitous promoter can be a CpG-depleted murine CMV enhancer/human elongation factor-1 alpha promoter (mCMV/hEF1α).

In an aspect, a disclosed vector can comprise a tissue-specific promoter operably linked to the isolated nucleic acid molecule. In an aspect, the term “operably linked” means joined as part of the same nucleic acid molecule, suitably positioned and oriented for transcription to be initiated from the promoter. In an aspect, a disclosed tissue-specific promoter can be a liver-specific promoter, a muscle-specific promoter, a neuron-specific promoter (such as, for example, a synapsin I promoter), or a combination thereof. In an aspect, a disclosed liver-specific promoter can be α1-microglobulin/bikunin enhancer/thyroid hormone-binding globulin promoter.

In an aspect, a disclosed liver-specific promoter can comprise any liver-specific promoter known to the art. In an aspect, a liver-specific promoter can comprise about 845-bp and comprise the thyroid hormone-binding globulin promoter sequences (2382 to 13), two copies of α1-microglobulinybikunin enhancer sequences (22,804 through 22,704), and a 71-bp leader sequence as described by Ill C R, et al. (1997). In an aspect, a disclosed liver-specific promoter can comprise the sequence set froth in SEQ ID NO:34. In an aspect, a disclosed liver-specific promoter can comprise a sequence having at least 40%, 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:34. In an aspect, a disclosed liver-specific promoter can comprise a sequence having at least 40%-60%, at least 60%-80%, at least 80%-90%, or at least 90%-100% identity to the sequence set forth in SEQ ID NO:34.

In an aspect, a disclosed promoter can be an endogenous promoter. In an aspect, a disclosed endogenous promoter can generally be obtained from a non-coding region upstream of a transcription initiation site of a gene (such as, for example, a disclosed GBE or some other enzyme involved in the glycogen signaling pathway). In an aspect, a disclosed engoengous promoter can be used for constitutive and efficient expression of a disclosed transgene (e.g., a nucleic acid sequence encoding a polypeptide capable of preventing glycogen accumulation and/or degrading accumulated glycogen). The skilled person is familiar with the methods and tools to identify an endogenous promoter such as, for example, the endogenous promoter for GBE.

In an aspect, a disclosed vector can comprise an immunotolerant dual promoter comprising a liver-specific promoter and a ubiquitous promoter. In an aspect, an immunotolerant dual promoter can comprise a liver-specific promoter and another tissue specific promoter (such as, for example, a muscle-specific promoter, a neuron-specific promoter (such as, for example, a synapsin I promoter), a skeletal muscle-specific promoter, and a heart-specific promoter). In an aspect, a disclosed immunotolerant dual promoter can comprise a α1-microglobulin/bikunin enhancer/thyroid hormone-binding globulin promoter and a CB promoter. In an aspect, a disclosed immunotolerant dual promoter can comprise a al-microglobulin/bikunin enhancer/thyroid hormone-binding globulin promoter and a CpG-depleted mCMV/hEF1α promoter.

In an aspect, a disclosed immunotolerant dual promoter can comprise the sequence set forth in SEQ ID NO:05 or SEQ ID NO:06. In an aspect, a disclosed immunotolerant dual promoter can comprise a sequence having at least 40%, 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:05 or SEQ ID NO:06. In an aspect, a disclosed dual promoter can be engineered into a gene expression cassette such that the 3′ end of the liver-specific promoter is operably linked to the 5′ end of the ubiquitous promoter or the 3′ end of the ubiquitous promoter is operably linked to the 5′ end of the liver-specific promoter. Delivering a therapeutic gene under the control of a disclosed dual promoter has the surprising advantage of preventing a transgene-induced T cell response of a therapeutic transgene product for gene therapy of human genetic diseases that affect multiple tissues (such as GSD IV and/or APBD).

In an aspect, a disclosed viral vector can comprise a gene expression cassette comprising the one or more promoters, the isolated nucleic acid molecule comprising the CpG-depleted and codon-optimized nucleic acid sequence encoding the polypeptide, and a polyadenylation sequence. In an aspect, the nucleic acid sequence can have a coding sequence that is less than about 4.5 kilobases.

Disclosed herein is an AAV vector comprising an isolated nucleic acid molecule, wherein the isolated nucleic acid sequence encodes a human glycogen branching enzyme, and wherein the isolated nucleic acid sequence is CpG-depleted and codon-optimized for expression in a human or a mammalian cell. In an aspect, a mammalian cell can be a cell from any non-human species, such as, for example, a cell from a gorilla, a chimpanzee, a Rhesus monkey, a dog, a cow, a mouse, and a rat.

In an aspect, a disclosed human glycogen branching enzyme can comprise the sequence set forth in SEQ ID NO:01. In an aspect, a disclosed human glycogen branching enzyme can comprise a sequence having at least 30%, at least 40%, at least 50%, at least 60%, at least 70%, at least 80%, or at least 90% identity to the sequence set forth in SEQ ID NO:01.

In an aspect, a disclosed human glycogen branching enzyme can comprise the sequence set forth in SEQ ID NO:03 or SEQ ID NO:04. In an aspect, a disclosed human glycogen branching enzyme can comprise a sequence having at least 30%, at least 40%, at least 50%, at least 60%, at least 70%, at least 80%, or at least 90% identity to the sequence set forth in SEQ ID NO:03 or SEQ ID NO:04. In an aspect, a disclosed human glycogen branching enzyme can comprise a sequence having at least 50% identity to the sequence set forth in SEQ ID NO:03, or SEQ ID NO:04. In an aspect, a disclosed nucleic acid sequence can comprise a sequence having at least 80% identity to the sequence set forth in SEQ ID NO:03 or SEQ ID NO:04. In an aspect, the original (non-CpG-depleted) polynucleotide open reading frame (ORF) sequence of a disclosed human glycogen branching enzyme can comprise the sequence set forth in SEQ ID NO:2. In an aspect, the original (non-CpG-depleted) polynucleotide open reading frame (ORF) sequence of a disclosed human glycogen branching enzyme can comprise a sequence having at least 50-69%, at least 70-89%, or at least 90-99% identity to the sequence set forth in SEQ ID NO:02. In an aspect, a mammalian cell can be a cell from any non-human species, such as, for example, a cell from a gorilla, a chimpanzee, a Rhesus monkey, a dog, a cow, a mouse, and a rat.

In an aspect, a disclosed vector can comprise a ubiquitous promoter. In an aspect, a ubiquitous promoter can be a CB promoter or a CpG-depleted mCMV/hEF1α promoter. In an aspect, a disclosed vector can comprise a tissue-specific promoter. In an aspect, a tissue-specific promoter can be a liver-specific promoter, a muscle-specific promoter, a neuron-specific promoter (such as, for example, a synapsin I promoter), or a combination thereof.

In an aspect, a disclosed liver-specific promoter can comprise any liver-specific promoter known to the art. In an aspect, a liver-specific promoter can be a α1-microglobulin/bikunin enhancer/thyroid hormone-binding globulin promoter. In an aspect, a liver specific promoter can comprise about 845-bp and comprise the thyroid hormone-binding globulin promoter sequences (2382 to 13), two copies of α1-microglobulinybikunin enhancer sequences (22,804 through 22,704), and a 71-bp leader sequence as described by Ill C R, et al. (1997). In an aspect, a disclosed liver-specific promoter can comprise the sequence set froth in SEQ ID NO:34. In an aspect, a disclosed liver-specific promoter can comprise a sequence having at least 40%, 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:34. In an aspect, a disclosed liver-specific promoter can comprise a sequence having at least 40%-60%, at least 60%-80%, at least 80%-90%, or at least 90%-100% identity to the sequence set forth in SEQ ID NO:34.

In an aspect, a disclosed promoter can be an endogenous promoter. In an aspect, a disclosed endogenous promoter can generally be obtained from a non-coding region upstream of a transcription initiation site of a gene (such as, for example, a disclosed GBE or some other enzyme involved in the glycogen signaling pathway). In an aspect, a disclosed engoengous promoter can be used for constitutive and efficient expression of a disclosed transgene (e.g., a nucleic acid sequence encoding a polypeptide capable of preventing glycogen accumulation and/or degrading accumulated glycogen). The skilled person is familiar with the methods and tools to identify an endogenous promoter such as, for example, the endogenous promoter for GBE.

In an aspect, a disclosed vector can comprise an immunotolerant dual promoter comprising a liver-specific promoter and a ubiquitous promoter. In an aspect, an immunotolerant dual promoter can comprise a liver-specific promoter and another tissue specific promoter (such as, for example, a muscle-specific promoter, a neuron-specific promoter (such as, for example, a synapsin I promoter), a skeletal muscle-specific promoter, and a heart-specific promoter). In an aspect, an immunotolerant dual promoter can comprise a al-microglobulin/bikunin enhancer/thyroid hormone-binding globulin promoter and a CB promoter. In an aspect, an immunotolerant dual promoter can comprise a α1-microglobulin/bikunin enhancer/thyroid hormone-binding globulin promoter and a CpG-depleted mCMV/hEF1α promoter. In an aspect, an immunotolerant dual promoter can comprise the nucleic acid sequence set forth in SEQ ID NO:05 or SEQ ID NO:06. In an aspect, an immunotolerant dual promoter can comprise a nucleic acid sequence having at least 40%, 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:05 or SEQ ID NO:06. In an aspect of a disclosed vector, the isolated nucleic acid molecule can have a nucleotide sequence having about 4.5 kb or less.

Disclosed herein is vector comprising a gene expression cassette comprising an isolated nucleic acid molecule comprising a nucleic acid sequence encoding a polypeptide for preventing glycogen accumulation and/or degrading accumulated glycogen under the control of a ubiquitous promoter, a tissue-specific promoter, or an immunotolerant dual promoter comprising a liver-specific promoter and a ubiquitous promoter, wherein the nucleic acid sequence is CpG-depleted and codon-optimized for expression in a human or a mammalian cell. In an aspect, a mammalian cell can be a cell from any non-human species, such as, for example, a cell from a gorilla, a chimpanzee, a Rhesus monkey, a dog, a cow, a mouse, and a rat.

In an aspect, a disclosed vector can be a viral vector or a non-viral vector. In an aspect, a disclosed viral vector can be an adenovirus vector, an adeno-associated virus vector, a herpes simplex virus vector, a retrovirus vector, a lentivirus vector, and alphavirus vector, a flavivirus vector, a rhabdovirus vector, a measles virus vector, a Newcastle disease viral vector, a poxvirus vector, or a picomavirus vector. In an aspect, a disclosed non-viral vector can be a polymer based vector, a peptide based vector, a lipid nanoparticle, a solid lipid nanoparticle, or a cationic lipid based vector.

In an aspect, a disclosed viral vector can be an AAV vector. AAV vectors include, but are not limited to, AAV1, AAV2, AAV3 (including 3a and 3b), AAV4, AAV5, AAV6, AAV7, AAV8, AAVrh8, AAV9, AAV10, AAVrh10, AAV11, AAV12, AAV13, AAVrh39, AAVrh43, AAVcy.7 as well as bovine AAV, caprine AAV, canine AAV, equine AAV, ovine AAV, avian AAV, primate AAV, non-primate AAV, and any other virus classified by the International Committee on Taxonomy of Viruses (ICTV) as an AAV. In an aspect, AAV capsids can be chimeras either created by capsid evolution or by rational capsid engineering from the naturally isolated AAV variants to capture desirable serotype features such as enhanced or specific tissue tropism and host immune response escape, including but not limited to AAV-DJ, AAV-HAE1, AAV-HAE2, AAVM41, AAV-1829, AAV2 Y/F, AAV2 T/V, AAV2i8, AAV2.5, AAV9.45, AAV9.61, AAV-B1, AAV-AS, AAV9.45A-String (e.g., AAV9.45-AS), AAV9.45Angiopep, AAV9.47-Angiopep, and AAV9.47-AS, AAV-PHP.B, AAV-PHP.eB, AAV-PHP.S, AAV-F, AAVcc.47, and AAVcc.81. In an aspect, an AAV vector can be AAV9, AAVF, AAVcc.47, or AAVcc.81. In an aspect, a disclosed AAV vector can be AAV-Rh74 or a related variant (e.g., capsid variants like RHM4-1).

In an aspect, a disclosed ubiquitous promoter can be operably linked to the isolated nucleic acid molecule. In an aspect, a disclosed ubiquitous promoter can be a CMV enhancer/chicken β-actin promoter.

In an aspect, a disclosed dual promoter can be an immunotolerant dual promoter. In an aspect, an immunotolerant dual promoter can comprise a liver-specific promoter and another tissue specific promoter (such as, for example, a muscle-specific promoter, a neuron-specific promoter (such as, for example, a synapsin I promoter), a skeletal muscle-specific promoter, and a heart-specific promoter). In an aspect, a disclosed immunotolerant dual promoter can comprise a α1-microglobulin/bikunin enhancer/thyroid hormone-binding globulin promoter and a CMV enhancer/beta-actin (CB) promoter. In an aspect, a disclosed immunotolerant dual promoter can comprise the nucleic acid sequence set forth in SEQ ID NO:05 or SEQ ID NO:06. In an aspect, a disclosed immunotolerant dual promoter can comprise a nucleic acid sequence having at least 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:05 or SEQ ID NO:06.

In an aspect, a disclosed encoded polypeptide can degrade insoluble amylopectin-like glycogen, Lafora bodies, polyglucosan bodies, or any form of accumulated glycogen. In an aspect, a disclosed encoded polypeptide can be derived from plant, bacteria, or another microorganism. In an aspect, a disclosed encoded polypeptide can be derived from any non-human species, such as, for example, gorilla, chimpanzee, Rhesus monkey, dog, cow, mouse, rat, chicken, zebrafish, fruit fly, mosquito, C. elegans, and frog. In an aspect, a disclosed encoded polypeptide can be a human glycogen branching enzyme. In an aspect, a disclosed encoded polypeptide can be a human salivary or pancreatic amylase. In an aspect, a disclosed encoded polypeptide can comprise the sequence set forth in SEQ ID NO:01. In an aspect, a disclosed encoded polypeptide can comprise a sequence having at least 50% or at least 90% identity to the sequence set forth in SEQ ID NO:01.

In an aspect, a disclosed nucleic acid sequence encoding a polypeptide can comprise the sequence set forth in SEQ ID NO:03 or SEQ ID NO:04. In an aspect, human glycogen branching enzyme can comprise a sequence having at least 30%, at least 40%, at least 50%, at least 60%, at least 70%, at least 80%, or at least 90% identity to the sequence set forth in SEQ ID NO:03 or SEQ ID NO:04. In an aspect, a disclosed nucleic acid sequence encoding a polypeptide can comprise a sequence having at least 50% identity to the sequence set forth in SEQ ID NO:03, or SEQ ID NO:04. In an aspect, a disclosed nucleic acid sequence can comprise a sequence having at least 80% identity to the sequence set forth in SEQ ID NO:03 or SEQ ID NO:04. In an aspect, a disclosed nucleic acid sequence can comprise a sequence having at least 50-69%, at least 70-89%, or at least 90-99% identity to the sequence set forth in SEQ ID NO:02. In an aspect, a disclosed isolated nucleic acid molecule can have a nucleotide sequence having about 4.5 kb or less.

### 3. Formulations

Disclosed herein is a pharmaceutical formulation comprising a disclosed vector or a disclosed isolated nucleic acid molecule.

Disclosed herein is a pharmaceutical formulation comprising a vector comprising an isolated nucleic acid molecule comprising a nucleic acid sequence encoding a polypeptide for preventing glycogen accumulation and/or degrading accumulated glycogen, wherein the nucleic acid sequence is CpG-depleted and codon-optimized for expression in a human or a mammalian cell, wherein the vector is in a pharmaceutically acceptable carrier.

Disclosed herein is a pharmaceutical formulation comprising a disclosed isolated nucleic acid molecule comprising a nucleic acid sequence encoding a polypeptide for preventing glycogen accumulation and/or degrading accumulated glycogen, wherein the nucleic acid sequence is CpG-depleted and codon-optimized for expression in a human or a mammalian cell, wherein the vector is in a pharmaceutically acceptable carrier.

In an aspect, a mammalian cell can be a cell from any non-human species, such as, for example, a cell from a gorilla, a chimpanzee, a Rhesus monkey, a dog, a cow, a mouse, and a rat.

In an aspect, a disclosed formulation can comprise (i) one or more active agents, (ii) biologically active agents, (iii) one or more pharmaceutically active agents, (iv) one or more immune-based therapeutic agents, (v) one or more clinically approved agents, or (vi) a combination thereof. In an aspect, a disclosed composition can comprise one or more proteasome inhibitors. In an aspect, a disclosed composition can comprise one or more immunosuppressives or immunosuppressive agents. In an aspect, an immunosuppressive agent can be anti-thymocyte globulin (ATG), cyclosporine (CSP), mycophenolate mofetil (MMF), or a combination thereof. In an aspect, a disclosed formulation can comprise an anaplerotic agent (such as, for example, C7 compounds like triheptanoin).

In an aspect, a disclosed formulation can comprise a RNA therapeutic. A RNA therapeutic can comprise RNA-mediated interference (RNAi) and/or antisense oligonucleotides (ASO). In an aspect, a disclosed RNA therapeutic can be directed at GYS1 and/or GYS2. A RNA therapeutic can comprise therapy delivered via LNPs.

In an aspect, a disclosed formulation can comprise a disclosed small molecule. In an aspect, a disclosed small molecule can inhibit and/or reduce the expression level and/or the activity level of glycogen synthase. In an aspect, a disclosed small molecule can, for example, inhibit glycogen synthase (i.e., GYS1 and/or GYS2) in a cell or a subject to reduce glycogen synthesis and/or glycogen accumulation in cells and tissues (e.g., skeletal muscle, lung tissue, liver tissue, brain tissue, or any other tissue having glycogen accumulation) when GAA and/or GBE activity and/or expression levels are reduced (e.g., SRT). In an aspect, a disclosed small molecule can be guaiacol. In an aspect, a disclosed small molecule that inhibits glycogen synthase (GYS1) can be BBB permeable or BBB non-permeable.

In an aspect, a disclosed formulation can comprise an inhibitor of phosphorylation. For example, a disclosed formulation can comprise a modulator of the enzyme activity of GYS1 whereby the modulator acts through inhibitory phosphorylation (e.g., reduced phosphorylation of GYS1 kinase AMPK).

In an aspect, a disclosed formulation can comprise an enzyme or enzyme precursor for enzyme replacement therapy (ERT).

### 4. Plasmids

Disclosed herein is a plasmid comprising a nucleic acid sequence encoding a CMV enhancer/chicken β-actin (CB) promoter and mGBE. In an aspect, a plasmid comprising a nucleic acid sequence encoding a CB promoter and mGBE can comprise the sequence set forth in SEQ ID NO:23 or a sequence having at least 30%, at least 40%, at least 50%, at least 60%, at least 70%, at least 80%, or at least 90% identity to the sequence set forth in SEQ ID NO:23 or a sequence having at least 40-59%, at least 50-69%, or at least 80-99% identity to the sequence set forth in SEQ ID NO:23.

Disclosed herein is a plasmid comprising a nucleic acid sequence encoding a CMV enhancer/chicken β-actin (CB) promoter and hGBE. In an aspect, a plasmid comprising a nucleic acid sequence encoding a CB promoter and hGBE can comprise the sequence set forth in SEQ ID NO:24 or a sequence having at least 30%, at least 40%, at least 50%, at least 60%, at least 70%, at least 80%, or at least 90% identity to the sequence set forth in SEQ ID NO:24 or a sequence having at least 40-59%, at least 50-69%, or at least 80-99% identity to the sequence set forth in SEQ ID NO:24.

Disclosed herein is a plasmid comprising a nucleic acid sequence encoding a tandem LSP-CB fusion promoter and hGBE. In an aspect, a plasmid comprising a nucleic acid sequence encoding a tandem LSP-CB fusion promoter and hGBE can comprise the sequence set forth in SEQ ID NO:25 or a sequence having at least 30%, at least 40%, at least 50%, at least 60%, at least 70%, at least 80%, or at least 90% identity to the sequence set forth in SEQ ID NO:25 or a sequence having at least 40-59%, at least 50-69%, or at least 80-99% identity to the sequence set forth in SEQ ID NO:25.

Disclosed herein is a plasmid comprising a nucleic acid sequence encoding a tandem LSP-hEF1α fusion promoter and hGBE^{CpG-free}. In an aspect, a plasmid comprising a nucleic acid sequence encoding a tandem LSP-hEF1α fusion promoter and hGBE^{CpG-free} can comprise the sequence set forth in SEQ ID NO:26 or a sequence having at least 30%, at least 40%, at least 50%, at least 60%, at least 70%, at least 80%, or at least 90% identity to the sequence set forth in SEQ ID NO:26 or a sequence having at least 40-59%, at least 50-69%, or at least 80-99% identity to the sequence set forth in SEQ ID NO:26.

Disclosed herein is a plasmid comprising a nucleic acid sequence encoding a synapsin promoter and hGBE^{CpG-free}-WPRE. In an aspect, a plasmid comprising a nucleic acid sequence encoding a synapsin promoter and hGBE^{CpG-free}-WPRE. can comprise the sequence set forth in SEQ ID NO:27 or a sequence having at least 30%, at least 40%, at least 50%, at least 60%, at least 70%, at least 80%, or at least 90% identity to the sequence set forth in SEQ ID NO:27 or a sequence having at least 40-59%, at least 50-69%, or at least 80-99% identity to the sequence set forth in SEQ ID NO:27.

Disclosed herein is a plasmid comprising a nucleic acid sequence encoding a hEF1α promoter and hGBE^{CpG-free}. In an aspect, a plasmid comprising a nucleic acid sequence encoding a hEF1α promoter and hGBE^{CpG-free} can comprise the sequence set forth in SEQ ID NO:30 or a sequence having at least 30%, at least 40%, at least 50%, at least 60%, at least 70%, at least 80%, or at least 90% identity to the sequence set forth in SEQ ID NO:30 or a sequence having at least 40-59%, at least 50-69%, or at least 80-99% identity to the sequence set forth in SEQ ID NO:30.

Disclosed herein is a plasmid comprising a nucleic acid sequence encoding a CB promoter and hGBE^{CpG-free}. In an aspect, a plasmid comprising a nucleic acid sequence encoding a CB promoter and hGBE^{CpG-free} can comprise the sequence set forth in SEQ ID NO:31 or a sequence having at least 30%, at least 40%, at least 50%, at least 60%, at least 70%, at least 80%, or at least 90% identity to the sequence set forth in SEQ ID NO:31 or a sequence having at least 40-59%, at least 50-69%, or at least 80-99% identity to the sequence set forth in SEQ ID NO:31.

Disclosed herein is a plasmid comprising a nucleic acid sequence encoding a tandem LSP-hEF1α fusion promoter and hGBE^{CpG-free}. In an aspect, a plasmid comprising a nucleic acid sequence encoding a tandem LSP-hEF1α fusion promoter and hGBE^{CpG-free} can comprise the sequence set forth in SEQ ID NO:32 or a sequence having at least 30%, at least 40%, at least 50%, at least 60%, at least 70%, at least 80%, or at least 90% identity to the sequence set forth in SEQ ID NO:32 or a sequence having at least 40-59%, at least 50-69%, or at least 80-99% identity to the sequence set forth in SEQ ID NO:32.

Disclosed herein is a plasmid comprising a nucleic acid sequence encoding a tandem LSP-CB fusion promoter and hGBE^{CpG-free}. In an aspect, a plasmid comprising a nucleic acid sequence encoding a tandem LSP-CB fusion promoter and hGBE can comprise the sequence set forth in SEQ ID NO:33 or a sequence having at least 30%, at least 40%, at least 50%, at least 60%, at least 70%, at least 80%, or at least 90% identity to the sequence set forth in SEQ ID NO:33 or a sequence having at least 40-59%, at least 50-69%, or at least 80-99% identity to the sequence set forth in SEQ ID NO:33.

### 5. Cells

Disclosed herein are cells comprising a disclosed isolated nucleic acid molecule, a disclosed vector, and/or a disclosed plasmid. Cells are known to the art. In an aspect, a disclosed cell can comprise the plasmid set forth in any one of SEQ ID NO:23-SEQ ID NO:27 or in any one of SEQ ID NO:30-SEQ ID NO:33.

### 6. Animals

Disclosed herein are animals treated with one or more disclosed isolated nucleic acid molecules, disclosed vectors, disclosed pharmaceutical formulations, and/or disclosed plasmids. Cells are known to the art. In an aspect, a disclosed animal has been treated with a vector comprising the plasmid set forth in any one of SEQ ID NO:23-SEQ ID NO:27 or SEQ ID NO:30-SEQ ID NO:33.

### C. Methods for Treating and/or Preventing GSD IV and/or APBD Disease Progression

Disclosed herein is a method of treating and/or preventing GSD IV and/or APBD disease progression comprising administering to a subject in need thereof a therapeutically effective amount of a disclosed isolated nucleic acid molecule, a disclosed vector, disclosed pharmaceutical formulation, or a combination thereof.

Disclosed herein is a method of treating and/or preventing GSD IV and/or APBD disease progression comprising administering to a subject in need thereof a therapeutically effective amount of a disclosed isolated nucleic acid molecule, a disclosed vector, disclosed pharmaceutical formulation, or a combination thereof, and restoring the level of glycogen synthase (GYS1) and/or GBE to normal or near normal in the subject or in a tissue and/or organ in the subject.

Disclosed herein is a method of treating and/or preventing GSD IV and/or APBD disease progression comprising administering to a subject in need thereof a therapeutically effective amount of a vector comprising an isolated nucleic acid molecule comprising a nucleic acid sequence encoding a polypeptide for preventing glycogen accumulation and/or degrading accumulated glycogen, wherein the nucleic acid sequence is CpG-depleted and codon-optimized for expression in a human or a mammalian cell, wherein glycogen accumulation is prevented and/or accumulated glycogen is degraded in the subject.

Disclosed herein is a method of treating and/or preventing GSD IV and/or APBD disease progression comprising preventing glycogen accumulation and/or degrading accumulated glycogen in a subject in need thereof by administering to the subject a therapeutically effective amount of a vector comprising an isolated nucleic acid molecule comprising a nucleic acid sequence encoding a polypeptide for preventing glycogen accumulation and/or degrading accumulated glycogen, wherein the nucleic acid sequence is CpG-depleted and codon-optimized for expression in a human or a mammalian cell.

Disclosed herein is a method of treating and/or preventing GSD IV and/or APBD disease progression comprising administering to a subject in need thereof a therapeutically effective amount of a vector comprising an isolated nucleic acid molecule comprising a nucleic acid sequence encoding a polypeptide for preventing glycogen accumulation and/or degrading accumulated glycogen, wherein the nucleic acid sequence is CpG-depleted and codon-optimized for expression in a human or a mammalian cell, thereby preventing glycogen accumulation and/or degrading accumulated glycogen in the subject.

Disclosed herein is a method of preventing glycogen accumulation and/or degrading accumulated glycogen comprising administering to a subject having GSD IV a therapeutically effective amount of a vector comprising an isolated nucleic acid molecule comprising a nucleic acid sequence encoding a polypeptide for preventing glycogen accumulation and/or degrading accumulated glycogen, wherein the nucleic acid sequence is CpG-depleted and codon-optimized for expression in a human or a mammalian cell.

Disclosed herein is a method of treating and/or preventing GSD IV and/or APBD disease progression comprising administering to a subject in need thereof a therapeutically effective amount of a disclosed isolated nucleic acid molecule, a disclosed vector, and/or a disclosed pharmaceutical formulation, and a therapeutically effective amount of an agent for reducing the expression level and/or activity level of glycogen synthase.

Disclosed herein is a method of treating and/or preventing GSD IV and/or APBD disease progression comprising administering to a subject in need thereof a therapeutically effective amount of a vector comprising an isolated nucleic acid molecule comprising a nucleic acid sequence encoding a polypeptide for preventing glycogen accumulation and/or degrading accumulated glycogen, wherein the nucleic acid sequence is CpG-depleted and codon-optimized for expression in a human or a mammalian cell, a therapeutically effective amount of an agent for reducing the expression level and/or activity level of glycogen synthase, wherein glycogen accumulation is prevented and/or accumulated glycogen is degraded in the subject.

Disclosed herein is a method of treating and/or preventing GSD IV and/or APBD disease progression comprising preventing glycogen accumulation and/or degrading accumulated glycogen in a subject in need thereof by administering to the subject a therapeutically effective amount of a vector comprising an isolated nucleic acid molecule comprising a nucleic acid sequence encoding a polypeptide for preventing glycogen accumulation and/or degrading accumulated glycogen, wherein the nucleic acid sequence is CpG-depleted and codon-optimized for expression in a human or a mammalian cell, and a therapeutically effective amount of an agent for reducing the expression level and/or activity level of glycogen synthase.

Disclosed herein is a method of treating and/or preventing GSD IV and/or APBD disease progression comprising administering to a subject in need thereof a therapeutically effective amount of a vector comprising an isolated nucleic acid molecule comprising a nucleic acid sequence encoding a polypeptide for preventing glycogen accumulation and/or degrading accumulated glycogen, wherein the nucleic acid sequence is CpG-depleted and codon-optimized for expression in a human or a mammalian cell, and a therapeutically effective amount of an agent for reducing the expression level and/or activity level of glycogen synthase, thereby preventing glycogen accumulation and/or degrading accumulated glycogen in the subject.

Disclosed herein is a method of preventing glycogen accumulation and/or degrading accumulated glycogen comprising administering to a subject having GSD IV disease progression a therapeutically effective amount of a vector comprising an isolated nucleic acid molecule comprising a nucleic acid sequence encoding a polypeptide for preventing glycogen accumulation and/or degrading accumulated glycogen, wherein the nucleic acid sequence is CpG-depleted and codon-optimized for expression in a human or a mammalian cell, and a therapeutically effective amount of an agent for reducing the expression level and/or activity level of glycogen synthase.

In an aspect, the encoded polypeptide can degrade glycogen, polyglucosan bodies, amylopectin-like glycogen, Lafora bodies, or any combination thereof. In an aspect, the encoded polypeptide can degrade any form of accumulated glycogen.

In an aspect, “glycogen” can refer to glycogen, polyglucosan bodies, amylopectin-like glycogen, or any combination thereof. For example, the term “glycogen accumulation” can comprise accumulation of polyglucosan bodies and/or amylopectin-like glycogen in addition to the accumulation of glycogen. In an aspect, accumulation can refer to accumulation of glycogen, polyglucosan bodies, amylopectin-like glycogen, or any combination thereof.

In an aspect, a disclosed encoded polypeptide can be a human glycogen branching enzyme. In an aspect, a disclosed encoded polypeptide can be a human salivary or pancreatic amylase. In an aspect, a disclosed encoded polypeptide can be derived from plant, bacteria, or another microorganism. In an aspect, a disclosed encoded polypeptide can be derived from any non-human species, such as, for example, gorilla, chimpanzee, Rhesus monkey, dog, cow, mouse, rat, chicken, zebrafish, fruit fly, mosquito, C. elegans, and frog. In an aspect, a mammalian cell can be a cell from any non-human species, such as, for example, a cell from a gorilla, a chimpanzee, a Rhesus monkey, a dog, a cow, a mouse, and a rat.

In an aspect, a disclosed encoded polypeptide can comprise the sequence set forth in SEQ ID NO:01. In an aspect, a disclosed encoded polypeptide can comprise a sequence having at least 30%, at least 40%, at least 50%, at least 60%, at least 70%, at least 80%, or at least 90% identity to the sequence set forth in SEQ ID NO:01. In an aspect, a disclosed encoded polypeptide can comprise the sequence set forth in SEQ ID NO:07. In an aspect, a disclosed encoded polypeptide can comprise a sequence having at least 30%, at least 40%, at least 50%, at least 60%, at least 70%, at least 80%, or at least 90% identity to the sequence set forth in SEQ ID NO:07. In an aspect, a disclosed encoded polypeptide can comprise the sequence set forth in SEQ ID NO:08. In an aspect, a disclosed encoded polypeptide can comprise a sequence having at least 30%, at least 40%, at least 50%, at least 60%, at least 70%, at least 80%, or at least 90% identity to the sequence set forth in SEQ ID NO:08.

In an aspect, a disclosed nucleic acid sequence encoding a polypeptide can comprise the sequence set forth in SEQ ID NO:03 or SEQ ID NO:04. In an aspect, a disclosed nucleic acid sequence encoding a polypeptide can comprise a sequence having at least 30%, at least 40%, at least 50%, at least 60%, at least 70%, at least 80%, or at least 90% identity to the sequence set forth in SEQ ID NO:03 or SEQ ID NO:04. In an aspect, a disclosed nucleic acid sequence can comprise a sequence having at least 80% identity to the sequence set forth in SEQ ID NO:03 or SEQ ID NO:04. In an aspect, the original (non-CpG-depleted) polynucleotide open reading frame (ORF) sequence of human glycogen branching enzyme can comprise the sequence set forth in SEQ ID NO:2. In an aspect, the original (non-CpG-depleted) polynucleotide open reading frame (ORF) sequence of human glycogen branching enzyme can comprise a sequence having at least 50-69%, at least 70-89%, or at least 90-99% identity to the sequence set forth in SEQ ID NO:02.

In an aspect, a disclosed vector can be a viral vector or non-viral vector. In an aspect, a disclosed viral vector can be an adenovirus vector, an adeno-associated virus vector, a herpes simplex virus vector, a retrovirus vector, a lentivirus vector, and alphavirus vector, a flavivirus vector, a rhabdovirus vector, a measles virus vector, a Newcastle disease viral vector, a poxvirus vector, or a picomavirus vector. In an aspect, a disclosed non-viral vector can be a polymer-based vector, a peptide-based vector, a lipid nanoparticle, a solid lipid nanoparticle, or a cationic lipid based vector.

In an aspect, a disclosed viral vector can be an AAV vector. AAV vectors include, but are not limited to, AAV1, AAV2, AAV3 (including 3a and 3b), AAV4, AAV5, AAV6, AAV7, AAV8, AAVrh8, AAV9, AAV10, AAVrh10, AAV11, AAV12, AAV13, AAVrh39, AAVrh43, AAVcy.7 as well as bovine AAV, caprine AAV, canine AAV, equine AAV, ovine AAV, avian AAV, primate AAV, non-primate AAV, and any other virus classified by the International Committee on Taxonomy of Viruses (ICTV) as an AAV. In an aspect, AAV capsids can be chimeras either created by capsid evolution or by rational capsid engineering from the naturally isolated AAV variants to capture desirable serotype features such as enhanced or specific tissue tropism and host immune response escape, including but not limited to AAV-DJ, AAV-HAE1, AAV-HAE2, AAVM41, AAV-1829, AAV2 Y/F, AAV2 TN, AAV2i8, AAV2.5, AAV9.45, AAV9.61, AAV-B1, AAV-AS, AAV9.45A-String (e.g., AAV9.45-AS), AAV9.45Angiopep, AAV9.47-Angiopep, and AAV9.47-AS, AAV-PHP.B, AAV-PHP.eB, AAV-PHP.S, AAV-F, AAVcc.47, and AAVcc.81. In an aspect, an AAV vector can be AAV9, AAVF, AAVcc.47, or AAVcc.81. In an aspect, a disclosed AAV vector can be AAV-Rh74 or a related variant (e.g., capsid variants like RHM4-1).

In an aspect, a disclosed vector can be administered via intravenous, intraarterial, intramuscular, intraperitoneal, subcutaneous, intra-CSF, intrathecal, intraventricular, or in utero administration. In an aspect, a disclosed vector can be administered via intra-CSF administration in combination with a disclosed nucleic acid molecule, a disclosed vector, and/or a disclosed pharmaceutical formulation. In an aspect, a disclosed vector can be administered via intra-CSF administration in combination with RNAi, antisense oligonucleotides, miRNA, one or more small molecules, one or more therapeutic agents, one or more proteasome inhibitors, one or more immune modulators, and/or a gene editing system. In an aspect, a disclosed vector can be administered via LNP administration. In an aspect, a subject can be a human subject. In an aspect, a disclosed vector can be delivered to the subject's liver, heart, skeletal muscle, smooth muscle, CNS, PNS, or a combination thereof. In an aspect, a disclosed vector can be concurrently and/or serially administered to a subject via multiple routes of administration. For example, in an aspect, administering a disclosed vector can comprise intravenous administration and intra-cistern magna (ICM) administration. In an aspect, administering a disclosed vector can comprise IV administration and intrathecal (ITH) administration.

In an aspect, a therapeutically effective amount of disclosed vector can be delivered via intravenous (IV) administration and can comprise a range of 1×10¹⁰ to 2×10¹⁴·vg/kg. In an aspect, a therapeutically effective amount of disclosed vector can be delivered via intra-cistem magna (ICM) administration and can comprise a range of 1×10⁹ to 2×10¹⁴·vg. In an aspect, a therapeutically effective amount of disclosed vector can be delivered via intrathecal (ITH) administration and can comprise a range of 1×10⁹ to 2×10¹⁴·vg. In an aspect, a therapeutically effective amount of disclosed vector can be delivered via intracerebroventricular (ICV) administration and can comprise a range of 1×10⁹ to 2×10¹⁴·vg. In an aspect, a therapeutically effective amount of a disclosed vector can comprise a single dose or a series of doses totalling the desired effective amount.

In an aspect, a disclosed vector can comprise a ubiquitous promoter. In an aspect, a disclosed ubiquitous promoter can be a CMV enhancer/chicken β-actin promoter or a CpG-depleted mCMV/hEF1α promoter. In an aspect, a disclosed vector can comprise a tissue-specific promoter. In an aspect, a disclosed tissue-specific promoter can be a liver-specific promoter, a muscle-specific promoter, a neuron-specific promoter (such as, for example, a synapsin I promoter), or a combination thereof. In an aspect, a disclosed liver-specific promoter can be a α1-microglobulin/bikunin enhancer/thyroid hormone-binding globulin promoter.

In an aspect, a disclosed liver-specific promoter can comprise any liver-specific promoter known to the art. In an aspect, a liver specific promoter can comprise about 845-bp and comprise the thyroid hormone-binding globulin promoter sequences (2382 to 13), two copies of α1-microglobulinybikunin enhancer sequences (22,804 through 22,704), and a 71-bp leader sequence as described by Ill C R, et al. (1997). In an aspect, a disclosed liver-specific promoter can comprise the sequence set froth in SEQ ID NO:34. In an aspect, a disclosed liver-specific promoter can comprise a sequence having at least 40%, 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:34. In an aspect, a disclosed liver-specific promoter can comprise a sequence having at least 40%-60%, at least 60%-80%, at least 80%-90%, or at least 90%-100% identity to the sequence set forth in SEQ ID NO:34.

In an aspect, a disclosed promoter can be an endogenous promoter. In an aspect, a disclosed endogenous promoter can generally be obtained from a non-coding region upstream of a transcription initiation site of a gene (such as, for example, a disclosed GBE or some other enzyme involved in the glycogen signaling pathway). In an aspect, a disclosed engoengous promoter can be used for constitutive and efficient expression of a disclosed transgene (e.g., a nucleic acid sequence encoding a polypeptide capable of preventing glycogen accumulation and/or degrading accumulated glycogen). The skilled person is familiar with the methods and tools to identify an endogenous promoter such as, for example, the endogenous promoter for GBE.

In an aspect, a disclosed vector can comprise an immunotolerant dual promoter comprising a liver-specific promoter and a ubiquitous promoter. In an aspect, an immunotolerant dual promoter can comprise a liver-specific promoter and another tissue specific promoter (such as, for example, a muscle-specific promoter, a neuron-specific promoter (such as, for example, a synapsin I promoter), a skeletal muscle-specific promoter, and a heart-specific promoter). In an aspect, a disclosed immunotolerant dual promoter can comprise a α1-microglobulin/bikunin enhancer/thyroid hormone-binding globulin promoter and a CB promoter. In an aspect, a disclosed immunotolerant dual promoter can comprise a al-microglobulin/bikunin enhancer/thyroid hormone-binding globulin promoter and a CpG-depleted mCMV/hEF1α promoter. In an aspect, a disclosed immunotolerant dual promoter can comprise the sequence set forth in SEQ ID NO:05 or SEQ ID NO:06. In an aspect, a disclosed immunotolerant dual promoter can comprise a sequence having at least 40%, 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:05 or SEQ ID NO:06.

In an aspect, a disclosed method can comprise administering a oligonucleotide therapeutic agent. A disclosed oligonucleotide therapeutic agent can comprise a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed oligonucleotide therapeutic agent can be an ASO or an RNAi. In an aspect, a disclosed oligonucleotide therapeutic agent can comprise one or more modifications at any position applicable.

In an aspect, a disclosed oligonucleotide therapeutic agent can comprise a CRISPR-based endonuclease. In an aspect, a disclosed endonuclease can be Cas9. In an aspect, a disclosed Cas9 can be from Staphylococcus aureus or Streptococcus pyogenes. Cas9 can have the sequence set forth in SEQ ID NO:09 or a fragment thereof. In an aspect, a disclosed Cas9 can have a sequence having at least 75%, at least 80%, at least 85%, at least 90%, or at least 95% identity to the sequence set forth in SEQ ID NO:09 or a fragment thereof. Cas9 can have the sequence set forth in SEQ ID NO:10 or a fragment thereof. In an aspect, a disclosed Cas9 can have a sequence having at least 75%, at least 80%, at least 85%, at least 90%, or at least 95% identity to the sequence set forth in SEQ ID NO:10 or a fragment thereof. In an aspect, a disclosed nucleic acid sequence for Cas9 can comprise the sequence set forth in SEQ ID NO:11 or a fragment thereof. In an aspect, a disclosed nucleic acid sequence for Cas9 can comprise a sequence having at least 80%, at least 85%, at least 90%, or at least 95% identity to the sequence set forth in SEQ ID NO:11 or a fragment thereof.

In an aspect, a disclosed method can comprise reducing the expression level, activity level, or both of glycogen synthase. In an aspect, reducing the expression level, activity, or both of glycogen synthase comprises administering a therapeutically effective amount of an agent for reducing the expression level and/or activity level of glycogen synthase. In an aspect, a glycogen synthase can be GYS1 (muscle glycogen synthase) or GYS2 (liver glycogen synthase) or both. In an aspect, a disclosed method of reducing the expression level, activity level, or both of glycogen synthase can comprise administering an RNA therapeutic. RNA therapeutics are known to the art and include double stranded RNA-mediated interference (RNAi) and antisense oligonucleotides (ASO). Thus, in an aspect, a disclosed method can comprise administering RNAi or administering ASO or both. In an aspect, a disclosed method can comprise administering RNAi or administering ASO or both directed at GYS1 and/or GYS2.

In an aspect, a disclosed method of reducing the expression level and/or activity level of glycogen synthase can comprise SRT. For example, in an aspect, SRT can comprise inhibiting glycogen synthase (i.e., GYS1 and/or GYS2) in a cell or a subject to reduce glycogen synthesis and/or glycogen accumulation in cells and tissues (e.g., skeletal muscle, lung tissue, liver tissue, brain tissue, or any other tissue having glycogen accumulation) when GAA and/or GBE activity and/or expression levels are reduced. In an aspect, SRT can comprise siRNA-based therapies, shRNA-based therapies, antisense therapies, gene-editing therapies, and therapies using one or more small molecules or peptide drugs.

In an aspect, a disclosed method of reducing the expression level, activity level, or both of glycogen synthase can comprise administering a small molecule. In an aspect, a disclosed small molecule can reduce activity and/or expression of GYS1 in view of the reduced activity and/or expression level of GAA, GBE, or one or more other enzymes in the metabolic pathways of glycogen metabolism and glycolysis. In an aspect, a disclosed small molecule can traverse the blood-brain-barrier. In an aspect, a disclosed small molecule can be guaiacol. In an aspect, a disclosed small molecule that inhibits glycogen synthase (GYS1) can be orally delivered.

In an aspect, a disclosed method of reducing the expression level and/or activity level of glycogen synthase can comprise using a gene editing system. In an aspect, a gene editing system can comprise CRISPR/Cas9, or can comprise zinc finger nucleases (ZFNs), transcription activator-like effector nucleases (TALENs), and/or homing endonucleases.

In an aspect, a disclosed method can further comprise administering to the subject a therapeutically effective amount of a therapeutic agent. In an aspect, a disclosed method can comprise reducing glycogen levels by administering a glycogen synthase inhibitor (e.g., RNAi, ASO, etc.) to the subject, or modifying the subject's diet, for example, by using cornstarch or another slow release starch to prevent hypoglycemia, or modifying the subject's diet, for example, by consuming a high amount of protein, fat, or other anaplerotic agents (such as, for example, C7 compounds like triheptanoin), exercise or a combination thereof. In an aspect, a disclosed method can comprise gene editing one or more relevant genes (such as, for example, genes in the glycogenolysis pathway), wherein editing includes but is not limited to single gene knockout, loss of function screening of multiple genes at one, gene knockin, or a combination thereof.

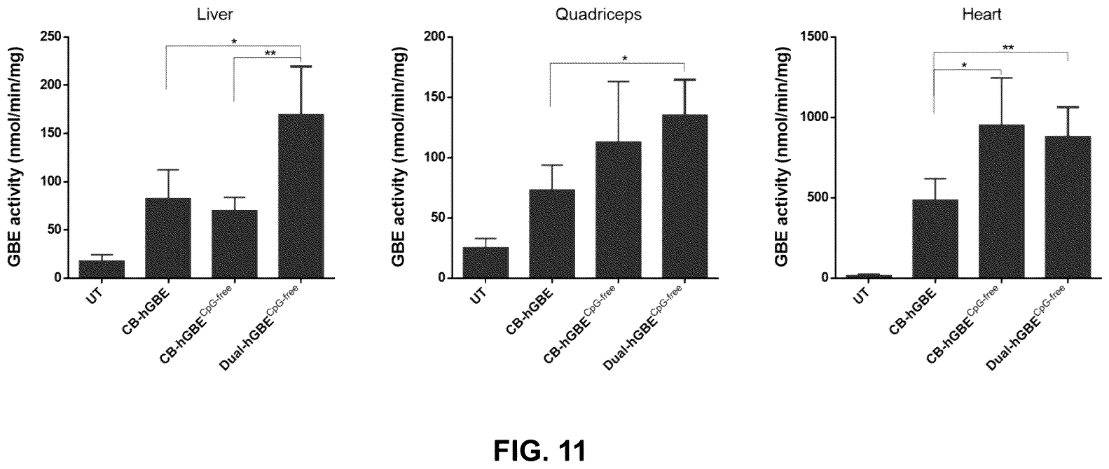

In an aspect, a disclosed method can further comprise administering to the subject a therapeutically effective amount of an agent that can correct one or more aspects of a dysregulated glycogen metabolism pathway, such as glycogen synthesis or glycogenolysis. In an aspect, such an agent can comprise an enzyme for enzyme replacement therapy. In an aspect, a disclosed enzyme can replace a mutated or dysfunction or nonexistence product of the GYG1, RBCK1, PRKAG2, or GBE gene, or a combination thereof. In an aspect, a disclosed enzyme can replace any enzyme in a dysregulated or dysfunctional glycogen metabolism pathway (FIG. 11).

As known to the art, glycogen synthesis and breakdown is regulated according to the energy state of the cell determined by the ratio of ATP to ADP. When glucose is abundant the amount of ATP is higher and that of AMP is low so that AMPK remains unphosphorylated and inactive. However, when glucose concentrations are low, ATP production decreases, while ATP is converted to ADP and AMP by cellular processes that use ATP as an energy source.

Higher concentrations of ADP and AMP activate AMPK, or more specifically, its a subunit (AMPKα). Active AMPKα triggers catabolic metabolism, which prevents the synthesis of glycogen, lipids, and most proteins while activating glycogen breakdown, oxidative phosphorylation, and mitochondrial biogenesis.

In an aspect, a disclosed method can comprise restoring the level of glycogen synthase (GYS1) to normal or near normal in a subject or in a tissue and/or organ in a subject. In an aspect, a disclosed method can comprise restoring the level of GBE to normal or near normal in a subject or in a tissue and/or organ in a subject. In an aspect, a disclosed method can comprise restoring the ratio of GYS1 and GBE to normal or near normal in a subject or in a tissue and/or organ in a subject.

In an aspect, a disclosed method can comprise restoring glucose homeostasis. In an aspect of a disclosed method, techniques to monitor, measure, and/or assess the restoring glucose homeostasis can comprise qualitative (or subjective) means as well as quantitative (or objective) means. These means are known to the skilled person.

In an aspect, a disclosed method can restore one or more aspects of the glycogen signaling pathway, restore one or more aspects of the glycogenolysis signaling pathway, can restore one or more aspects of the glycogenesis signaling pathway, or any combination thereof. In an aspect, a restoring one or more aspects of a disclosed signaling pathway can comprise restoring the activity and/or functionality of one or more enzymes identified in FIG. 2. In an aspect, restoration can be a 10%, 20%, 30%, 40%, 50%, 60%, 70%, 80%, 90%, 100%, or any amount of restoration when compared to a pre-existing level such as, for example, a pre-treatment level. In an aspect, the amount of restoration can be 10-20%, 20-30%, 30-40%, 40-50%, 50-60%, 60-70%, 70-80%, 80-90%, or 90-100% more than a pre-existing level such as, for example, a pre-treatment level. In an aspect, restoration can be measured against a control level (e.g., a level in a subject not having a GSD). In an aspect, restoration can be a partial or incomplete restoration. In an aspect, restoration can be complete or near complete restoration such that the level of expression, activity and/or functionality is similar to that of a wild-type or control level.

In an aspect, a disclosed method can comprise restoring one or more aspects of cellular homeostasis and/or cellular functionality. In an aspect, restoring one or more aspects of cellular homeostasis and/or cellular functionality can comprise one or more of the following: (i) correcting cell starvation in one or more cell types (such as, for example, liver cells, muscle cells, cells in the PNS, and cells in the CNS); (ii) normalizing aspects of autophagy pathway (correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) preventing, slowing, and/or eliminating hypoglycemia, ketosis, and/or other liver abnormalities related to liver disease; (vi) improving, preventing, and/or reversing neurogenic bladder, gait disturbances, and/or neuropathy; (vii) inhibiting, preventing, stabilizing, and/or slowing the rate of progression of the multi-systemic manifestations of GSD IV including cardiomyopathy, hepatic, and musculoskeletal dysfunction, (viii) inhibiting, preventing, stabilizing, and/or slowing the rate of progression of the multi-systemic manifestations of APBD; (ix) inhibiting, preventing, and/or slowing the formation and/or cellular inclusion of polyglucosan bodies; (x) inhibiting, preventing, stabilizing, and/or slowing the rate of progression of the onset of CNS- and PNS-related manifestations including neurogenic bladder, peripheral neuropathy, motor neuron disease, gait disturbances, cognitive decline, and other CNS/PNS manifestations as known to the skilled person in the art, (xi) inhibiting, preventing, stabilizing, and/or slowing the rate of progression of liver disease including fibrosis, cirrhosis, hepatic adenomas, and liver hepatocellular carcinoma, or (xi) any combination thereof. In an aspect, restoring one or more aspects of cellular homeostasis can comprise improving, enhancing, restoring, and/or preserving one or more aspects of cellular structural and/or functional integrity.

In an aspect of a disclosed method, techniques to monitor, measure, and/or assess the restoring one or more aspects of cellular homeostasis and/or cellular functionality can comprise qualitative (or subjective) means as well as quantitative (or objective) means. These means are known to the skilled person.

In an aspect, a disclosed method can comprise administering one or more immune modulators. In an aspect, a disclosed immune modulator can be methotrexate, rituximab, intravenous gamma globulin, or bortezomib, or a combination thereof. In an aspect, a disclosed immune modulator can be bortezomib or SVP-Rapamycin.

In an aspect, a disclosed method can comprise plasmapheresis to remove, for example, antibodies to one or more administered treatments.

In an aspect, a disclosed immune modulator such as methotrexate can be administered at a transient low to high-dose. In an aspect, a disclosed immune modulator can be administered at a dose of about 0.1 mg/kg body weight to about 0.6 mg/kg body weight. In an aspect, a disclosed immune modulator can be administered at a dose of about 0.4 mg/kg body weight. In an aspect, a disclosed immune modulator can be administered at about a daily dose of 0.4 mg/kg body weight for 3 to 5 or greater cycles, with up to three days per cycle. In an aspect, a disclosed immune modulator can be administered at about a daily dose of 0.4 mg/kg body weight for a minimum of 3 cycles, with three days per cycle. In an aspect, a person skilled in the art can determine the appropriate number of cycles. In an aspect, a disclosed immune modulator can be administered as many times as necessary to achieve a desired clinical effect.

In an aspect, a disclosed immune modulator can be administered orally about one hour before a disclosed therapeutic agent. In an aspect, a disclosed immune modulator can be administered subcutaneously about 15 minutes before a disclosed therapeutic agent. In an aspect, a disclosed immune modulator can be administered concurrently with or prior to a disclosed therapeutic agent. In an aspect, a disclosed immune modulator can be administered orally about one hour or a few days before a disclosed isolated nucleic acid molecule, a disclosed vector, a disclosed pharmaceutical formulation, or a combination thereof. In an aspect, a disclosed immune modulator can be administered subcutaneously about 15 minutes before or a few days before a disclosed isolated nucleic acid molecule, a disclosed vector, a disclosed pharmaceutical formulation, or a combination thereof. In an aspect, a disclosed immune modulator can be administered concurrently with a disclosed isolated nucleic acid molecule, a disclosed vector, a disclosed pharmaceutical formulation, or a combination thereof.

In an aspect, a disclosed method can comprise administering one or more proteasome inhibitors (e.g., bortezomib, carfilzomib, marizomib, ixazomib, and oprozomib). In an aspect, a proteasome inhibitor can be an agent that acts on plasma cells (e.g., daratumumab). In an aspect, an agent that acts on a plasma cell can be melphalan hydrochloride, melphalan, pamidronate disodium, carmustine, carfilzomib, carmustine, cyclophosphamide, daratumumab, doxorubicin hydrochloride liposome, doxorubicin hydrochloride liposome, elotuzumab, melphalan hydrochloride, panobinostat, ixazomib citrate, carfilzomib, lenalidomide, melphalan, melphalan hydrochloride, plerixafor, ixazomib citrate, pamidronate disodium, panobinostat, plerixafor, pomalidomide, pomalidomide, lenalidomide, selinexor, thalidomide, thalidomide, bortezomib, selinexor, zoledronic acid, or zoledronic acid.

In an aspect, a disclosed method can comprise administering one or more proteasome inhibitors or agents that act on plasma cells prior to administering a disclosed isolated nucleic acid molecule, a disclosed vector, or a disclosed pharmaceutical formulation. In an aspect, a disclosed method can comprise administering one or more proteasome inhibitors or one or more agents that act on plasma cells concurrently with administering a disclosed isolated nucleic acid molecule, a disclosed vector, or a disclosed pharmaceutical formulation. In an aspect, a disclosed method can comprise administering one or more proteasome inhibitors or one or more agents that act on plasma cells subsequent to administering a disclosed isolated nucleic acid molecule, a disclosed vector, or a disclosed pharmaceutical formulation.

In an aspect, a disclosed method can comprise administering one or more proteasome inhibitors more than 1 time. In an aspect, a disclosed method can comprise administering one or more proteasome inhibitors repeatedly over time.

In an aspect, a disclosed method can comprise administering one or more immunosuppressive agents. In an aspect, an immunosuppressive agent can be, but is not limited to, azathioprine, methotrexate, sirolimus, anti-thymocyte globulin (ATG), cyclosporine (CSP), mycophenolate mofetil (MMF), steroids, or a combination thereof. In an aspect, a disclosed method can comprise administering one or more immunosuppressive agents more than 1 time.

In an aspect, a disclosed method can comprise administering one or more one or more immunosuppressive agents repeatedly over time. In an aspect, a disclosed method can comprise administering a compound that targets or alters antigen presentation or humoral or cell mediated or innate immune responses.

In an aspect, a disclosed method can comprise administering a compound that exerts a therapeutic effect against B cells and/or a compound that targets or alters antigen presentation or humoral or cell mediated immune response. In an aspect, a disclosed compound can be rituximab, methotrexate, intravenous gamma globulin, anti CD4 antibody, anti CD2, an anti-FcRN antibody, a BTK inhibitor, an anti-IGF1R antibody, a CD19 antibody (e.g., inebilizumab), an anti-IL6 antibody (e.g., tocilizumab), an antibody to CD40, an IL2 mutein, or a combination thereof. Also disclosed herein are Treg infusions that can be administered as a way to help with immune tolerance (e.g., antigen specific Treg cells to AAV).

In an aspect, a disclosed method comprises administering lipid nanoparticles (LNPs). In an aspect, LNPs can be organ-targeted. In an aspect, LNPs can be liver-targeted or skeletal muscle targeted. For example, in an aspect, mRNA therapy with lipid nanoparticle encapsulation for systemic delivery to hepatocytes has the potential to restore metabolic enzymatic activity for one or more glycogen storage diseases such as GSD IV and/or APBD, Lafora disease (including those diseases caused by mutations in the EPM2A gene (glucan phosphatase, laforin) or the NHLRC1 gene (NHL repeat containing E3 ubiquitin protein ligase 1 or EPM2B)), polyglucosan body myopathy-1, polyglucosan body myopathy-2, or any disease or pathology caused by a mutation in a GYG1 gene, a RBCK1 gene, or a PRKAG2 gene. In an aspect, the mRNA therapy focuses on a GBE gene, a GYG1 gene, a RBCK1 gene, a PRKAG2 gene, or a combination thereof. In an aspect, the mRNA therapy focuses on one or more genes in the glycogenolysis pathway.

In an aspect, a disclosed method can comprise treating a subject that has developed or is likely to develop neutralizing antibodies (ABs) to the vector, capsid, and/or transgene. In an aspect, treating a subject that has developed or is likely to develop neutralizing antibodies can comprise plasmapheresis and immunosuppression. In an aspect, a disclosed method can comprise using immunosuppression to decrease the T cell, B cell, and/or plasma cell population, decrease the innate immune response, inflammatory response, and antibody levels in general. In an aspect, a disclosed method can comprise administering an IgG-degrading agent that depletes pre-existing neutralizing antibodies. In an aspect, a disclosed method can comprise administering to the subject IdeS or IdeZ, rapamycin, and/or SVP-Rapamycin. In an aspect, a disclosed IgG-degrading agent is bacteria-derived IdeS or IdeZ.

In an aspect, a disclosed method can comprise plasmapheresis to remove, for example, antibodies to one or more administered treatments.

In an aspect, a disclosed method can further comprise administering to the subject an effective amount an isolated nucleic acid encoding a protein that is deficient or absent in the subject. In an aspect, a disclosed encoded protein can comprise a recombinant human protein such as, for example, recombinant alpha-glucosidase (GAA). In an aspect, a disclosed GAA can comprise the amino acid sequence set forth in any one of SEQ ID NO:15-SEQ ID NO:19 or a fragment thereof. In an aspect, a disclosed GAA can comprise a sequence having at least 30%, at least 40%, at least 50%, at least 60%, at least 70%, at least 80%, or at least 90% identity to the amino acid sequence set forth in any one of SEQ ID NO:15-SEQ ID NO:19 or a fragment thereof. In an aspect, a disclosed GAA can be Myozyme or Lumizyme. In an aspect of a disclosed method, a disclosed isolated nucleic acid sequence for GAA can comprise the sequence set forth in SEQ ID NO:20, SEQ ID NO:21 or a fragment thereof. In an aspect, a disclosed isolated nucleic acid sequence for GAA can comprise a sequence having at least 30%, at least 40%, at least 50%, at least 60%, at least 70%, at least 80%, or at least 90% identity to the nucleotide sequence set forth in SEQ ID NO:20, SEQ ID NO:21, or a fragment thereof. In an aspect, a disclosed isolated nucleic acid encoding a recombinant protein such as, for example, GAA, can be present in a disclosed viral vector including, for example, an AAV vector or a self-complementary AAV vector. In an aspect, a disclosed immune modulator and a disclosed therapeutic agent can be concurrently administered. In an aspect, a disclosed composition comprising GAA or a disclosed vector comprising a disclosed isolated nucleic acid molecule encoding GAA can be administered prior to, concurrent with, or after the administration of a disclosed vector comprising a disclosed isolated nucleic acid molecule comprising a nucleic acid sequence encoding a polypeptide for preventing glycogen accumulation and/or degrading accumulated glycogen.

Disclosed herein is a method of treating and/or preventing GSD IV and/or APBD disease progression comprising administering to a subject in need thereof a therapeutically effective amount of a disclosed isolated nucleic acid molecule.

Disclosed herein is a method of treating and/or preventing GSD IV and/or APBD disease progression comprising administering to a subject in need thereof an isolated nucleic acid molecule comprising a nucleic acid sequence encoding a therapeutic polypeptide, wherein glycogen accumulation is prevented and/or accumulated a glycogen is degraded in the subject.

Disclosed herein is a method of treating and/or preventing GSD IV and/or APBD disease progression comprising preventing glycogen accumulation and/or degrading accumulated glycogen in a subject in need thereof by administering to the subject an isolated nucleic acid molecule comprising a nucleic acid sequence encoding a therapeutic polypeptide.

Disclosed herein is a method of treating and/or preventing GSD IV and/or APBD disease progression comprising administering to a subject in need thereof an isolated nucleic acid molecule comprising a nucleic acid sequence encoding a therapeutic polypeptide, thereby preventing glycogen accumulation and/or degrading accumulated glycogen in the subject.

Disclosed herein is a method of preventing glycogen accumulation or degrading accumulated glycogen comprising administering to a subject having GSD IV and/or APBD an isolated nucleic acid molecule comprising a nucleic acid sequence encoding a therapeutic polypeptide.

Disclosed herein is a method of treating and/or preventing GSD IV and/or APBD disease progression comprising administering to a subject in need thereof an isolated nucleic acid molecule comprising a nucleic acid sequence encoding a therapeutic polypeptide, and a therapeutically effective amount of an agent for reducing the expression level and/or activity level of glycogen synthase, wherein glycogen accumulation is prevented and/or accumulated glycogen is degraded in the subject.

Disclosed herein is a method of treating and/or preventing GSD IV and/or APBD disease progression comprising preventing glycogen accumulation and/or degrading accumulated glycogen in a subject in need thereof by administering to the subject an isolated nucleic acid molecule comprising a nucleic acid sequence encoding a therapeutic polypeptide, and a therapeutically effective amount of an agent for reducing the expression level and/or activity level of glycogen synthase.

Disclosed herein is a method of treating and/or preventing GSD IV and/or APBD disease progression comprising administering to a subject in need thereof an isolated nucleic acid molecule comprising a nucleic acid sequence encoding a therapeutic polypeptide, and a therapeutically effective amount of an agent for reducing the expression level and/or activity level of glycogen synthase, thereby preventing glycogen accumulation and/or degrading accumulated glycogen in the subject.

Disclosed herein is a method of preventing glycogen accumulation and/or degrading accumulated glycogen comprising administering to a subject having GSD IV and/or ABPD an isolated nucleic acid molecule comprising a nucleic acid sequence encoding a therapeutic polypeptide, and a therapeutically effective amount of an agent for reducing the expression level and/or activity level of glycogen synthase.

In an aspect, a disclosed nucleic acid sequence can be CpG-depleted and codon-optimized for expression in a human or a mammalian cell. In an aspect, a mammalian cell can be a cell from any non-human species, such as, for example, a cell from a gorilla, a chimpanzee, a Rhesus monkey, a dog, a cow, a mouse, and a rat.

In an aspect, the encoded polypeptide can degrade amylopectin-like glycogen. In an aspect, the encoded polypeptide can degrade Lafora bodies, polyglucosan bodies, or any form of accumulated glycogen.

In an aspect, a disclosed encoded polypeptide can be a human glycogen branching enzyme. In an aspect, a disclosed encoded polypeptide can be a human salivary or pancreatic amylase. In an aspect, a disclosed encoded polypeptide can be derived from plant, bacteria, or another microorganism. In an aspect, a disclosed encoded polypeptide can be derived from any non-human species, such as, for example, gorilla, chimpanzee, Rhesus monkey, dog, cow, mouse, rat, chicken, zebrafish, fruit fly, mosquito, C. elegans, and frog.

In an aspect, a disclosed encoded polypeptide can comprise the sequence set forth in SEQ ID NO:01. In an aspect, a disclosed encoded polypeptide can comprise a sequence having at least 30%, at least 40%, at least 50%, at least 60%, at least 70%, at least 80%, or at least 90% identity to the sequence set forth in SEQ ID NO:01. In an aspect, a disclosed encoded polypeptide can comprise the sequence set forth in SEQ ID NO:07. In an aspect, a disclosed encoded polypeptide can comprise a sequence having at least 30%, at least 40%, at least 50%, at least 60%, at least 70%, at least 80%, or at least 90% identity to the sequence set forth in SEQ ID NO:07. In an aspect, a disclosed encoded polypeptide can comprise the sequence set forth in SEQ ID NO:08. In an aspect, a disclosed encoded polypeptide can comprise a sequence having at least 30%, at least 40%, at least 50%, at least 60%, at least 70%, at least 80%, or at least 90% identity to the sequence set forth in SEQ ID NO:08.

In an aspect, a disclosed nucleic acid sequence encoding a polypeptide can comprise the sequence set forth in SEQ ID NO:03 or SEQ ID NO:04. In an aspect, a disclosed nucleic acid sequence encoding a polypeptide can comprise a sequence having at least 30%, at least 40%, at least 50%, at least 60%, at least 70%, at least 80%, or at least 90% identity to the sequence set forth in SEQ ID NO:03 or SEQ ID NO:04. In an aspect, a disclosed nucleic acid sequence can comprise a sequence having at least 80% identity to the sequence set forth in SEQ ID NO:03 or SEQ ID NO:04. In an aspect, the original (non-CpG-depleted) polynucleotide open reading frame (ORF) sequence of human glycogen branching enzyme can comprise the sequence set forth in SEQ ID NO:2. In an aspect, the original (non-CpG-depleted) polynucleotide open reading frame (ORF) sequence of human glycogen branching enzyme can comprise a sequence having at least 50-69%, at least 70-89%, or at least 90-99% identity to the sequence set forth in SEQ ID NO:02.

In an aspect, a disclosed isolated nucleic acid molecule can be administered via intravenous, intraarterial, intramuscular, intraperitoneal, subcutaneous, intra-CSF, intrathecal, intraventricular, or in utero administration. In an aspect, a disclosed isolated nucleic acid molecule can be administered via intra-CSF administration in combination with a disclosed nucleic acid molecule, a disclosed vector, and/or a disclosed pharmaceutical formulation. In an aspect, a disclosed isolated nucleic acid molecule can be administered via intra-CSF administration in combination with RNAi, antisense oligonucleotides, miRNA, one or more small molecules, one or more therapeutic agents, one or more proteasome inhibitors, one or more immune modulators, and/or a gene editing system. In an aspect, a disclosed isolated nucleic acid molecule can be administered via LNP administration. In an aspect, a subject can be a human subject. In an aspect, disclosed isolated nucleic acid molecule can be delivered to the subject's liver, heart, skeletal muscle, smooth muscle, CNS, PNS, or a combination thereof. In an aspect, a disclosed vector can be concurrently and/or serially administered to a subject via multiple routes of administration. For example, in an aspect, administering a disclosed vector can comprise intravenous administration and intra-cistern magna (ICM) administration. In an aspect, administering a disclosed vector can comprise IV administration and intrathecal (ITH) administration.

In an aspect, a therapeutically effective amount of disclosed vector can be delivered via intravenous (IV) administration and can comprise a range of 1×10¹⁰ to 2×10¹⁴·vg/kg. In an aspect, a therapeutically effective amount of disclosed vector can be delivered via intra-cistem magna (ICM) administration and can comprise a range of 1×10⁹ to 2×10¹⁴·vg. In an aspect, a therapeutically effective amount of disclosed vector can be delivered via intrathecal (ITH) administration and can comprise a range of 1×10⁹ to 2×10¹⁴·vg. In an aspect, a therapeutically effective amount of disclosed vector can be delivered via intracerebroventricular (ICV) administration and can comprise a range of 1×10⁹ to 2×10¹⁴·vg. In an aspect, a therapeutically effective amount of a disclosed vector can comprise a single dose or a series of doses totalling the desired effective amount.

In an aspect, a disclosed isolated nucleic acid molecule can be present in a vector. In an aspect, a disclosed vector can be a viral vector or non-viral vector. In an aspect, a disclosed viral vector can be an adenovirus vector, an adeno-associated virus vector, a herpes simplex virus vector, a retrovirus vector, a lentivirus vector, and alphavirus vector, a flavivirus vector, a rhabdovirus vector, a measles virus vector, a Newcastle disease viral vector, a poxvirus vector, or a picornavirus vector. In an aspect, a disclosed non-viral vector can be a polymer based vector, a peptide based vector, a lipid nanoparticle, a solid lipid nanoparticle, or a cationic lipid based vector.

In an aspect, a disclosed viral vector can be an AAV vector. AAV vectors include, but are not limited to, AAV1, AAV2, AAV3 (including 3a and 3b), AAV4, AAV5, AAV6, AAV7, AAV8, AAVrh8, AAV9, AAV10, AAVrh10, AAV11, AAV12, AAV13, AAVrh39, AAVrh43, AAVcy.7 as well as bovine AAV, caprine AAV, canine AAV, equine AAV, ovine AAV, avian AAV, primate AAV, non-primate AAV, and any other virus classified by the International Committee on Taxonomy of Viruses (ICTV) as an AAV. In an aspect, AAV capsids can be chimeras either created by capsid evolution or by rational capsid engineering from the naturally isolated AAV variants to capture desirable serotype features such as enhanced or specific tissue tropism and host immune response escape, including but not limited to AAV-DJ, AAV-HAE1, AAV-HAE2, AAVM41, AAV-1829, AAV2 Y/F, AAV2 T/V, AAV2i8, AAV2.5, AAV9.45, AAV9.61, AAV-B1, AAV-AS, AAV9.45A-String (e.g., AAV9.45-AS), AAV9.45Angiopep, AAV9.47-Angiopep, and AAV9.47-AS, AAV-PHP.B, AAV-PHP.eB, AAV-PHP.S, AAV-F, AAVcc.47, and AAVcc.81. In an aspect, an AAV vector can be AAV9, AAVF, AAVcc.47, or AAVcc.81. In an aspect, a disclosed AAV vector can be AAV-Rh74 or a related variant (e.g., capsid variants like RHM4-1).

In an aspect, a disclosed vector can comprise a ubiquitous promoter. In an aspect, a disclosed ubiquitous promoter can be a CMV enhancer/chicken β-actin promoter or a CpG-depleted mCMV/hEF1α promoter. In an aspect, a disclosed vector can comprise a tissue-specific promoter. In an aspect, a disclosed tissue-specific promoter can be a liver-specific promoter, a muscle-specific promoter, a neuron-specific promoter (such as, for example, a synapsin I promoter), or a combination thereof. In an aspect, a disclosed liver-specific promoter can be a α1-microglobulin/bikunin enhancer/thyroid hormone-binding globulin promoter.

In an aspect, a disclosed liver-specific promoter can comprise any liver-specific promoter known to the art. In an aspect, a liver specific promoter can comprise about 845-bp and comprise the thyroid hormone-binding globulin promoter sequences (2382 to 13), two copies of α1-microglobulinybikunin enhancer sequences (22,804 through 22,704), and a 71-bp leader sequence as described by Ill C R, et al. (1997). In an aspect, a disclosed liver-specific promoter can comprise the sequence set froth in SEQ ID NO:34. In an aspect, a disclosed liver-specific promoter can comprise a sequence having at least 40%, 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:34. In an aspect, a disclosed liver-specific promoter can comprise a sequence having at least 40%-60%, at least 60%-80%, at least 80%-90%, or at least 90%-100% identity to the sequence set forth in SEQ ID NO:34.

In an aspect, a disclosed promoter can be an endogenous promoter. In an aspect, a disclosed endogenous promoter can generally be obtained from a non-coding region upstream of a transcription initiation site of a gene (such as, for example, a disclosed GBE or some other enzyme involved in the glycogen signaling pathway). In an aspect, a disclosed engoengous promoter can be used for constitutive and efficient expression of a disclosed transgene (e.g., a nucleic acid sequence encoding a polypeptide capable of preventing glycogen accumulation and/or degrading accumulated glycogen). The skilled person is familiar with the methods and tools to identify an endogenous promoter such as, for example, the endogenous promoter for GBE.

In an aspect, a disclosed vector can comprise an immunotolerant dual promoter comprising a liver-specific promoter and a ubiquitous promoter. In an aspect, an immunotolerant dual promoter can comprise a liver-specific promoter and another tissue specific promoter (such as, for example, a muscle-specific promoter, a neuron-specific promoter (such as, for example, a synapsin I promoter), a skeletal muscle-specific promoter, and a heart-specific promoter). In an aspect, a disclosed immunotolerant dual promoter can comprise a α1-microglobulin/bikunin enhancer/thyroid hormone-binding globulin promoter and a CB promoter. In an aspect, a disclosed immunotolerant dual promoter can comprise a al-microglobulin/bikunin enhancer/thyroid hormone-binding globulin promoter and a CpG-depleted mCMV/hEF1α promoter. In an aspect, a disclosed immunotolerant dual promoter can comprise the sequence set forth in SEQ ID NO:05 or SEQ ID NO:06. In an aspect, a disclosed immunotolerant dual promoter can comprise a sequence having at least 40%, 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:05 or SEQ ID NO:06.

In an aspect, a disclosed method can comprise reducing the expression level, activity level, or both of glycogen synthase. In an aspect, reducing the expression level, activity, or both of glycogen synthase comprises administering a therapeutically effective amount of an agent for reducing the expression level and/or activity level of glycogen synthase. In an aspect, a glycogen synthase can be GYS1 (muscle glycogen synthase) or GYS2 (liver glycogen synthase) or both. In an aspect, a disclosed method of reducing the expression level, activity level, or both of glycogen synthase can comprise administering an RNA therapeutic. RNA therapeutics are known to the art and include double stranded RNA-mediated interference (RNAi) and antisense oligonucleotides (ASO). Thus, in an aspect, a disclosed method can comprise administering RNAi or administering ASO or both. In an aspect, a disclosed method can comprise administering RNAi or administering ASO or both directed at GYS1 and/or GYS2.

In an aspect, a disclosed method can further comprise administering to the subject a therapeutically effective amount of a therapeutic agent. In an aspect, a disclosed method can comprise reducing glycogen levels by administering a glycogen synthase inhibitor (e.g., RNAi, ASO, etc.) to the subject, or modifying the subject's diet, for example, by using cornstarch or another slow release starch to prevent hypoglycemia, or modifying the subject's diet, for example, by consuming a high amount of protein, fat, or other anaplerotic agents (such as, for example, C7 compounds like triheptanoin), exercise or a combination thereof. In an aspect, a disclosed method can comprise gene editing one or more relevant genes (such as, for example, genes in the glycogenolysis pathway), wherein editing includes but is not limited to single gene knockout, loss of function screening of multiple genes at one, gene knockin, or a combination thereof).

In an aspect, a disclosed method can further comprise administering to the subject a therapeutically effective amount of an agent that can correct one or more aspects of a dysregulated glycogen metabolism pathway, such as glycogen synthesis or glycogenolysis. In an aspect, such an agent can comprise an enzyme for enzyme replacement therapy. In an aspect, a disclosed enzyme can replace a mutated or dysfunction or nonexistence product of the GYG1, RBCK1, PRKAG2, or GBE gene, or a combination thereof. In an aspect, a disclosed enzyme can replace any enzyme in a dysregulated or dysfunctional glycogen metabolism pathway (FIG. 2).

In an aspect, a disclosed method can comprise administering one or more immune modulators. In an aspect, a disclosed immune modulator can be methotrexate, rituximab, intravenous gamma globulin, or bortezomib, or a combination thereof. In an aspect, a disclosed immune modulator can be bortezomib or SVP-Rapamycin.

In an aspect, a disclosed method can comprise plasmapheresis to remove, for example, antibodies to one or more administered treatments.

In an aspect, a disclosed immune modulator such as methotrexate can be administered at a transient low to high-dose. In an aspect, a disclosed immune modulator can be administered at a dose of about 0.1 mg/kg body weight to about 0.6 mg/kg body weight. In an aspect, a disclosed immune modulator can be administered at a dose of about 0.4 mg/kg body weight. In an aspect, a disclosed immune modulator can be administered at about a daily dose of 0.4 mg/kg body weight for 3 to 5 or greater cycles, with up to three days per cycle. In an aspect, a disclosed immune modulator can be administered at about a daily dose of 0.4 mg/kg body weight for a minimum of 3 cycles, with three days per cycle. In an aspect, a person skilled in the art can determine the appropriate number of cycles. In an aspect, a disclosed immune modulator can be administered as many times as necessary to achieve a desired clinical effect.

In an aspect, a disclosed immune modulator can be administered orally about one hour before a disclosed therapeutic agent. In an aspect, a disclosed immune modulator can be administered subcutaneously about 15 minutes before a disclosed therapeutic agent. In an aspect, a disclosed immune modulator can be administered concurrently with a disclosed therapeutic agent.

In an aspect, a disclosed immune modulator can be administered orally about one hour or a few days before a disclosed isolated nucleic acid molecule, a disclosed vector, a disclosed pharmaceutical formulation, or a combination thereof. In an aspect, a disclosed immune modulator can be administered subcutaneously about 15 minutes before or a few days before a disclosed isolated nucleic acid molecule, a disclosed vector, a disclosed pharmaceutical formulation, or a combination thereof. In an aspect, a disclosed immune modulator can be administered concurrently with a disclosed isolated nucleic acid molecule, a disclosed vector, a disclosed pharmaceutical formulation, or a combination thereof.

In an aspect, a disclosed method can comprise administering one or more proteasome inhibitors (e.g., bortezomib, carfilzomib, marizomib, ixazomib, and oprozomib). In an aspect, a proteasome inhibitor can be an agent that acts on plasma cells (e.g., daratumumab). In an aspect, an agent that acts on a plasma cell can be melphalan hydrochloride, melphalan, pamidronate disodium, carmustine, carfilzomib, carmustine, cyclophosphamide, daratumumab, doxorubicin hydrochloride liposome, doxorubicin hydrochloride liposome, elotuzumab, melphalan hydrochloride, panobinostat, ixazomib citrate, carfilzomib, lenalidomide, melphalan, melphalan hydrochloride, plerixafor, ixazomib citrate, pamidronate disodium, panobinostat, plerixafor, pomalidomide, pomalidomide, lenalidomide, selinexor, thalidomide, thalidomide, bortezomib, selinexor, zoledronic acid, or zoledronic acid.

In an aspect, a disclosed method can comprise administering one or more proteasome inhibitors or agents that act on plasma cells prior to administering a disclosed isolated nucleic acid molecule, a disclosed vector, or a disclosed pharmaceutical formulation. In an aspect, a disclosed method can comprise administering one or more proteasome inhibitors or one or more agents that act on plasma cells concurrently with administering a disclosed isolated nucleic acid molecule, a disclosed vector, or a disclosed pharmaceutical formulation. In an aspect, a disclosed method can comprise administering one or more proteasome inhibitors or one or more agents that act on plasma cells subsequent to administering a disclosed isolated nucleic acid molecule, a disclosed vector, or a disclosed pharmaceutical formulation.

In an aspect, a disclosed method can comprise administering one or more proteasome inhibitors more than 1 time. In an aspect, a disclosed method can comprise administering one or more proteasome inhibitors repeatedly over time.

In an aspect, a disclosed method can comprise administering one or more immunosuppressive agents. In an aspect, an immunosuppressive agent can be, but is not limited to, azathioprine, methotrexate, sirolimus, anti-thymocyte globulin (ATG), cyclosporine (CSP), mycophenolate mofetil (MMF), steroids, or a combination thereof. In an aspect, a disclosed method can comprise administering one or more immunosuppressive agents more than 1 time.

In an aspect, a disclosed method can comprise administering one or more one or more immunosuppressive agents repeatedly over time. In an aspect, a disclosed method can comprise administering a compound that targets or alters antigen presentation or humoral or cell mediated or innate immune responses.

In an aspect, a disclosed method can comprise administering a compound that exerts a therapeutic effect against B cells and/or a compound that targets or alters antigen presentation or humoral or cell mediated immune response. In an aspect, a disclosed compound can be rituximab, methotrexate, intravenous gamma globulin, anti CD4 antibody, anti CD2, an anti-FcRN antibody, a BTK inhibitor, an anti-IGF1R antibody, a CD19 antibody (e.g., inebilizumab), an anti-IL6 antibody (e.g., tocilizumab), an antibody to CD40, an IL2 mutein, or a combination thereof. Also disclosed herein are Treg infusions that can be administered as a way to help with immune tolerance (e.g., antigen specific Treg cells to AAV).

In an aspect, a disclosed method comprises administering lipid nanoparticles (LNPs). In an aspect, LNPs can be organ-targeted. In an aspect, LNPs can be liver-targeted or skeletal muscle targeted. For example, in an aspect, mRNA therapy with lipid nanoparticle encapsulation for systemic delivery to hepatocytes has the potential to restore metabolic enzymatic activity for one or more glycogen storage diseases such as GSD IV, Lafora disease (including those diseases caused by mutations in the EPM2A gene (glucan phosphatase, laforin) or the NHLRC1 gene (NHL repeat containing E3 ubiquitin protein ligase 1 or EPM2B)), polyglucosan body myopathy-1, polyglucosan body myopathy-2, APBD, or any disease or pathology caused by a mutation in a GYG1 gene, a RBCK1 gene, or a PRKAG2 gene. In an aspect, the mRNA therapy focuses on a GBE gene, aGYG1 gene, a RBCK1 gene, a PRKAG2 gene, or a combination thereof. In an aspect, the mRNA therapy focuses on one or more genes in the glycogenolysis pathway.

In an aspect, a disclosed method can comprise treating a subject that has developed or is likely to develop neutralizing antibodies (ABs) to the vector, capsid, and/or transgene. In an aspect, treating a subject that has developed or is likely to develop neutralizing antibodies can comprise plasmapheresis and immunosuppression. In an aspect, a disclosed method can comprise using immunosuppression to decrease the T cell, B cell, and/or plasma cell population, decrease the innate immune response, inflammatory response, and antibody levels in general. In an aspect, a disclosed method can comprise administering an IgG-degrading agent that depletes pre-existing neutralizing antibodies. In an aspect, a disclosed method can comprise administering to the subject IdeS or IdeZ, rapamycin, and/or SVP-Rapamycin. In an aspect, a disclosed IgG-degrading agent is bacteria-derived IdeS or IdeZ.

In an aspect, a disclosed method can comprise plasmapheresis to remove, for example, antibodies to one or more administered treatments.

### D. Methods for Treating and/or Preventing a Disease

Disclosed herein is a method of treating and/or preventing a disease comprising administering to a subject in need thereof a therapeutically effective amount of a disclosed isolated nucleic acid molecule, a disclosed vector, disclosed pharmaceutical formulation, or a combination thereof, wherein the disease is a GSD (such as GSD IV and/or APBD), Lafora disease (including those diseases caused by mutations in the EPM2A gene (glucan phosphatase, laforin) or the NHLRC1 gene (NHL repeat containing E3 ubiquitin protein ligase 1 or EPM2B)), polyglucosan body myopathy-1, polyglucosan body myopathy-2, or any disease or pathology caused by a mutation in a GYG1 gene, a RBCK1 gene, or a PRKAG2 gene.

Disclosed herein is a method of treating and/or preventing a disease comprising administering to a subject in need thereof a therapeutically effective amount of a disclosed isolated nucleic acid molecule, a disclosed vector, disclosed pharmaceutical formulation, or a combination thereof, and restoring the level of glycogen synthase (GYS1) and/or GBE to normal or near normal in the subject or in a tissue and/or organ in the subject.

Disclosed herein is a method of treating and/or preventing a disease comprising administering to a subject in need thereof a therapeutically effective amount of a vector comprising an isolated nucleic acid molecule comprising a nucleic acid sequence encoding a polypeptide for preventing glycogen accumulation and/or degrading accumulated glycogen, wherein the nucleic acid sequence is CpG-depleted and codon-optimized for expression in a human or a mammalian cell, and wherein glycogen accumulation is prevented and/or accumulated glycogen is degraded in the subject, wherein the disease is a GSD (such as GSD IV and/or APBD), Lafora disease (including those diseases caused by mutations in the EPM2A gene (glucan phosphatase, laforin) or the NHLRC1 gene (NHL repeat containing E3 ubiquitin protein ligase 1 or EPM2B)), polyglucosan body myopathy-1, polyglucosan body myopathy-2, or any disease or pathology caused by a mutation in a GYG1 gene, a RBCK1 gene, or a PRKAG2 gene.

Disclosed herein is a method of treating and/or preventing a disease comprising preventing glycogen accumulation and/or degrading accumulated glycogen in a subject in need thereof by administering to the subject a therapeutically effective amount of a vector comprising an isolated nucleic acid molecule comprising a nucleic acid sequence encoding a polypeptide for preventing glycogen accumulation and/or degrading accumulated glycogen, wherein the nucleic acid sequence is CpG-depleted and codon-optimized for expression in a human or a mammalian cell, wherein the disease is a GSD (such as GSD IV and/or APBD), Lafora disease (including those diseases caused by mutations in the EPM2A gene (glucan phosphatase, laforin) or the NHLRC1 gene (NHL repeat containing E3 ubiquitin protein ligase 1 or EPM2B)), polyglucosan body myopathy-1, polyglucosan body myopathy-2, or any disease or pathology caused by a mutation in a GYG1 gene, a RBCK1 gene, or a PRKAG2 gene.

Disclosed herein is a method of treating and/or preventing a disease comprising administering to a subject in need thereof a therapeutically effective amount of a vector comprising an isolated nucleic acid molecule comprising a nucleic acid sequence encoding a polypeptide for preventing glycogen accumulation and/or degrading accumulated glycogen, wherein the nucleic acid sequence is CpG-depleted and codon-optimized for expression in a human or a mammalian cell, thereby preventing glycogen accumulation and/or degrading accumulated glycogen in the subject, wherein the disease is a GSD (such as GSD IV and/or APBD), Lafora disease (including those diseases caused by mutations in the EPM2A gene (glucan phosphatase, laforin) or the NHLRC1 gene (NHL repeat containing E3 ubiquitin protein ligase 1 or EPM2B)), polyglucosan body myopathy-1, polyglucosan body myopathy-2, or any disease or pathology caused by a mutation in a GYG1 gene, a RBCK1 gene, or a PRKAG2 gene.

Disclosed herein is a method of preventing glycogen accumulation and/or degrading accumulated glycogen comprising administering to a subject having a disease a therapeutically effective amount of a vector comprising an isolated nucleic acid molecule comprising a nucleic acid sequence encoding a polypeptide for preventing glycogen accumulation and/or degrading accumulated glycogen, wherein the nucleic acid sequence is CpG-depleted and codon-optimized for expression in a human or a mammalian cell, wherein the disease is a GSD (such as GSD IV and/or APBD), Lafora disease (including those diseases caused by mutations in the EPM2A gene (glucan phosphatase, laforin) or the NHLRC1 gene (NHL repeat containing E3 ubiquitin protein ligase 1 or EPM2B)), polyglucosan body myopathy-1, polyglucosan body myopathy-2, or any disease or pathology caused by a mutation in a GYG1 gene, a RBCK1 gene, or a PRKAG2 gene.

Disclosed herein is a method of treating and/or preventing a disease comprising administering to a subject in need thereof a therapeutically effective amount of a disclosed isolated nucleic acid molecule, a disclosed vector, and/or a disclosed pharmaceutical formulation, and a therapeutically effective amount of an agent for reducing the expression level and/or activity level of glycogen synthase, wherein the disease is a GSD (such as GSD IV and/or APBD), Lafora disease (including those diseases caused by mutations in the EPM2A gene (glucan phosphatase, laforin) or the NHLRC1 gene (NHL repeat containing E3 ubiquitin protein ligase 1 or EPM2B)), polyglucosan body myopathy-1, polyglucosan body myopathy-2, or any disease or pathology caused by a mutation in a GYG1 gene, a RBCK1 gene, or a PRKAG2 gene.

Disclosed herein is a method of treating and/or preventing a disease comprising administering to a subject in need thereof a therapeutically effective amount of a vector comprising an isolated nucleic acid molecule comprising a nucleic acid sequence encoding a polypeptide for preventing glycogen accumulation and/or degrading accumulated glycogen, wherein the nucleic acid sequence is CpG-depleted and codon-optimized for expression in a human or a mammalian cell, and a therapeutically effective amount of an agent for reducing the expression level and/or activity level of glycogen synthase, wherein glycogen accumulation is prevented and/or accumulated glycogen is degraded in the subject, wherein the disease is a GSD (such as GSD IV and/or APBD), Lafora disease (including those diseases caused by mutations in the EPM2A gene (glucan phosphatase, laforin) or the NHLRC1 gene (NHL repeat containing E3 ubiquitin protein ligase 1 or EPM2B)), polyglucosan body myopathy-1, polyglucosan body myopathy-2, or any disease or pathology caused by a mutation in a GYG1 gene, a RBCK1 gene, or a PRKAG2 gene.

Disclosed herein is a method of treating and/or preventing a disease comprising preventing glycogen accumulation and/or degrading accumulated glycogen in a subject in need thereof by administering to the subject a therapeutically effective amount of a vector comprising an isolated nucleic acid molecule comprising a nucleic acid sequence encoding a polypeptide for preventing glycogen accumulation and/or degrading accumulated glycogen, wherein the nucleic acid sequence is CpG-depleted and codon-optimized for expression in a human or a mammalian cell, and a therapeutically effective amount of an agent for reducing the expression level and/or activity level of glycogen synthase, wherein the disease is a GSD (such as GSD IV and/or APBD), Lafora disease (including those diseases caused by mutations in the EPM2A gene (glucan phosphatase, laforin) or the NHLRC1 gene (NHL repeat containing E3 ubiquitin protein ligase 1 or EPM2B)), polyglucosan body myopathy-1, polyglucosan body myopathy-2, or any disease or pathology caused by a mutation in a GYG1 gene, a RBCK1 gene, or a PRKAG2 gene.

Disclosed herein is a method of treating and/or preventing a disease comprising administering to a subject in need thereof a therapeutically effective amount of a vector comprising an isolated nucleic acid molecule comprising a nucleic acid sequence encoding a polypeptide for preventing glycogen accumulation and/or degrading accumulated glycogen, wherein the nucleic acid sequence is CpG-depleted and codon-optimized for expression in a human or a mammalian cell, and a therapeutically effective amount of an agent for reducing the expression level and/or activity level of glycogen synthase, thereby preventing glycogen accumulation and/or degrading accumulated glycogen in the subject, wherein the disease is a GSD (such as GSD IV and/or APB), Lafora disease (including those diseases caused by mutations in the EPM2A gene (glucan phosphatase, laforin) or the NHLRC1 gene (NHL repeat containing E3 ubiquitin protein ligase 1 or EPM2B)), polyglucosan body myopathy-1, polyglucosan body myopathy-2, or any disease or pathology caused by a mutation in a GYG1 gene, a RBCK1 gene, or a PRKAG2 gene.

In an aspect, the encoded polypeptide can degrade glycogen, polyglucosan bodies, amylopectin-like glycogen, Lafora bodies, or any combination thereof. In an aspect, the encoded polypeptide can degrade any form of accumulated glycogen.

In an aspect, “glycogen” can refer to glycogen, polyglucosan bodies, amylopectin-like glycogen, or any combination thereof. For example, the term “glycogen accumulation” can comprise accumulation of polyglucosan bodies and/or amylopectin-like glycogen in addition to the accumulation of glycogen. In an aspect, accumulation can refer to accumulation of glycogen, polyglucosan bodies, amylopectin-like glycogen, or any combination thereof.

In an aspect, a disclosed encoded polypeptide can be a human glycogen branching enzyme. In an aspect, a disclosed encoded polypeptide can be a human salivary or pancreatic amylase. In an aspect, a disclosed encoded polypeptide can be derived from plant, bacteria, or another microorganism. In an aspect, a disclosed encoded polypeptide can be derived from any non-human species, such as, for example, gorilla, chimpanzee, Rhesus monkey, dog, cow, mouse, rat, chicken, zebrafish, fruit fly, mosquito, C. elegans, and frog. In an aspect, a mammalian cell can be a cell from any non-human species, such as, for example, a cell from a gorilla, a chimpanzee, a Rhesus monkey, a dog, a cow, a mouse, and a rat.

In an aspect, a disclosed encoded polypeptide can comprise the sequence set forth in SEQ ID NO:01. In an aspect, a disclosed encoded polypeptide can comprise a sequence having at least 30%, at least 40%, at least 50%, at least 60%, at least 70%, at least 80%, or at least 90% identity to the sequence set forth in SEQ ID NO:01. In an aspect, a disclosed encoded polypeptide can comprise the sequence set forth in SEQ ID NO:07. In an aspect, a disclosed encoded polypeptide can comprise a sequence having at least 30%, at least 40%, at least 50%, at least 60%, at least 70%, at least 80%, or at least 90% identity to the sequence set forth in SEQ ID NO:07. In an aspect, a disclosed encoded polypeptide can comprise the sequence set forth in SEQ ID NO:08. In an aspect, a disclosed encoded polypeptide can comprise a sequence having at least 30%, at least 40%, at least 50%, at least 60%, at least 70%, at least 80%, or at least 90% identity to the sequence set forth in SEQ ID NO:08.

In an aspect, a disclosed nucleic acid sequence encoding a polypeptide can comprise the sequence set forth in SEQ ID NO:03 or SEQ ID NO:04. In an aspect, a disclosed nucleic acid sequence encoding a polypeptide can comprise a sequence having at least 30%, at least 40%, at least 50%, at least 60%, at least 70%, at least 80%, or at least 90% identity to the sequence set forth in SEQ ID NO:03 or SEQ ID NO:04. In an aspect, a disclosed nucleic acid sequence can comprise a sequence having at least 80% identity to the sequence set forth in SEQ ID NO:03 or SEQ ID NO:04. In an aspect, the original (non-CpG-depleted) polynucleotide open reading frame (ORF) sequence of human glycogen branching enzyme can comprise the sequence set forth in SEQ ID NO:2. In an aspect, the original (non-CpG-depleted) polynucleotide open reading frame (ORF) sequence of human glycogen branching enzyme can comprise a sequence having at least 50-69%, at least 70-89%, or at least 90-99% identity to the sequence set forth in SEQ ID NO:02.

In an aspect, a disclosed nucleic acid molecule can be in a vector. In an aspect, a disclosed vector can be a viral vector or non-viral vector. In an aspect, a disclosed viral vector can be an adenovirus vector, an adeno-associated virus vector, a herpes simplex virus vector, a retrovirus vector, a lentivirus vector, and alphavirus vector, a flavivirus vector, a rhabdovirus vector, a measles virus vector, a Newcastle disease viral vector, a poxvirus vector, or a picornavirus vector. In an aspect, a disclosed non-viral vector can be a polymer-based vector, a peptide-based vector, a lipid nanoparticle, a solid lipid nanoparticle, or a cationic lipid based vector.

In an aspect, a disclosed viral vector can be an AAV vector. AAV vectors include, but are not limited to, AAV1, AAV2, AAV3 (including 3a and 3b), AAV4, AAV5, AAV6, AAV7, AAV8, AAVrh8, AAV9, AAV10, AAVrh10, AAV11, AAV12, AAV13, AAVrh39, AAVrh43, AAVcy.7 as well as bovine AAV, caprine AAV, canine AAV, equine AAV, ovine AAV, avian AAV, primate AAV, non-primate AAV, and any other virus classified by the International Committee on Taxonomy of Viruses (ICTV) as an AAV. In an aspect, AAV capsids can be chimeras either created by capsid evolution or by rational capsid engineering from the naturally isolated AAV variants to capture desirable serotype features such as enhanced or specific tissue tropism and host immune response escape, including but not limited to AAV-DJ, AAV-HAE1, AAV-HAE2, AAVM41, AAV-1829, AAV2 Y/F, AAV2 TN, AAV2i8, AAV2.5, AAV9.45, AAV9.61, AAV-B1, AAV-AS, AAV9.45A-String (e.g., AAV9.45-AS), AAV9.45Angiopep, AAV9.47-Angiopep, and AAV9.47-AS, AAV-PHP.B, AAV-PHP.eB, AAV-PHP.S, AAV-F, AAVcc.47, and AAVcc.81. In an aspect, an AAV vector can be AAV9, AAVF, AAVcc.47, or AAVcc.81. In an aspect, a disclosed AAV vector can be AAV-Rh74 or a related variant (e.g., capsid variants like RHM4-1).

In an aspect, a disclosed isolated nucleic acid molecule and/or a disclosed vector can be administered via intravenous, intraarterial, intramuscular, intraperitoneal, subcutaneous, intra-CSF, intrathecal, intraventricular, or in utero administration. In an aspect, a disclosed isolated nucleic acid molecule and/or a disclosed vector can be administered via intra-CSF administration in combination with a disclosed nucleic acid molecule, a disclosed vector, and/or a disclosed pharmaceutical formulation. In an aspect, a disclosed isolated nucleic acid molecule and/or a disclosed vector can be administered via intra-CSF administration in combination with RNAi, antisense oligonucleotides, miRNA, one or more small molecules, one or more therapeutic agents, one or more proteasome inhibitors, one or more immune modulators, and/or a gene editing system. In an aspect, a disclosed isolated nucleic acid molecule and/or a disclosed vector can be administered via LNP administration. In an aspect, a subject can be a human subject. In an aspect, a disclosed vector can be delivered to the subject's liver, heart, skeletal muscle, smooth muscle, CNS, PNS, or a combination thereof. In an aspect, a disclosed vector can be concurrently and/or serially administered to a subject via multiple routes of administration. For example, in an aspect, administering a disclosed vector can comprise intravenous administration and intra-cistern magna (ICM) administration. In an aspect, administering a disclosed vector can comprise IV administration and intrathecal (ITH) administration.

In an aspect, a therapeutically effective amount of disclosed vector can be delivered via intravenous (IV) administration and can comprise a range of 1×10¹⁰ to 2×10¹⁴·vg/kg. In an aspect, a therapeutically effective amount of disclosed vector can be delivered via intra-cistem magna (ICM) administration and can comprise a range of 1×10⁹ to 2×10¹⁴·vg. In an aspect, a therapeutically effective amount of disclosed vector can be delivered via intrathecal (ITH) administration and can comprise a range of 1×10⁹ to 2×10¹⁴·vg. In an aspect, a therapeutically effective amount of disclosed vector can be delivered via intracerebroventricular (ICV) administration and can comprise a range of 1×10⁹ to 2×10¹⁴·vg. In an aspect, a therapeutically effective amount of a disclosed vector can comprise a single dose or a series of doses totalling the desired effective amount.

In an aspect, a disclosed vector can comprise a ubiquitous promoter. In an aspect, a disclosed ubiquitous promoter can be a CMV enhancer/chicken β-actin promoter or a CpG-depleted mCMV/hEF1α promoter. In an aspect, a disclosed vector can comprise a tissue-specific promoter. In an aspect, a disclosed tissue-specific promoter can be a liver-specific promoter, a muscle-specific promoter, a neuron-specific promoter (such as, for example, a synapsin I promoter), or a combination thereof. In an aspect, a disclosed liver-specific promoter can be a α1-microglobulin/bikunin enhancer/thyroid hormone-binding globulin promoter.

In an aspect, a disclosed liver-specific promoter can comprise any liver-specific promoter known to the art. In an aspect, a liver specific promoter can comprise about 845-bp and comprise the thyroid hormone-binding globulin promoter sequences (2382 to 13), two copies of α1-microglobulinybikunin enhancer sequences (22,804 through 22,704), and a 71-bp leader sequence as described by Ill C R, et al. (1997). In an aspect, a disclosed liver-specific promoter can comprise the sequence set froth in SEQ ID NO:34. In an aspect, a disclosed liver-specific promoter can comprise a sequence having at least 40%, 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:34. In an aspect, a disclosed liver-specific promoter can comprise a sequence having at least 40%-60%, at least 60%-80%, at least 80%-90%, or at least 90%-100% identity to the sequence set forth in SEQ ID NO:34.

In an aspect, a disclosed promoter can be an endogenous promoter. In an aspect, a disclosed endogenous promoter can generally be obtained from a non-coding region upstream of a transcription initiation site of a gene (such as, for example, a disclosed GBE or some other enzyme involved in the glycogen signaling pathway). In an aspect, a disclosed engoengous promoter can be used for constitutive and efficient expression of a disclosed transgene (e.g., a nucleic acid sequence encoding a polypeptide capable of preventing glycogen accumulation and/or degrading accumulated glycogen). The skilled person is familiar with the methods and tools to identify an endogenous promoter such as, for example, the endogenous promoter for GBE.

In an aspect, a disclosed vector can comprise an immunotolerant dual promoter comprising a liver-specific promoter and a ubiquitous promoter. In an aspect, an immunotolerant dual promoter can comprise a liver-specific promoter and another tissue specific promoter (such as, for example, a muscle-specific promoter, a neuron-specific promoter (such as, for example, a synapsin I promoter), a skeletal muscle-specific promoter, and a heart-specific promoter). In an aspect, a disclosed immunotolerant dual promoter can comprise a α1-microglobulin/bikunin enhancer/thyroid hormone-binding globulin promoter and a CB promoter. In an aspect, a disclosed immunotolerant dual promoter can comprise a al-microglobulin/bikunin enhancer/thyroid hormone-binding globulin promoter and a CpG-depleted mCMV/hEF1α promoter. In an aspect, a disclosed immunotolerant dual promoter can comprise the sequence set forth in SEQ ID NO:05 or SEQ ID NO:06. In an aspect, a disclosed immunotolerant dual promoter can comprise a sequence having at least 40%, 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:05 or SEQ ID NO:06.

In an aspect, a disclosed method can comprise reducing the expression level, activity level, or both of glycogen synthase. In an aspect, reducing the expression level, activity, or both of glycogen synthase comprises administering a therapeutically effective amount of an agent for reducing the expression level and/or activity level of glycogen synthase. In an aspect, a glycogen synthase can be GYS1 (muscle glycogen synthase) or GYS2 (liver glycogen synthase) or both. In an aspect, a disclosed method of reducing the expression level, activity level, or both of glycogen synthase can comprise administering an RNA therapeutic. RNA therapeutics are known to the art and include double stranded RNA-mediated interference (RNAi) and antisense oligonucleotides (ASO). Thus, in an aspect, a disclosed method can comprise administering RNAi or administering ASO or both. In an aspect, a disclosed method can comprise administering RNAi or administering ASO or both directed at GYS1 and/or GYS2.

In an aspect, a disclosed method of reducing the expression level, activity level, or both of glycogen synthase can comprise SRT. For example, in an aspect, SRT can comprise inhibiting glycogen synthase (i.e., GYS1 and/or GYS2) in a cell or a subject to reduce glycogen synthesis and/or glycogen accumulation in cells and tissues (e.g., skeletal muscle, lung tissue, liver tissue, brain tissue, or any other tissue having glycogen accumulation) when GAA and/or GBE activity and/or expression levels are reduced. In an aspect, SRT can comprise siRNA-based therapies, shRNA-based therapies, antisense therapies, gene-editing therapies, and therapies using one or more small molecules or peptide drugs.

In an aspect, a disclosed method can comprise restoring the level of glycogen synthase (GYS1) to normal or near normal in a subject or in a tissue and/or organ in a subject. In an aspect, a disclosed method can comprise restoring the level of GBE to normal or near normal in a subject or in a tissue and/or organ in a subject. In an aspect, a disclosed method can comprise restoring the ratio of GYS1 and GBE to normal or near normal in a subject or in a tissue and/or organ in a subject.

In an aspect, a disclosed method can comprise restoring glucose homeostasis. In an aspect of a disclosed method, techniques to monitor, measure, and/or assess the restoring glucose homeostasis can comprise qualitative (or subjective) means as well as quantitative (or objective) means. These means are known to the skilled person.

In an aspect, a disclosed method can restore one or more aspects of the glycogen signaling pathway, restore one or more aspects of the glycogenolysis signaling pathway, can restore one or more aspects of the glycogenesis signaling pathway, or any combination thereof. In an aspect, a restoring one or more aspects of a disclosed signaling pathway can comprise restoring the activity and/or functionality of one or more enzymes identified in FIG. 2. In an aspect, restoration can be a 10%, 20%, 30%, 40%, 50%, 60%, 70%, 80%, 90%, 100%, or any amount of restoration when compared to a pre-existing level such as, for example, a pre-treatment level. In an aspect, the amount of restoration can be 10-20%, 20-30%, 30-40%, 40-50%, 50-60%, 60-70%, 70-80%, 80-90%, or 90-100% more than a pre-existing level such as, for example, a pre-treatment level. In an aspect, restoration can be measured against a control level (e.g., a level in a subject not having a GSD (such as GSD IV and/or APBD)). In an aspect, restoration can be a partial or incomplete restoration. In an aspect, restoration can be complete or near complete restoration such that the level of expression, activity and/or functionality is similar to that of a wild-type or control level.

In an aspect, a disclosed method can comprise restoring one or more aspects of cellular homeostasis and/or cellular functionality. In an aspect, restoring one or more aspects of cellular homeostasis and/or cellular functionality can comprise one or more of the following: (i) correcting cell starvation in one or more cell types (such as, for example, liver cells, muscle cells, cells in the PNS, and cells in the CNS); (ii) normalizing aspects of autophagy pathway (correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) preventing, slowing, and/or eliminating hypoglycemia, ketosis, and/or other liver abnormalities related to liver disease; (vi) improving, preventing, and/or reversing neurogenic bladder, gait disturbances, and/or neuropathy; (vii) inhibiting, preventing, stabilizing, and/or slowing the rate of progression of the multi-systemic manifestations of GSD IV including cardiomyopathy, hepatic and musculoskeletal dysfunction, (viii) inhibiting, preventing, stabilizing, and/or slowing the rate of progression of the multi-systemic manifestations of APBD; (ix) inhibiting, preventing, and/or slowing the formation and/or cellular inclusion of polyglucosan bodies; (x) inhibiting, preventing, stabilizing, and/or slowing the rate of progression of the onset of CNS- and PNS-related manifestations including neurogenic bladder, peripheral neuropathy, motor neuron disease, gait disturbances, cognitive decline, and other CNS/PNS manifestations as known to the skilled person in the art; (xi) inhibiting, preventing, stabilizing, and/or slowing the rate of progression of liver disease including fibrosis, cirrhosis, hepatic adenomas, and liver hepatocellular carcinoma, or (xii) any combination thereof. In an aspect, restoring one or more aspects of cellular homeostasis can comprise improving, enhancing, restoring, and/or preserving one or more aspects of cellular structural and/or functional integrity.

In an aspect of a disclosed method, techniques to monitor, measure, and/or assess the restoring one or more aspects of cellular homeostasis and/or cellular functionality can comprise qualitative (or subjective) means as well as quantitative (or objective) means. These means are known to the skilled person.

In an aspect, a disclosed method of reducing the expression level, activity level, or both of glycogen synthase can comprise administering a small molecule. In an aspect, a disclosed small molecule can reduce activity and/or expression of GYS1 in view of the reduced activity and/or expression level of GAA, GBE, or one or more other enzymes in the metabolic pathways of glycogen metabolism and glycolysis. In an aspect, a disclosed small molecule can traverse the blood-brain-barrier. In an aspect, a disclosed small molecule can be guaiacol. In an aspect, a disclosed small molecule that inhibits glycogen synthase (GYS1) can be orally delivered.

In an aspect, a disclosed method of reducing the expression level and/or activity level of glycogen synthase can comprise using a gene editing system. In an aspect, a gene editing system can comprise CRISPR/Cas9, or can comprise zinc finger nucleases (ZFNs), transcription activator-like effector nucleases (TALENs), and/or homing endonucleases.

In an aspect, a disclosed method can further comprise administering to the subject a therapeutically effective amount of a therapeutic agent. In an aspect, a disclosed method can comprise reducing glycogen levels by administering a glycogen synthase inhibitor (e.g., RNAi, ASO, etc.) to the subject, or modifying the subject's diet, for example, by using cornstarch or another slow release starch to prevent hypoglycemia, or modifying the subject's diet, for example, by consuming a high amount of protein, fat, or other anaplerotic agents (such as, for example, C7 compounds like triheptanoin), exercise or a combination thereof. In an aspect, a disclosed method can comprise gene editing one or more relevant genes (such as, for example, genes in the glycogenolysis pathway), wherein editing includes but is not limited to single gene knockout, loss of function screening of multiple genes at one, gene knockin, or a combination thereof).

In an aspect, a disclosed method can further comprise administering to the subject a therapeutically effective amount of an agent that can correct one or more aspects of a dysregulated glycogen metabolism pathway, such as glycogen synthesis or glycogenolysis. In an aspect, such an agent can comprise an enzyme for enzyme replacement therapy. In an aspect, a disclosed enzyme can replace a mutated or dysfunction or nonexistence product of the GYG1, RBCK1, PRKAG2, or GBE gene, or a combination thereof. In an aspect, a disclosed enzyme can replace any enzyme in a dysregulated or dysfunctional glycogen metabolism pathway (see, e.g., FIG. 2).

In an aspect, a disclosed method can comprise administering one or more immune modulators. In an aspect, a disclosed immune modulator can be methotrexate, rituximab, intravenous gamma globulin, or bortezomib, or a combination thereof. In an aspect, a disclosed immune modulator can be bortezomib or SVP-Rapamycin.

In an aspect, a disclosed method can comprise plasmapheresis to remove, for example, antibodies to one or more administered treatments.

In an aspect, a disclosed immune modulator such as methotrexate can be administered at a transient low to high-dose. In an aspect, a disclosed immune modulator can be administered at a dose of about 0.1 mg/kg body weight to about 0.6 mg/kg body weight. In an aspect, a disclosed immune modulator can be administered at a dose of about 0.4 mg/kg body weight. In an aspect, a disclosed immune modulator can be administered at about a daily dose of 0.4 mg/kg body weight for 3 to 5 or greater cycles, with up to three days per cycle. In an aspect, a disclosed immune modulator can be administered at about a daily dose of 0.4 mg/kg body weight for a minimum of 3 cycles, with three days per cycle. In an aspect, a person skilled in the art can determine the appropriate number of cycles. In an aspect, a disclosed immune modulator can be administered as many times as necessary to achieve a desired clinical effect.

In an aspect, a disclosed immune modulator can be administered orally about one hour before a disclosed therapeutic agent. In an aspect, a disclosed immune modulator can be administered subcutaneously about 15 minutes before a disclosed therapeutic agent. In an aspect, a disclosed immune modulator can be administered concurrently with a disclosed therapeutic agent.

In an aspect, a disclosed immune modulator can be administered orally about one hour or a few days before a disclosed isolated nucleic acid molecule, a disclosed vector, a disclosed pharmaceutical formulation, or a combination thereof. In an aspect, a disclosed immune modulator can be administered subcutaneously about 15 minutes before or a few days before a disclosed isolated nucleic acid molecule, a disclosed vector, a disclosed pharmaceutical formulation, or a combination thereof. In an aspect, a disclosed immune modulator can be administered concurrently with a disclosed isolated nucleic acid molecule, a disclosed vector, a disclosed pharmaceutical formulation, or a combination thereof.

In an aspect, a disclosed method can comprise administering one or more proteasome inhibitors (e.g., bortezomib, carfilzomib, marizomib, ixazomib, and oprozomib). In an aspect, a proteasome inhibitor can be an agent that acts on plasma cells (e.g., daratumumab). In an aspect, an agent that acts on a plasma cell can be melphalan hydrochloride, melphalan, pamidronate disodium, carmustine, carfilzomib, carmustine, cyclophosphamide, daratumumab, doxorubicin hydrochloride liposome, doxorubicin hydrochloride liposome, elotuzumab, melphalan hydrochloride, panobinostat, ixazomib citrate, carfilzomib, lenalidomide, melphalan, melphalan hydrochloride, plerixafor, ixazomib citrate, pamidronate disodium, panobinostat, plerixafor, pomalidomide, pomalidomide, lenalidomide, selinexor, thalidomide, thalidomide, bortezomib, selinexor, zoledronic acid, or zoledronic acid.

In an aspect, a disclosed method can comprise administering one or more proteasome inhibitors or agents that act on plasma cells prior to administering a disclosed isolated nucleic acid molecule, a disclosed vector, or a disclosed pharmaceutical formulation. In an aspect, a disclosed method can comprise administering one or more proteasome inhibitors or one or more agents that act on plasma cells concurrently with administering a disclosed isolated nucleic acid molecule, a disclosed vector, or a disclosed pharmaceutical formulation. In an aspect, a disclosed method can comprise administering one or more proteasome inhibitors or one or more agents that act on plasma cells subsequent to administering a disclosed isolated nucleic acid molecule, a disclosed vector, or a disclosed pharmaceutical formulation.

In an aspect, a disclosed method can comprise administering one or more proteasome inhibitors more than 1 time. In an aspect, a disclosed method can comprise administering one or more proteasome inhibitors repeatedly over time.

In an aspect, a disclosed method can comprise administering one or more immunosuppressive agents. In an aspect, an immunosuppressive agent can be, but is not limited to, azathioprine, methotrexate, sirolimus, anti-thymocyte globulin (ATG), cyclosporine (CSP), mycophenolate mofetil (MMF), steroids, or a combination thereof. In an aspect, a disclosed method can comprise administering one or more immunosuppressive agents more than 1 time.

In an aspect, a disclosed method can comprise administering one or more one or more immunosuppressive agents repeatedly over time. In an aspect, a disclosed method can comprise administering a compound that targets or alters antigen presentation or humoral or cell mediated or innate immune responses.

In an aspect, a disclosed method can comprise administering a compound that exerts a therapeutic effect against B cells and/or a compound that targets or alters antigen presentation or humoral or cell mediated immune response. In an aspect, a disclosed compound can be rituximab, methotrexate, intravenous gamma globulin, anti CD4 antibody, anti CD2, an anti-FcRN antibody, a BTK inhibitor, an anti-IGF1R antibody, a CD19 antibody (e.g., inebilizumab), an anti-IL6 antibody (e.g., tocilizumab), an antibody to CD40, an IL2 mutein, or a combination thereof. Also disclosed herein are Treg infusions that can be administered as a way to help with immune tolerance (e.g., antigen specific Treg cells to AAV).

In an aspect, a disclosed method comprises administering lipid nanoparticles (LNPs). In an aspect, LNPs can be organ-targeted. In an aspect, LNPs can be liver-targeted or skeletal muscle targeted. For example, in an aspect, mRNA therapy with lipid nanoparticle encapsulation for systemic delivery to hepatocytes has the potential to restore metabolic enzymatic activity for one or more glycogen storage diseases such as GSD IV, Lafora disease (including those diseases caused by mutations in the EPM2A gene (glucan phosphatase, laforin) or the NHLRC1 gene (NHL repeat containing E3 ubiquitin protein ligase 1 or EPM2B)), polyglucosan body myopathy-1, polyglucosan body myopathy-2, APBD, or any disease or pathology caused by a mutation in a GYG1 gene, a RBCK1 gene, or a PRKAG2 gene. In an aspect, the mRNA therapy focuses on a GBE gene, aGYG1 gene, a RBCK1 gene, a PRKAG2 gene, or a combination thereof. In an aspect, the mRNA therapy focuses on one or more genes in the glycogenolysis pathway.

In an aspect, a disclosed method can comprise treating a subject that has developed or is likely to develop neutralizing antibodies (ABs) to the vector, capsid, and/or transgene. In an aspect, treating a subject that has developed or is likely to develop neutralizing antibodies can comprise plasmapheresis and immunosuppression. In an aspect, a disclosed method can comprise using immunosuppression to decrease the T cell, B cell, and/or plasma cell population, decrease the innate immune response, inflammatory response, and antibody levels in general. In an aspect, a disclosed method can comprise administering an IgG-degrading agent that depletes pre-existing neutralizing antibodies. In an aspect, a disclosed method can comprise administering to the subject IdeS or IdeZ, rapamycin, and/or SVP-Rapamycin. In an aspect, a disclosed IgG-degrading agent is bacteria-derived IdeS or IdeZ.

In an aspect, a disclosed method can comprise plasmapheresis to remove, for example, antibodies to one or more administered treatments.

In an aspect, a disclosed method can further comprise administering to the subject an effective amount an isolated nucleic acid encoding a protein that is deficient or absent in the subject. In an aspect, a disclosed encoded protein can comprise a recombinant human protein such as, for example, recombinant alpha-glucosidase (GAA). In an aspect, a disclosed GAA can comprise the amino acid sequence set forth in any one of SEQ ID NO:15-SEQ ID NO: 19 or a fragment thereof. In an aspect, a disclosed GAA can comprise a sequence having at least 30%, at least 40%, at least 50%, at least 60%, at least 70%, at least 80%, or at least 90% identity to the amino acid sequence set forth in any one of SEQ ID NO:15-SEQ ID NO:19 or a fragment thereof. In an aspect, a disclosed GAA can be Myozyme or Lumizyme. In an aspect of a disclosed method, a disclosed isolated nucleic acid sequence for GAA can comprise the sequence set forth in SEQ ID NO:20, SEQ ID NO:21 or a fragment thereof. In an aspect, a disclosed isolated nucleic acid sequence for GAA can comprise a sequence having at least 30%, at least 40%, at least 50%, at least 60%, at least 70%, at least 80%, or at least 90% identity to the nucleotide sequence set forth in SEQ ID NO:20, SEQ ID NO:21, or a fragment thereof. In an aspect, a disclosed isolated nucleic acid encoding a recombinant protein such as, for example, GAA, can be present in a disclosed viral vector including, for example, an AAV vector or a self-complementary AAV vector. In an aspect, a disclosed immune modulator and a disclosed therapeutic agent can be concurrently administered. In an aspect, a disclosed composition comprising GAA or a disclosed vector comprising a disclosed isolated nucleic acid molecule encoding GAA can be administered prior to, concurrent with, or after the administration of a disclosed vector comprising a disclosed isolated nucleic acid molecule comprising a nucleic acid sequence encoding a polypeptide for preventing glycogen accumulation and/or degrading accumulated glycogen.

### E. Methods for Preventing Accumulation of Glycogen, Polyglucosan Bodies, and/or Amylopectin-Like Glycogen

Disclosed herein is a method of preventing glycogen accumulation and/or degrading accumulated glycogen comprising administering to a subject in need thereof a therapeutically effective amount of a disclosed isolated nucleic acid molecule, a disclosed vector, disclosed pharmaceutical formulation, or a combination thereof.

Disclosed herein is a method of preventing glycogen accumulation and/or degrading accumulated glycogen comprising administering to a subject in need thereof a therapeutically effective amount of a disclosed isolated nucleic acid molecule, a disclosed vector, disclosed pharmaceutical formulation, or a combination thereof, and restoring the level of glycogen synthase (GYS1) and/or GBE to normal or near normal in the subject or in a tissue and/or organ in the subject.

Disclosed herein is a method of preventing glycogen accumulation and/or degrading accumulated glycogen comprising administering to a subject in need thereof a therapeutically effective amount of a disclosed isolated nucleic acid molecule, a disclosed vector, a disclosed pharmaceutical formulation, or a combination thereof, and a therapeutically effective amount of an agent for reducing the expression level and/or activity level of glycogen synthase.

Disclosed herein is a method of preventing glycogen accumulation and/or degrading accumulated glycogen comprising administering to a subject having a disease a therapeutically effective amount of a vector comprising an isolated nucleic acid molecule comprising a nucleic acid sequence encoding a polypeptide for preventing glycogen accumulation and/or degrading accumulated glycogen, wherein the nucleic acid sequence is CpG-depleted and codon-optimized for expression in a human or a mammalian cell.

Disclosed herein is a method of preventing glycogen accumulation and/or degrading accumulated glycogen comprising administering to the subject a therapeutically effective amount of a vector comprising an isolated nucleic acid molecule comprising a nucleic acid sequence encoding a polypeptide for preventing glycogen accumulation and/or degrading accumulated glycogen, wherein the nucleic acid sequence is CpG-depleted and codon-optimized for expression in a human or a mammalian cell.

Disclosed herein is a method of preventing glycogen accumulation and/or degrading accumulated glycogen comprising administering to a subject in need thereof a therapeutically effective amount of a vector comprising an isolated nucleic acid molecule comprising a nucleic acid sequence encoding a polypeptide for preventing glycogen accumulation and/or degrading accumulated glycogen, wherein the nucleic acid sequence is CpG-depleted and codon-optimized for expression in a human or a mammalian cell, wherein glycogen accumulation is prevented and/or accumulated glycogen is degraded in the subject.

Disclosed herein is a method of preventing glycogen accumulation and/or degrading accumulated glycogen comprising administering to a subject in need thereof a therapeutically effective amount of a vector comprising an isolated nucleic acid molecule comprising a nucleic acid sequence encoding a polypeptide for preventing glycogen accumulation and/or degrading accumulated glycogen, wherein the nucleic acid sequence is CpG-depleted and codon-optimized for expression in a human or a mammalian cell, thereby preventing glycogen accumulation and/or degrading accumulated glycogen in the subject.

Disclosed herein is a method of preventing glycogen accumulation and/or degrading accumulated glycogen comprising administering to a subject having GSD IV a therapeutically effective amount of a vector comprising an isolated nucleic acid molecule comprising a nucleic acid sequence encoding a polypeptide for preventing glycogen accumulation and/or degrading accumulated glycogen, wherein the nucleic acid sequence is CpG-depleted and codon-optimized for expression in a human or a mammalian cell.

Disclosed herein is a method of preventing glycogen accumulation and/or degrading accumulated glycogen on comprising administering to a subject in need thereof a therapeutically effective amount of a vector comprising an isolated nucleic acid molecule comprising a nucleic acid sequence encoding a polypeptide for preventing glycogen accumulation and/or degrading accumulated glycogen, wherein the nucleic acid sequence is CpG-depleted and codon-optimized for expression in a human or a mammalian cell, and a therapeutically effective amount of an agent for reducing the expression level and/or activity level of glycogen synthase, wherein glycogen accumulation is prevented and/or accumulated glycogen is degraded in the subject.

Disclosed herein is a method of preventing glycogen accumulation and/or degrading accumulated glycogen comprising administering to the subject a therapeutically effective amount of a vector comprising an isolated nucleic acid molecule comprising a nucleic acid sequence encoding a polypeptide for preventing glycogen accumulation and/or degrading accumulated glycogen, wherein the nucleic acid sequence is CpG-depleted and codon-optimized for expression in a human or a mammalian cell, and a therapeutically effective amount of an agent for reducing the expression level and/or activity level of glycogen synthase.

Disclosed herein is a method of preventing glycogen accumulation and/or degrading accumulated glycogen comprising administering to a subject in need thereof a therapeutically effective amount of a vector comprising an isolated nucleic acid molecule comprising a nucleic acid sequence encoding a polypeptide for preventing glycogen accumulation and/or degrading accumulated glycogen, wherein the nucleic acid sequence is CpG-depleted and codon-optimized for expression in a human or a mammalian cell, and a therapeutically effective amount of an agent for reducing the expression level and/or activity level of glycogen synthase, thereby preventing glycogen accumulation and/or degrading accumulated glycogen in the subject.

In an aspect, “glycogen” can refer to glycogen, polyglucosan bodies, amylopectin-like glycogen, Lafora bodies, or any combination thereof. For example, the term “glycogen accumulation” can comprise accumulation of glycogen, Lafora bodies, polyglucosan bodies, amylopectin-like glycogen, or any combination thereof. In an aspect, accumulation can refer to accumulation of glycogen, polyglucosan bodies, Lafora bodies, amylopectin-like glycogen, or any combination thereof. In an aspect, a disclosed encoded polypeptide can degrade glycogen, polyglucosan bodies, amylopectin-like glycogen, Lafora bodies, or any combination thereof. In an aspect, a disclosed encoded polypeptide can degrade any form of accumulated glycogen.

In an aspect, a subject can have a GSD (such as GSD IV and/or APBD), Lafora disease (including those diseases caused by mutations in the EPM2A gene (glucan phosphatase, laforin) or the NHLRC1 gene (NHL repeat containing E3 ubiquitin protein ligase 1 or EPM2B)), polyglucosan body myopathy-1, polyglucosan body myopathy-2, or any disease or pathology caused by a mutation in a GYG1 gene, a RBCK1 gene, or a PRKAG2 gene.

In an aspect, a disclosed encoded polypeptide can be a human glycogen branching enzyme. In an aspect, a disclosed encoded polypeptide can be a human salivary or pancreatic amylase. In an aspect, a disclosed encoded polypeptide can be derived from plant, bacteria, or another microorganism. In an aspect, a disclosed encoded polypeptide can be derived from any non-human species, such as, for example, gorilla, chimpanzee, Rhesus monkey, dog, cow, mouse, rat, chicken, zebrafish, fruit fly, mosquito, C. elegans, and frog. In an aspect, a mammalian cell can be a cell from any non-human species, such as, for example, a cell from a gorilla, a chimpanzee, a Rhesus monkey, a dog, a cow, a mouse, and a rat.

In an aspect, a disclosed encoded polypeptide can comprise the sequence set forth in SEQ ID NO:01. In an aspect, a disclosed encoded polypeptide can comprise a sequence having at least 30%, at least 40%, at least 50%, at least 60%, at least 70%, at least 80%, or at least 90% identity to the sequence set forth in SEQ ID NO:01. In an aspect, a disclosed encoded polypeptide can comprise the sequence set forth in SEQ ID NO:07. In an aspect, a disclosed encoded polypeptide can comprise a sequence having at least 30%, at least 40%, at least 50%, at least 60%, at least 70%, at least 80%, or at least 90% identity to the sequence set forth in SEQ ID NO:07. In an aspect, a disclosed encoded polypeptide can comprise the sequence set forth in SEQ ID NO:08. In an aspect, a disclosed encoded polypeptide can comprise a sequence having at least 30%, at least 40%, at least 50%, at least 60%, at least 70%, at least 80%, or at least 90% identity to the sequence set forth in SEQ ID NO:08.

In an aspect, a disclosed nucleic acid sequence encoding a polypeptide can comprise the sequence set forth in SEQ ID NO:03 or SEQ ID NO:04. In an aspect, a disclosed nucleic acid sequence encoding a polypeptide can comprise a sequence having at least 30%, at least 40%, at least 50%, at least 60%, at least 70%, at least 80%, or at least 90% identity to the sequence set forth in SEQ ID NO:03 or SEQ ID NO:04. In an aspect, a disclosed nucleic acid sequence can comprise a sequence having at least 80% identity to the sequence set forth in SEQ ID NO:03 or SEQ ID NO:04. In an aspect, the original (non-CpG-depleted) polynucleotide open reading frame (ORF) sequence of human glycogen branching enzyme can comprise the sequence set forth in SEQ ID NO:2. In an aspect, the original (non-CpG-depleted) polynucleotide open reading frame (ORF) sequence of human glycogen branching enzyme can comprise a sequence having at least 50-69%, at least 70-89%, or at least 90-99% identity to the sequence set forth in SEQ ID NO:02.

In an aspect, a disclosed nucleic acid molecule can be in a disclosed vector. In an aspect, a disclosed vector can be a disclosed viral vector or a disclosed non-viral vector (discussed supra). In an aspect, a disclosed viral vector can be a disclosed AAV vector such as, for example, can be AAV9, AAVF, AAVcc.47, or AAVcc.81. In an aspect, a disclosed AAV vector can be AAV-Rh74 or a related variant (e.g., capsid variants like RHM4-1).

In an aspect, a disclosed isolated nucleic acid molecule and/or a disclosed vector can be administered via any disclosed method of administration such as, for example, intravenous, intraarterial, intramuscular, intraperitoneal, subcutaneous, intra-CSF, intrathecal, intraventricular, or in utero administration.

In an aspect, a disclosed isolated nucleic acid molecule and/or a disclosed vector can be administered via intra-CSF administration in combination with a disclosed nucleic acid molecule, a disclosed vector, and/or a disclosed pharmaceutical formulation. In an aspect, a disclosed vector can be delivered to the subject's liver, heart, skeletal muscle, smooth muscle, CNS, PNS, or a combination thereof. In an aspect, a disclosed vector can be concurrently and/or serially administered to a subject via multiple routes of administration. For example, in an aspect, administering a disclosed vector can comprise intravenous administration and intra-cistern magna (ICM) administration. In an aspect, administering a disclosed vector can comprise IV administration and intrathecal (ITH) administration.

In an aspect, a therapeutically effective amount of disclosed vector can be delivered via intravenous (IV) administration and can comprise a range of 1×10¹⁰ to 2×10¹⁴·vg/kg. In an aspect, a therapeutically effective amount of disclosed vector can be delivered via intra-cistem magna (ICM) administration and can comprise a range of 1×10⁹ to 2×10¹⁴·vg. In an aspect, a therapeutically effective amount of disclosed vector can be delivered via intrathecal (ITH) administration and can comprise a range of 1×10⁹ to 2×10¹⁴·vg. In an aspect, a therapeutically effective amount of disclosed vector can be delivered via intracerebroventricular (ICV) administration and can comprise a range of 1×10⁹ to 2×10¹⁴·vg. In an aspect, a therapeutically effective amount of a disclosed vector can comprise a single dose or a series of doses totalling the desired effective amount.

In an aspect, a disclosed vector can comprise a ubiquitous promoter. In an aspect, a disclosed ubiquitous promoter can be a CMV enhancer/chicken β-actin promoter or a CpG-depleted mCMV/hEF1α promoter. In an aspect, a disclosed vector can comprise a tissue-specific promoter. In an aspect, a disclosed tissue-specific promoter can be a liver-specific promoter, a muscle-specific promoter, a neuron-specific promoter (such as, for example, a synapsin I promoter), or a combination thereof. In an aspect, a disclosed liver-specific promoter can be a α1-microglobulin/bikunin enhancer/thyroid hormone-binding globulin promoter.

In an aspect, a disclosed liver-specific promoter can comprise any liver-specific promoter known to the art. In an aspect, a liver specific promoter can comprise about 845-bp and comprise the thyroid hormone-binding globulin promoter sequences (2382 to 13), two copies of α1-microglobulinybikunin enhancer sequences (22,804 through 22,704), and a 71-bp leader sequence as described by Ill C R, et al. (1997). In an aspect, a disclosed liver-specific promoter can comprise the sequence set froth in SEQ ID NO:34. In an aspect, a disclosed liver-specific promoter can comprise a sequence having at least 40%, 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:34. In an aspect, a disclosed liver-specific promoter can comprise a sequence having at least 40%-60%, at least 60%-80%, at least 80%-90%, or at least 90%-100% identity to the sequence set forth in SEQ ID NO:34.

In an aspect, a disclosed promoter can be an endogenous promoter. In an aspect, a disclosed endogenous promoter can generally be obtained from a non-coding region upstream of a transcription initiation site of a gene (such as, for example, a disclosed GBE or some other enzyme involved in the glycogen signaling pathway). In an aspect, a disclosed engoengous promoter can be used for constitutive and efficient expression of a disclosed transgene (e.g., a nucleic acid sequence encoding a polypeptide capable of preventing glycogen accumulation and/or degrading accumulated glycogen). The skilled person is familiar with the methods and tools to identify an endogenous promoter such as, for example, the endogenous promoter for GBE.

In an aspect, a disclosed vector can comprise an immunotolerant dual promoter comprising a liver-specific promoter and a ubiquitous promoter. In an aspect, an immunotolerant dual promoter can comprise a liver-specific promoter and another tissue specific promoter (such as, for example, a muscle-specific promoter, a neuron-specific promoter (such as, for example, a synapsin I promoter), a skeletal muscle-specific promoter, and a heart-specific promoter). In an aspect, a disclosed immunotolerant dual promoter can comprise a α1-microglobulin/bikunin enhancer/thyroid hormone-binding globulin promoter and a CB promoter. In an aspect, a disclosed immunotolerant dual promoter can comprise a al-microglobulin/bikunin enhancer/thyroid hormone-binding globulin promoter and a CpG-depleted mCMV/hEF1α promoter. In an aspect, a disclosed immunotolerant dual promoter can comprise the sequence set forth in SEQ ID NO:05 or SEQ ID NO:06. In an aspect, a disclosed immunotolerant dual promoter can comprise a sequence having at least 40%, 50%, 60%, 70%, 80%, 85%, 86%, 87%, 88%, 89%, 90%, 91%, 92%, 93%, 94%, 95%, 96%, 97%, 98%, or 99% identity to the sequence set forth in SEQ ID NO:05 or SEQ ID NO:06.

In an aspect, a disclosed method can comprise reducing the expression level, activity level, or both of glycogen synthase. In an aspect, reducing the expression level, activity, or both of glycogen synthase comprises administering a therapeutically effective amount of an agent for reducing the expression level and/or activity level of glycogen synthase. In an aspect, a glycogen synthase can be GYS1 (muscle glycogen synthase) or GYS2 (liver glycogen synthase) or both. In an aspect, a disclosed method of reducing the expression level, activity level, or both of glycogen synthase can comprise administering an RNA therapeutic. RNA therapeutics are known to the art and include double stranded RNA-mediated interference (RNAi) and antisense oligonucleotides (ASO). Thus, in an aspect, a disclosed method can comprise administering RNAi or administering ASO or both. In an aspect, a disclosed method can comprise administering RNAi or administering ASO or both directed at GYS1 and/or GYS2.

In an aspect, a disclosed method of reducing the expression level, activity level, or both of glycogen synthase can comprise SRT. For example, in an aspect, SRT can comprise inhibiting glycogen synthase (i.e., GYS1 and/or GYS2) in a cell or a subject to reduce glycogen synthesis and/or glycogen accumulation in cells and tissues (e.g., skeletal muscle, lung tissue, liver tissue, brain tissue, or any other tissue having glycogen accumulation) when GAA and/or GBE activity and/or expression levels are reduced. In an aspect, SRT can comprise siRNA-based therapies, shRNA-based therapies, antisense therapies, gene-editing therapies, and therapies using one or more small molecules or peptide drugs.

In an aspect, a disclosed method can comprise restoring the level of glycogen synthase (GYS1) to normal or near normal in a subject or in a tissue and/or organ in a subject. In an aspect, a disclosed method can comprise restoring the level of GBE to normal or near normal in a subject or in a tissue and/or organ in a subject. In an aspect, a disclosed method can comprise restoring the ratio of GYS1 and GBE to normal or near normal in a subject or in a tissue and/or organ in a subject.

In an aspect, a disclosed method can comprise restoring glucose homeostasis. In an aspect of a disclosed method, techniques to monitor, measure, and/or assess the restoring glucose homeostasis can comprise qualitative (or subjective) means as well as quantitative (or objective) means. These means are known to the skilled person.

In an aspect, a disclosed method can restore one or more aspects of the glycogen signaling pathway, restore one or more aspects of the glycogenolysis signaling pathway, can restore one or more aspects of the glycogenesis signaling pathway, or any combination thereof. In an aspect, a restoring one or more aspects of a disclosed signaling pathway can comprise restoring the activity and/or functionality of one or more enzymes identified in FIG. 2. In an aspect, restoration can be a 10%, 20%, 30%, 40%, 50%, 60%, 70%, 80%, 90%, 100%, or any amount of restoration when compared to a pre-existing level such as, for example, a pre-treatment level. In an aspect, the amount of restoration can be 10-20%, 20-30%, 30-40%, 40-50%, 50-60%, 60-70%, 70-80%, 80-90%, or 90-100% more than a pre-existing level such as, for example, a pre-treatment level. In an aspect, restoration can be measured against a control level (e.g., a level in a subject not having a GSD). In an aspect, restoration can be a partial or incomplete restoration. In an aspect, restoration can be complete or near complete restoration such that the level of expression, activity and/or functionality is similar to that of a wild-type or control level.

In an aspect, a disclosed method can comprise restoring one or more aspects of cellular homeostasis and/or cellular functionality. In an aspect, restoring one or more aspects of cellular homeostasis and/or cellular functionality can comprise one or more of the following: (i) correcting cell starvation in one or more cell types (such as, for example, liver cells, muscle cells, cells in the PNS, and cells in the CNS); (ii) normalizing aspects of autophagy pathway (correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) preventing, slowing, and/or eliminating hypoglycemia, ketosis, and/or other liver abnormalities related to liver disease; (vi) improving, preventing, and/or reversing neurogenic bladder, gait disturbances, and/or neuropathy; (vii) inhibiting, preventing, stabilizing, and/or slowing the rate of progression of the multi-systemic manifestations of GSD IV including cardiomyopathy, hepatic and musculoskeletal dysfunction, (viii) inhibiting, preventing, stabilizing, and/or slowing the rate of progression of the multi-systemic manifestations of APBD; (ix) inhibiting, preventing, and/or slowing the formation and/or cellular inclusion of polyglucosan bodies; (x) inhibiting, preventing, stabilizing, and/or slowing the rate of progression of the onset of CNS- and PNS-related manifestations including neurogenic bladder, peripheral neuropathy, motor neuron disease, gait disturbances, cognitive decline, and other CNS/PNS manifestations as known to the skilled person in the art, or (xi) inhibiting, preventing, stabilizing, and/or slowing the rate of progression of liver disease including fibrosis, cirrhosis, hepatic adenomas, and liver hepatocellular carcinoma, or (xii) any combination thereof. In an aspect, restoring one or more aspects of cellular homeostasis can comprise improving, enhancing, restoring, and/or preserving one or more aspects of cellular structural and/or functional integrity.

In an aspect of a disclosed method, techniques to monitor, measure, and/or assess the restoring one or more aspects of cellular homeostasis and/or cellular functionality can comprise qualitative (or subjective) means as well as quantitative (or objective) means. These means are known to the skilled person.

In an aspect, a disclosed method of reducing the expression level, activity level, or both of glycogen synthase can comprise administering a small molecule. In an aspect, a disclosed small molecule can reduce activity and/or expression of GYS1 in view of the reduced activity and/or expression level of GAA, GBE, or one or more other enzymes in the metabolic pathways of glycogen metabolism and glycolysis. In an aspect, a disclosed small molecule can traverse the blood-brain-barrier. In an aspect, a disclosed small molecule can be guaiacol. In an aspect, a disclosed small molecule that inhibits glycogen synthase (GYS1) can be orally delivered.

In an aspect, a disclosed method of reducing the expression level and/or activity level of glycogen synthase can comprise using a gene editing system. In an aspect, a gene editing system can comprise CRISPR/Cas9, or can comprise zinc finger nucleases (ZFNs), transcription activator-like effector nucleases (TALENs), and/or homing endonucleases.

In an aspect, a disclosed method can further comprise administering to the subject a therapeutically effective amount of a therapeutic agent. In an aspect, a disclosed method can comprise reducing glycogen levels by administering a glycogen synthase inhibitor (e.g., RNAi, ASO, etc.) to the subject, or modifying the subject's diet, for example, by using cornstarch or another slow release starch to prevent hypoglycemia, or modifying the subject's diet, for example, by consuming a high amount of protein, fat, or other anaplerotic agents (such as, for example, C7 compounds like triheptanoin), exercise or a combination thereof. In an aspect, a disclosed method can comprise gene editing one or more relevant genes (such as, for example, genes in the glycogenolysis pathway), wherein editing includes but is not limited to single gene knockout, loss of function screening of multiple genes at one, gene knockin, or a combination thereof).

In an aspect, a disclosed method can further comprise administering to the subject a therapeutically effective amount of an agent that can correct one or more aspects of a dysregulated glycogen metabolism pathway, such as glycogen synthesis or glycogenolysis. In an aspect, such an agent can comprise an enzyme for enzyme replacement therapy. In an aspect, a disclosed enzyme can replace a mutated or dysfunction or nonexistence product of the GYG1, RBCK1, PRKAG2, or GBE gene, or a combination thereof. In an aspect, a disclosed enzyme can replace any enzyme in a dysregulated or dysfunctional glycogen metabolism pathway (see, e.g., FIG. 2).

In an aspect, a disclosed method can comprise administering one or more immune modulators such as, for example, methotrexate, rituximab, intravenous gamma globulin, or bortezomib, SVP-Rapamycin, or a combination thereof. In an aspect, a disclosed method can comprise plasmapheresis to remove, for example, antibodies to one or more administered treatments.

In an aspect, a disclosed immune modulator such as methotrexate can be administered at a transient low to high-dose as discussed supra. In an aspect, a person skilled in the art can determine the appropriate number of cycles. In an aspect, a disclosed immune modulator can be administered as many times as necessary to achieve a desired clinical effect.

In an aspect, a disclosed immune modulator can be administered orally about one hour before a disclosed therapeutic agent. In an aspect, a disclosed immune modulator can be administered subcutaneously about 15 minutes before a disclosed therapeutic agent. In an aspect, a disclosed immune modulator can be administered concurrently with a disclosed therapeutic agent.

In an aspect, a disclosed immune modulator can be administered orally about one hour or a few days before a disclosed isolated nucleic acid molecule, a disclosed vector, a disclosed pharmaceutical formulation, or a combination thereof. In an aspect, a disclosed immune modulator can be administered subcutaneously about 15 minutes before or a few days before a disclosed isolated nucleic acid molecule, a disclosed vector, a disclosed pharmaceutical formulation, or a combination thereof. In an aspect, a disclosed immune modulator can be administered concurrently with a disclosed isolated nucleic acid molecule, a disclosed vector, a disclosed pharmaceutical formulation, or a combination thereof.

In an aspect, a disclosed method can comprise administering one or more disclosed proteasome inhibitors (e.g., bortezomib, carfilzomib, marizomib, ixazomib, and oprozomib and others discussed supra) and discussed supra. In an aspect, a proteasome inhibitor can be a disclosed agent that acts on plasma cells (e.g., daratumumab). In an aspect, an agent that acts on a plasma cell can be melphalan hydrochloride, melphalan, pamidronate disodium, carmustine, carfilzomib, carmustine, cyclophosphamide, daratumumab, doxorubicin hydrochloride liposome, doxorubicin hydrochloride liposome, elotuzumab, melphalan hydrochloride, panobinostat, ixazomib citrate, carfilzomib, lenalidomide, melphalan, melphalan hydrochloride, plerixafor, ixazomib citrate, pamidronate disodium, panobinostat, plerixafor, pomalidomide, pomalidomide, lenalidomide, selinexor, thalidomide, thalidomide, bortezomib, selinexor, zoledronic acid, or zoledronic acid.

In an aspect, a disclosed method can comprise administering one or more proteasome inhibitors or agents that act on plasma cells prior to administering a disclosed isolated nucleic acid molecule, a disclosed vector, or a disclosed pharmaceutical formulation. In an aspect, a disclosed method can comprise administering one or more proteasome inhibitors or one or more agents that act on plasma cells concurrently with administering a disclosed isolated nucleic acid molecule, a disclosed vector, or a disclosed pharmaceutical formulation. In an aspect, a disclosed method can comprise administering one or more proteasome inhibitors or one or more agents that act on plasma cells subsequent to administering a disclosed isolated nucleic acid molecule, a disclosed vector, or a disclosed pharmaceutical formulation.

In an aspect, a disclosed method can comprise administering one or more proteasome inhibitors more than 1 time. In an aspect, a disclosed method can comprise administering one or more proteasome inhibitors repeatedly over time.

In an aspect, a disclosed method can comprise administering one or more immunosuppressive agents. In an aspect, an immunosuppressive agent can be, but is not limited to, azathioprine, methotrexate, sirolimus, anti-thymocyte globulin (ATG), cyclosporine (CSP), mycophenolate mofetil (MMF), steroids, or a combination thereof. In an aspect, a disclosed method can comprise administering one or more immunosuppressive agents more than 1 time.

In an aspect, a disclosed method can comprise administering one or more one or more immunosuppressive agents repeatedly over time. In an aspect, a disclosed method can comprise administering a compound that targets or alters antigen presentation or humoral or cell mediated or innate immune responses.

In an aspect, a disclosed method can comprise administering a compound that exerts a therapeutic effect against B cells and/or a compound that targets or alters antigen presentation or humoral or cell mediated immune response. In an aspect, a disclosed compound can be rituximab, methotrexate, intravenous gamma globulin, anti CD4 antibody, anti CD2, an anti-FcRN antibody, a BTK inhibitor, an anti-IGF1R antibody, a CD19 antibody (e.g., inebilizumab), an anti-IL6 antibody (e.g., tocilizumab), an antibody to CD40, an IL2 mutein, or a combination thereof. Also disclosed herein are Treg infusions that can be administered as a way to help with immune tolerance (e.g., antigen specific Treg cells to AAV).

In an aspect, a disclosed method comprises administering lipid nanoparticles (LNPs). In an aspect, LNPs can be organ-targeted. In an aspect, LNPs can be liver-targeted or skeletal muscle targeted. For example, in an aspect, mRNA therapy with lipid nanoparticle encapsulation for systemic delivery to hepatocytes has the potential to restore metabolic enzymatic activity for one or more glycogen storage diseases such as GSD IV and/or APBD, Lafora disease (including those diseases caused by mutations in the EPM2A gene (glucan phosphatase, laforin) or the NHLRC1 gene (NHL repeat containing E3 ubiquitin protein ligase 1 or EPM2B)), polyglucosan body myopathy-1, polyglucosan body myopathy-2, or any disease or pathology caused by a mutation in a GYG1 gene, a RBCK1 gene, or a PRKAG2 gene. In an aspect, the mRNA therapy focuses on a GBE gene, aGYG1 gene, a RBCK1 gene, a PRKAG2 gene, or a combination thereof. In an aspect, the mRNA therapy focuses on one or more genes in the glycogenolysis pathway.

In an aspect, a disclosed method can comprise treating a subject that has developed or is likely to develop neutralizing antibodies (ABs) to the vector, capsid, and/or transgene. In an aspect, treating a subject that has developed or is likely to develop neutralizing antibodies can comprise plasmapheresis and immunosuppression. In an aspect, a disclosed method can comprise using immunosuppression to decrease the T cell, B cell, and/or plasma cell population, decrease the innate immune response, inflammatory response, and antibody levels in general. In an aspect, a disclosed method can comprise administering an IgG-degrading agent that depletes pre-existing neutralizing antibodies. In an aspect, a disclosed method can comprise administering to the subject IdeS or IdeZ, rapamycin, and/or SVP-Rapamycin. In an aspect, a disclosed IgG-degrading agent is bacteria-derived IdeS or IdeZ.

In an aspect, a disclosed method can comprise plasmapheresis to remove, for example, antibodies to one or more administered treatments.

In an aspect, a disclosed method can further comprise administering to the subject an effective amount an isolated nucleic acid encoding a protein that is deficient or absent in the subject. In an aspect, a disclosed encoded protein can comprise a recombinant human protein such as, for example, recombinant alpha-glucosidase (GAA). In an aspect, a disclosed GAA can comprise the amino acid sequence set forth in any one of SEQ ID NO:15-SEQ ID NO:19 or a fragment thereof. In an aspect, a disclosed GAA can comprise a sequence having at least 30%, at least 40%, at least 50%, at least 60%, at least 70%, at least 80%, or at least 90% identity to the amino acid sequence set forth in any one of SEQ ID NO:15-SEQ ID NO:19 or a fragment thereof. In an aspect, a disclosed GAA can be Myozyme or Lumizyme. In an aspect of a disclosed method, a disclosed isolated nucleic acid sequence for GAA can comprise the sequence set forth in SEQ ID NO:20, SEQ ID NO:21 or a fragment thereof. In an aspect, a disclosed isolated nucleic acid sequence for GAA can comprise a sequence having at least 30%, at least 40%, at least 50%, at least 60%, at least 70%, at least 80%, or at least 90% identity to the nucleotide sequence set forth in SEQ ID NO:20, SEQ ID NO:21, or a fragment thereof. In an aspect, a disclosed isolated nucleic acid encoding a recombinant protein such as, for example, GAA, can be present in a disclosed viral vector including, for example, an AAV vector or a self-complementary AAV vector. In an aspect, a disclosed immune modulator and a disclosed therapeutic agent can be concurrently administered. In an aspect, a disclosed composition comprising GAA or a disclosed vector comprising a disclosed isolated nucleic acid molecule encoding GAA can be administered prior to, concurrent with, or after the administration of a disclosed vector comprising a disclosed isolated nucleic acid molecule comprising a nucleic acid sequence encoding a polypeptide for preventing glycogen accumulation and/or degrading accumulated glycogen.

### F. Agents

### 1. Biologically Active Agents

As used herein, the term “biologically active agent” or “biologic active agent” or “bioactive agent” means an agent that is capable of providing a local or systemic biological, physiological, or therapeutic effect in the biological system to which it is applied. For example, the bioactive agent can act to control infection or inflammation, enhance cell growth and tissue regeneration, control tumor growth, act as an analgesic, promote anti-cell attachment, and enhance bone growth, among other functions. Other suitable bioactive agents can include anti-viral agents, vaccines, hormones, antibodies (including active antibody fragments sFv, Fv, and Fab fragments), aptamers, peptide mimetics, functional nucleic acids, therapeutic proteins, peptides, or nucleic acids. Other bioactive agents include prodrugs, which are agents that are not biologically active when administered but, upon administration to a subject are converted to bioactive agents through metabolism or some other mechanism. Additionally, any of the compositions of the invention can contain combinations of two or more bioactive agents. It is understood that a biologically active agent can be used in connection with administration to various subjects, for example, to humans (i.e., medical administration) or to animals (i.e., veterinary administration). As used herein, the recitation of a biologically active agent inherently encompasses the pharmaceutically acceptable salts thereof.

### 2. Pharmaceutically Active Agents

As used herein, the term “pharmaceutically active agent” includes a “drug” or a “vaccine” and means a molecule, group of molecules, complex or substance administered to an organism for diagnostic, therapeutic, preventative medical, or veterinary purposes. This term includes externally and internally administered topical, localized and systemic human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term may also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. This term can also specifically include nucleic acids and compounds comprising nucleic acids that produce a bioactive effect, for example deoxyribonucleic acid (DNA) or ribonucleic acid (RNA). Pharmaceutically active agents include the herein disclosed categories and specific examples. It is not intended that the category be limited by the specific examples. Those of ordinary skill in the art will recognize also numerous other compounds that fall within the categories and that are useful according to the invention. Examples include a radiosensitizer, the combination of a radiosensitizer and a chemotherapeutic, a steroid, a xanthine, a beta-2-agonist bronchodilator, an anti-inflammatory agent, an analgesic agent, a calcium antagonist, an angiotensin-converting enzyme inhibitors, a beta-blocker, a centrally active alpha-agonist, an alpha-1-antagonist, carbonic anhydrase inhibitors, prostaglandin analogs, a combination of an alpha agonist and a beta blocker, a combination of a carbonic anhydrase inhibitor and a beta blocker, an anticholinergic/antispasmodic agent, a vasopressin analogue, an antiarrhythmic agent, an antiparkinsonian agent, an antiangina/antihypertensive agent, an anticoagulant agent, an antiplatelet agent, a sedative, an ansiolytic agent, a peptidic agent, a biopolymeric agent, an antineoplastic agent, a laxative, an antidiarrheal agent, an antimicrobial agent, an antifungal agent, or a vaccine. In a further aspect, the pharmaceutically active agent can be coumarin, albumin, bromolidine, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenterol, clenbuterol, bambuterol, salmeterol, fenoterol; antiinflammatory agents, including antiasthmatic anti-inflammatory agents, antiarthritis antiinflammatory agents, and non-steroidal antiinflammatory agents, examples of which include but are not limited to sulfides, mesalamine, budesonide, salazopyrin, diclofenac, pharmaceutically acceptable diclofenac salts, nimesulide, naproxene, acetominophen, ibuprofen, ketoprofen and piroxicam; analgesic agents such as salicylates; calcium channel blockers such as nifedipine, amlodipine, and nicardipine; angiotensin-converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, timol hemihydrate, levobunolol hydrochloride, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists (i.e., alpha adrenergic receptor agonist) such as clonidine, brimonidine tartrate, and apraclonidine hydrochloride; alpha-1-antagonists such as doxazosin and prazosin; anticholinergic/antispasmodic agents such as dicyclomine hydrochloride, scopolamine hydrobromide, glycopyrrolate, clidinium bromide, flavoxate, and oxybutynin; vasopressin analogues such as vasopressin and desmopressin; prostaglandin analogs such as latanoprost, travoprost, and bimatoprost; cholinergics (i.e., acetylcholine receptor agonists) such as pilocarpine hydrochloride and carbachol; glutamate receptor agonists such as the N-methyl D-aspartate receptor agonist memantine; anti-Vascular endothelial growth factor (VEGF) aptamers such as pegaptanib; anti-VEGF antibodies (including but not limited to anti-VEGF-A antibodies) such as ranibizumab and bevacizumab; carbonic anhydrase inhibitors such as methazolamide, brinzolamide, dorzolamide hydrochloride, and acetazolamide; antiarrhythmic agents such as quinidine, lidocaine, tocainide hydrochloride, mexiletine hydrochloride, digoxin, verapamil hydrochloride, propafenone hydrochloride, flecaimide acetate, procainamide hydrochloride, moricizine hydrochloride, and diisopyramide phosphate; antiparkinsonian agents, such as dopamine, L-Dopa/Carbidopa, selegiline, dihydroergocryptine, pergolide, lisuride, apomorphine, and bromocryptine; antiangina agents and antihypertensive agents such as isosorbide mononitrate, isosorbide dinitrate, propranolol, atenolol and verapamil; anticoagulant and antiplatelet agents such as coumadin, warfarin, acetylsalicylic acid, and ticlopidine; sedatives such as benzodiazapines and barbiturates; ansiolytic agents such as lorazepam, bromazepam, and diazepam; peptidic and biopolymeric agents such as calcitonin, leuprolide and other LHRH agonists, hirudin, cyclosporin, insulin, somatostatin, protirelin, interferon, desmopressin, somatotropin, thymopentin, pidotimod, erythropoietin, interleukins, melatonin, granulocyte/macrophage-CSF, and heparin; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxine hydrochloride, loperamide hydrochloride, furazolidone, diphenoxylate hydrochloride, and microorganisms; vaccines such as bacterial and viral vaccines; antimicrobial agents such as penicillins, cephalosporins, and macrolides, antifungal agents such as imidazolic and triazolic derivatives; and nucleic acids such as DNA sequences encoding for biological proteins, and antisense oligonucleotides. It is understood that a pharmaceutically active agent can be used in connection with administration to various subjects, for example, to humans (i.e., medical administration) or to animals (i.e., veterinary administration). As used herein, the recitation of a pharmaceutically active agent inherently encompasses the pharmaceutically acceptable salts thereof.

### 3. Anti-Bacterial Agents

As used herein, anti-bacterial agents are known to the art. For example, the art generally recognizes several categories of anti-bacterial agents including (1) penicillins, (2) cephalosporins, (3) quinolones, (4) aminoglycosides, (5) monobactams, (6) carbapenems, (7) macrolides, and (8) other agents. For example, as used herein, an anti-bacterial agent can comprise Afenide, Amikacin, Amoxicillin, Ampicillin, Arsphenamine, Augmentin, Azithromycin, Azlocillin, Aztreonam, Bacampicillin, Bacitracin, Balofloxacin, Besifloxacin, Capreomycin, Carbacephem (loracarbef), Carbenicillin, Cefacetrile (cephacetrile), Cefaclomezine, Cefaclor, Cefadroxil (cefadroxyl), Cefalexin (cephalexin), Cefaloglycin (cephaloglycin), Cefalonium (cephalonium), Cefaloram, Cefaloridine (cephaloradine), Cefalotin (cephalothin), Cefamandole, Cefaparole, Cefapirin (cephapirin), Cefatrizine, Cefazaflur, Cefazedone, Cefazolin (cephazolin), Cefcanel, Cefcapene, Cefclidine, Cefdaloxime, Cefdinir, Cefditoren, Cefedrolor, Cefempidone, Cefepime, Cefetamet, Cefetrizole, Cefivitril, Cefixime, Cefluprenam, Cefmatilen, Cefmenoxime, Cefmepidium, Cefmetazole, Cefodizime, Cefonicid, Cefoperazone, Cefoselis, Cefotaxime, Cefotetan, Cefovecin, Cefoxazole, Cefoxitin, Cefozopran, Cefpimizole, Cefpirome, Cefpodoxime, Cefprozil (cefproxil), Cefquinome, Cefradine (cephradine), Cefrotil, Cefroxadine, Cefsumide, Ceftaroline, Ceftazidime, Ceftazidime/Avibactam, Cefteram, Ceftezole, Ceftibuten, Ceftiofur, Ceftiolene, Ceftioxide, Ceftizoxime, Ceftobiprole, Ceftriaxone, Cefuracetime, Cefuroxime, Cefuzonam, Cephalexin, Chloramphenicol, Chlorhexidine, Ciprofloxacin, Clarithromycin, Clavulanic Acid, Clinafloxacin, Clindamycin, Cloxacillin, Colimycin, Colistimethate, Colistin, Crysticillin, Cycloserine 2, Demeclocycline, Dicloxacillin, Dirithromycin, Doripenem, Doxycycline, Efprozil, Enoxacin, Ertapenem, Erythromycin, Ethambutol, Flucloxacillin, Flumequine, Fosfomycin, Furazolidone, Gatifloxacin, Geldanamycin, Gemifloxacin, Gentamicin, Glycopeptides, Grepafloxacin, Herbimycin, Imipenem, Isoniazid, Kanamycin, Levofloxacin, Lincomycin, Linezolid, Lipoglycopeptides, Lomefloxacin, Meropenem, Meticillin, Metronidazole, Mezlocillin, Minocycline, Mitomycin, Moxifloxacin, Mupirocin, Nadifloxacin, Nafcillin, Nalidixic Acid, Neomycin, Netilmicin, Nitrofurantoin, Norfloxacin, Ofloxacin, Oxacillin, Oxazolidinones, Oxolinic Acid, Oxytetracycline, Oxytetracycline, Paromomycin, Pazufloxacin, Pefloxacin, Penicillin G, Penicillin V, Pipemidic Acid, Piperacillin, Piromidic Acid, Pivampicillin, Pivmecillinam, Platensimycin, Polymyxin B, Pristinamycin, Prontosil, Prulifloxacin, Pvampicillin, Pyrazinamide, Quinupristin/dalfopristin, Rifabutin, Rifalazil, Rifampin, Rifamycin, Rifapentine, Rosoxacin, Roxithromycin, Rufloxacin, Sitafloxacin, Sparfloxacin, Spectinomycin, Spiramycin, Streptomycin, Sulbactam, Sulfacetamide, Sulfamethizole, Sulfamethoxazole, Sulfanilimide, Sulfisoxazole, Sulphonamides, Sultamicillin, Teicoplanin, Telavancin, Telithromycin, Temafloxacin, Tetracycline, Thiamphenicol, Ticarcillin, Tigecycline, Tinidazole, Tobramycin, Tosufloxacin, Trimethoprim, Trimethoprim-Sulfamethoxazole, Troleandomycin, Trovafloxacin, Tuberactinomycin, Vancomycin, Viomycin, or pharmaceutically acceptable salts thereof (e.g., such as, for example, chloride, bromide, iodide, and periodate), or a combination thereof. As used herein, the recitation of an anti-bacterial agent inherently encompasses the pharmaceutically acceptable salts thereof.

### 4. Anti-Fungal Agents

Anti-fungal agents are known to the art. The art generally recognizes several categories of anti-fungal agents including (1) azoles (imidazoles), (2) antimetabolites, (3) allylamines, (4) morpholine, (5) glucan synthesis inhibitors (echinocandins), (6) polyenes, (7) benoxaaborale; (8) other antifungal/onychomycosis agents, and (9) new classes of antifungal/onychomycosis agents. For example, as used herein, an anti-fungal agent can comprise Abafungin, Albaconazole, Amorolfin, Amphotericin B, Anidulafungin, Bifonazole, Butenafine, Butoconazole, Candicidin, Caspofungin, Ciclopirox, Clotrimazole, Econazole, Fenticonazole, Filipin, Fluconazole, Flucytosine, Griseofulvin, Haloprogin, Hamycin, Isavuconazole, Isoconazole, Itraconazole, Ketoconazole, Micafungin, Miconazole, Naftifine, Natamycin, Nystatin, Omoconazole, Oxiconazole, Polygodial, Posaconazole, Ravuconazole, Rimocidin, Sertaconazole, Sulconazole, Terbinafine, Terconazole, Tioconazole, Tolnaftate, Undecylenic Acid, Voriconazole, or pharmaceutically acceptable salts thereof, or a combination thereof. In an aspect, an anti-fungal agent can be an azole. Azoles include, but are not limited to, the following: clotrimazole, econazole, fluconazole, itraconazole, ketoconazole, miconazole, oxiconazole, sulconazole, and voriconazole. As used herein, the recitation of an anti-fungal agent inherently encompasses the pharmaceutically acceptable salts thereof.

### 5. Anti-Viral Agents

Anti-viral agents are known to the art. As used herein, for example, an anti-viral can comprise Abacavir, Acyclovir (Aciclovir), Adefovir, Amantadine, Ampligen, Amprenavir (Agenerase), Umifenovir (Arbidol), Atazanavir, Atripla, Baloxavir marboxil (Xofluza), Biktarvy, Boceprevir, Bulevirtide, Cidofovir, Cobicistat (Tybost), Combivir, Daclatasvir (Daklinza), Darunavir, Delavirdine, Descovy, Didanosine, Docosanol, Dolutegravir, Doravirine (Pifeltro), Edoxudine, Efavirenz, Elvitegravir, Emtricitabine, Enfuvirtide, Entecavir, Etravirine (Intelence), Famciclovir, Fomivirsen, Fosamprenavir, Foscamet, Ganciclovir (Cytovene), Ibacitabine, Ibalizumab (Trogarzo), Idoxuridine, Imiquimod, Imunovir, Indinavir, Lamivudine, Letermovir (Prevymis), Lopinavir, Loviride, Maraviroc, Methisazone, Moroxydine, Nelfinavir, Nevirapine, Nexavir (formerly Kutapressin), Nitazoxanide, Norvir, Oseltamivir (Tamiflu), Penciclovir, Peramivir, Penciclovir, Peramivir (Rapivab), Pleconaril, Podophyllotoxin, Raltegravir, Remdesivir, Ribavirin, Rilpivirine (Edurant), Rilpivirine, Rimantadine, Ritonavir, Saquinavir, Simeprevir (Olysio), Sofosbuvir, Stavudine, Taribavirin (Viramidine), Telaprevir, Telbivudine (Tyzeka), Tenofovir alafenamide, Tenofovir disoproxil, Tenofovir, Tipranavir, Trifluridine, Trizivir, Tromantadine, Truvada, Umifenovirk, Valaciclovir, Valganciclovir (Valtrex), Vicriviroc, Vidarabine, Zalcitabine, Zanamivir (Relenza), Zidovudine, and combinations thereof. As used herein, the recitation of any anti-viral agent inherently encompasses the pharmaceutically acceptable salts thereof.

### 6. Corticosteroids

Corticosteroids are well-known in the art. Corticosteroids mimic the effects of hormones that the body produces naturally in your adrenal glands. Corticosteroids can suppress inflammation and can reduce the signs and symptoms of inflammatory conditions (e.g., arthritis and asthma). Corticosteroids can also suppress the immune system. Corticosteroids can act on a number of different cells (e.g., mast cells, neutrophils, macrophages and lymphocytes) and a number of different mediators (e.g., histamine, leukotriene, and cytokine subtypes).

Steroids include, but are not limited to, the following: triamcinolone and its derivatives (e.g., diacetate, hexacetonide, and acetonide), betamethasone and its derivatives (e.g., dipropionate, benzoate, sodium phosphate, acetate, and valerate), dexamethasone and its derivatives (e.g., dipropionate and valerate), flunisolide, prednisone and its derivatives (e.g., acetate), prednisolone and its derivatives (e.g., acetate, sodium phosphate, and tebutate), methylprednisolone and its derivatives (e.g., acetate and sodium succinate), fluocinolone and its derivatives (e.g., acetonide), diflorasone and its derivatives (e.g., diacetate), halcinonide, desoximetasone (desoxymethasone), diflucortolone and its derivatives (e.g., valerate), flucloronide (fluclorolone acetonide), fluocinonide, fluocortolone, fluprednidene and its derivatives (e.g., acetate), flurandrenolide (flurandrenolone), clobetasol and its derivatives (e.g., propionate), clobetasone and its derivatives (e.g., butyrate), alclometasone, flumethasone and its derivatives (e.g., pivalate), fluocortolone and its derivatives (e.g., hexanoate), amcinonide, beclometasone and its derivatives (e.g., dipropionate), fluticasone and its derivatives (e.g., propionate), difluprednate, prednicarbate, flurandrenolide, mometasone, and desonide. As used herein, the recitation of a corticosteroid inherently encompasses the pharmaceutically acceptable salts thereof.

### 7. Analgesics

The compositions of the present disclosure can also be used in combination therapies with opioids and other analgesics, including narcotic analgesics, Mu receptor antagonists, Kappa receptor antagonists, non-narcotic (i.e., non-addictive) analgesics, monoamine uptake inhibitors, adenosine regulating agents, cannabinoid derivatives, Substance P antagonists, neurokinin-1 receptor antagonists and sodium channel blockers, among others. Preferred combination therapies comprise a composition useful in methods described herein with one or more compounds selected from aceclofenac, acemetacin, .alpha.-acetamidocaproic acid, acetaminophen, acetaminosalol, acetanilide, acetylsalicylic acid (aspirin), S-adenosylmethionine, alclofenac, alfentanil, allylprodine, alminoprofen, aloxiprin, alphaprodine, aluminum bis (acetylsalicylate), amfenac, aminochlorthenoxazin, 3-amino-4-hydroxybutyric acid, 2-atnino-4-picoline, aminopropylon, aminopyrine, amixetrine, ammonium salicylate, ampiroxicam, amtolmetin guacil, anileridine, antipyrine, antipyrine salicylate, antrafenine, apazone, bendazac, benorylate, benoxaprofen, benzpiperylon, benzydamine, benzylmorphine, bermoprofen, bezitramide, .alpha.-bisabolol, bromfenac, p-bromoacetanilide, 5-bromosalicylic acid acetate, bromosaligenin, bucetin, bucloxic acid, bucolome, bufexamac, bumadizon, buprenorphine, butacetin, butibufen, butophanol, calcium acetylsalicylate, carbamazepine, carbiphene, carprofen, carsalam, chlorobutanol, chlorthenoxazin, choline salicylate, cinchophen, cinmetacin, ciramadol, clidanac, clometacin, clonitazene, clonixin, clopirac, clove, codeine, codeine methyl bromide, codeine phosphate, codeine sulfate, cropropamide, crotethamide, desomorphine, dexoxadrol, dextromoramide, dezocine, diampromide, diclofenac sodium, difenamizole, difenpiramide, diflunisal, dihydrocodeine, dihydrocodeinone enol acetate, dihydromorphine, dihydroxyalutninum acetylsalicylate, dimenoxadol, dimepheptanol, dimethylthiambutene, dioxaphetyl butyrate, dipipanone, diprocetyl, dipyrone, ditazol, droxicam, emorfazone, enfenamic acid, epirizole, eptazocine, etersalate, ethenzamide, ethoheptazine, ethoxazene, ethylmethylthiambutene, ethylmorphine, etodolac, etofenamate, etonitazene, eugenol, felbinac, fenbufen, fenclozic acid, fendosal, fenoprofen, fentanyl, fentiazac, fepradinol, feprazone, floctafenine, flufenamic acid, flunoxaprofen, fluoresone, flupirtine, fluproquazone, flurbiprofen, fosfosal, gentisic acid, glafenine, glucametacin, glycol salicylate, guaiazulene, hydrocodone, hydromorphone, hydroxypethidine, ibufenac, ibuprofen, ibuproxam, imidazole salicylate, indomethacin, indoprofen, isofezolac, isoladol, isomethadone, isonixin, isoxepac, isoxicam, ketobemidone, ketoprofen, ketorolac, p-lactophenetide, lefetamine, levorphanol, lofentanil, lonazolac, lomoxicam, loxoprofen, lysine acetylsalicylate, magnesium acetylsalicylate, meclofenamic acid, mefenamic acid, meperidine, meptazinol, mesalamine, metazocine, methadone hydrochloride, methotrimeprazine, metiazinic acid, metofoline, metopon, mofebutazone, mofezolac, morazone, morphine, morphine hydrochloride, morphine sulfate, morpholine salicylate, myrophine, nabumetone, nalbuphine, 1-naphthyl salicylate, naproxen, narceine, nefopam, nicomorphine, nifenazone, niflumic acid, nimesulide, 5′-nitro-2′-propoxyacetanilide, norlevorphanol, normethadone, normorphine, norpipanone, olsalazine, opium, oxaceprol, oxametacine, oxaprozin, oxycodone, oxymorphone, oxyphenbutazone, papaveretum, paranyline, parsalmide, pentazocine, perisoxal, phenacetin, phenadoxone, phenazocine, phenazopyridine hydrochloride, phenocoll, phenoperidine, phenopyrazone, phenyl acetylsalicylate, phenylbutazone, phenyl salicylate, phenyramidol, piketoprofen, piminodine, pipebuzone, piperylone, piprofen, pirazolac, piritramide, piroxicam, pranoprofen, proglumetacin, proheptazine, promedol, propacetamol, propiram, propoxyphene, propyphenazone, proquazone, protizinic acid, ramifenazone, remifentanil, rimazolium metilsulfate, salacetamide, salicin, salicylamide, salicylamide o-acetic acid, salicylsulfuric acid, salsalte, salverine, simetride, sodium salicylate, sufentanil, sulfasalazine, sulindac, superoxide dismutase, suprofen, suxibuzone, talniflumate, tenidap, tenoxicam, terofenamate, tetrandrine, thiazolinobutazone, tiaprofenic acid, tiaramide, tilidine, tinoridine, tolfenamic acid, tolmetin, tramadol, tropesin, viminol, xenbucin, ximoprofen, zaltoprofen and zomepirac. Analgesics are well known in the art. See, for example, The Merck Index, 12th Edition (1996), Therapeutic Category and Biological Activity Index, and the lists provided under “Analgesic”, “Anti-inflammatory” and “Antipyretic”. As used herein, the recitation of an analgesic inherently encompasses the pharmaceutically acceptable salts thereof.

### 8. Immunostimulants

The term “immunostimulant” is used herein to describe a substance which evokes, increases, and/or prolongs an immune response to an antigen. Immunomodulatory agents modulate the immune system, and, as used herein, immunostimulants are also referred to as immunomodulatory agents, where it is understood that the desired modulation is to stimulate the immune system. There are two main categories of immunostimulants, specific and non-specific. Specific immunostimulants provide antigenic specificity in immune response, such as vaccines or any antigen, and non-specific immunostimulants act irrespective of antigenic specificity to augment immune response of other antigen or stimulate components of the immune system without antigenic specificity, such as adjuvants and non-specific immunostimulators. Immunostimulants can include, but are not limited to, levamisole, thalidomide, erythema nodosum leprosum, BCG, cytokines such as interleukins or interferons, including recombinant cytokines and interleukin 2 (aldeslukin), 3D-MPL, QS21, CpG ODN 7909, miltefosine, anti-PD-1 or PD-1 targeting drugs, and acid (DCA, a macrophage stimulator), imiquimod and resiquimod (which activate immune cells through the toll-like receptor 7), chlorooxygen compounds such as tetrachlorodecaoxide (TCDO), agonistic CD40 antibodies, soluble CD40L, 4-1BB:4-1BBL agonists, OX40 agonists, TLR agonists, moieties that deplete regulatory T cells, arabinitol-ceramide, glycerol-ceramide, 6-deoxy and 6-sulfono-myo-insitolceramide, iNKT agonists, and TLR agonists. As used herein, the recitation of an immunostimulant inherently encompasses the pharmaceutically acceptable salts thereof.

### 9. Immune-Based Product

As used herein, immune-based products include, but are not limited to, toll-like receptors modulators such as tlr1, tlr2, tlr3, tlr4, tlr5, tlr6, tlr7, tlr8, tlr9, tlr10, tlr11, tlr12, and tlr13; programmed cell death protein 1 (Pd-1) modulators; programmed death-ligand 1 (Pd-L1) modulators; IL-15 agonists; DermaVir; interleukin-7; plaquenil (hydroxychloroquine); proleukin (aldesleukin, IL-2); interferon alfa; interferon alfa-2b; interferon alfa-n3; pegylated interferon alfa; interferon gamma; hydroxyurea; mycophenolate mofetil (MPA) and its ester derivative mycophenolate mofetil (MMF); ribavirin; rintatolimod, polymer polyethyleneimine (PEI); gepon; rintatolimod; IL-12; WF-10; VGV-1; MOR-22; BMS-936559; CYT-107, interleukin-15/Fc fusion protein, normferon, peginterferon alfa-2a, peginterferon alfa-2b, recombinant interleukin-15, RPI-MN, GS-9620, and IR-103. As used herein, the recitation of an immune-based product inherently encompasses the pharmaceutically acceptable salts thereof.

### G. Kits

Disclosed herein is a kit comprising a disclosed isolated nucleic acid molecule, a disclosed vector, a disclosed pharmaceutical formulation, and/or a combination thereof. In an aspect, a kit can comprise a disclosed isolated nucleic acid molecule, a disclosed vector, a disclosed pharmaceutical formulation, and/or a combination thereof, and one or more agents. “Agents” are known to the art and are described supra. In an aspect, the one or more agents can treat, prevent, inhibit, and/or ameliorate one or more comorbidities in a subject. In an aspect, one or more active agents can treat, inhibit, prevent, and/or ameliorate a GSD symptom or a GSD related complication. In an aspect, one or more active agents can treat, inhibit, prevent, and/or ameliorate a symptom of GSD IV and/or APBD, Lafora disease (including those diseases caused by mutations in the EPM2A gene (glucan phosphatase, laforin) or the NHLRC1 gene (NHL repeat containing E3 ubiquitin protein ligase 1 or EPM2B)), polyglucosan body myopathy-1, polyglucosan body myopathy-2, or any disease or pathology caused by a mutation in a GYG1 gene, a RBCK1 gene, or a PRKAG2 gene symptom or a complication related to Lafora disease (including those diseases caused by mutations in the EPM2A gene (glucan phosphatase, laforin) or the NHLRC1 gene (NHL repeat containing E3 ubiquitin protein ligase 1 or EPM2B)), polyglucosan body myopathy-1, polyglucosan body myopathy-2, or any disease or pathology caused by a mutation in a GYG1 gene, a RBCK1 gene, or a PRKAG2 gene. In an aspect, one or more active agents can reduce the expression level and/or activity level of glycogen synthase.

In an aspect, a disclosed kit can comprise at least two components constituting the kit. Together, the components constitute a functional unit for a given purpose (such as, for example, treating a subject diagnosed with or suspected of having GSD IV and/or APBD). Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation. In an aspect, a kit for use in a disclosed method can comprise one or more containers holding a disclosed isolated nucleic acid molecule, a disclosed vector, a disclosed pharmaceutical formulation, or a combination thereof, and a label or package insert with instructions for use. In an aspect, suitable containers include, for example, bottles, vials, syringes, blister pack, etc. The containers can be formed from a variety of materials such as glass or plastic. The container can hold a disclosed isolated nucleic acid molecule, a disclosed vector, a disclosed pharmaceutical formulation, or a combination thereof, and can have a sterile access port (for example the container may be an intravenous solution bag or a vial having a stopper pierceable by a hypodermic injection needle). The label or package insert can indicate that a disclosed isolated nucleic acid molecule, a disclosed vector, a disclosed pharmaceutical formulation, or a combination thereof can be used for treating, preventing, inhibiting, and/or ameliorating GSD IV and/or APBD or complications and/or symptoms associated with GSD IV and/or APBD. A kit can comprise additional components necessary for administration such as, for example, other buffers, diluents, filters, needles, and syringes.

### VII. EXAMPLES

In the past decade, AAV has become the most commonly used gene therapy vector in clinical trials for a broad range of human genetic diseases. (Rittie L, et al. (2019) Mol Ther. 27(10):1706-1717). Gene therapy with AAV9 provides a suitable treatment option for APBD because AAV9 can reliably transduce liver and muscle tissues with high efficiency and can cross the blood-brain barrier to deliver the therapeutic genes to the CNS following systemic injection. (Samaranch L, et al. (2012) Hum Gene Ther. 23(4):382-389; Manfredsson F P, et al (2009) Mol Ther. 17(3):403-405). In May 2019, the U.S. Food and Drug Administration approved Zolgensma (AVXS-101), an AAV9-based gene therapy, for treatment of pediatric patients with spinal muscular atrophy type 1 based upon its clinical success in improving the overall survival and motor function. (Mendell J R, et al. (2017) N Engl J Med. 377(18):1713-1722). However, acute liver failure resulted from the high-dose vector regimen was reported in two patients within 8 weeks of receiving AVXS-101, a development that underscored the need for the development of a more potent vector for gene delivery to the CNS (Feldman A G, et al. (2020) J Pediatr. 225:252-258). An AAV9 vector (AAV-CB-hGBE) expressing hGBE driven by the universally active CMV enhancer/chicken β-actin (CB) promoter completely prevented PB formation in the skeletal muscles in infant Gbe1^{ys/ys} mice and partially corrected PB accumulation in the brain for up to 9 months of age. (Yi H, et al. (2017) Hum Gene Ther. 28(3):286-294). The same AAV vector, however, also elicited strong cytotoxic T lymphocytes (CTL) response to the human protein in adult Gbe1^{ys/ys} mice, resulting in a rapid loss of hGBE expression. Thus, there is a need for an AAV vector with enhanced CNS transduction potency to deliver an optimal hGBE expression cassette.

The Examples that follow are illustrative of specific embodiments of the invention, and various uses thereof. They set forth for explanatory purposes only and are not to be taken as limiting the invention.

### Example 1

### Construction of the LSP-CB Dual Promoter

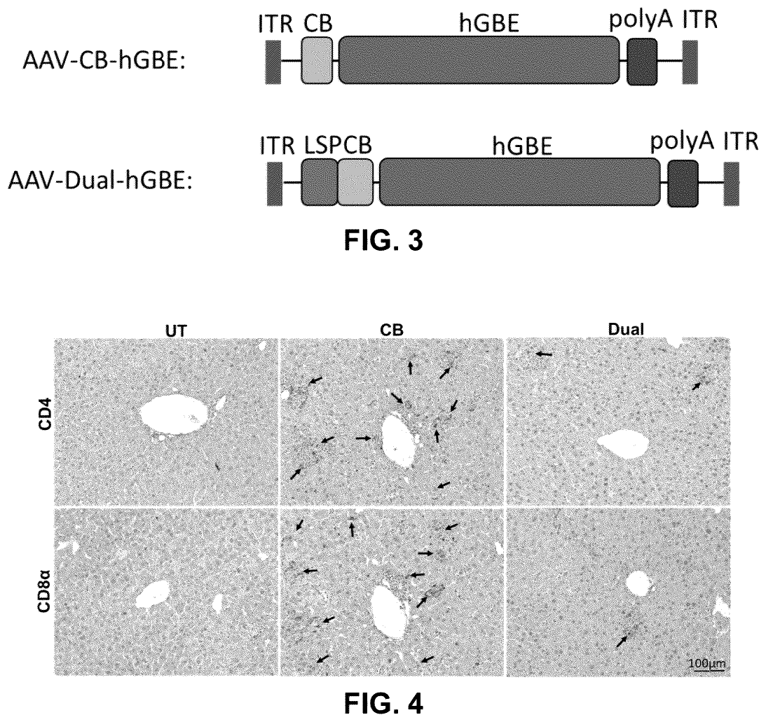

A new Dual promoter was designed and constructed to determine whether a dual promoter would retain the ability of a liver-specific promoter (LSP) to induce immunotolerance and enable expression of the therapeutic transgene in all affected tissues by a ubiquitous promoter. FIG. 3 shows two AAV constructs—AAV-CB-hGBE (having the ubiquitous CMV enhancer/chicken β-actin (CB) promoter) (SEQ ID NO:24) and AAV-Dual-hGBE (having a tandem LSP and CB fusion promoter) (SEQ ID NO:25). ITR indicates inverted repeats and polyA indicates human growth hormone polyA signal sequence. Both vectors shown in FIG. 3 carried an unmodified hGBE.

### Example 2

### The LSP-CB Dual Promoter Prevented hGBE-Related CTL Response in GSD IV Mice

The Dual promoter retained the ability of the liver-specific promoter to induce immunotolerance to hGBE. The AAV vectors in FIG. 3 were packaged in AAV9 and then intravenously injected into 10-week-old GSD IV mice (Gbe1^{ys/ys}) at the same dose of 2.5×10¹³ vg/kg. Mice were euthanized after two weeks. To examine transgene-induced T cell responses, liver sections from untreated (UT) mice (n=3), AAV-CB-hGBE treated mice (n=3) (CB), and AAV-Dual-hGBE treated mice (n=3) (Dual) were immunohistochemically stained with an anti-CD4 or anti-CD8α monoclonal antibody (Abcam). FIG. 4 shows liver sections from untreated (UT), AAV-CB-hGBE treated (CB), and AAV-Dual-hGBE treated (Dual) mice were stained with an anti-CD4 or anti-CD8α monoclonal antibody (Abcam) that infiltrations of CD4⁺ and CD8⁺ lymphocytes (black arrows) were abundant in the CB-treated livers but barely detectable in the Dual-treated livers (Dual). The images represent n=3 mice in each group. The Dual promoter functioned to prevent a cytotoxic T cell mediated immune reaction.

### Example 3

### Dual Promoter Resulted in Higher hGBE Expression than CB Promoter in Major Tissues of GSD IV Mice

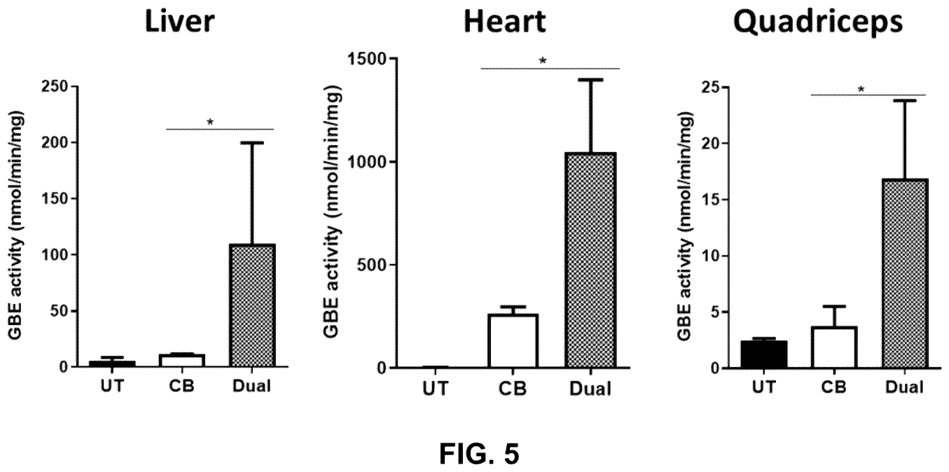

While inducing immunotolerance as described above, the Dual (LSP/CB) promoter also enabled expression of the therapeutic transgene. FIG. 5 shows that the level of GBE enzyme activities in the liver (left panel), heart (middle panel), and quadriceps muscle (right panel) of Dual-treated mice (n=3) was significantly higher (p<0.01) in each tissue type than the level of enzymatic activities in the CB-treated mice (n=3) two (2) weeks after AAV injection (2.5×10¹³ vg/kg). The Dual promoter functioned to drive transgene expression.

### Example 4

### AAV-Dual-hGBE Resulted in Persistent hGBE Expression and Glycogen Reduction in Major Tissues of GSD IV Mice

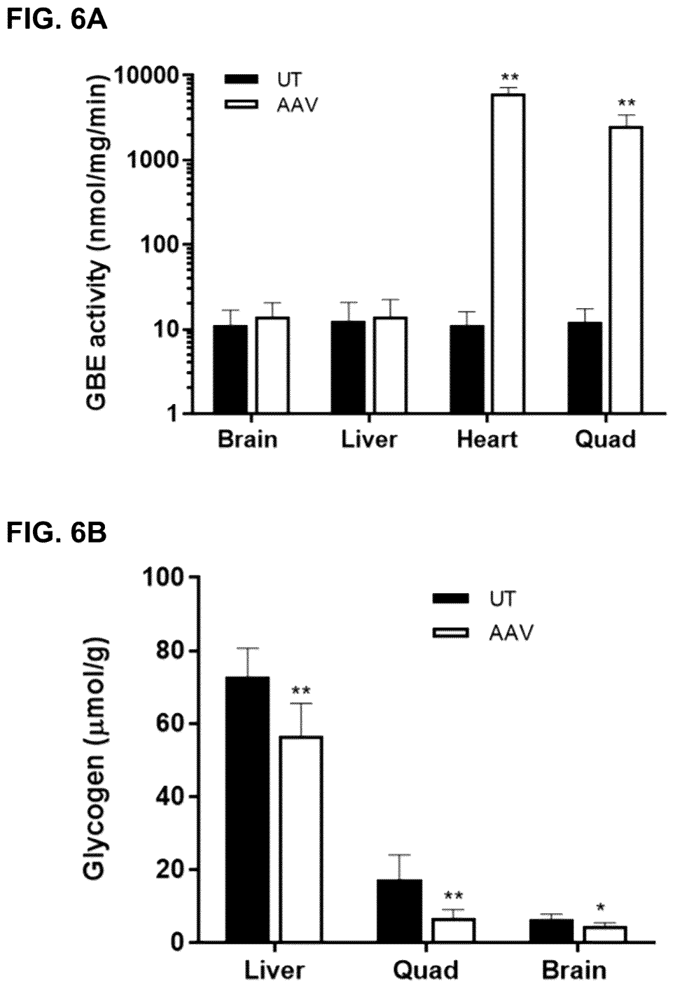

The AAV-Dual-hGBE (LSP/CB) packaged in AAV9 was intravenously injected into 6-week-old GSD IV mice at a dose of 1×10¹⁴ vg/kg. Tissues were collected 6 weeks later. FIG. 6A shows significantly elevated GBE activities in the heart and quadriceps of AAV-treated mice compared with that in the untreated age-matched mice (**p<0.01). FIG. 6B shows significantly reduced glycogen levels in the quadriceps (**p<0.01) and brain (*p<0.05) of the AAV-treated mice compared with that in the untreated age-matched mice (n=8 mice for each group).

### Example 5

### AAV-Dual-hGBE Treatment Improved Muscle Function in GSD IV Mice

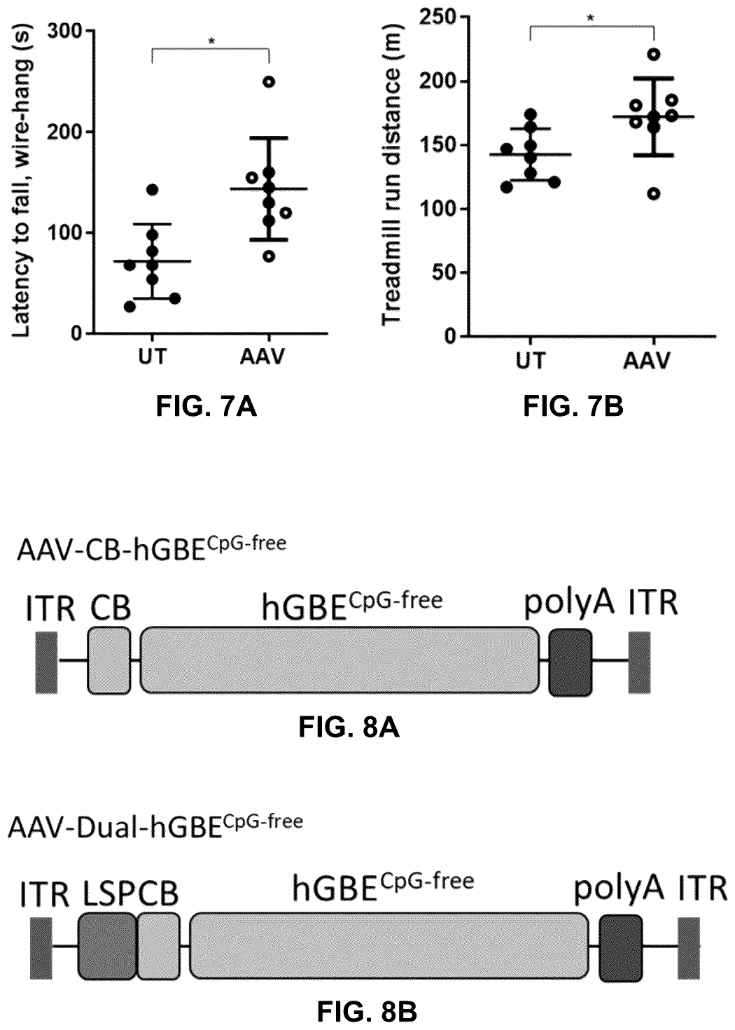

While reducing glycogen accumulation in tissues as described above in Example 4, the AAV-Dual-hGBE treated also significantly improved muscle function in GSD IV mice (Gbe1^{ys/ys}). Mice were intravenously injected at 6 weeks of age with 1×10¹⁴ vg/kg AAV9-Dual-hGBE vector. Six (6) weeks later, muscle functions were determined by the significantly (*p<0.05) increased performance in wire-hang test (FIG. 7A) and treadmill test (FIG. 7B). UT represents age-matched untreated mice (n=8).

### Example 6

### Depletion of CpG Motifs from hGBE ORF Did not Reduce or Impair hGBE Expression in Cultured HEK293T Cells and in GSD IV Mice

Depletion of CpG motifs from transgene cassettes is an effective approach to reduce CTL responses to the AAV capsids and therapeutic transgene products (Faust S M, et al. (2013) J Clin Invest. 123(7):2994-3001; Wright J F, (2020) Mol Ther. 28(3):701-703). To confirm that the depletion of CpG motifs from transgene ORF was an effective approach to reduce CTL responses to the disclosed AAV constructs, a 2.1-kb CpG-free hGBE ORF (hGBE^{CpG-free}) DNA was synthesized by removing all 51 CpG motifs from the hGBE ORF (GenScript, Piscataway, NJ) and cloning it into the AAV-CB-hGBE and AAV-Dual-hGBE vectors to replace the unmodified hGBE ORF. The resulting vector plasmids were the new AAV-CB-hGBE^{CpG-free}(FIG. 8A) and AAV-Dual-hGBE^{CpG-free} (FIG. 8B).

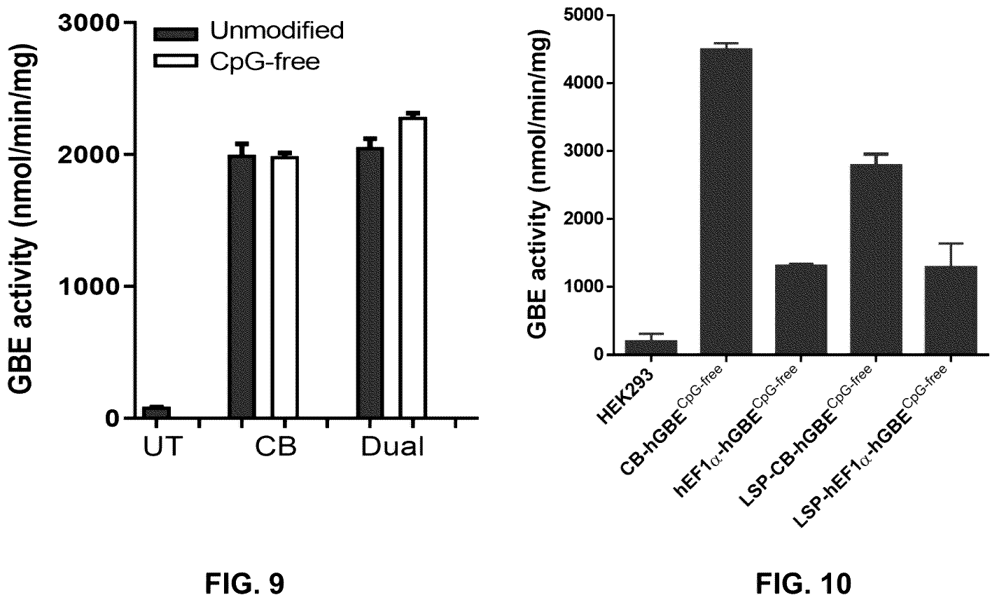

FIG. 9 shows that transfection of HEK293T cells with equal amount of the four AAV vector plasmids discussed above (FIG. 3 and FIG. 8) resulted in comparative levels of hGBE expression between the CpG-free and unmodified hGBE ORFs driven by either the CB or the Dual promoter. HEK293 cells growing on 10-cm dishes (˜70% confluent) were transfected in duplicates with 10 μg of each AAV vector (AAV-CB-hGBE, AAV-CB-hGBE^{CpG-free}, AAV-Dual-hGBE, and AAV-Dual-hGBE^{CpG-free}). The data provided in FIG. 9 represent an equal amount of the vector plasmids transfected to HEK293T cells in triplicates and GBE enzyme activity that was analyzed in cell lysates after 48 hours. Data represent the mean±SD. There is no significant difference in hGBE expression between the unmodified (hGBE) and CpG-depleted (hGBE^{CpG-free}) ORFs under the control of either the CB or the Dual promoter. Two additional AAV constructs containing hGBE^{CpG-free} were also created: AAV-hEF1α-hGBE^{CpG-free} (having a CpG-free human EF1α promoter (Invitrogen) rather than the CB promoter) and AAV-LSP-hEF1α-hGBE^{CpG-free} (having a new LSP and hEF1α dual promoter) (SEQ ID NO:26). FIG. 10 shows the resulting GBE expression following transfection of HEK293T cells with an equal amount (10 μg plasmid per 10 cm dish) of the 4 AAV vector plasmids having the CpG-free hGBE ORF driven by either the CB promoter, a CpG-free hEF1α (human translation elongation factor 1 alpha) promoter, a new dual promoter comprising the LSP and hEF1α, or the original dual promoter comprising LPS and CB. The GBE expression driven by the CB promoter was markedly higher than that by the hEF1α promoter; similarly, the GBE expression driven by the LSP-CB dual promoter was markedly higher than that by the new LSP-hEF1α dual promoter. These data indicate that the CpG-free hEF1α is a much weaker promoter than the CB. The non-transfected HEK293T generated the lowest level of GBE activity. The data provided in FIG. 10 represent equal amount of the vector plasmids transfected to HEK293T cells in triplicates and GBE enzyme activity that was analyzed in cell lysates after 48 hrs. Data represent the mean±SD.

To test in vivo expression of hGBE from CpG-free hGBE cassettes, AAV-CB-hGBE^{CpG-free} and AAV-Dual-hGBE^{CpG-free} (LSP-CB) vectors at a dose of 2.5×10¹³ vg/kg were injected into 3-month-old GSD IV mice via tail vein. Untreated mice and AAV-CB-hGBE treated mice were included as controls. All AAV vectors were packaged in AAV9 (n=5 mice per each group). One month later, tissues were homogenized in cold water and GBE activities were measured in tissue lysates of liver, skeletal muscle (quadriceps), and heart. As shown in FIG. 11, the AAV-Dual-hGBE^{CpG-free} resulted in significantly (*p<0.05 and **p<0.01) higher GBE activities in all the tissues tested (liver, quadriceps, and heart) than the AAV-CB-hGBE. The AAV-CB-hGBE^{CpG-free} resulted in a similar level of GBE activity to AAV-CB-hGBE in liver (left panel) but higher levels of GBE expression in the quadriceps (middle panel) and heart (right panel) than the AAV-CB-hGBE. These data demonstrate that the depletion of CpG motifs from the hGBE ORF as in the sequence presented did not impair hGBE expression in mammalian cells in vitro and in vivo.

### Example 7

### Optimization of hGBE Expression Cassette to Minimize Gene Therapy Related Immune Response

To identify the optimal hGBE expression cassette, the performance of various constructs having the Dual promoter is compared in GSD IV mice. Table 1 summarizes the groups for this experiment. All four AAV vectors are packaged in AAV9 and intravenously injected into 6-week-old GSD IV mice at the same dose of 1×10¹³ vg/kg. The transgene-related CTL response in adult GSD IV mice of hGBE CpG-free ORF is compared to that of the unmodified ORF under the control of the CB promoter; that is, Group 2 compared to Group 3. The transgene-related CTL response in adult GSD IV mice of hGBE CpG-free ORF is also compared to that of the unmodified ORF for under the control of the Dual promoter (having LSP and CB); that is, Group 4 is compared to Group 5.

TABLE 1

| Group | Treatment | Promoters | Capsid | Dose (vg/kg) |
|---|---|---|---|---|
| 1 | Vehicle | — | X | X |
| 2 | AAV-CB-hGBE | CB | AAV9 | 1 × 10¹³ |
| 3 | AAV-CB-hGBE^{CpG-free} | CB | AAV9 | 1 × 10¹³ |
| 4 | AAV-Dual-hGBE | LSP-CB | AAV9 | 1 × 10¹³ |
| 5 | AAV-Dual-hGBE^{CpG-free} | LSP-CB | AAV9 | 1 × 10¹³ |

Table 2 summarizes additional experimental groups to identify the optimal hGBE expression cassette in which a CpG-free EF1α (a murine CMV enhancer/human elongation factor-1 alpha promoter or mCMV/hEF1α) promoter replaces the CB promoter. All four new AAV vectors are packaged in AAV9 and intravenously injected into 6-week-old GSD IV mice at the same dose of 1×10¹³ vg/kg. The transgene-related CTL response in adult GSD IV mice of hGBE CpG-free ORF is compared to that of the unmodified ORF under the control of the EF1α promoter; that is, Group 2 compared to Group 3. The transgene-related CTL response in adult GSD IV mice of hGBE CpG-free ORF is also compared to that of the unmodified ORF under the control of the Dual promoter (having EF1α and LSP); that is, Group 4 is compared to Group 5.

TABLE 2

| | | | | Dose |
|---|---|---|---|---|
| Group | Treatment | Promoters | Capsid | (vg/kg) |
| 1 | Vehicle | — | X | X |
| 2 | AAV- EF1α-hGBE | EF1α | AAV9 | 1 × 10¹³ |
| 3 | AAV- EF1α-hGBE^{CpG-free} | EF1α | AAV9 | 1 × 10¹³ |
| 4 | AAV-Dual-hGBE | LSP-EF1α | AAV9 | 1 × 10¹³ |
| 5 | AAV-Dual-hGBE^{CpG-free} | LSP-EF1α | AAV9 | 1 × 10¹³ |

All mice are euthanized 4 weeks after AAV injection. Fresh tissue specimens including liver, heart, skeletal muscles (quadriceps, gastrocnemius, and diaphragm), bladder, spinal cord, and brain are immediately frozen on dry ice and stored at −80° C. until used for biochemical analyses, or fixed in 10% neutral-buffered formalin for histology. Human GBE expression is analyzed in tissue lysates by GBE enzyme activity assay and Western blot (Yi H, et al. (2017)). AAV vector bio-distribution is examined by quantitative PCR as described in Yi H, et al. (2017)). Transgene-related CTL response is determined by immunohistochemical staining of CD8⁺ and CD4⁺ lymphocytes in the liver sections.

### Example 8

### Evaluation of Efficacy of Novel AAV-F Vector Carrying the Optimal hGBE Cassette in GSD IV Mice

AAV-F previously demonstrated enhanced CNS transduction potency (Hanlon K S, et al. (2019) Mol Ther Methods Clin Dev. 15:320-332). AAV-CB-GFP packaged as either AAV9 or AAV-F was intravenously injected into adult C57BL/6 mice at the same dose of 3.2×10¹³ vector genomes (vg)/kg. As shown in FIG. 12, after 3 weeks, the AAV-F capsid displayed robust transgene expression in the brain and spinal cord in adult C57BL/6 mice (modified from Hanlon K S, et al. (2019)). Similar levels of GFP expression were reported for the liver and muscle.

To confirm that AAV-F carrying the optimal hGBE expression cassette enhances CNS transduction and reduces the effective vector dose, various AAF constructs carrying the LSP-CB Dual promoter and the CpG-Free hGBE ORF are constructed. Table 3 shows these AAV constructs.

TABLE 3

| Group | Treatment | Promoters | Capsid | Dose (vg/kg) |
|---|---|---|---|---|
| 1 | Vehicle | — | X | X |
| 2 | AAV-Dual-hGBE^{CpG-free} | LSP-CB | AAV-F | 1 × 10¹³ |
| 3 | AAV-Dual-hGBE^{CpG-free} | LSP-CB | AAV-F | 5 × 10¹³ |
| 4 | AAV-Dual-hGBE^{CpG-free} | LSP-CB | AAV9 | 5 × 10¹³ |

Table 4 shows additional AAV constructs to evaluate the efficacy of a novel AAV-F vector carrying the optimal hGBE cassette. In the AAV constructs of Table 4, a CpG-free EF1α promoter replaces the CB promoter to generate anew LSP-EF1α dual promoter.

TABLE 4

| Group | Treatment | Promoters | Capsid | Dose (vg/kg) |
|---|---|---|---|---|
| 1 | Vehicle | — | X | X |
| 2 | AAV-Dual-hGBE^{CpG-free} | LSP-EF1α | AAV-F | 1 × 10¹³ |
| 3 | AAV-Dual-hGBE^{CpG-free} | LSP-EF1α | AAV-F | 5 × 10¹³ |
| 4 | AAV-Dual-hGBE^{CpG-free} | LSP-EF1α | AAV9 | 5 × 10¹³ |

The AAV-Dual-hGBE^{CpG-free} vector (having either LSP-CB or LSP-EF1α dual promoter) is packaged into AAV-F or AAV9 (as control) and intravenously injected into 6-week-old Gbe1^{ys/ys} mice according to Table 3 (LSP-CB) or Table 4 (LSP-EF1α). Comprehensive behavioral tests are performed to monitor the improvement of neuromuscular and neurological functions induced by or related to the AAV treatments. A set of behavioral tests that can detect early neurological deficits resulting from extensive lysosomal glycogen accumulation in the CNS has been described. (Lim J A, et al. (2018) Mol Ther. 26(5):382-383).

A routine treadmill exhaustion test to assess exercise tolerance and a wire hang test to assess muscle strength are performed (Zhang P, et al. (2012) Hum Gene Ther. 23(5):460-72; Lim J A, et al. (2020) Mol Ther Methods Clin Dev. 18:240-249). Three (3) months after vector injection, additional tests are performed. For example, a urination frequency test to assess bladder function is performed by placing mice individually in metabolic cages with clean VSOP paper (Bio-Rad). Urine output (voiding frequency and volume) is measured by evaluating the surface area of the stained paper. (Wang Z, et al. (2012) Diabetes. 61(8):2134-2145).

To assess the sensory impairment (peripheral neuropathy or pain test), the mechanical sensitivity based on the thresholds of hind paw withdrawal in response to Von Frey filament stimulus using the up and down method is measured. (Pitcher G M, et al. (1999) J Neurosci Methods. 87(2):185-193; Berta T, et al. (2014) J Clin Invest. 124(3):1173-1186). To assess coordination and balance, the time that a mouse spends crossing a narrow beam from one end to the other is measured. (Lim J A, et al. (2018) Mol Ther. 26(5):382-383).

Upon the completion of these functional tests, all mice are euthanized to collect blood samples for testing plasma liver enzyme activities (e.g., AST and ALT). (Yi H, et al. (2017)). Tissue collections and analyses are performed as described above. PB reduction is assayed by glycogen content assay in tissue lysates and by Periodic acid-Schiff (PAS)-staining of formalin-fixed tissue sections. (Yi H, et al. (2017) Hum Gene Ther. 28(3):286-294).

For analysis of data from multiple groups, one-way ANOVA with post hoc test (Tukey) is performed using Prism software (Graphpad). For a two-group comparison, equal variance, unpaired, two-tailed Student's t-test are performed. (Yi et al., 2017; Lim et al., 2018).

### Example 9

### Expression of Mouse GBE Reduced Glycogen Levels in Liver and Muscle of GSD IV Mice

A new construct was designed to examine the long-term effects of GBE expression in a mouse model of GSD IV mice (Gbe1^{ys/ys}). The generation of Gbe1^{ys/ys} mice is discussed in Akman H O, et al. (2015) Hum Mol Genet. 24(23):6801-6810, which is incorporated herein for its teachings of the generation and characterization of the GSD IV mouse model. An AAV9 construct carrying a ubiquitous CMV enhancer/chicken β-actin (CB) promoter and murine GBE (mGBE) was used. Murine GBE is about 98% identical to human GBE. Mature 6-week-old Gbe1^{ys/ys} mice were intravenously injected via the tail with three different doses (Table 5). The low dose was 5×10¹² vg/kg (n=5). The mid (or medium) dose was 2.5×10¹³ vg/kg (n=5). The high dose was 1×10¹⁴ vg/kg (n=5). Mice were euthanized after 20 weeks of treatment at 26 weeks of age and tissues were collected.

TABLE 5

| Group | Treatment | Promoters | Capsid | Dose (vg/kg) |
|---|---|---|---|---|
| 1 | Vehicle | — | X | X |
| 2 | AAV-CB-mGBE | CB | AAV9 | 5.0 × 10¹² |
| 3 | AAV-CB-mGBE | CB | AAV9 | 2.5 × 10¹³ |
| 4 | AAV-CB-mGBE | CB | AAV9 | 1.0 × 10¹⁴ |

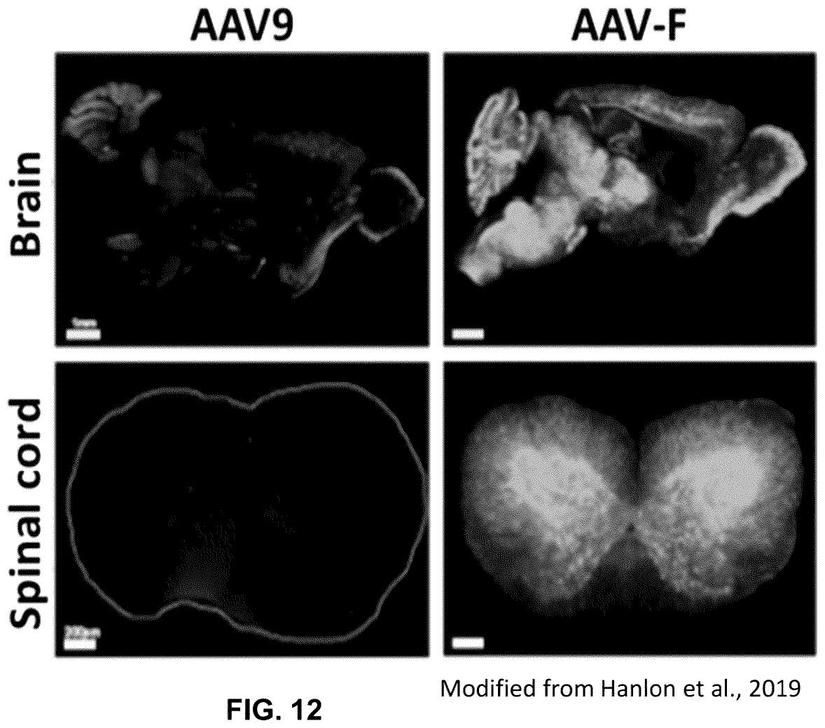

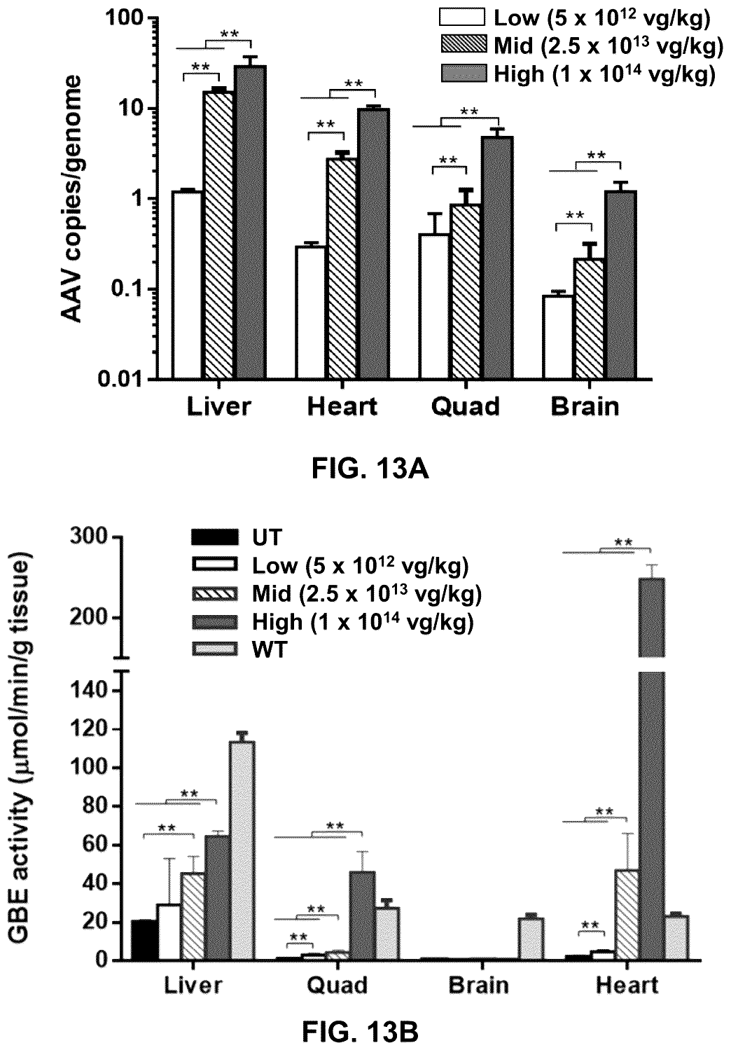

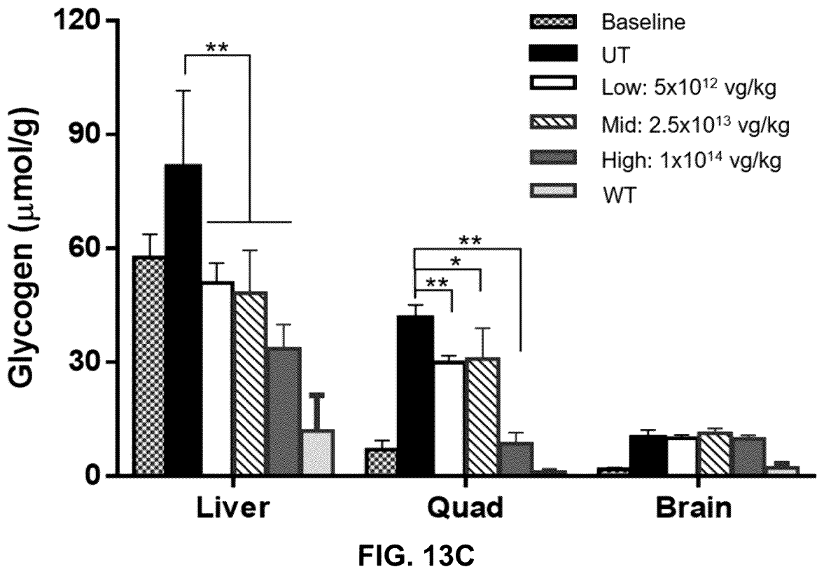

FIG. 13A shows that the AAV copies/genomes was highest following IV administration of the high dose (1×10¹⁴ vg/kg). In all four tissue types examined (i.e., liver, heart, quadriceps, and brain), the mid dose (2.5×10¹³ vg/kg) and the high dose resulted in significantly more AAV copies/genome than the low dose (5×10¹² vg/kg). The high dose also had significantly more AAV copies/genome than did the mid dose. ** indicates p<0.01.

FIG. 13B shows the level of GBE activity following IV administration of the low, mid, and high dose of AAV9-CB-mGBE in Gbe1^{ys/ys} mice as well as in untreated mice (n=8) and wild-type mice (n=8). The high AAV dose produced the highest level of GBE activity in the heart and in the quadriceps while the wild-type animals had the highest level of GBE activity in the brain and the liver. While the brain likely had no response due to low dose penetration from the systemic AAV injections, the work performed herein did not assess GBE activities in the CNS (brain). In turn, FIG. 13C shows that the elevated levels of GBE activity in the liver and the quadriceps resulted in clinically meaningful reductions in the levels of glycogen. UT mice had the highest glycogen levels in the liver and quadriceps. In FIG. 13A-FIG. 13C, UT means untreated Gbe1^{ys/ys} mice, WT means wild-type mice, and baseline refers to 6-week-old Gbe1^{ys/ys} mice (prior to the treatment protocol).

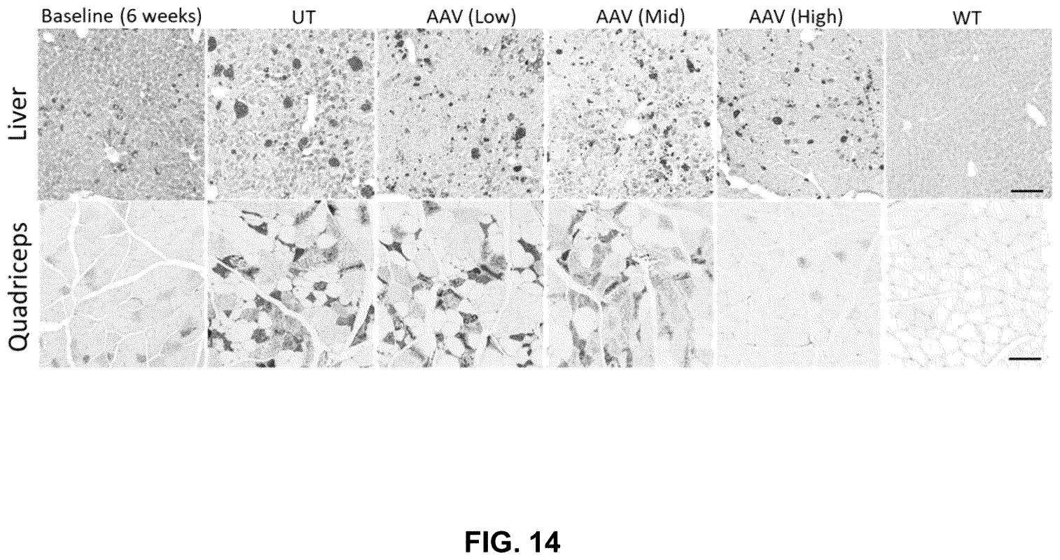

FIG. 14 shows reduced levels of polyglucosan body accumulation in AAV-treated animals. Here, PAS-D staining of the liver and muscle confirmed the measured glycogen content data in liver and muscle (FIG. 13C). In the liver of 6-week-old mice (baseline), polyglucosan body particles were fine and dispersed in hepatocytes. In 6-month-old untreated mice (UT), some cells contained large aggregates while others had lighter stain. Treatment with AAV reduced the size and number of glycogen particles in a dose-dependent manner. There were no visible polyglucosan bodies in the liver or the quadriceps muscle of WT animals. In the quadriceps muscle, there was weak PAS-D staining in cells, while large patches were present in 6-month-old UT mice. Thus, FIG. 14 shows that treatment with AAV9-CB-mGBE reduced polyglucosan bodies in a dose-dependent manner in muscle. The scale bars are 100 μm.

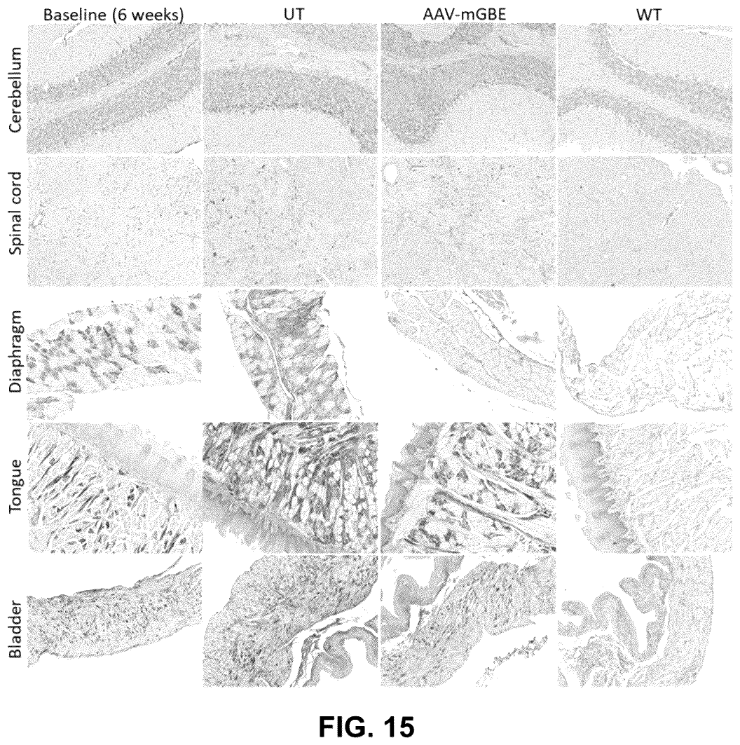

FIG. 15 shows reduced polyglucosan body accumulation in the spinal cord and other muscular tissues following the high-dose (1×10¹⁴ vg/kg) AAV treatment. The amount of polyglucosan bodies in the spinal cord was reduced in AAV-treated mice when compared to both WT and UT mice. The high-dose AAV treatment also resulted in significantly less polyglucosan body accumulation in diaphragm, tongue, and bladder tissue. The scale bars are 100 μm.

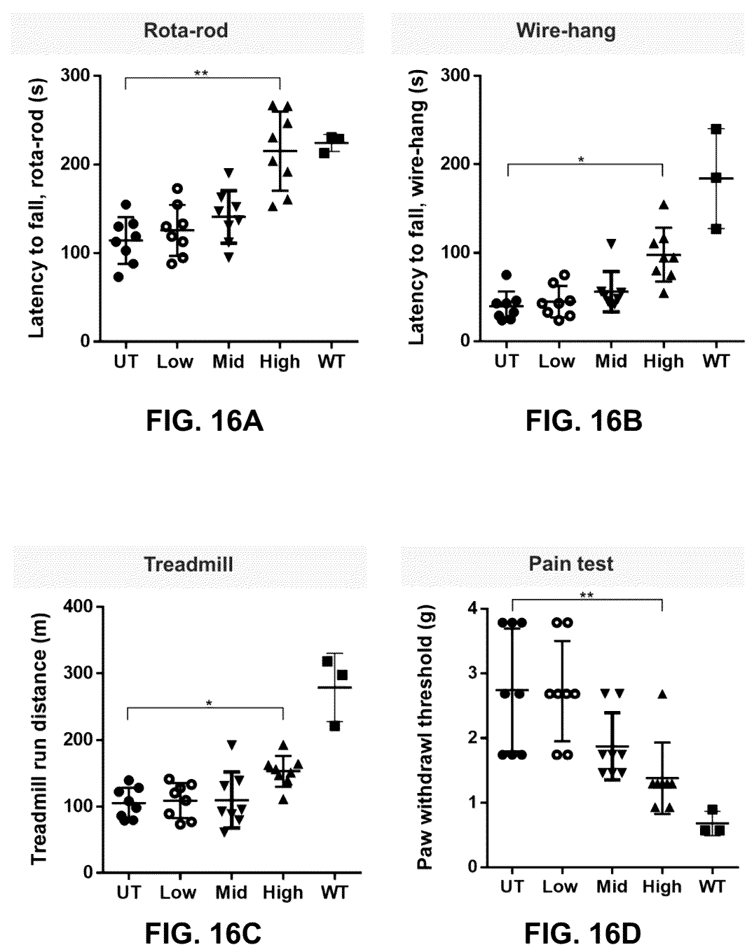

FIG. 16A-FIG. 16D show that treatment with AAV9-CB-mGBE rescued neurological and neuromuscular symptoms in GSD IV (Gbe1^{ys/ys}) mice. There were a dose-dependent improvement of neurological and neuromuscular phenotypes following the systemic administration of AAV9-CB-mGBE (SEQ ID NO:23). The tests included the Rota-rod test (FIG. 16A), the wire-hang test (FIG. 16B), the treadmill test (FIG. 16C), and the pain test (FIG. 16D). These results confirmed that a focus on the CNS/PNS aspects of disease provided lasting benefit and validated the rationale for exploring intra-CSF dosing regimen to target neurological and motor neuron related disease.

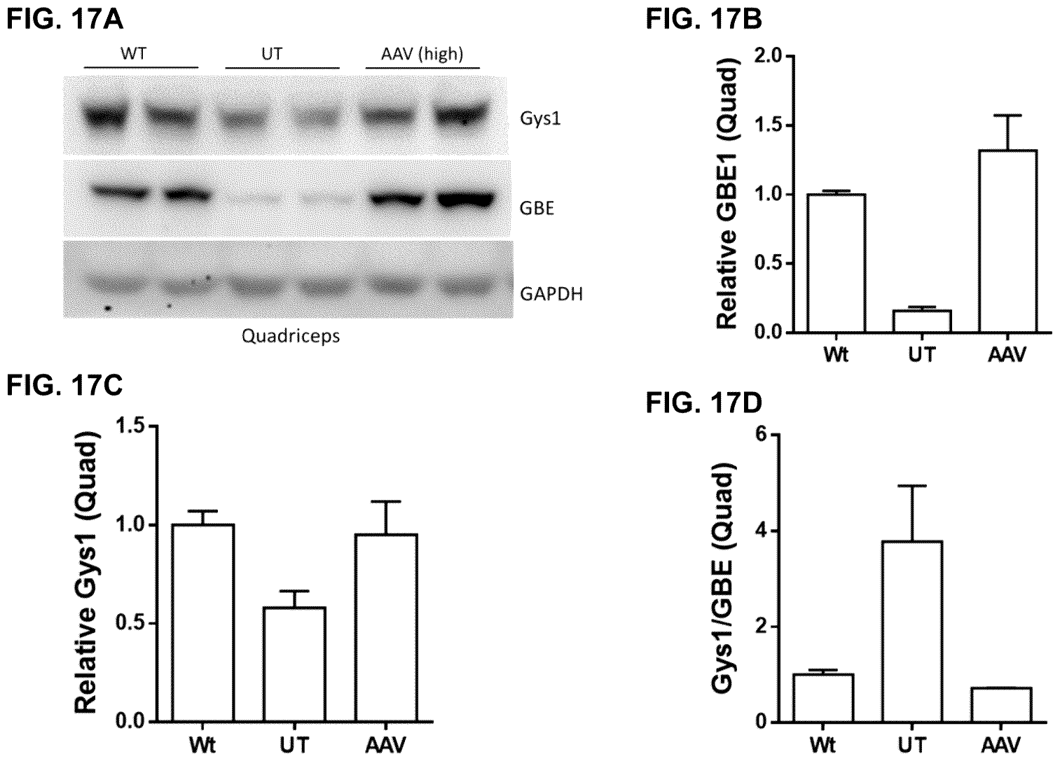

FIG. 17A-FIG. 17D show that AAV9-CB-mGBE treatment restored GBE and GYS1 expression. The resulting Western blots in FIG. 17A demonstrated that the high dose of AAV9-CB-mGBE restored the level of GYS1 to near wild-type levels. WT means normal mice at 6 months of age while UT means untreated Gbe1^{ys/ys} mice at 6 months of age. The antibodies used for this Western blot included rabbit anti-glycogen synthase (GS) (Cell Signaling mAb #3886, 1:3000), rabbit anti-GBE1 (Abcam ab180596, 1:2000), and goat anti-GAPDH (Novus NB300-320, 1:3000). FIG. 17B shows GBE protein levels in the quadriceps of untreated mice, wild-type mice, and AAV-treated mice. The AAV treated mice had near wild-type levels of GBE. FIG. 17C shows relative Gys1 protein levels in the quadriceps of untreated mice, wild-type mice, and AAV-treated mice, and demonstrated that the AAV-treated mice and wild-type mice had similar Gys1 expression levels. FIG. 17D shows the ratio of Gys1/GBE in untreated mice, wild-type mice, and AAV-treated mice, and demonstrated that wild-type mice and AAV-treated mice had a similar ratio of Gys1/GBE. This indicates that AAV treatment restored the Gys1/GBE ratio. For all mice (whether treated or not), the levels of GBE and Gys1 in the brain were low.

### Example 10

### Novel AAV-F Capsid Enhanced CNS Transduction

Next, as part of a broader CNS-targeted gene therapy strategy, whether the use of AAV-F capsid could enhance CNS transduction in Gbe1^{ys/ys} mice was examined. First, the AAV-CB-mGBE vector (SEQ ID NO:23) was packaged into AAV-F capsid. Second, adult 6-week-old Gbe1^{ys/ys} mice (n=9) were intravenously injected via the tail with 2.5×10¹³ vg/kg of the AAVF-CB-mGBE viral vector (Table 6). Mice were euthanized after 20 weeks of treatment at 26 weeks of age and tissues were collected.

TABLE 6

| Group | Treatment | Promoters | Capsid | Dose (vg/kg) |
|---|---|---|---|---|
| 1 | Vehicle | — | — | — |
| 2 | AAV-CB-mGBE | CB | AAV-F | 2.5 × 10¹³ |

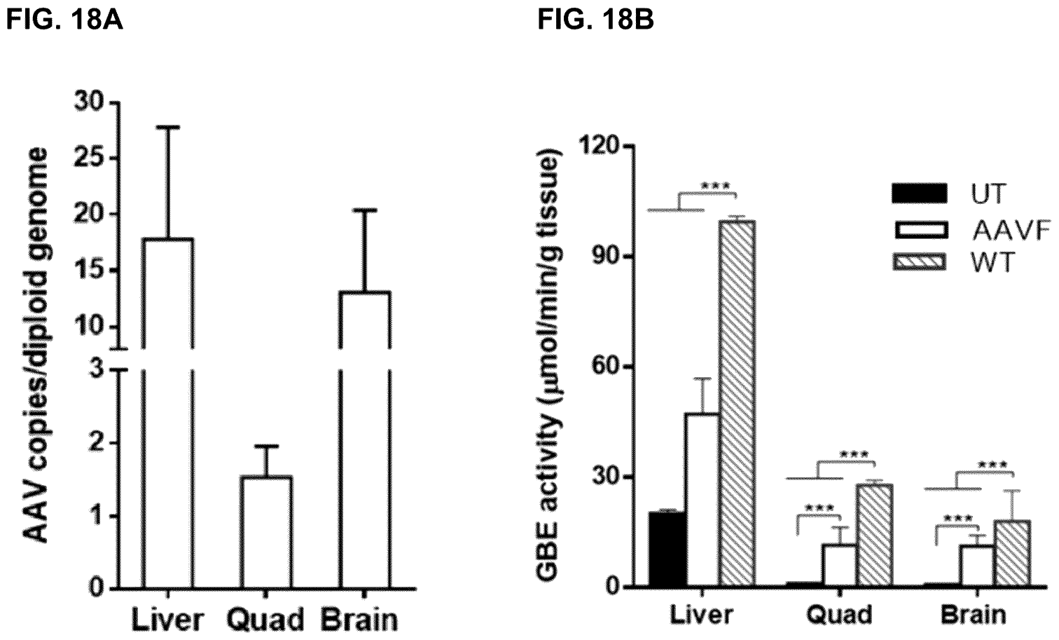

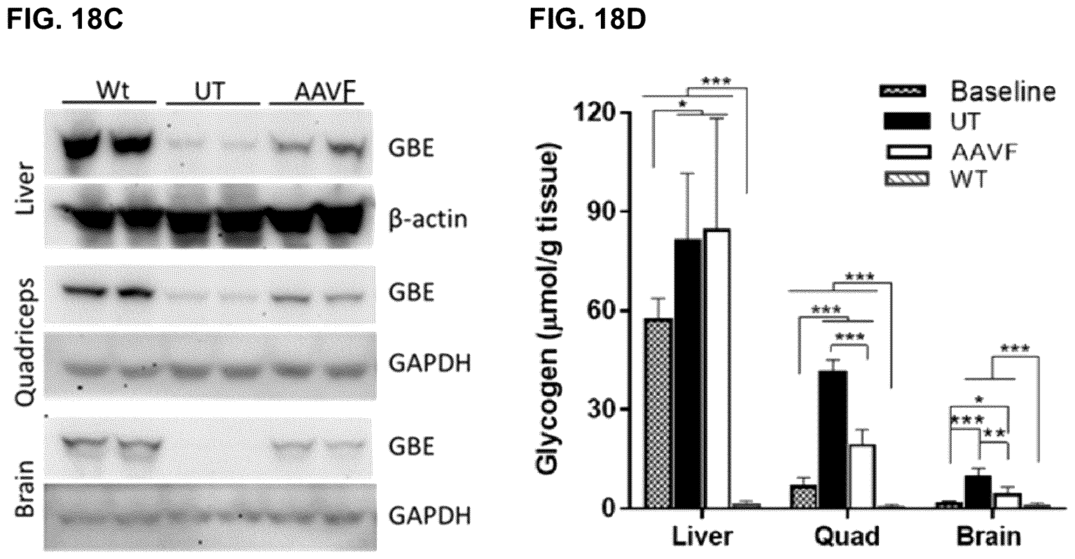

Treatment of Gbe1^{ys/ys} mice (n=9) with AAVF-CB-mGBE for 20 weeks increased GBE expression and reduced glycogen levels in tissues. FIG. 18A shows the number of AAV copies (genomes) in the liver, quadriceps, and brain of Gbe1^{ys/ys} mice treated with AAVF-CB-mGBE. FIG. 18B shows the level of GBE activity in the liver, quadriceps, and brain of untreated mice, wild-type mice, and AAVF-treated Gbe1^{ys/ys} mice. While the AAVF-CB-mGBE treated mice had more GBE activity than the untreated mice, the level of GBE activity was greatest in wild-type mice. FIG. 18C shows a series of Western blots demonstrating that AAVF-CB-mGBE increased GBE expression in the liver, the quadriceps, and the brain of Gbe1^{ys/ys} mice when compared to the untreated 26-week-old Gbe1^{ys/ys} mice. Finally, FIG. 18D shows that AAVF-CB-mGBE treatment decreased the level of glycogen in the quadriceps and brain when compared to the untreated Gbe1^{ys/ys} mice. In FIG. 18D, baseline means untreated 6-week-old Gbe1^{ys/ys} mice. Here, *p<0.05, **p<0.01, and ***p<0.001.

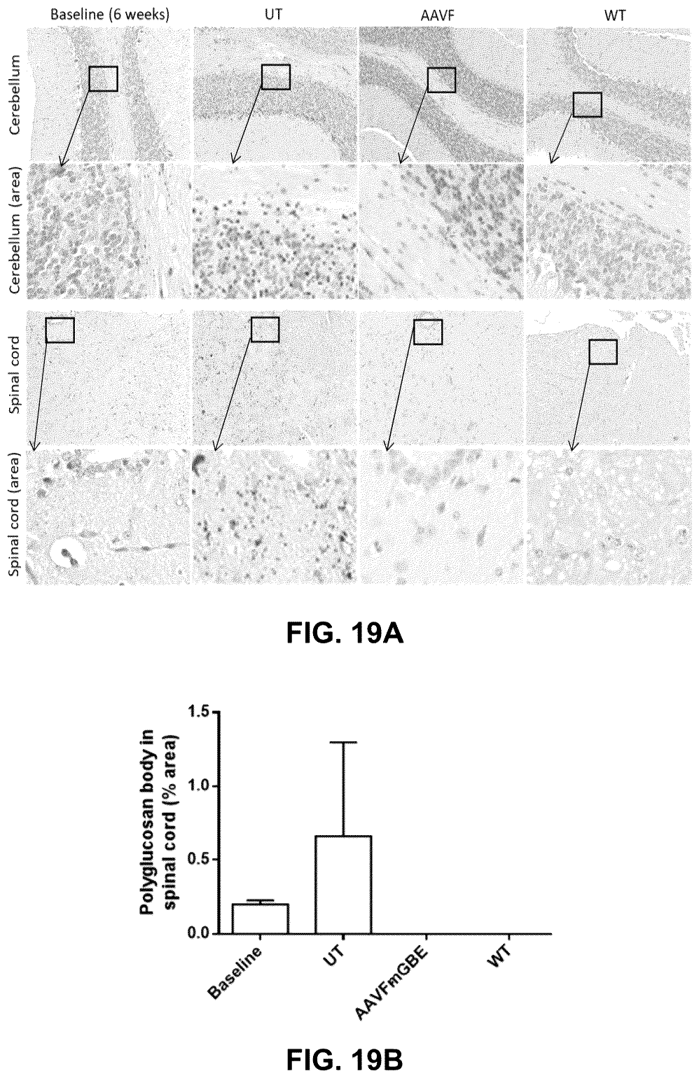

As shown in FIG. 19A, AAVF-CB-mGBE treatment significantly reduced polyglucosan body accumulation in the CNS (brain and spinal cord). In AAVF-CB-mGBE treated mice, very few Pas-D⁺ particles existed in the cerebellum or spinal cord sections from baseline and AAVF-treated mice. Conversely, there were abundant Pas-D⁺ particles seen in the cerebellum and the spinal cord in untreated mice. FIG. 19B shows the quantified polyglucosan data in spinal cords of UT, WT, baseline, and AAVF-treated mice.

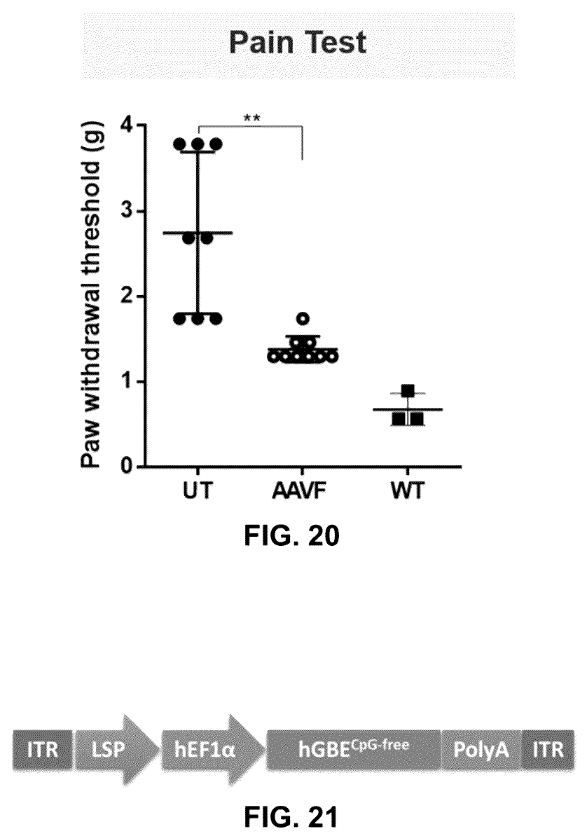

AAVF-CB-mGBE treated mice also showed neurological improvement. Following systemic treatment with AAVF-CB-mGBE for 20 weeks (n=9), there were significant improvements on the pain test (despite the low dose for systemic administration (2.5×10¹³ vg/kg). FIG. 20 shows the pain test in UT (n=8), WT (n=8), and AAVF-treated mice. These data demonstrated that CNS-targeted gene therapy was a viable strategy and supported the evaluation of intra-CSF administration.

### Example 11

### AAVF Having a Dual Promoter Comprising hEF1α Did not Raise GBE Activity in the Brain

To examine the effect of a novel dual promoter, the AAV-LSP-hEF1α-hGBE^{CpG-free} vector (SEQ ID NO:27) was packaged as AAV9 (AAV9-LSP-hEF1α-hGBE^{CpG-free}) or AAV-F (AAVF-LSP-hEF1α-hGBE^{CpG-free}). As shown in FIG. 21, the new dual promoter comprised LSP (human alfa-antitrypsin (hAAT)-derived) and hEF1α (a CpG-free mCMV enhancer/human EF1α promoter) while ITR indicates inverted repeats and polyA indicates human growth hormone polyA signal sequence. Both AAV vectors were intravenously injected via the tail at 2.5×10¹³ vg/kg into 6-week-old Gbe1^{ys/ys} mice (n=4 for AAV9, n=5 for AAVF; Table 7). Treatment lasted 6 weeks and mice were euthanized at 12 weeks of age. These studies aimed to assess a new ubiquitous promoter and to quantify liver expression with this dual promoter.

TABLE 7

| Group | Treatment | Promoters | Capsid | Dose (vg/kg) |
|---|---|---|---|---|
| 1 | Vehicle | — | X | X |
| 2 | AAV-Dual-hGBE^{CpG-free} | LSP-EF1α | AAV9 | 2.5 × 10¹³ |
| 3 | AAV-Dual-hGBE^{CpG-free} | LSP-EF1α | AAV-F | 2.5 × 10¹³ |

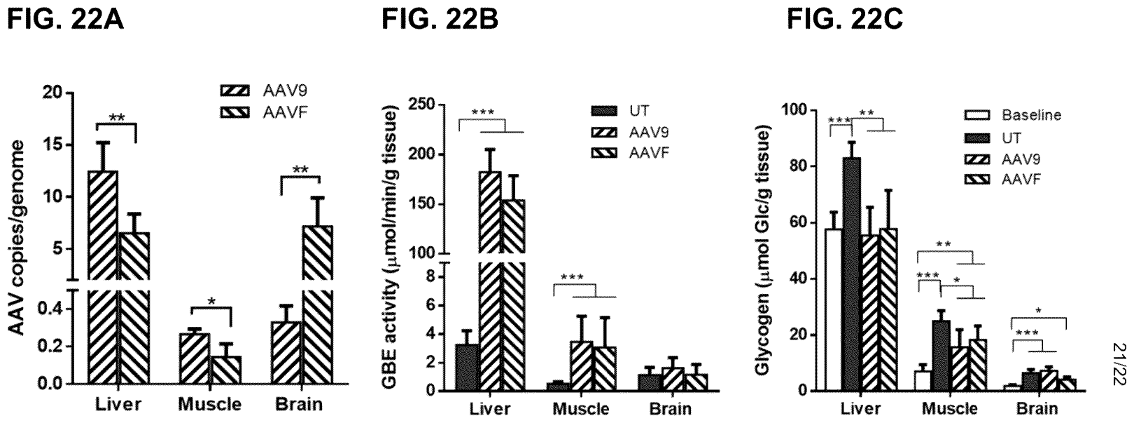

FIG. 22A shows the AAV biodistribution in the liver, the muscle, and the brain of AAV9-treated mice (n=4) and of AAVF-treated mice (n=5) compared to untreated (UT) mice (n=5). FIG. 22B shows the level of GBE activity in the liver, the muscle, and the brain of AAV9-treated mice and of AAVF-treated mice while FIG. 22C shows the resulting level of glycogen in the liver, the muscle, and the brain of these AAV-treated mice. Here, AAVF treatment did not enhance GBE activity in brain. Moreover, GBE activity in the muscle was also very low following treatment with either AAV9 or AAVF. These results indicated that the activity of the hEF1α promoter is too low to drive hGBE expression in these tissues.

### Example 12

### Crafting a Gene Therapy Approach Using a Neuron-Specific Promoter

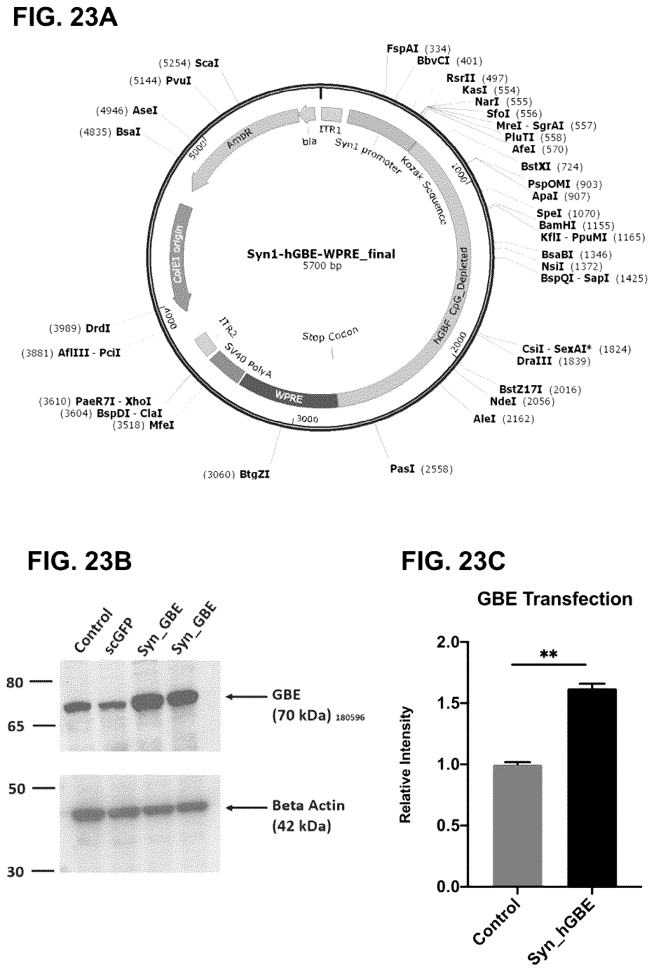

FIG. 23A shows the generation of an AAV construct of CpG-depleted human GBE with a neuron-specific promoter (i.e., synapsin). FIG. 23B shows Western blots of HEK293 cells, which demonstrated increased expression of hGBE with synapsin plasmid transfection. Synapsin plasmid transfection is compared to transfection with GFP plasmid and no plasmid (negative control), with β-actin as the housekeeping protein. FIG. 23C shows the quantification of the Western blots of FIG. 23B, which confirmed the increased expression of hGBE via transfection with the synapsin plasmid compared to the negative control.

One skilled in the art will readily appreciate that the present invention is well adapted to carry out the objects and obtain the ends and advantages mentioned as well as those inherent therein. The present examples along with the methods, procedures, treatments, molecules, constructs, therapies, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

1. An isolated nucleic acid molecule, comprising: a nucleic acid sequence encoding a polypeptide capable of preventing glycogen accumulation and/or degrading accumulated glycogen, wherein the nucleic acid sequence is a codon-optimized for expression in a human or mammalian cell, and wherein the polypeptide capable of preventing glycogen accumulation and/or degrading accumulated glycogen comprises a glycogen branching enzyme (GBE).

2. (canceled)

3. The isolated nucleic acid molecule of claim 1, wherein the nucleic acid sequence is CpG-depleted.

4. (canceled)

5. The isolated nucleic acid molecule of claim 1, wherein the nucleic acid sequence comprises a sequence having at least 50-69% identity to the sequence set forth in SEQ ID NO:03 or SEQ ID NO:04.

6. The isolated nucleic acid molecule of claim 1, wherein accumulated glycogen comprises polyglucosan bodies, Lafora bodies, amylopectin-like glycogen, or any combination thereof.

7. A vector comprising the isolated nucleic acid molecule of claim 1.

8. The vector of claim 7, wherein the vector comprises a ubiquitous or tissue-specific promoter.

9. (canceled)

10. The vector of claim 8, wherein the tissue-specific promoter comprises a synapsin I promoter or a α1-microglobulin/bikunin enhancer/thyroid hormone-binding globulin promoter.

11. The vector of claim 8, wherein the ubiquitous promoter comprises a CMV enhancer/chicken β-actin (CB) promoter or a murine CMV enhancer/human elongation factor-1 alpha (mCMV/hEF1α) promoter.

12. The vector of claim 7, wherein the vector is an adeno-associated virus (AAV) vector.

13.-14. (canceled)

15. A pharmaceutical formulation, comprising: the vector of claim 7 in a pharmaceutically acceptable carrier.

16. A method of treating and/or preventing glycogen storage disease type IV (GSD IV) and/or adult polyglucosan body disease (APBD) disease progression, the method comprising:
administering to a subject in need thereof a therapeutically effective amount of the vector of claim 7, wherein, following administration, glycogen accumulation is prevented and/or accumulated glycogen is degraded in the subject.

17.-20. (canceled)

21. The method of claim 16, wherein administering the vector comprises
(i) intravenous administration and the therapeutically effective amount of the vector comprises 1×10¹⁰ vg to 2×10¹⁴ vg; or
(ii) intrathecal, intracerebroventricular, or intra-cisterna magna administration and the therapeutically effective amount of the vector comprises 1×10⁹ vg to 2×10¹⁴ vg.

22. (canceled)

23. The method of claim 16, wherein the vector is delivered to the subject's liver, heart, skeletal muscle, smooth muscle, central nervous system, peripheral nervous system, or a combination thereof.

24. The method of claim 16, wherein one or more aspects of cellular homeostasis and/or cellular functionality are restored.

25. The method of claim 24, wherein restoration of one or more aspects of cellular homeostasis and/or cellular functionality comprises
(i) correction of cell starvation in one or more cell types;
(ii) normalization of aspects of the autophagy pathway;
(iii) improvement in and/or preservation of mitochondrial functionality and/or structural integrity;
(iv) improvement in and/or preservation of organelle functionality and/or structural integrity;
(v) preventing, slowing, and/or eliminating hypoglycemia, ketosis, and/or other liver abnormalities related to liver disease;
(vi) improvement in and/or reversal of neurogenic bladder, gait disturbances, and/or neuropathy;
(vii) prevention of and/or stabilization of the rate of progression of the multi-systemic manifestations of GSD IV and/or APBD, wherein the multi-systemic manifestations comprise cardiomyopathy, hepatic, and musculoskeletal dysfunction;
(viii) prevention of and/or slowing of the formation and/or cellular inclusion of polyglucosan bodies;
(ix) prevention of and/or slowing of the rate of progression of the onset of central nervous system (CNS)- and peripheral nervous system (PNS)-related manifestations, wherein the CNS- and PNS-related manifestations comprise neurogenic bladder, peripheral neuropathy, motor neuron disease, gait disturbances, and/or cognitive decline;
(x) prevention of and/or slowing of the rate of progression of liver disease, wherein the level of fibrosis, cirrhosis, hepatic adenomas, and liver hepatocellular carcinoma is decreased and/or reduced; or
(x) any combination thereof.

26. The method of claim 16, further comprising administering to the subject a therapeutically effective amount of an agent capable of reducing the expression level and/or activity level of glycogen synthase (GYS), wherein the agent is a gene therapy, RNA interference (RNAi), microRNA, antisense oligonucleotide (ASO), gene editing, or small molecule.

27. (canceled)

28. The method of claim 26, wherein the GYS/GBE ratio is restored to normal or near normal.

29. (canceled)

30. The method of claim 26, wherein the agent is guaiacol.

31. The method of claim 16, further comprising administering to the subject one or more immune modulators.

32. The method of claim 31, wherein the one or more immune modulators comprise methotrexate, rituximab, intravenous gamma globulin, synthetic vaccine particles containing rapamycin (SVP-Rapamycin), bortezomib, or a combination thereof.

33.-37. (canceled)
